(19) United States
(12) Patent Application Publication
(10) Pub. No.: US 2026/0035356 A1
(43) Pub. Date: Feb. 5, 2026

(54) MODULATORS OF TYK2 PROTEOLYSIS AND ASSOCIATED METHODS OF USE

(71) Applicant: Arvinas Operations, Inc., New Haven, CT (US)

(72) Inventors: David Langley, Meriden, CT (US); Lawrence B. Snyder, Killingworth, CT (US); Kurt Zimmermann, Durham, CT (US)

(21) Appl. No.: 18/997,207

(22) PCT Filed: Jul. 21, 2023
(86) PCT No.: PCT/US2023/028405

§ 371 Date: Jan. 21, 2025

Related U.S. Application Data
(60) Provisional application No. 63/391,216, filed on Jul. 21, 2022.

(51) Int. Cl. C07D 401/14; A61K 31/501; A61K 31/506; A61K 45/06; A61K 47/55

(52) U.S. Cl. CPC C07D 401/14; A61K 31/501; A61K 31/506; A61K 45/06; A61K 47/55

(57) ABSTRACT

Bifunctional compounds, which find utility as modulators of non-receptor tyrosine kinase 2 (TYK2), are described herein. In particular, the bifunctional compounds of the present disclosure contain on one end a moiety that binds to the cereblon E3 ubiquitin ligase and on the other end a moiety which binds TYK2, such that the target protein is placed in proximity to the ubiquitin ligase to effect degradation (and inhibition) of target protein. The bifunctional compounds of the present disclosure exhibit a broad range of pharmacological activities associated with degradation/inhibition of target protein. Diseases or disorders that result from aberrant regulation of the target protein are treated or prevented with compounds and compositions of the present disclosure.

### RELATED APPLICATIONS

This application claims the benefit of priority to U.S. provisional application No. 63/391,026, filed Jul. 21, 2023.xc

### INCORPORATION BY REFERENCE

All cited references are hereby incorporated herein by reference in their entirety, including U.S. patent application Ser. No. 14/686,640, filed on Apr. 14, 2015, published as U.S. Patent Application Publication No. 2015/0291562; and U.S. patent application Ser. No. 14/792,414, filed on Jul. 6, 2015, published as U.S. Patent Application Publication No. 2016/0058872; and U.S. patent application Ser. No. 15/953,108, filed on Apr. 13, 2018, published as U.S. Patent Application Publication No. 2018/0228907; U.S. patent application Ser. No. 14/441,183, filed on Nov. 7, 2013, published as U.S. Patent Application Publication No. 2015/0299183A1, issued as U.S. Pat. No. 9,505,748; and U.S. patent application Ser. No. 15/289,437 filed 10 Oct. 2016, published as U.S. Patent Application Publication No. 2017/0022192A1, issued as U.S. Pat. No. 10,000,480; and U.S. patent application Ser. No. 15/034,915 filed 16 Jan. 2014, published as U.S. Patent Application Publication No. 2016/0280649A1, issued as U.S. Pat. No. 9,663,467; and U.S. patent application Ser. No. 15/480,787, filed on Apr. 6, 2017, published as U.S. Patent Application Publication No. 2017/0209426A1, issued as U.S. Pat. No. 9,987,266; and International Patent Application No. PCT/US2014/011769, filed Jan. 16, 2014, published as International Patent Application Publication No. WO2015/069310A1; and International Patent Application No. PCT/US2013/068846, filed Nov. 7, 2013, published as International Patent Application Publication No. WO2014/074661A1.

### FIELD

The description provides hetero-bifunctional compounds comprising a target protein binding moiety and a E3 ubiquitin ligase binding moiety, and associated methods of use. The bifunctional compounds are useful as modulators of targeted ubiquitination of tyrosine kinase 2 (TYK2), which is then degraded and/or inhibited.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore, are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands which bind to these ligases. For example, since the discovery of nutlins, the first small molecule E3 ligase inhibitors, additional compounds have been reported that target E3 ligases.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubiquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

Bifunctional compounds such as those described in U.S. Patent Application Publications 2015/0291562 and 2014/0356322 (incorporated herein by reference), function to recruit endogenous proteins to an E3 ubiquitin ligase for ubiquitination and subsequent degradation in the proteasome degradation pathway. In particular, the publications cited above describe bifunctional or proteolysis-targeting chimeric (PROTAC®) protein degrader compounds, which find utility as modulators of targeted ubiquitination of a variety of polypeptides and other proteins, which are then degraded and/or inhibited by the bifunctional compounds.

TYK2 is the first member of the Janus family of kinases (JAK) that was described and which functions in multiple immune signaling pathways. The JAK family, which also includes JAK1, JAK2, and JAK3, is known to mediate the signaling of numerous cytokines that cause inflammation. TYK2 has been implicated in interferon (IFN)-α, interleukin (IL)-6, IL-10, IL-12 and IL-23 signaling. TYK2 is a drug candidate for multiple diseases of the immune system. Selectively targeting TYK2 using a small molecule is a significant challenge due to the high homology of the ATP active site within the JAK family. See Strobl et al. (2011) Tyrosine kinase 2 (TYK2) in cytokine signaling and host immunity. Front. Biosci. (Landmark Ed). 16:3214-3232. However, TYK2 has a pseudokinase domain that has been implicated in altered susceptibility to infection and immunological disorders. As such, a class of small molecules targeting the TYK2 pseudokinase domain, e.g., BMS-986165, are reported to be useful in the modulation of IL-12, IL-23 and/or IFNα signaling cascades, and are disclosed in U.S. Pat. Nos. 9,505,748; 10,000,480; 9,663,467; 9,987,266; and International Patent Application Publications WO2015/069310A1 and WO2014/074661A1, which are incorporated herein by reference.

An ongoing need exists in the art for effective treatments for disease, e.g., immunological or inflammatory disorders. Numerous studies in humans propose a link between TYK2 genetic variants and several autoimmune diseases, inflammatory diseases and tumors. Thus, TYK2 appears as an attractive target for therapeutic intervention. However, non-specific effects, and the inability to target and modulate TYK2, remain as obstacles to the development of effective treatments. As such, additional small-molecule therapeutic agents that target TYK2, and which leverage or potentiate E3 ubiquitin ligase (e.g., cereblon) substrate specificity and targeted protein degradation of would be very useful.

### SUMMARY

The present disclosure describes hetero-bifunctional compounds that function to recruit non-receptor tyrosine protein kinase TYK2 to an E3 ubiquitin ligase for targeted ubiquitination and subsequent proteasomal degradation, and methods of making and using the same. In addition, the description provides methods of using an effective amount of the compounds of the invention as described herein for the treatment or amelioration of a disease condition, such as an auto immune disease, e.g., multiple sclerosis, psoriasis, Crohn's disease, rheumatoid arthritis, biliary cirrhosis, or ankylosing spondylitis.

As such, in one aspect the disclosure provides hetero-bifunctional compounds, which comprise an E3 ubiquitin ligase binding moiety (i.e., a ligand for an E3 ubiquitin ligase (a “ULM” group)), and a moiety that binds TYK2 (i.e., a protein targeting moiety or “PTM” group, that is, a TYK2 targeting ligand or a “TTM” group) such that the TYK2 protein is thereby placed in proximity to the ubiquitin ligase to effect ubiquitination and subsequent degradation (and/or inhibition) of the TYK2 protein. In a preferred embodiment, the ULM (ubiquitination ligase binding moiety) is a cereblon E3 ubiquitin ligase binding moiety (CLM). For example, the structure of the bifunctional compound can be depicted as:

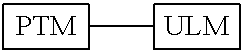

The respective positions of the PTM and ULM moieties (e.g., CLM), as well as their number as illustrated herein, is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as:

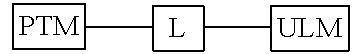

where PTM is a TYK2-targeting moiety (TTM), L is a linker, e.g., a bond or a chemical linking group coupling PTM to ULM, and ULM is a cereblon E3 ubiquitin ligase binding moiety (CLM).

For example, the structure of the bifunctional compound can be depicted as:

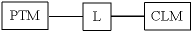

wherein: PTM is a TYK2-targeting moiety (TTM); “L” is a linker (e.g. a bond or a chemical linking group) coupling the PTM and CLM; and CLM is cereblon E3 ubiquitin ligase binding moiety that binds to cereblon.

In certain embodiments, the compounds as described herein comprise multiple independently selected ULMs, multiple PTMs, multiple chemical linkers or a combination thereof.

In any of the aspects or embodiments described herein, the PTM is a small molecule that binds the pseudokinase domain of TYK2 or a mutant thereof. In any of the aspects or embodiments described herein, the PTM is a small molecule capable of binding TYK2. In any of the aspects or embodiments described herein, the PTM is a small molecule that binds the pseudokinase or JAK homology-2 (JH2) domain of TYK2. In certain embodiments, the small molecule that binds the pseudokinase domain of TYK2 is as described herein.

In an embodiment, the CLM comprises a chemical group derived from an imide, a thioimide, an amide, or a thioamide. In a particular embodiment, the chemical group is a phthalimido group, or an analog or derivative thereof. In a certain embodiment, the CLM is selected from thalidomide, lenalidomide, pomalidomide, analogs thereof, isosteres thereof, and derivatives thereof. Other contemplated CLMs are described in U.S. Patent Application Publication No. 2015/0291562, which is incorporated herein by reference in its entirety.

In certain embodiments, “L” is a bond. In additional embodiments, the linker “L” is a connector with a linear non-hydrogen atom number in the range of 1 to 20. The connector “L” can contain, but is not limited to one or more functional groups such as ether, amide, alkane, alkene, alkyne, ketone, hydroxyl, carboxylic acid, thioether, sulfoxide, and sulfone. The linker can contain aromatic, heteroaromatic, cyclic, bicyclic or tricyclic moieties. Substitution with halogen, such as Cl, F, Br and I can be included in the linker. In the case of fluorine substitution, single or multiple fluorines can be included.

In certain embodiments, CLM is a derivative of piperidine-2,6-dione, where piperidine-2,6-dione can be substituted at the 3-position, and the 3-substitution can be bicyclic hetero-aromatics with the linkage as C—N bond or C—C bond. Examples of CLM can be, but are not limited to, pomalidomide, lenalidomide and thalidomide and their analogs.

In an additional aspect, the description provides therapeutic compositions comprising an effective amount of a compound as described herein, or a salt form thereof, and a pharmaceutically acceptable carrier. The therapeutic compositions can be used to trigger targeted degradation of TYK2 and/or inhibition of TYK2 in a patient or subject, for example, an animal such as a human, and can be used for treating or ameliorating one or more disease states, conditions, or symptoms causally related to TYK2 which treatment is accomplished through degradation or inhibition of the TYK2 protein or controlling or lowering TYK2 protein levels in a patient or subject. In certain embodiments, the therapeutic compositions as described herein may be used to effectuate the degradation of TYK2 for the treatment or amelioration of a disease such as, e.g., an infection, an inflammatory or immunological disorder, or cancer.

In yet another aspect, the present disclosure provides a method of ubiquitinating TYK2 in a cell. In certain embodiments, the method comprises administering a hetero-bifunctional compound as described herein comprising a PTM that binds TYK2, and a CLM, preferably linked through a chemical linker moiety, as described herein, to effectuate degradation of the TYK2 protein. Though not wanting to be limited by theory, the inventors believe that, pursuant to the invention, poly-ubiquitination of the TYK2 protein will occur when it is placed in proximity to the E3 ubiquitin ligase via use of the hetero-bifunctional compound, thereby triggering subsequent degradation of the TYK2 protein via the proteasomal pathway and control or reduction of TYK2 protein levels in cells of the subject. The control or reduction in TYK2 protein levels afforded by the present disclosure provides treatment of a disease state, condition or related symptom, as modulated through a lowering of the amount of TYK2 protein in cells of the subject.

In still another aspect, the description provides methods for treating or ameliorating a disease, condition, or symptom thereof in a subject or a patient, e.g., an animal such as a human, comprising administering to a subject in need thereof a composition comprising an effective amount, e.g., a therapeutically effective amount, of a hetero-bifunctional compound as described herein or salt form thereof, and a pharmaceutically acceptable carrier, wherein the composition is effective for treating or ameliorating the disease or disorder or symptom thereof in the subject.

In another aspect, the description provides methods for identifying the effects of the degradation of TYK2 protein in a biological system using compounds according to the present disclosure.

In another aspect, the description provides processes and intermediates for making a hetero-bifunctional compound of the invention capable of targeted ubiquitination and degradation of the TYK2 protein in a cell.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the disclosure, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating embodiments of the disclosure and are not to be construed as limiting the disclosure. Further objects, features and advantages of the disclosure will become apparent from the following detailed description taken in conjunction with the accompanying figures showing illustrative embodiments of the disclosure.

FIGS. 1A and 1B. Illustration of general principle for PROTAC function. FIG. 1A. Exemplary PROTACs comprise a protein targeting moiety (PTM; darkly shaded rectangle), a ubiquitin ligase binding moiety (ULM; lightly shaded triangle), and optionally a linker moiety (L; black line) coupling or tethering the PTM to the ULM. FIG. 1B Illustrates the functional use of the hetero-bifunctional protein degrading compounds (commercially known as PROTAC® brand compounds) as described herein. Briefly, the ULM (triangle) recognizes and binds to a specific E3 ubiquitin ligase, and the PTM (large rectangle) binds and recruits a target protein bringing it into close proximity to the E3 ubiquitin ligase. Typically, the E3 ubiquitin ligase is complexed with an E2 ubiquitin-conjugating protein (E2), and either alone or via the E2 protein catalyzes attachment of multiple ubiquitin molecules (black circles) to a lysine on the target protein via an isopeptide bond. The poly-ubiquitinated protein (far right) has thereby been targeted for degradation by the proteosomal machinery of the cell.

FIGS. 2A and 2B. FIG. 2A indicates the amount of TKY2 protein detected in cell lysate as a percent of TYK2 detected from cells treated with only dimethyl sulfoxide (DMSO), the vehicle, at various compound concentrations (in nanomolar (nM)). The figure shows that the exemplary hetero-bifunctional protein degrader compounds induce reductions in TYK2. FIG. 2B shows the amount of TYK2 protein, normalized to the amount of GADPH protein, detected in cell lysates after 24 hour treatment with DMSO (vehicle control), pomalidomide (a cereblon-binding, non-PROTAC control), a TYK2 hetero-bifunctional protein degrader compound as described herein (“PROTAC® example 1” or “PROTAC 1”), or PROTAC 1 plus pomalidomide. The data indicate that pomalidomide alone does not significantly reduce the amount of TYK2 protein. However, PROTAC 1 is effective at 100 nM in reducing the amount of TYK2 protein. The reversal of the PROTAC effect observed with both PROTAC 1 and pomalidomide treatment is likely due to competition for cereblon binding, and a reduction in the efficiency of cereblon-TYK2 interactions facilitated by PROTAC 1.

### DETAILED DESCRIPTION

The following is a detailed description provided to aid those skilled in the art in practicing the present invention. Those of ordinary skill in the art may make modifications and variations in the embodiments described herein without departing from the spirit or scope of the present disclosure. All publications, patent applications, patents, and other references mentioned herein are expressly incorporated by reference in their entirety.

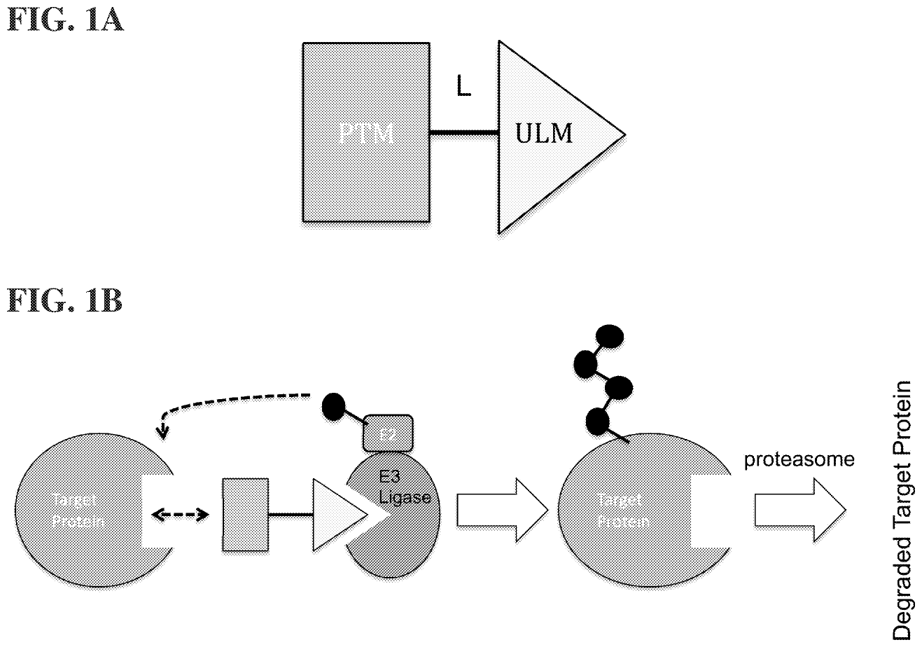

Presently described are compounds, compositions and methods that relate to the surprising and unexpected discovery that an E3 ubiquitin ligase (e.g., a cereblon E3 ubiquitin ligase) ubiquitinates the human TYK2 protein once the E3 ubiquitin ligase and the TYK2 protein are placed in proximity via a bifunctional compound that binds both the E3 ubiquitin ligase and the TYK2 protein at its pseudokinase domain. Accordingly the present disclosure provides compounds and compositions comprising an E3 ubiquitin ligase binding moiety (“ULM”) coupled by a bond or chemical linking group (L) to a protein targeting moiety (“PTM”) that targets the TYK2 protein, which results in the ubiquitination of the TYK2 protein, and which leads to degradation of the TYK2 protein by the proteasome (see FIG. 1).

TYK2 is a tyrosine kinase and member of the Janus kinases (JAKs) protein family, which also includes JAK1, JAK2, and JAK3. JAK proteins associate with the cytoplasmic domain of type I and type II cytokine receptors and promulgate cytokine signals by phosphorylating receptor subunits. JAKs share a common structure, comprising seven JAK homology domains (JH1-JH7). The N terminus of JAKs (JH7-JH3) is the most divergent and contains important domains for association with the intracellular tails of cytokine receptors and nuclear localization, whereas the C-terminus contains tandem kinase domains (JH1 and JH2). See Strobl et al. (2011) Tyrosine kinase 2 (TYK2) in cytokine signaling and host immunity. Front. Biosci. (Landmark Ed). 16:3214-3232. JH1 is an active kinase (carboxyl-terminal JH1) and JH2 is catalytically inactive and referred to as kinase-like or pseudokinase domain. Within JH1, conserved dual tyrosine residues are located in the activation-loop, a general characteristic of protein tyrosine kinases. Auto- and/or transphosphorylation by other JAKs is thought to induce conformational changes that usually positively regulate kinase activity and facilitate substrate binding

The TYK2 protein has around 1200 amino acids and a theoretical molecular weight of approximately 130 kDa. TYK2 functions primarily in IL-12 and type I-IFN signaling. However, in addition to IFN-α and -β and IL-12 signaling, TYK2 has major effects on the transduction of IL-23, IL-10, and IL-6 signals. See Strobl et al. ibid.

The pseudokinase domain of TYK2 exerts critical regulatory functions, and it has been demonstrated that certain mutations in the pseudokinase domain or pharmacologic targeting of the pseudokinase domain can alter receptor interactions as well as abolish TYK2 catalytic activity. See, e.g., Strobl et al. (2011) ibid.; Shaw et al.; Wrobleski et al, Highly Selective Inhibition of Tyrosine Kinase 2 (TYK2) for the Treatment of Autoimmune Diseases: Discovery of the Allosteric Inhibitor BMS-986165, J. Med. Chem. 2019, 62:8973-8995; Burke et al., Autoimmune pathways in mice and humans are blocked by pharmacological stabilization of the TYK2 pseudokinase domain, Sci. Transl. Med. 11:eaaw1736 (2019); Olli et al., New insights into the structure and function of the pseudokinase domain in JAK2, Biochemical Society Transactions (2013), 41(4):1002-1007; Boudeau et al., Emerging roles of pseudokinases, Trends in Cell Biology (2006), 16(9):443-452. A natural mutation in the TYK2 pseudokinase domain underlies altered susceptibility of B10.Q/J mice to infection and autoimmunity. PNAS 100(20):11594-11599. Therefore, while direct inhibition of the catalytic domain of TYK2 might be expected to result in off-target or non-specific effects, targeting of the pseudokinase domain can provide a means to specifically target TYK2.

In one aspect, the description provides compounds in which the PTM preferably binds to the pseudokinase site on the TYK2 protein. The present disclosure also provides a library of compositions and the use thereof to produce targeted degradation of the TYK2 protein in a cell.

In certain aspects, the present disclosure provides hetero-bifunctional compounds which comprise a ligand, e.g., a small molecule ligand (i.e., having a molecular weight of below 2,000, 1,000, 500, or 200 Daltons), which is capable of binding to an E3 ubiquitin ligase, such as cereblon. The compounds also comprise a small molecule moiety that is capable of binding to the pseudokinase site of TYK2 in such a way that the TYK2 protein is placed in proximity to the ubiquitin ligase to effect ubiquitination and degradation (and/or inhibition) of the TYK2 protein. “Small molecule” means, in addition to the above, that the molecule is non-peptidyl, that is, it is not considered a peptide, e.g., comprises fewer than 4, 3, or 2 amino acids. In accordance with the present description, each of the PTM, ULM and hetero-bifunctional molecule is a small molecule.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value in the range, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either/or both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element, unless otherwise indicated.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

It should also be understood that, in certain methods or processes described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more therapeutic agents at the same time) and time-varied administration (administration of one or more therapeutic agents at a time different from that of the administration of an additional therapeutic agent or agents), as long as the two or more therapeutic agents are present in the patient to some extent, preferably at effective amounts, at the same time. In certain preferred aspects, one or more of the hetero-bifunctional compounds described herein are coadministered with at least one additional bioactive agent, e.g., an anticancer agent. In particularly preferred aspects, the co-administration of such compounds results in synergistic activity and/or therapy such as, e.g., anticancer activity.

The term “compound”, as used herein, unless otherwise indicated, refers to any specific hetero-bifunctional compound disclosed herein, pharmaceutically acceptable salts and solvates thereof, and deuterated forms of any of the aforementioned molecules, where applicable. Deuterated compounds contemplated are those in which one or more of the hydrogen atoms contained in the drug molecule have been replaced by deuterium. Such deuterated compounds preferably have one or more improved pharmacokinetic or pharmacodynamic properties (e.g., longer half-life) compared to the equivalent “undeuterated” compound.

The term “ubiquitin ligase” refers to a family of proteins that facilitate the transfer of one or more ubiquitins to a specific substrate protein. Addition of a chain of several ubiquitins (poly-ubiquitination) targets the substrate protein for degradation. For example, cereblon is an E3 ubiquitin ligase that alone, or in combination with an E2 ubiquitin-conjugating enzyme, can ultimately cause the attachment of a chain of four ubiquitins to a lysine residue on the target protein, thereby targeting the protein for degradation by the proteasome. The ubiquitin ligase is involved in poly-ubiquitination such that a first ubiquitin is attached to a lysine on the target protein; a second ubiquitin is attached to the first; a third is attached to the second, and a fourth is attached to the third. Such poly-ubiquitination marks proteins for degradation by the proteasome.

The term “patient” or “subject” is used throughout the specification to describe an animal, preferably a human or a domesticated animal, to whom treatment, including prophylactic treatment, with the compositions according to the present disclosure is provided. For treatment of those diseases, conditions or symptoms that are specific for a specific animal, such as a human patient, the term “patient” refers to that specific animal, including a domesticated animal such as a dog or cat, or a farm animal such as a horse, cow, sheep, etc. In general, in the present disclosure, the terms “patient” and “subject” refer to a human patient unless otherwise stated or implied from the context of the use of the term.

The terms “effective” and “therapeutically effective” are used to describe an amount of a compound or composition which, when used within the context of its intended use, and either in a single dose or, more preferably after multiple doses within the context of a treatment regimen, effects an intended result such as an improvement in a disease or condition, or amelioration or reduction in one or more symptoms associated with a disease or condition. The terms “effective” and “therapeutically effective” subsume all other “effective amount” or “effective concentration” terms, which are otherwise described or used in the present application.

### Compounds and Compositions

In one aspect, the description provides hetero-bifunctional compounds comprising an E3 ubiquitin ligase binding moiety (“ULM”) that is a cereblon E3 ubiquitin ligase binding moiety (a “CLM”), The CLM is covalently coupled to a protein targeting moiety (PTM) that binds to the protein, which coupling is either directly by a bond or via a chemical linking group (L) according to the structure:

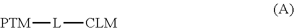

wherein L is the bond or chemical linking group, and PTM is a protein targeting moiety that binds to the protein TYK2, where the PTM is a TYK2 targeting moiety (TTM). The term CLM is inclusive of all cereblon binding moieties.

In certain embodiments, the CLM shows activity or binds to the E3 ubiquitin ligase (e.g., cereblon E3 ubiquitin ligase) with an IC₅₀ of less than about 200 μM. The IC₅₀ can be determined according to any suitable method known in the art, e.g., a fluorescent polarization assay.

In certain embodiments, the hetero-bifunctional compounds described herein demonstrate an activity with an IC₅₀ or DC₅₀ of less than about 100, 50, 10, 1, 0.5, 0.1, 0.05, 0.01, 0.005, 0.001 mM, or less than about 100, 50, 10, 1, 0.5, 0.1, 0.05, 0.01, 0.005, 0.001 μM, or less than about 100, 50, 10, 1, 0.5, 0.1, 0.05, 0.01, 0.005, 0.001 nM, or less than about 100, 50, 10, 1, 0.5, 0.1, 0.05, 0.01, 0.005, 0.001 pM.

The term “alkyl” shall mean within its context a linear, branch-chained or cyclic fully saturated hydrocarbon radical, preferably a C₁-C₁₀, preferably a C₁-C₆, or more preferably a C₁-C₃ alkyl group, which may be optionally substituted with any suitable functional group or groups. Examples of alkyl groups are methyl, ethyl, n-butyl, sec-butyl, n-hexyl, n-heptyl, n-octyl, n-nonyl, n-decyl, isopropyl, 2-methylpropyl, cyclopropyl, cyclopropylmethyl, cyclobutyl, cyclopentyl, cyclopentylethyl, cyclohexylethyl and cyclohexyl, among others. In certain embodiments, the alkyl group is end-capped with a halogen group (At, Br, Cl, F, or I).

The term “Alkenyl” refers to linear, branch-chained or cyclic C₂-C₁₀ (preferably C₂-C₆) hydrocarbon radicals containing at least one C═C bond.

The term “Alkynyl” refers to linear, branch-chained or cyclic C₂-C₁₀ (preferably C₂-C₆) hydrocarbon radicals containing at least one C≡C bond.

The term “alkylene” when used, refers to a —(CH₂)ₙ— group (n is an integer generally from 0-6), which may be optionally substituted. When substituted, the alkylene group preferably is substituted on one or more of the methylene groups with a C₁-C₆ alkyl group (including a cyclopropyl group or a t-butyl group), but may also be substituted with one or more halo groups, preferably from 1 to 3 halo groups or one or two hydroxyl groups, O—(C₁-C₆alkyl) groups or amino acid sidechains as otherwise disclosed herein. In certain embodiments, an alkylene group may be substituted with a urethane or alkoxy group (or other suitable functional group) which may be further substituted with a polyethylene glycol chain (of from 1 to 10, preferably 1 to 6, or more preferably 1 to 4 ethylene glycol units) to which is substituted (preferably, but not exclusively on the distal end of the polyethylene glycol chain) an alkyl chain substituted with a single halogen group, preferably a chlorine group. In still other embodiments, the alkylene (e.g., methylene) group, may be substituted with an amino acid sidechain group such as a sidechain group of a natural or unnatural amino acid, for example, alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan or tyrosine.

The term “unsubstituted” shall mean substituted only with hydrogen atoms. A range of carbon atoms which includes C₀ means that carbon is absent and is replaced with H. Thus, a range of carbon atoms which is C₀-C₆ includes carbons atoms of 1, 2, 3, 4, 5 and 6 and for C₀, H stands in place of carbon.

The term “substituted” or “optionally substituted” shall mean independently (i.e., where more than one substituent occurs, each substituent is selected independent of another substituent) one or more substituents (independently up to five substituents, preferably up to three substituents, more preferably 1 or 2 substituents on a moiety in a compound according to the present disclosure and may include substituents which themselves may be further substituted) at a carbon (or nitrogen) position anywhere on a molecule within context, and includes as possible substituents hydroxyl, thiol, carboxyl, cyano (C≡N), nitro (NO₂), halogen (preferably, 1, 2 or 3 halogens, especially on an alkyl, especially a methyl group such as a trifluoromethyl), an alkyl group (preferably, C₁-C₁₀, more preferably, C₁-C₆), aryl (especially phenyl and substituted phenyl, for example benzyl or benzoyl), alkoxy group (preferably, C₁-C₆alkyl or aryl, including phenyl and substituted phenyl), thioether (preferably, C₁-C₆ alkyl or aryl), acyl (preferably, C₁-C₆ acyl), ester or thioester (preferably, C₁-C₆ alkyl or aryl) including alkylene ester (such that attachment is on the alkylene group, rather than at the ester function which is preferably substituted with a C₁-C₆ alkyl or aryl group), halogen (preferably, F or Cl), amine (including a five- or six-membered cyclic alkylene amine, further including a C₁-C₆ alkyl amine or a C₁-C₆ dialkyl amine which alkyl groups may be substituted with one or two hydroxyl groups) or an optionally substituted —N(C₀-C₆ alkyl)C(O)(O—C₁-C₆ alkyl) group (which may be optionally substituted with a polyethylene glycol chain to which is further bound an alkyl group containing a single halogen, preferably chlorine substituent), hydrazine, amido, which are preferably independently substituted with one or two C₁-C₆ alkyl groups (including a carboxamide which is optionally substituted with one or two C₁-C₆ alkyl groups), alkanol (preferably, C₁-C₆ alkyl or aryl), or alkanoic acid (preferably, C₁-C₆ alkyl or aryl). Substituents according to the present disclosure may include, for example —SiR₁R₂R₃ groups where each of R₁ and R₂ is as otherwise described herein and R₃ is H or a C₁-C₆ alkyl group, preferably R₁, R₂, R₃ together is a C₁-C₃ alkyl group (including an isopropyl or t-butyl group). Each of the above-described groups may be linked directly to the substituted moiety or alternatively, the substituent may be linked to the substituted moiety (preferably in the case of an aryl or heteroaryl moiety) through an optionally substituted —(CH₂)ₘ or alternatively an optionally substituted —(OCH₂)ₘ—, —(OCH₂CH₂)ₘ or —(CH₂CH₂O)ₘ group, which may be substituted with any one or more of the above-described substituents. Alkylene groups —(CH₂)ₘ or —(CH₂)ₙ— groups or other chains such as ethylene glycol chains, as identified above, may be substituted anywhere on the chain. Preferred substituents on alkylene groups include halogen or C₁-C₆ (preferably C₁-C₃) alkyl groups, which may be optionally substituted with one or two hydroxyl groups, one or two ether groups (O—C₁-C₆ groups), up to three halo groups (preferably F), or a side chain of an amino acid as otherwise described herein and optionally substituted amide (preferably carboxamide substituted as described above) or urethane groups (often with one or two C₀-C₆ alkyl substituents, which group(s) may be further substituted). In certain embodiments, the alkylene group (often a single methylene group) is substituted with one or two optionally substituted C₁-C₆ alkyl groups, preferably C₁-C₄ alkyl group, most often methyl or O-methyl groups or a sidechain of an amino acid as otherwise described herein. In the present disclosure, a moiety in a molecule may be optionally substituted with up to five substituents, preferably up to three substituents. Most often, in the present disclosure moieties which are substituted are substituted with one or two substituents.

The term “substituted” (each substituent being independent of any other substituent) shall also mean within its context of use C₁-C₆ alkyl, C₁-C₆ alkoxy, halogen, amido, carboxamido, sulfone, including sulfonamide, keto, carboxy, C₁-C₆ ester (oxyester or carbonylester), C₁-C₆ keto, urethane —O—C(O)—NR₁R₂ or —N(R₁)—C(O)—O—R₁, nitro, cyano and amine (especially including a C₁-C₆ alkylene-NR₁R₂, a mono- or di-C₁-C₆ alkyl substituted amines which may be optionally substituted with one or two hydroxyl groups). Each of these groups contain unless otherwise indicated, within context, between 1 and 6 carbon atoms. In certain embodiments, preferred substituents will include for example, —NH—, —NHC(O)—, —O—, ═O, —(CH₂)ₘ (here, m and n are in context, 1, 2, 3, 4, 5 or 6), —S—, —S(O)—, SO₂— or —NH—C(O)—NH—, —(CH₂)ₙOH, —(CH₂)ₙSH, —(CH₂)ₙCOOH, C₁-C₆ alkyl, —(CH₂)ₙO—(C₁-C₆ alkyl), —(CH₂)ₙC(O)—(C₁-C₆ alkyl), —(CH₂)ₙOC(O)—(C₁-C₆ alkyl), —(CH₂)ₙC(O)O—(C₁-C₆ alkyl), —(CH₂)ₙNHC(O)—R₁, —(CH₂)ₙC(O)—NR₁R₂, —(OCH₂)ₙOH, —(CH₂O)ₙCOOH, C₁-C₆ alkyl, —(OCH₂)ₙO—(C₁-C₆ alkyl), —(CH₂O)ₙC(O)—(C₁-C₆ alkyl), —(OCH₂)ₙNHC(O)—R₁, —(CH₂O)ₙC(O)—NR₁R₂, —S(O)₂—R_{S}, —S(O)—R_{S} (R_{S} is C₁-C₆ alkyl or a —(CH₂)ₘ—NR₁R₂ group), NO₂, CN or halogen (F, Cl, Br, I, preferably F or Cl), depending on the context of the use of the substituent. R₁ and R₂ are each, within context, H or a C₁-C₆ alkyl group (which may be optionally substituted with one or two hydroxyl groups or up to three halogen groups, preferably fluorine). The term “substituted” shall also mean, within the chemical context of the compound defined and substituent used, an optionally substituted aryl or heteroaryl group or an optionally substituted heterocyclic group as otherwise described herein. Alkylene groups may also be substituted as otherwise disclosed herein, preferably with optionally substituted C₁-C₆ alkyl groups (methyl, ethyl or hydroxymethyl or hydroxyethyl is preferred, thus providing a chiral center), a sidechain of an amino acid group as otherwise described herein, an amido group as described hereinabove, or a urethane group O—C(O)—NR₁R₂ group where R₁ and R₂ are as otherwise described herein, although numerous other groups may also be used as substituents. Various optionally substituted moieties may be substituted with 3 or more substituents, preferably no more than 3 substituents and preferably with 1 or 2 substituents. It is noted that in instances where, in a compound at a particular position of the molecule substitution is required (principally, because of valency), but no substitution is indicated, then that substituent is construed or understood to be H, unless the context of the substitution suggests otherwise.

The term “aryl” or “aromatic”, in context, refers to a substituted (as otherwise described herein) or unsubstituted monovalent aromatic radical having a single ring (e.g., benzene, phenyl, benzyl) or condensed rings (e.g., naphthyl, anthracenyl, phenanthrenyl, etc.) and can be bound to the compound according to the present disclosure at any available stable position on the ring(s) or as otherwise indicated in the chemical structure presented. Other examples of aryl groups, in context, may include heterocyclic aromatic ring systems, “heteroaryl” groups having one or more nitrogen, oxygen, or sulfur atoms in the ring (moncyclic) such as imidazole, furyl, pyrrole, furanyl, thiene, thiazole, pyridine, pyrimidine, pyrazine, triazole, oxazole or fused ring systems such as indole, quinoline, indolizine, azaindolizine, benzofurazan, etc., among others, which may be optionally substituted as described above. Among the heteroaryl groups which may be mentioned include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, azaindolizine, purine, indazole, quinoline, dihydroquinoline, tetrahydroquinoline, isoquinoline, dihydroisoquinoline, tetrahydroisoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, pyrimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

The term “substituted aryl” refers to an aromatic carbocyclic group comprised of at least one aromatic ring or of multiple condensed rings at least one of which being aromatic, wherein the ring(s) are substituted with one or more substituents. For example, an aryl group can comprise a substituent(s) selected from: —(CH₂)ₙOH, —(CH₂)ₙ—O—(C₁-C₆)alkyl, —(CH₂)ₙ—O—(CH₂)ₙ—(C₁-C₆)alkyl, —(CH₂)ₙ—C(O)(C₀-C₆) alkyl, —(CH₂)ₙ—C(O)O(C₀-C₆)alkyl, —(CH₂)ₙ—OC(O)(C₀-C₆)alkyl, amine, mono- or di-(C₁-C₆ alkyl) amine wherein the alkyl group on the amine is optionally substituted with 1 or 2 hydroxyl groups or up to three halo (preferably F, Cl) groups, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is preferably connected to a PTM group, including a ULM group, via a linker group), and/or at least one of F, Cl, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methyl substituted isoxazole, an optionally substituted oxazole including a methyl substituted oxazole, an optionally substituted thiazole including a methyl substituted thiazole, an optionally substituted isothiazole including a methyl substituted isothiazole, an optionally substituted pyrrole including a methyl substituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methylsubstituted triazole group, an optionally substituted pyridine group, including a halo-(preferably, F) or methyl substituted pyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, and combinations thereof.

“Carboxyl” denotes the group —C(O)OR, where R is hydrogen, alkyl, substituted alkyl, aryl, substituted aryl, heteroaryl or substituted heteroaryl, whereas these generic substituents have meanings which are identical with definitions of the corresponding groups defined herein.

The term “heteroaryl” or “hetaryl” can mean but is in no way limited to an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ—C(O)—O—C₁-C₆ alkyl group), an optionally substituted pyridine (2-, 3, or 4-pyridine) or a group according to the chemical structure:

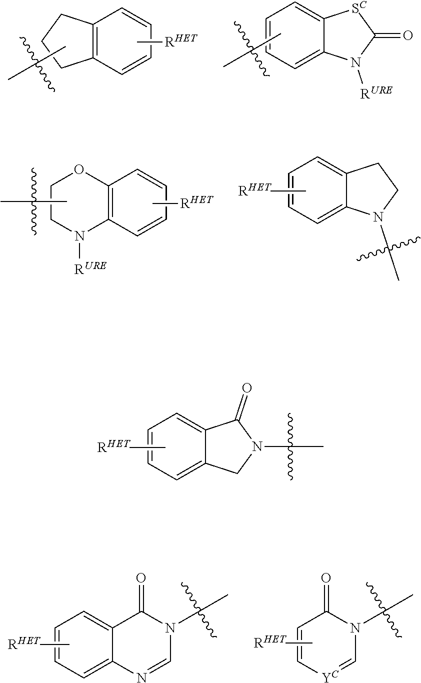

wherein:
S′ is CHR^{SS}, NR^{URE}, or O;
R^{HET} is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{C} is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl).

The terms “aralkyl” and “heteroarylalkyl” refer to groups that comprise both aryl or, respectively, heteroaryl as well as alkyl and/or heteroalkyl and/or carbocyclic and/or heterocycloalkyl ring systems according to the above definitions.

The term “arylalkyl” as used herein refers to an aryl group as defined above appended to an alkyl group defined above. The arylalkyl group is attached to the parent moiety through an alkyl group wherein the alkyl group is one to six carbon atoms. The aryl group in the arylalkyl group may be substituted as defined above.

The term “Heterocycle” refers to a cyclic group which contains at least one heteroatom, e.g., N, O or S, and may be aromatic (heteroaryl) or non-aromatic. Thus, the heteroaryl moieties are subsumed under the definition of heterocycle, depending on the context of its use. Exemplary heteroaryl groups are described hereinabove.

Exemplary heterocyclics include: azetidinyl, benzimidazolyl, 1,4-benzodioxanyl, 1,3-benzodioxolyl, benzoxazolyl, benzothiazolyl, benzothienyl, dihydroimidazolyl, dihydropyranyl, dihydrofuranyl, dioxanyl, dioxolanyl, ethyleneurea, 1,3-dioxolane, 1,3-dioxane, 1,4-dioxane, furyl, homopiperidinyl, inidazolyl, imidazolinyl, imidazolidinyl, indolinyl, indolyl, isoquinolinyl, isothiazolidinyl, isothiazolyl, isoxazolidinyl, isoxazolyl, morpholinyl, naphthyridinyl, oxazolidinyl, oxazolyl, pyridone, 2-pyrrolidone, pyridine, piperazinyl, N-methylpiperazinyl, piperidinyl, phthalimide, succinimide, pyrazinyl, pyrazolinyl, pyridyl, pyrinidinyl, pyrrolidinyl, pyrrolinyl, pyrrolyl, quilolinyl, tetrahydrofuranyl, tetrahydropyranyl, tetrahydroquinoline, thiazolidinyl, thiazolyl, thienyl, tetrahydrothiophene, oxane, oxetanyl, oxathiolanyl, thiane among others.

Heterocyclic groups can be optionally substituted with a member selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxy, carboxyalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SOaryl, —SO-heteroaryl, —SO2-alkyl, —SO2-substituted alkyl, —SO2-aryl, oxo (═O), and —SO2- heteroaryl. Such heterocyclic groups can have a single ring or multiple condensed rings. Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. The term “heterocyclic” also includes bicyclic groups in which any of the heterocyclic rings is fused to a benzene ring or a cyclohexane ring or another heterocyclic ring (for example, indolyl, quinolyl, isoquinolyl, tetrahydroquinolyl, and the like).

The term “cycloalkyl” can mean but is in no way limited to univalent groups derived from monocyclic or polycyclic alkyl groups or cycloalkanes, as defined herein, e.g., saturated monocyclic hydrocarbon groups having from three to twenty carbon atoms in the ring, including, but not limited to, cyclopropyl, cyclobutyl, cyclopentyl, cyclohexyl, cycloheptyl and the like. The term “substituted cycloalkyl” can mean but is in no way limited to a monocyclic or polycyclic alkyl group and being substituted by one or more substituents, for example, amino, halogen, alkyl, substituted alkyl, carbyloxy, carbylmercapto, aryl, nitro, mercapto or sulfo, whereas these generic substituent groups have meanings which are identical with definitions of the corresponding groups as defined in this legend.

“Heterocycloalkyl” refers to a monocyclic or polycyclic alkyl group in which at least one ring carbon atom of its cyclic structure being replaced with a heteroatom selected from the group consisting of N, O, S or P. “Substituted heterocycloalkyl” refers to a monocyclic or polycyclic alkyl group in which at least one ring carbon atom of its cyclic structure being replaced with a heteroatom selected from the group consisting of N, O, S or P and the group is containing one or more substituents selected from the group consisting of halogen, alkyl, substituted alkyl, carbyloxy, carbylmercapto, aryl, nitro, mercapto or sulfo, whereas these generic substituent group have meanings which are identical with definitions of the corresponding groups as defined in this legend.

The term “hydrocarbyl” shall mean a compound which contains carbon and hydrogen and which may be fully saturated, partially unsaturated or aromatic and includes aryl groups, alkyl groups, alkenyl groups and alkynyl groups.

The term “independently” is used herein to indicate that the variable, which is independently applied, varies independently from application to application.

The term “lower alkyl” refers to methyl, ethyl or propyl

The term “lower alkoxy” refers to methoxy, ethoxy or propoxy.

### Exemplary CLMs

### Neo-Imide Compounds

In one aspect the description provides CLMs useful for binding and recruiting cereblon. In certain embodiments, the CLM is selected from the group consisting of chemical structures:

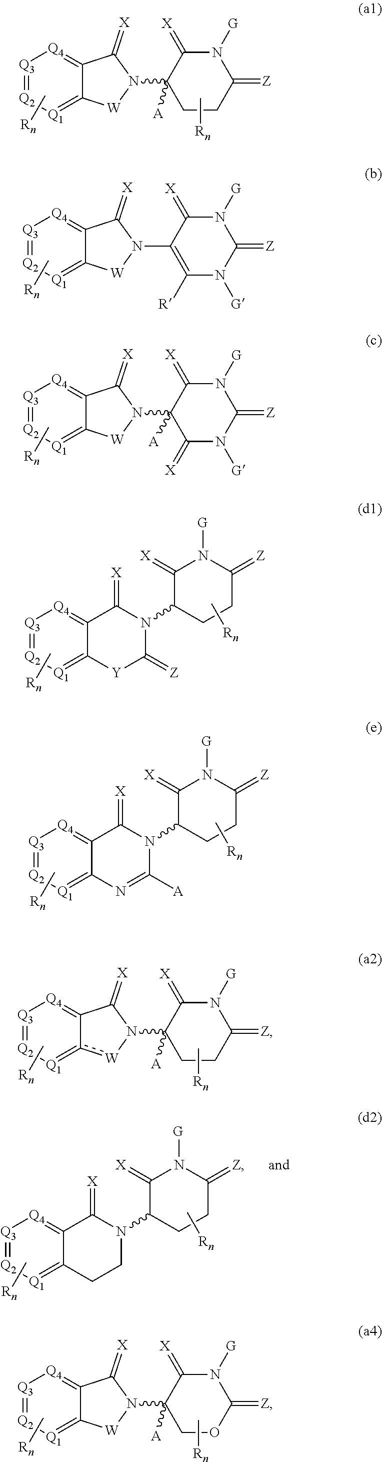

wherein:
W of Formulas (a) through (f) is independently selected from the group CH₂, O, CHR, C═O, SO₂, NH, N, optionally substituted cyclopropyl group, optionally substituted cyclobutyl group, and N-alkyl;
W₃ is selected from C or N;
X of Formulas (a) through (f) is independently selected from the group absent, O, S and CH₂;
Y of Formulas (a) through (f) is independently selected from the group CH₂, —C═CR′, NH, N-alkyl, N-aryl, N-heteroaryl, N-cycloalkyl, N-heterocyclyl, O, and S;
Z of Formulas (a) through (f) is independently selected from the group absent, O, and S or CH₂ except that both X and Z cannot be CH₂ or absent;
G and G′ of Formulas (a) through (f) are independently selected from the group H, optionally substituted linear or branched alkyl, OH, R′OCOOR, R′OCONRR″, CH₂-heterocyclyl optionally substituted with R′, and benzyl optionally substituted with R′;
Q1-Q4 of Formulas (a) through (f) represent a carbon C or N substituted with a group independently selected from H, R, N or N-oxide;
A of Formulas (a) through (f) is independently selected from the group H, optionally substituted linear or branched alkyl, cycloalkyl, Cl and F;
n of Formulas (a) through (f) represent an integer from 1 to 10 (e.g., 1-4, 1, 2, 3, 4, 5, 6, 7, 8, 9, or 10);
R of Formulas (a) through (f) comprises, but is not limited to: H, —C(═O)R′ (e.g., a carboxy group), —CONR′R″ (e.g., an amide group), —OR′ (e.g., OH), —NR′R″ (e.g., an amine group), —SR′, —SO₂R′, —SO₂NR′R″, —CR′R″—, —CR′NR′R″—, (—CR′O)_{n′}R″, optionally substituted heterocyclyl, optionally substituted aryl, (e.g., an optionally substituted C5-C7 aryl), optionally substituted alkyl-aryl (e.g., an alkyl-aryl comprising at least one of an optionally substituted C1-C6 alkyl, an optionally substituted C5-C7 aryl, or combinations thereof), optionally substituted heteroaryl, optionally substituted alkyl (e.g., a C1-C6 linear or branched alkyl optionally substituted with one or more halogen, cycloalkyl (e.g., a C3-C6 cycloalkyl), or aryl (e.g., C5-C7 aryl)), optionally substituted alkoxyl group (e.g., a methoxy, ethoxy, butoxy, propoxy, pentoxy, or hexoxy; wherein the alkoxyl may be substituted with one or more halogen, alkyl, haloalky, fluoroalkyl, cycloalkyl (e.g., a C3-C6 cycloalkyl), or aryl (e.g., C5-C7 aryl)), optionally substituted cycloalkyl, optionally substituted heterocyclyl, —P(O)(OR′)R″, —P(O)R′R″, —OP(O)(OR′)R″, —OP(O)R′R″, —Cl, —F, —Br, —I, —CF₃, —CN, —NR′SO₂NR′R″, —NR′CONR′R″, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —NO₂, —CO₂R′, —C(C═N—OR′)R″, —CR′═CR′R″, —CCR′, —S(C═O)(C═N—R′)R″, —SF₅ and —OCF₃, wherein at least one W, X, Y, Z, G, G′, R, R′, R″, Q1-Q4, or A is modified to be covalently joined to a PTM, a chemical linking group (L), a ULM, CLM, or combination thereof;
each of x, y, and z are independently 0, 1, 2, 3, 4, 5, or 6;
R′ and R″ of Formulas (a) through (f) are independently selected from H, optionally substituted linear or branched alkyl, optionally substituted cycloalkyl, optionally substituted aryl, optionally substituted heteroaryl, optionally substituted heterocyclic, —C(═O)R, optionally substituted heterocyclyl;
n′ of Formulas (a) through (f) is an integer from 1-10 (e.g. 1-4, 1, 2, 3, 4, 5, 6, 7, 8, 9, or 10);
[symbol] represents a single bond or a double bond; and
[symbol] of Formulas (a) through (f) represents a bond that may be stereospecific ((R) or (S)) or non-stereospecific.

In any of the compounds described herein, the CLM comprises a chemical structure selected from the group consisting of:

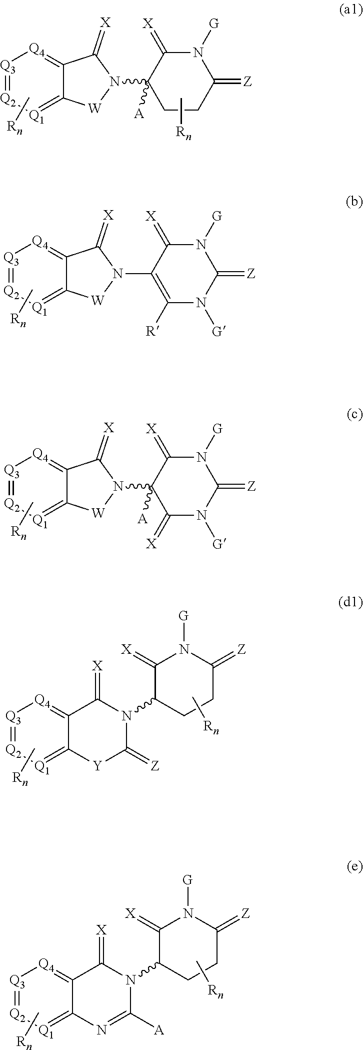

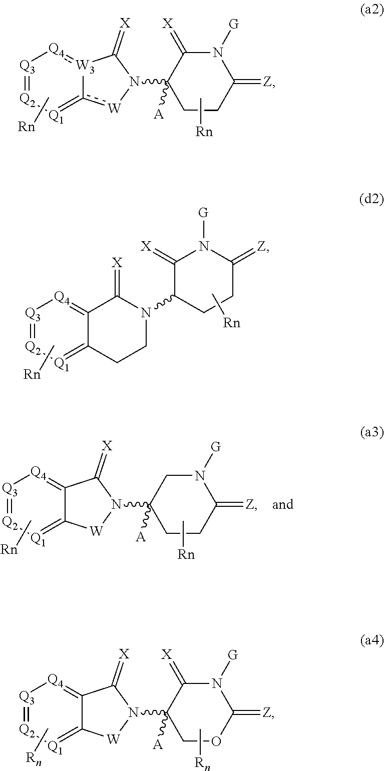

wherein:
W of Formulas (a) through (f) is independently selected from the group CH₂, O, CHR, C═O, SO₂, NH, N, optionally substituted cyclopropyl group, optionally substituted cyclobutyl group, and N-alkyl;
W₃ is selected from C or N;
X of Formulas (a) through (f) is independently selected from the group O, S and CH₂;
Y of Formulas (a) through (f) is independently selected from the group CH₂, —C═CR′, NH, N-alkyl, N-aryl, N-hetaryl, N-cycloalkyl, N-heterocyclyl, O, and S;
Z of Formulas (a) through (f) is independently selected from the group O, and S or CH2 except that both X and Z cannot be CH₂ or absent;
G and G′ of Formulas (a) through (f) are independently selected from the group H, optionally substituted linear or branched alkyl, OH, R′OCOOR, R′OCONRR″, CH₂-heterocyclyl optionally substituted with R′, and benzyl optionally substituted with R′;
Q1-Q4 of Formulas (a) through (f) represent a carbon C or N substituted with a group independently selected from H, R, N or N-oxide;
A of Formulas (a) through (f) is independently selected from the group H, optionally substituted linear or branched alkyl, cycloalkyl, Cl and F;
n of Formulas (a) through (f) represent an integer from 1 to 10 (e.g., 1-4, 1, 2, 3, 4, 5, 6, 7, 8, 9, or 10);
R of Formulas (a) through (f) comprises, but is not limited to: H, —C(═O)R′ (e.g., a carboxy group), —CONR′R″ (e.g., an amide group), —OR′ (e.g., OH), —NR′R″ (e.g. an amine group), —SR′, —SO2R′, —SO2NR′R″, —CR′R″—, —CR′NR′R″—, (—CR′O)_{n′}R″, optionally substituted aryl (e.g., an optionally substituted C5-C7 aryl), optionally substituted alkyl-aryl (e.g., an alkyl-aryl comprising at least one of an optionally substituted C1-C6 alkyl, an optionally substituted C5-C7 aryl, or combinations thereof), optionally substituted hetaryl, -optionally substituted linear or branched alkyl (e.g., a C1-C6 linear or branched alkyl optionally substituted with one or more halogen, cycloalkyl (e.g., a C3-C6 cycloalkyl), or aryl (e.g., C5-C7 aryl)), optionally substituted alkoxyl group (e.g., a methoxy, ethoxy, butoxy, propoxy, pentoxy, or hexoxy; wherein the alkoxyl may be substituted with one or more halogen, alkyl, haloalky, fluoroalkyl, cycloalkyl (e.g., a C3-C6 cycloalkyl), or aryl (e.g., C5-C7 aryl)), optionally substituted cycloalkyl, optionally substituted heterocyclyl, —P(O)(OR′)R″, —P(O)R′R″, —OP(O)(OR′)R″, —OP(O)R′R″, —Cl, —F, —Br, —I, —CF3, —CN, —NR′SO2NR′R″, —NR′CONR′R″, —CONR′COR″, —NR′C(═N—CN)NR′R″, —C(═N—CN)NR′R″, —NR′C(═N—CN)R″, —NR′C(═C—NO2)NR′R″, —SO2NR′COR″, —NO2, —CO2R′, —C(C═N—OR′)R″, —CR′═CR′R″, —CCR′, —S(C═O)(C═N—R′)R″, —SF5 and —OCF3, wherein at least one of W, X, Y, Z, G, G′, R, R′, R″, Q1-Q4, or A is covalently joined (directly or indirectly, e.g., via a functional group or an atom, such as O, S, N) to a PTM, a chemical linking group (L), a ULM, CLM, or combination thereof;
each of x, y, and z are independently 0, 1, 2, 3, 4, 5, or 6;
R′ and R″ of Formulas (a) through (f) are independently selected from a bond, H, optionally substituted linear or branched alkyl, optionally substituted cycloalkyl, optionally substituted aryl, optionally substituted heteroaryl, optionally substituted heterocyclic, —C(═O)R, optionally substituted heterocyclyl;
n′ of Formulas (a) through (f) is an integer from 1-10 (e.g., 1-4, 1, 2, 3, 4, 5, 6, 7, 8, 9, or 10); and
[symbol] of Formulas (a) through (f) represents a bond that may be stereospecific ((R) or (S)) or non-stereospecific.

In certain embodiments described herein, the CLM or ULM is selected from the structure of Formula (g):

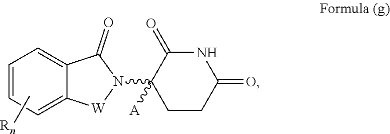

wherein:
W of Formula (g) is independently selected from the group CH₂, O, C═O, NH, and N-alkyl;
A of Formula (g) is selected from a H, methyl, or optionally substituted linear or branched alkyl;
n is an integer from 1 to 4;
R of Formula (g) is independently selected from a H, O, OH, N, NH, NH₂, methyl, optionally substituted linear or branched alkyl (e.g., optionally substituted linear or branched C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy), wherein at least one R or W is modified to be covalently joined to a PTM, a chemical linking group (L), a ULM, CLM, or combination thereof; and
[symbol] of Formula (g) represents a bond that may be stereospecific ((R) or (S)) or non-stereospecific.

In one embodiment described herein, R is selected from: O, OH, N, NH, NH₂, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy).

In one embodiment described herein, at least one R (e.g. an R group selected from the following O, OH, N, NH, NH₂, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy) or W is modified to be covalently joined to a PTM, a chemical linker group (L), a ULM, a CLM, or a combination thereof.

In any of the embodiments described herein, the W, X, Y, Z, G, G′, R, R′, R″, Q1-Q4, A, and Rn of Formulas (a) through (g) can independently be covalently coupled to a linker and/or a linker to which is attached one or more PTM, ULM, or CLM groups.

In any of the aspects or embodiments described herein, Rₙ comprises from 1 to 4 independently selected functional groups or atoms, for example, O, OH, N, C1-C6 alkyl, C1-C6 alkoxy, -alkyl-aryl (e.g., an -alkyl-aryl comprising at least one of C1-C6 alkyl, C4-C7 aryl, or a combination thereof), aryl (e.g., C5-C7 aryl), amine, amide, or carboxy, on the aryl or heteroaryl of the CLM, and optionally, one of which is modified to be covalently joined to a PTM, a chemical linker group (L), a ULM, CLM or combination thereof.

More specifically, non-limiting examples of CLMs include those shown below as well as those “hybrid” molecules that arise from the combination of one or more of the different features shown in the molecules below wherein at least one R or W is modified to be covalently joined to a PTM, a chemical linking group (L), a ULM, CLM, or combination thereof.

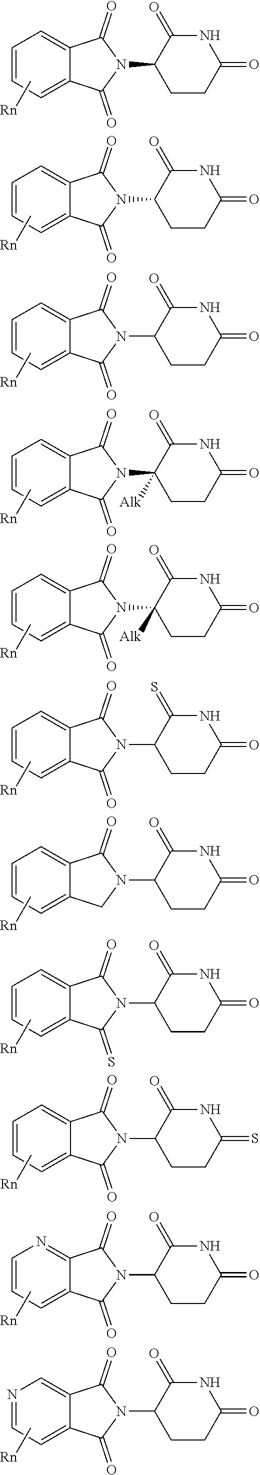

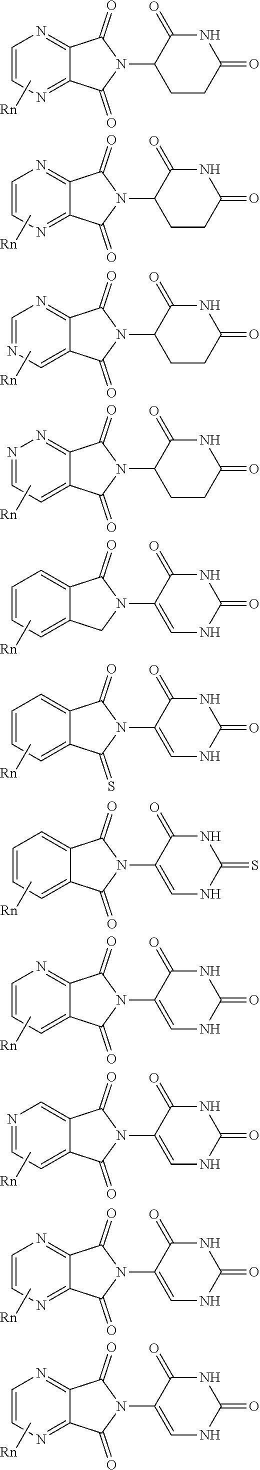

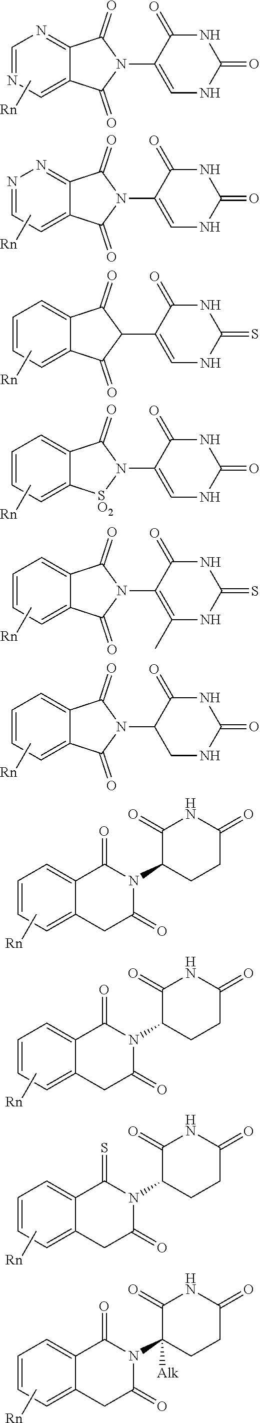

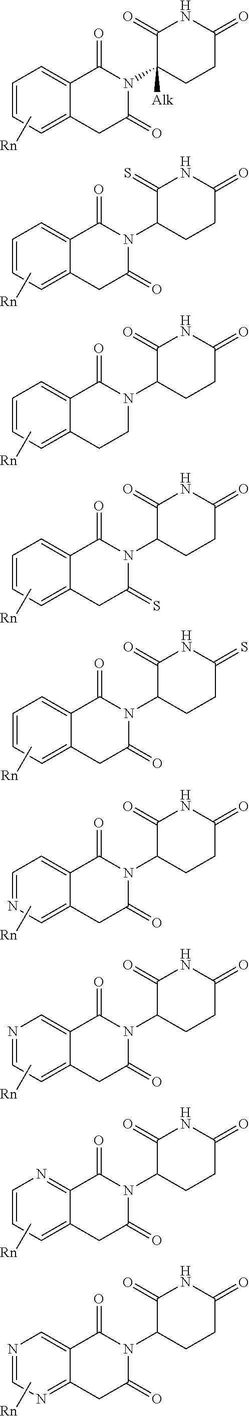

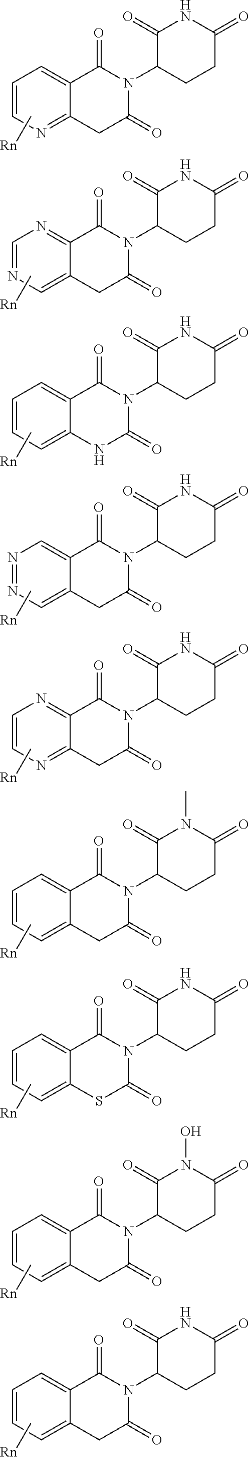

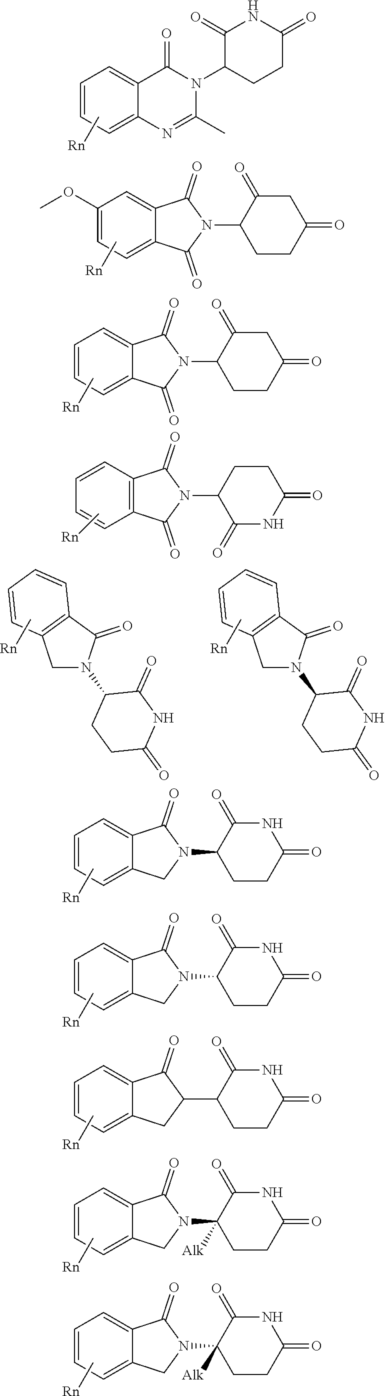

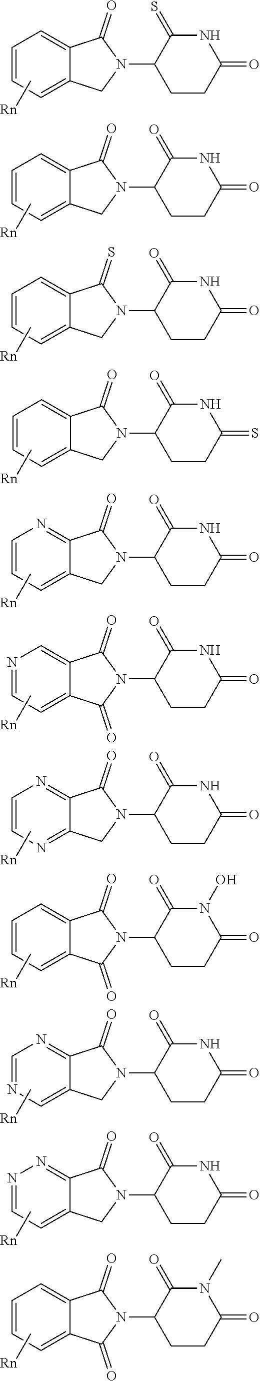

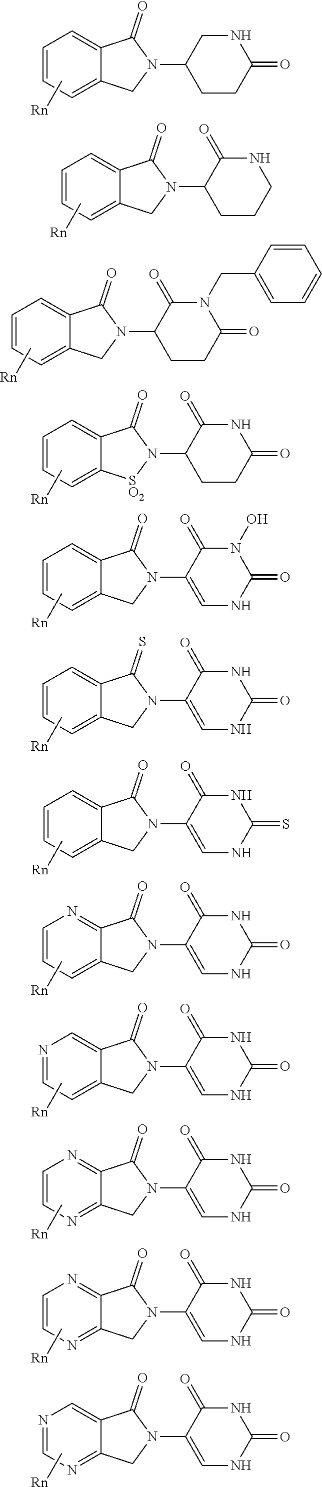

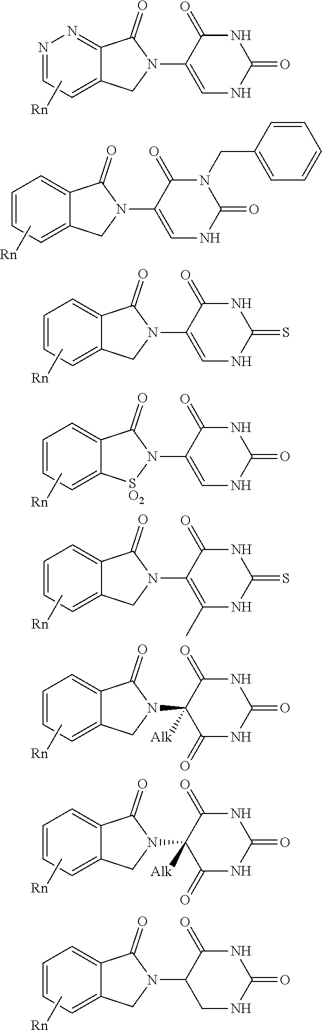

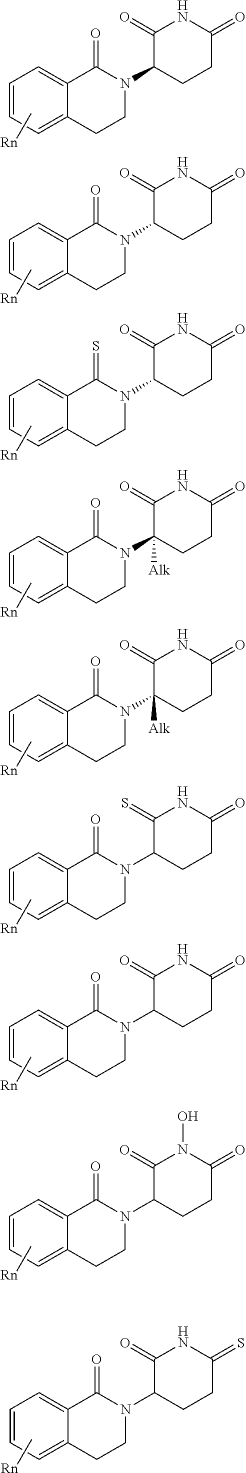

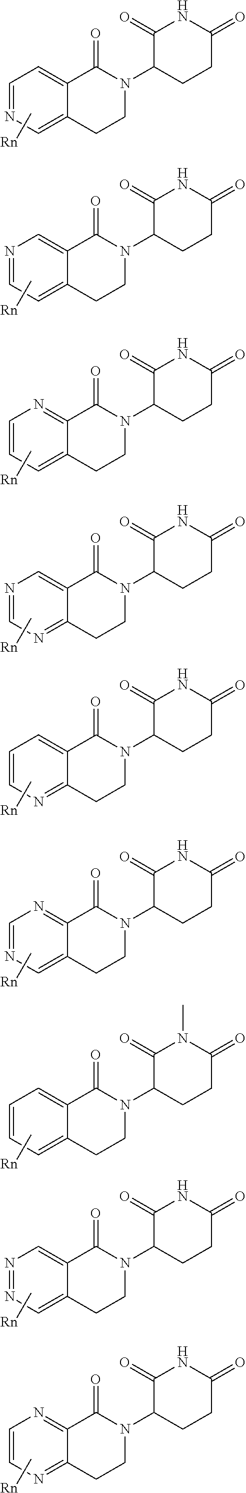

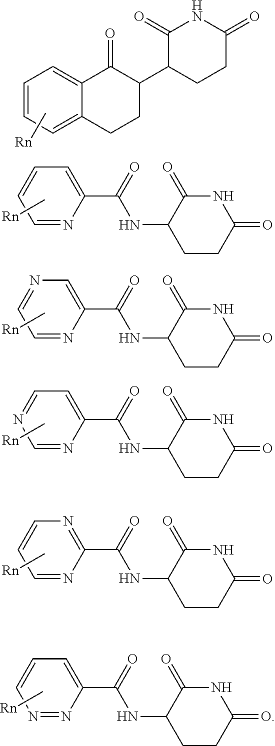

In any of the compounds described herein, the CLM comprises a chemical structure selected from the group:

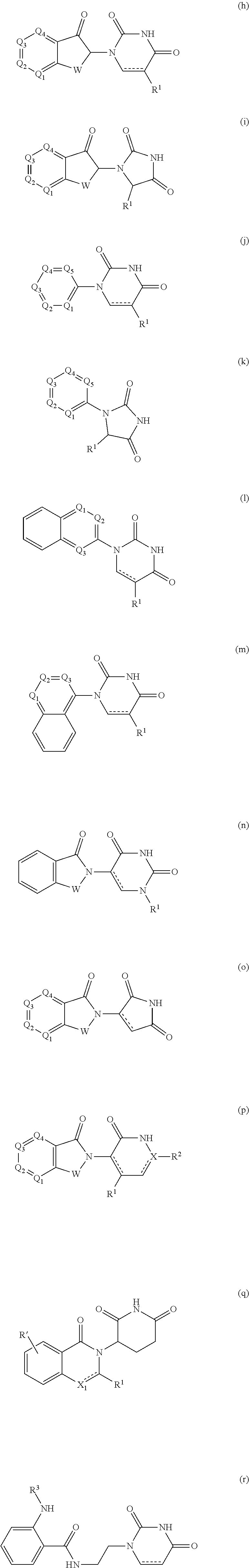

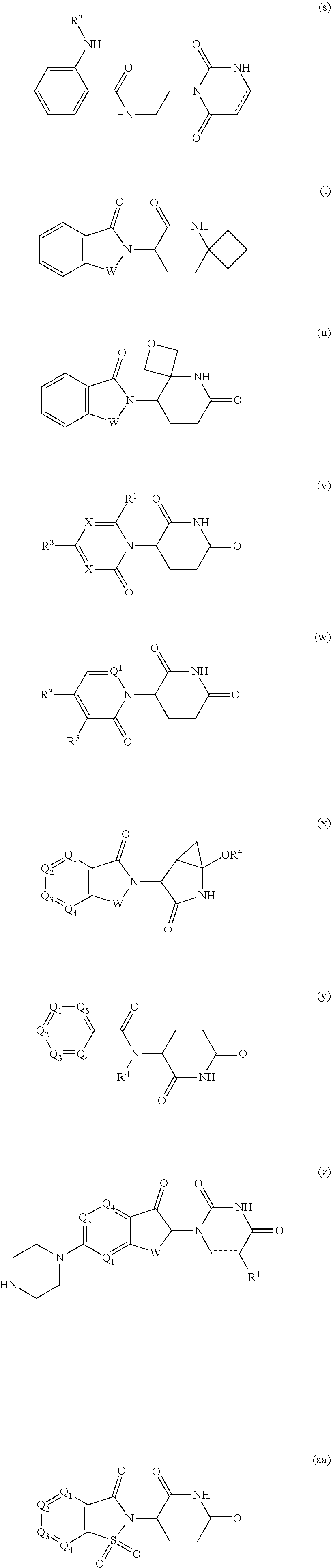

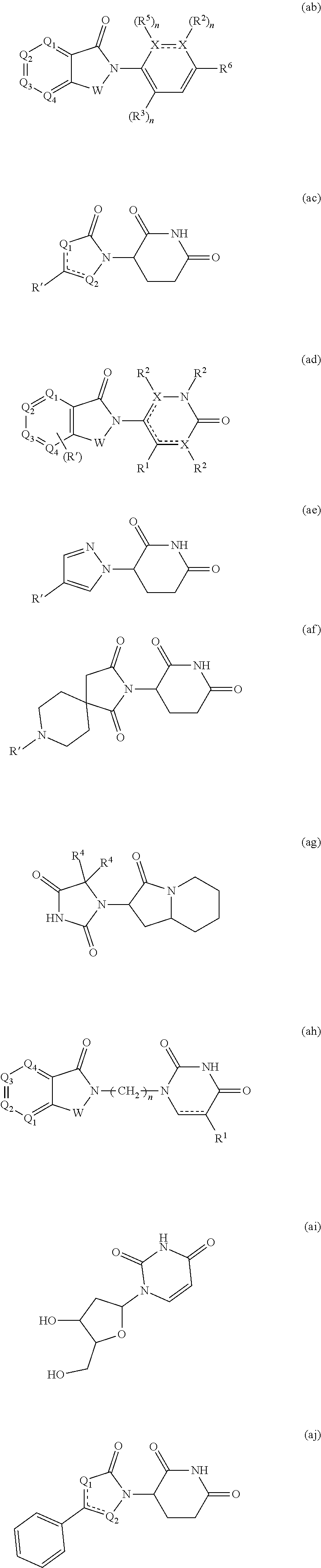

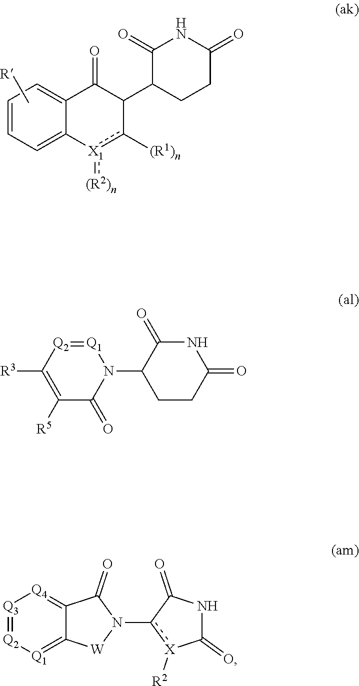

wherein:
W is independently selected from CH₂, O, CHR, C═O, SO₂, NH, N, optionally substituted cyclopropyl group, optionally substituted cyclobutyl group, and N-alkyl (e.g., CH₂, CHR, C═O, SO₂, NH, and N-alkyl);
Q₁, Q₂, Q₃, Q₄, Q₅ are each independently represent a carbon C or N substituted with a group independently selected from R′, N or N-oxide;
R¹ is selected from absent, H, OH, CN, C1-C3 alkyl, C═O;
R² is selected from the group absent, H, OH, CN, C1-C3 alkyl, CHF₂, CF₃, CHO, C(═O)NH₂;
R³ is selected from H, alkyl (e.g., C1-C6 or C1-C3 alkyl), substituted alkyl (e.g., substituted C1-C6 or C1-C3 alkyl), alkoxy (e.g., C1-C6 or C1-C3 alkoxyl), substituted alkoxy (e.g., substituted C1-C6 or C1-C3 alkoxyl);
R⁴ is selected from H, alkyl, substituted alkyl;
R⁵ and R⁶ are each independently H, halogen, C(═O)R′, CN, OH, CF₃;
X is C, CH, C═O, or N;
X₁ is C═O, N, CH, or CH₂;
R′ is selected from H, halogen, amine, alkyl (e.g., C1-C3 alkyl), substituted alkyl (e.g., substituted C1-C3 alkyl), alkoxy (e.g., C1-C3 alkoxyl), substituted alkoxy (e.g., substituted C1-C3 alkoxyl), NR²R³, C(═O)OR², optionally substituted phenyl;
n is 0-4;
[symbol] is a single or double bond; and
the CLM is covalently joined to a PTM, a chemical linker group (L), a ULM, CLM or combination thereof.

In one embodiment described herein, the CLM is covalently joined to a PTM, a chemical linker group (L), a ULM, a CLM, or a combination thereof via an R group (such as, R, R¹, R², R³, R⁴ or R′), W, X, or a Q group (such as, Q₁, Q₂, Q₃, Q₄, or Q₅).

In any of the embodiments described herein, the CLM is covalently joined to a PTM, a chemical linker group (L), a ULM, a CLM, or a combination thereof via W, X, R, R¹, R², R³, R⁴, R⁵, R′, Q₁, Q₂, Q₃, Q₄, and Q₅.

In any of the embodiments described herein, the W, X, R¹, R², R³, R⁴, R′, Q₁, Q₂, Q₃, Q₄, and Q₅ can independently be covalently coupled to a linker and/or a linker to which is attached to one or more PTM, ULM, ULM′, CLM groups.

More specifically, non-limiting examples of CLMs include those shown below as well as “hybrid” molecules or compounds that arise from combining one or more features of the following compounds:

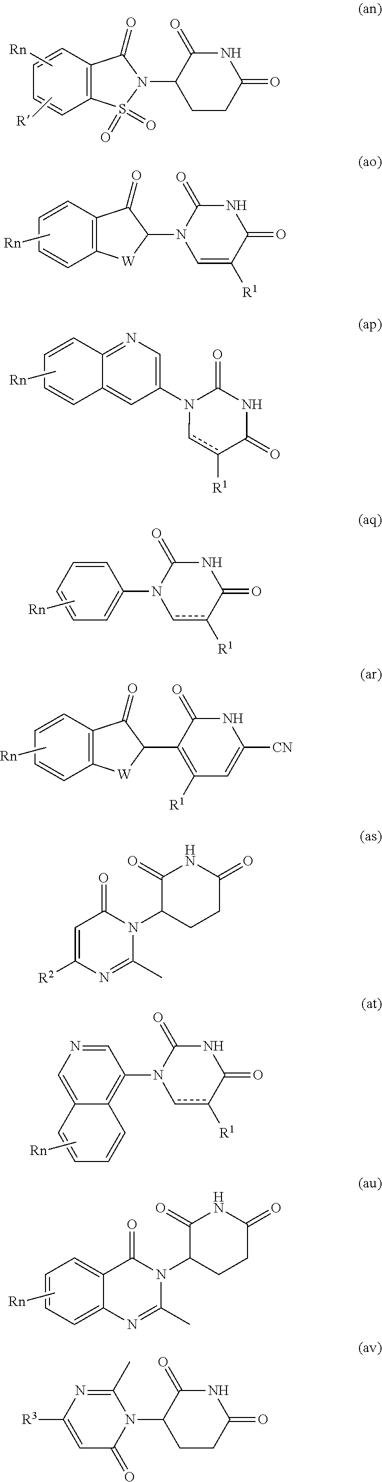

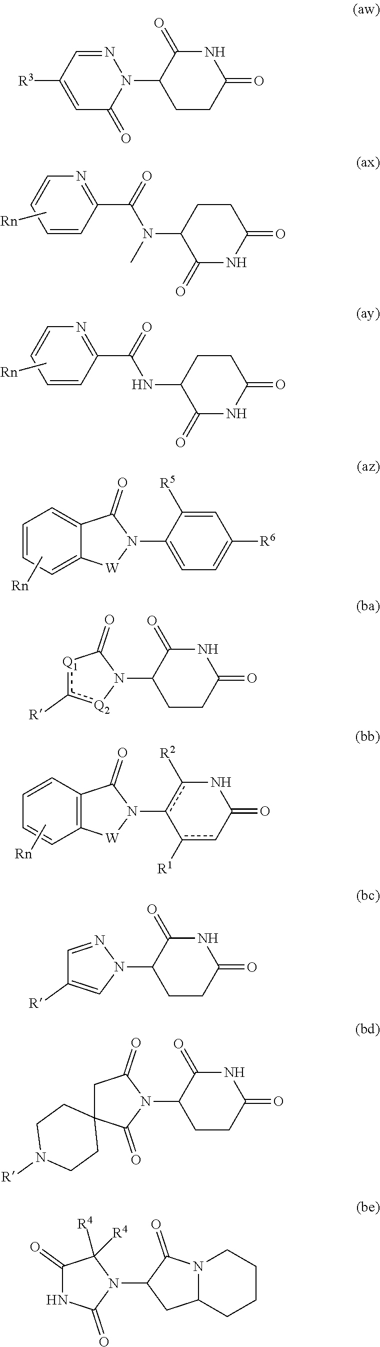

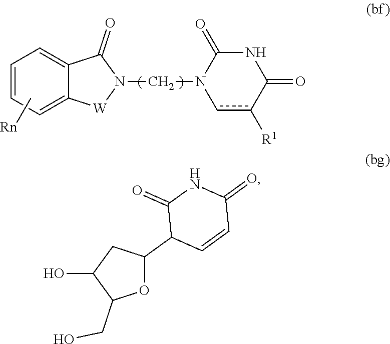

wherein:
W is independently selected from the group CH₂, CHR, C═O, SO₂, NH, and N-alkyl;
R¹ is selected from the group absent, H, CH, CN, C1-C3 alkyl;
R² is H or a C1-C3 alkyl;
R³ is selected from H, alkyl, substituted alkyl, alkoxy, substituted alkoxy;
R⁴ is methyl or ethyl;
R⁵ is H or halo;
R⁶ is H or halo;
R of the CLM is H;
R′ is H or an attachment point for a PTM, a chemical linker group (L), a ULM, a CLM,
Q₁ and Q₂ are each independently C or N substituted with a group independently selected from H or C1-C3 alkyl;
[symbol] is a single or double bond; and
R comprises a functional group or an atom, and n is an integer from 0-4.

In any of the embodiments described herein, the W, R¹, R², Q₁, Q₂, Q₃, Q₄, and R can independently be covalently coupled to a linker and/or a linker to which is attached one or more PTM, ULM, CLM groups.

In any of the embodiments described herein, the R¹, R², Q₁, Q₂, Q₃, Q₄, and R can independently be covalently coupled to a linker and/or a linker to which is attached one or more PTM, ULM, CLM groups.

In any of the embodiments described herein, the Q₁, Q₂, Q₃, Q₄, and R can independently be covalently coupled to a linker and/or a linker to which is attached one or more PTM, ULM, CLM groups.

In one embodiment described herein, R is modified to be covalently joined to the linker group (L), a PTM, a ULM, a second linker, or any multiple or combination thereof.

In one embodiment described herein, the CLM is selected from:

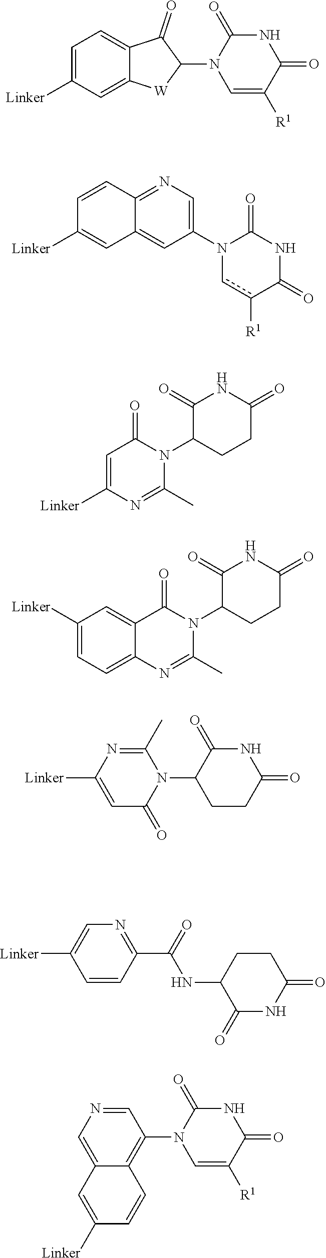

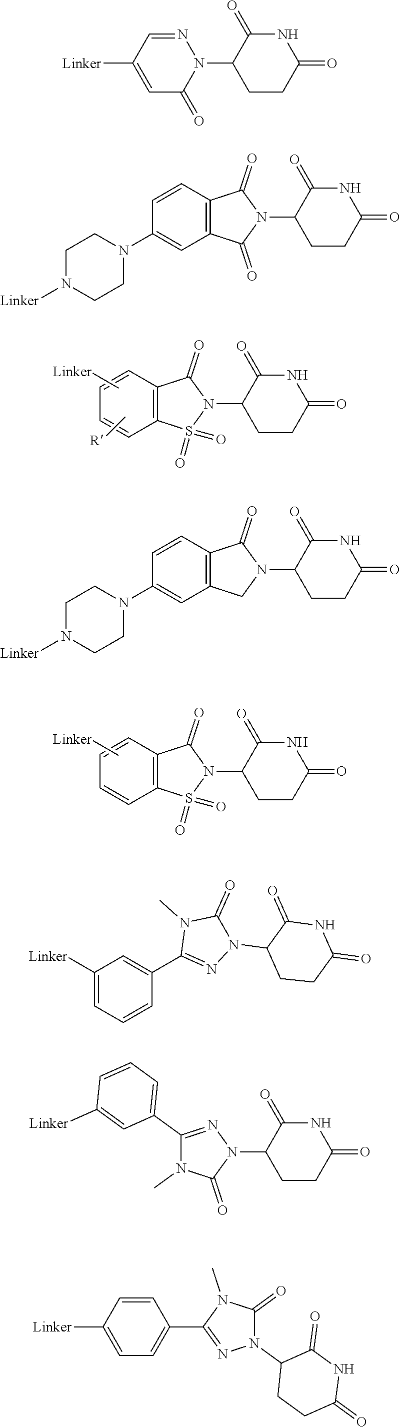

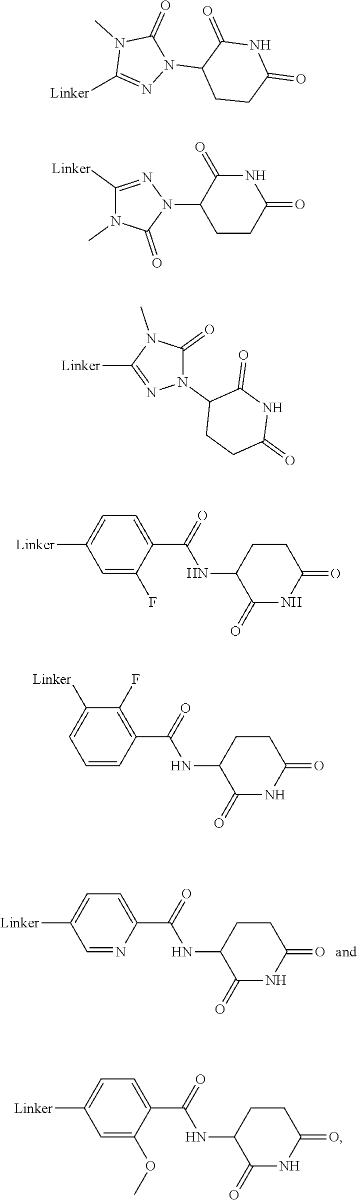

wherein R′ is a halogen and R¹ is as described in one embodiment described herein.

In certain cases, “CLM” can be an imide that binds to cereblon E3 ligase. These imides and linker attachment point can be, but not be limited to one of the following structures:

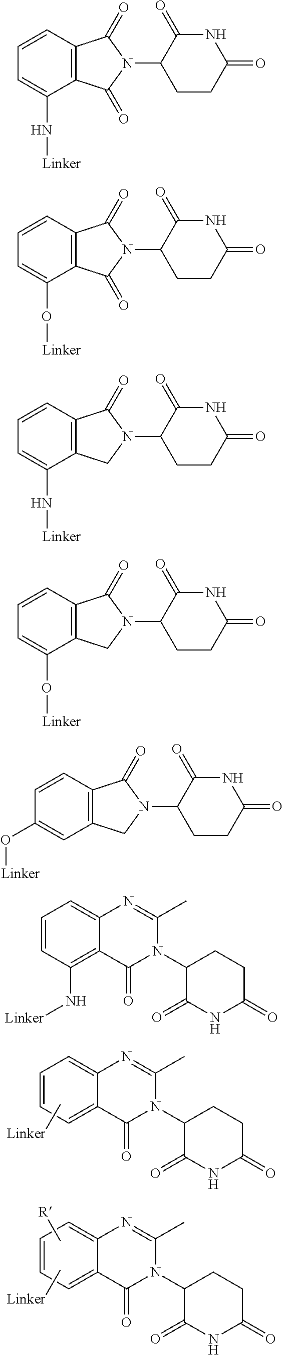

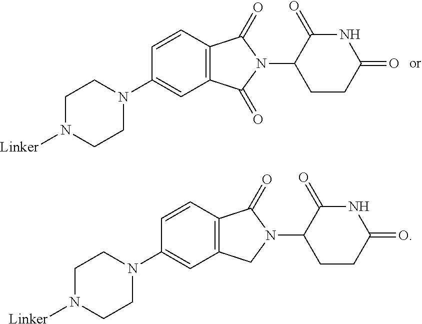

In one embodiment described herein, the ULM is selected from the group consisting of:

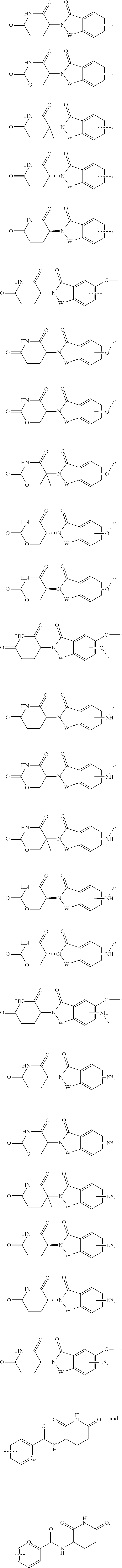

wherein:
[symbol] of the ULM indicates the point of attachment with a linker group or a PTM; and
N* is a nitrogen atom that is shared with the chemical linker group or PTM.

### Exemplary Linkers

In certain embodiments, the compounds as described herein include a PTM chemically linked to a ULM (e.g., CLM) via a chemical linker (L). In certain embodiments, the linker group L comprises one or more covalently connected structural units (e.g., -A^{L}_{1 . . .} (A^{L})_{q}- or -(A^{L})_{q}-), wherein A^{L} is a group coupled to PTM, and (A^{L})_{q} is a group coupled to ULM.

In one embodiment described herein, the linker (L) to a ULM (e.g., CLM) connection is a stable L-ULM connection. For example, in one embodiment described herein, when a linker (L) and a ULM are connected via a heteroatom (e.g., N, O, S), any additional heteroatom, if present, is separated by at least a carbon atom (e.g., —CH₂—), such as with an acetal or aminal group. By way of further example, in one embodiment described herein, when a linker (L) and a ULM are connected via a heteroatom, the heteroatom is not part of an ester.

In one embodiment described herein, the linker group L is a bond or a chemical linker group represented by the formula -(A^{L})_{q}-, wherein A is a chemical moiety and q is an integer from 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80), and wherein L is covalently bound to both the PTM and the ULM, and provides for binding of the PTM to the protein target and the ULM to an E3 ubiquitin ligase to effectuate target protein ubiquitination.

In one embodiment described herein, the linker group L is a bond or a chemical linker group represented by the formula -(A^{L})_{q}-, wherein A is a chemical moiety and q is an integer from 6-30 (e.g., 1, 2, 3, 4, 5, 6, 7, 8, 9, 10, 11, 12, 13, 14, 15, 16, 17, 18, 19, 20, 21, 22, 23, 24, or 25), and wherein L is covalently bound to both the PTM and the ULM, and provides for binding of the PTM to the protein target and the ULM to an E3 ubiquitin ligase in sufficient proximity to result in target protein ubiquitination.

In one embodiment described herein, the linker group L is -(A^{L})_{q}-, wherein:
(A^{L})_{q} is a group which connects a ULM (e.g., CLM), to PTM (TTM);
q of the linker is an integer greater than or equal to 1;
each A^{L} is independently selected from the group consisting of, a bond, CR^{L1}R^{L2}, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L1} and/or R^{L2} groups, C₃₋₁₁heterocyclyl optionally substituted with 0-6 R^{L1} and/or R^{L2} groups, C₅₋₁₃ spiroheterocyclyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂.

In certain embodiments, q is an integer greater than or equal to 1.

In one embodiment described herein, e.g., where q of the linker is greater than 2, (A^{L})_{q} is a group which is A^{L1} and (A^{L})_{q} wherein the linker couples a PTM to a ULM.

In one embodiment described herein, e.g., where q of the linker is 2, A^{L}₂ is a group which is connected to A^{L}₁ and to a ULM.

In one embodiment described herein, e.g., where q of the linker is 1, the structure of the linker group L is -A^{L}₁-, and A^{L}₁ is a group which connects a ULM moiety to a PTM moiety.

In one embodiment described herein, the unit A^{L} of linker (L) comprises a group represented by a general structure selected from the group consisting of:
—NR(CH₂)ₙ-(lower alkyl)-, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂—, —NR(CH₂)ₙ-(heterocycloalkyl)-, —NR(CH₂CH₂O)ₙ-(lower alkyl)-O—CH₂—, —NR(CH₂CH₂O)ₙ-(heterocycloalkyl)-O—CH₂—, —NR(CH₂CH₂O)ₙ-Aryl-O—CH₂—, —NR(CH₂CH₂O)ₙ-(heteroaryl)-O—CH₂—, —NR(CH₂CH₂O)ₙ-(cyclo alkyl)-O-(heteroaryl)-O—CH₂—, —NR(CH₂CH₂O)ₙ-(cyclo alkyl)-O-Aryl-O—CH₂—, —NR(CH₂CH₂O)ₙ-(lower alkyl)-NH-Aryl-O—CH₂—, —NR(CH₂CH₂O)ₙ-(lower alkyl)-O-Aryl-CH₂, —NR(CH₂CH₂O)ₙ-cycloalkyl-O-Aryl-, —NR(CH₂CH₂O)ₙ-cycloalkyl-O-(heteroaryl)l-, —NR(CH₂CH₂)ₙ-(cycloalkyl)-O-(heterocyclyl)-CH₂, —NR(CH₂CH₂)ₙ-(heterocyclyl)-(heterocyclyl)-CH₂, and —N(R1R2)-(heterocyclyl)-CH₂; where
n of the linker can be 0 to 10;
R of the linker can be H, or lower alkyl; and
R1 and R2 of the linker can form a ring with the connecting N.

In one embodiment described herein, the linker (L) includes an optionally substituted C₁-C₅₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkyl, and including all implied subranges, e.g., C₁-C₁₀, C₁-C₂₀; C₂-C₁₀, C₂₋₂₀; C₁₀-C₂₀, C₁₀-C₅₀ etc.), wherein each carbon is optionally independently substituted or replaced with (1) a heteroatom selected from N, O, S, P, or Si atoms that has an appropriate number of hydrogens, substitutions, or both to complete valency, (2) an optionally substituted cycloalkyl or bicyclic cycloalkly, (3) an optionally substituted heterocyloalkyl or bicyclic heterocyloalkyl, (4) an optionally substituted aryl or bicyclic aryl, or (5) optionally substituted heteroaryl or bicyclic heteroaryl. In one embodiment described herein, the linker (L) does not have heteroatom-heteroatom bonding (e.g., no heteroatoms are covalently linked or adjacently located).

In one embodiment described herein, the linker (L) includes an optionally substituted C₁-C₅₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkyl), wherein:
each carbon is optionally independently substituted or replaced with CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L1} and/or R^{L2} groups, C₃₋₁₁ heterocyclyl optionally substituted with 0-6 R^{L1} and/or R^{L2} groups, C₅₋₁₃ spiroheterocyclyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form a cycloalkyl and/or a heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂. In one embodiment described herein, the linker (L) does not have heteroatom-heteroatom bonding (e.g., no heteroatoms are covalently linked or adjacently located).

In one embodiment described herein, the unit A^{L} of the linker (L) comprises a structure selected from the group consisting of:

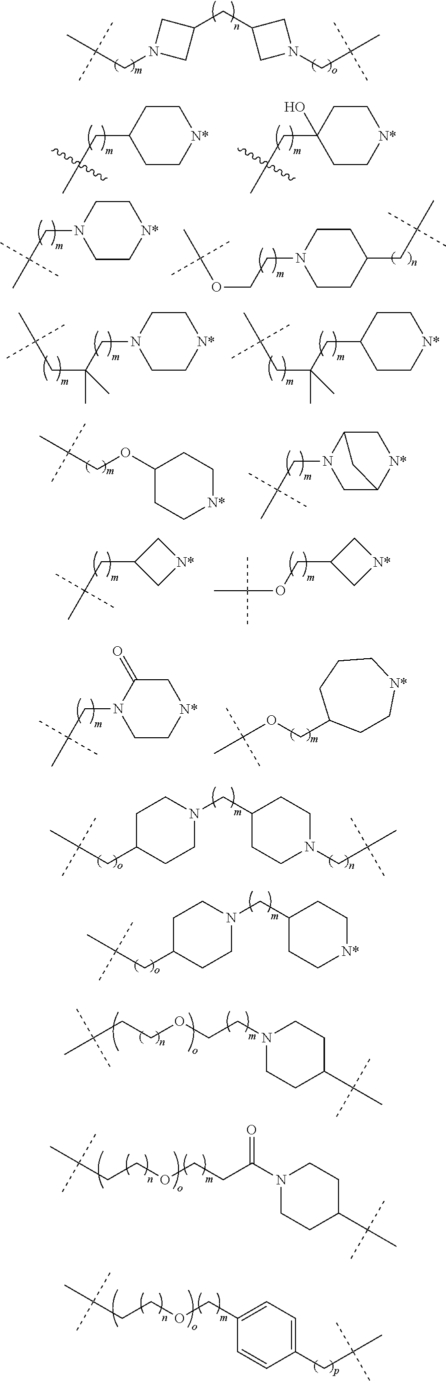

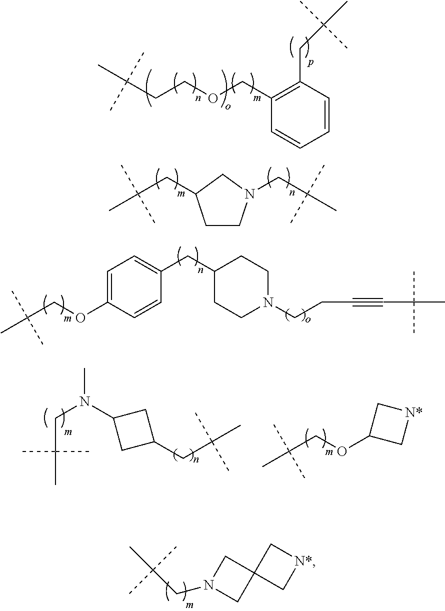

wherein:
N* is a nitrogen atom that is covalently linked to the ULM or PTM, or that is shared with the ULM or PTM; and
each m, n, o, p, q, and r is independently 0, 1, 2, 3, 4, 5, 6, 7, 8, 9, 10, 11, 12, 13, 14, 15, 16, 17, 18, 19, or 20.

In one embodiment described herein, the unit A^{L} of the linker (L) is selected from:

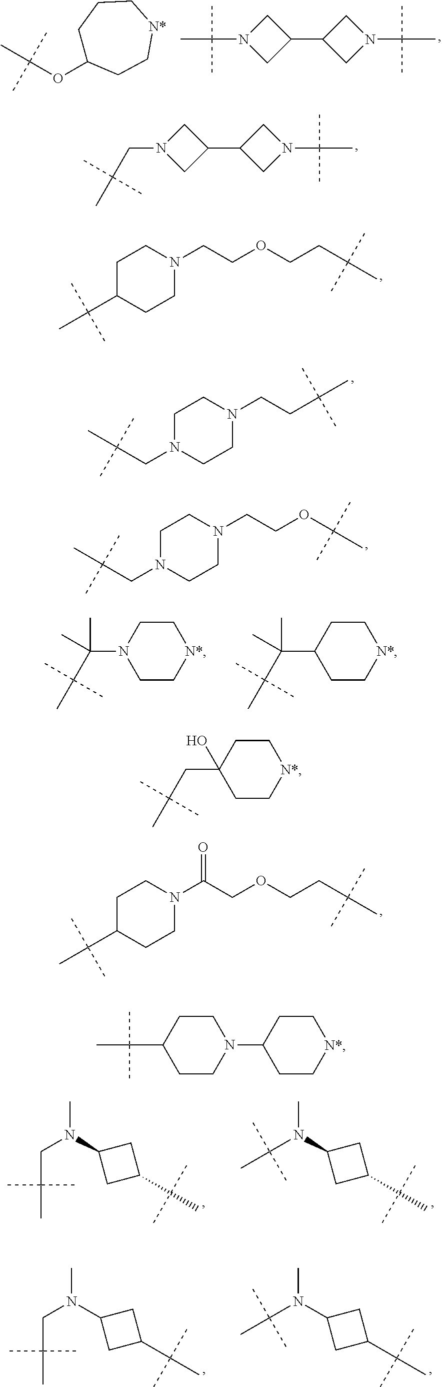

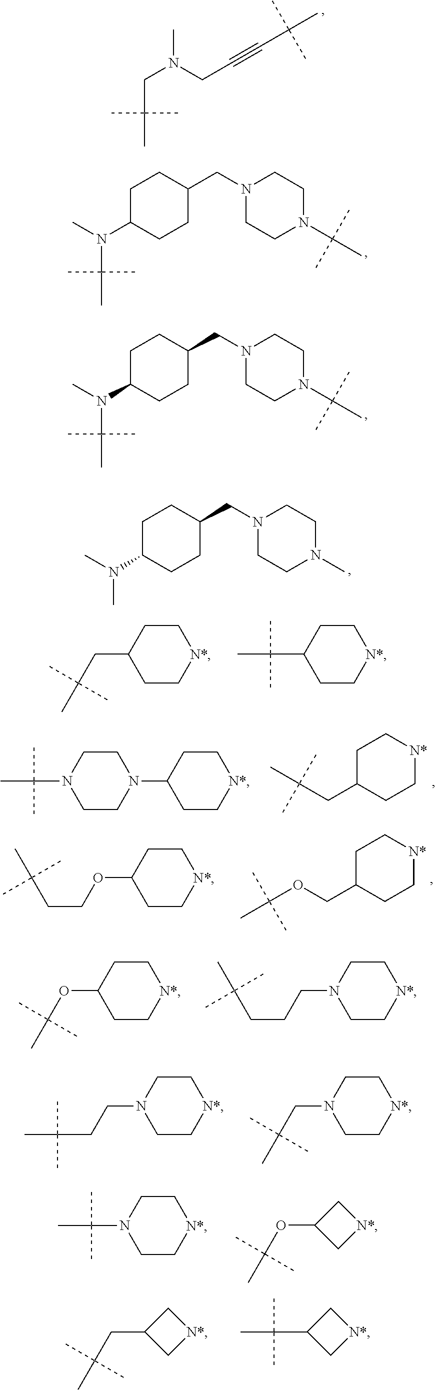

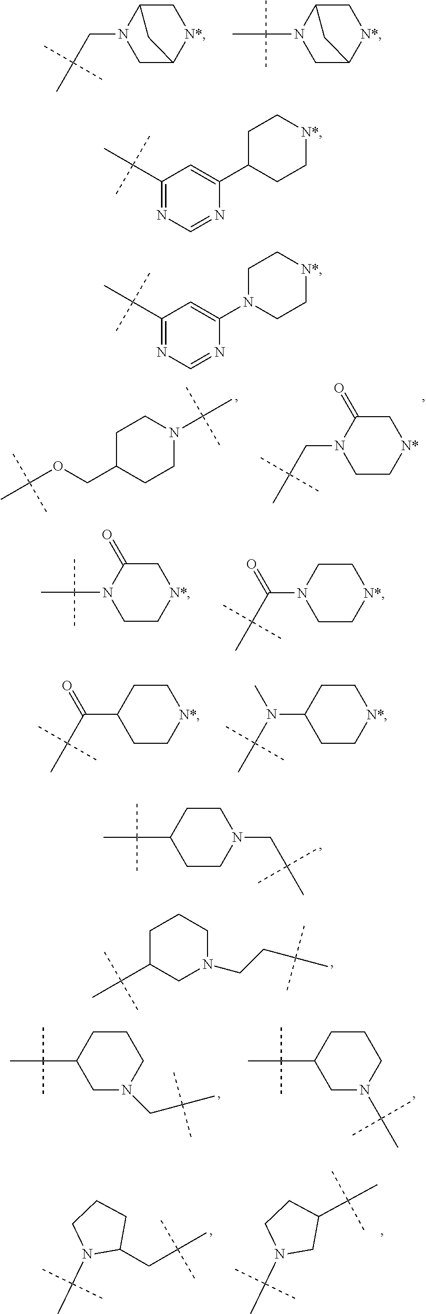

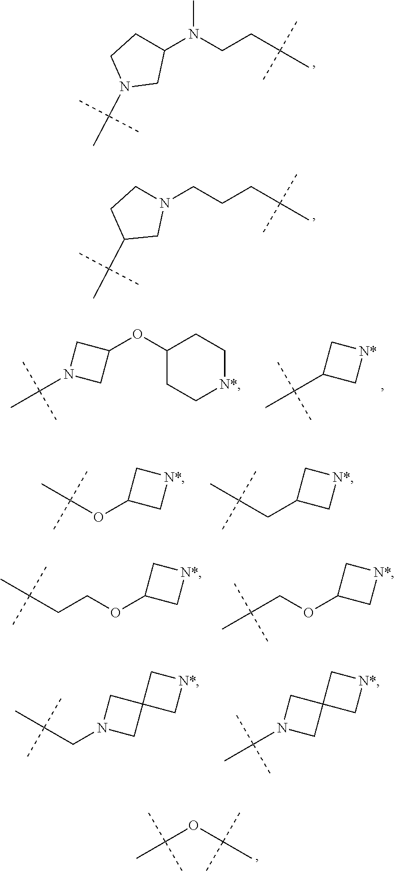

wherein N* is a nitrogen atom that is covalently linked to the ULM or PTM, or that is shared with the ULM or PTM.

In one embodiment described herein, the unit A^{L} of linker (L) comprises a group represented by a general structure selected from the group consisting of:

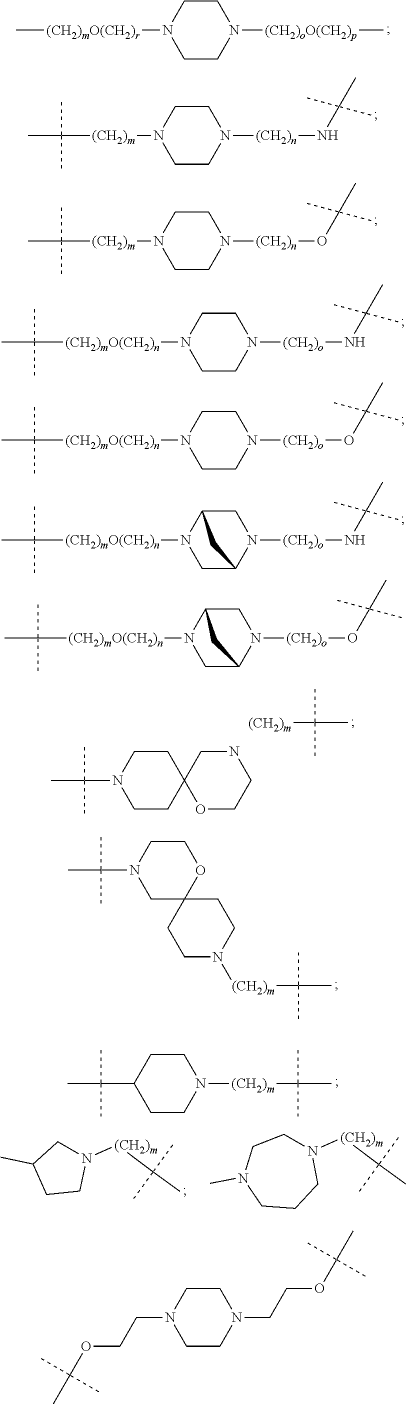

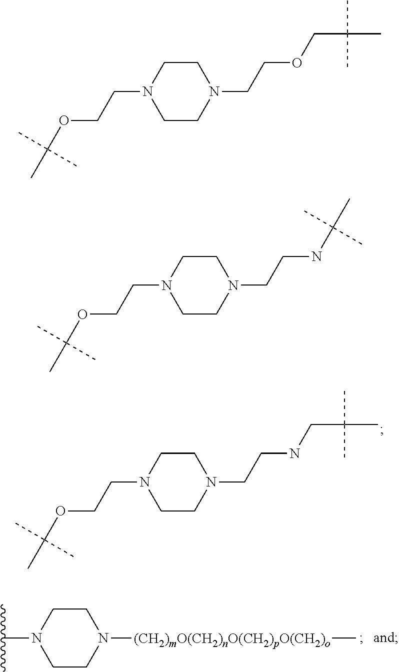

wherein
m, n, o, p, q, and r of the linker are independently 0, 1, 2, 3, 4, 5, 6, 7, 8, 9, 10, 11, 12, 13, 14, 15, 16, 17, 18, 19, or 20;
when m, n, o, p, q, and r are zero, N—O or O—O bond is absent,
X of the linker is H or F;

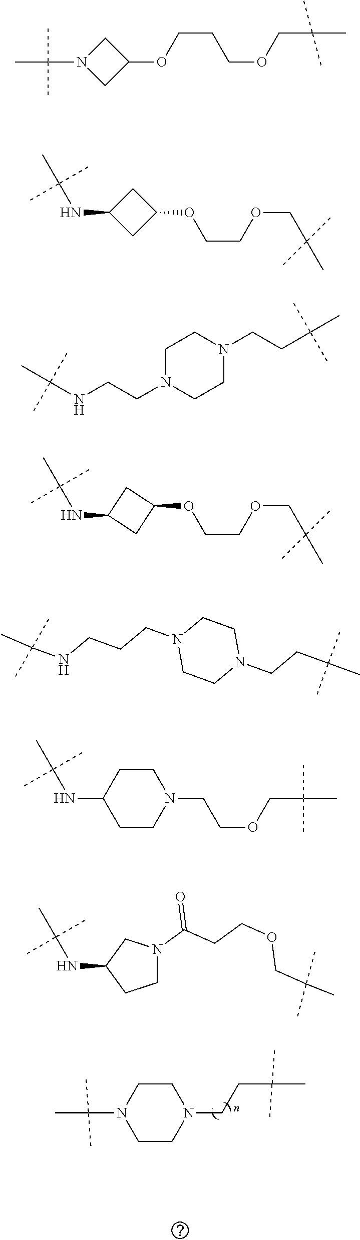

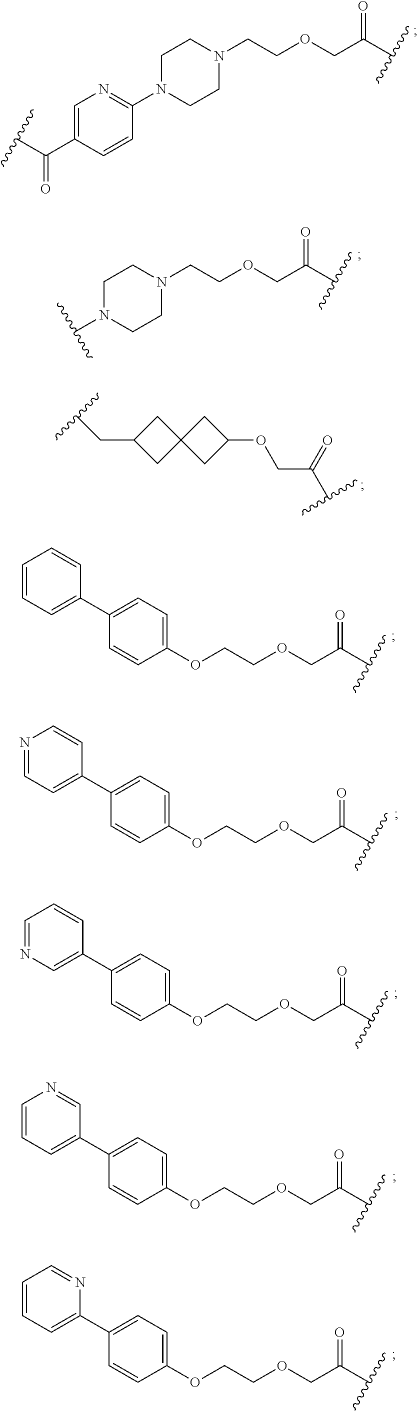

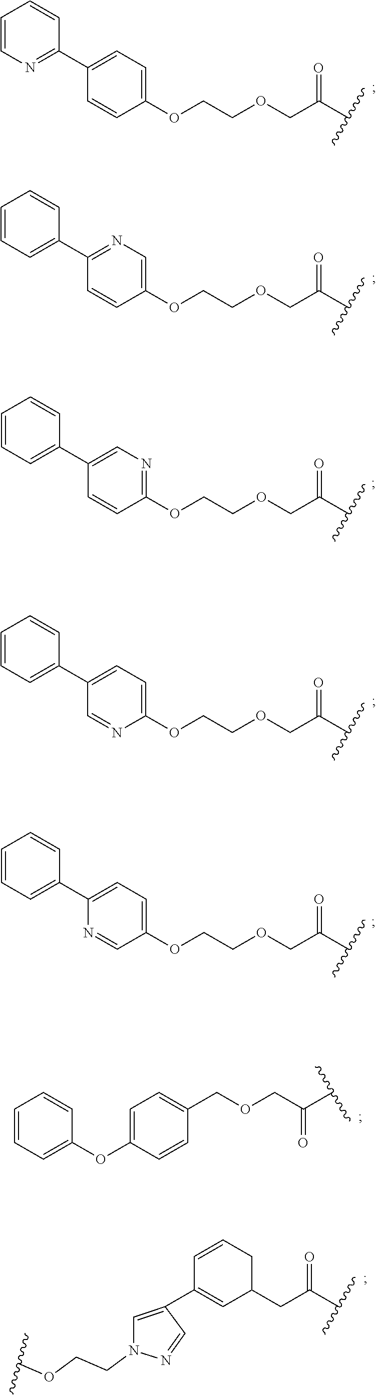

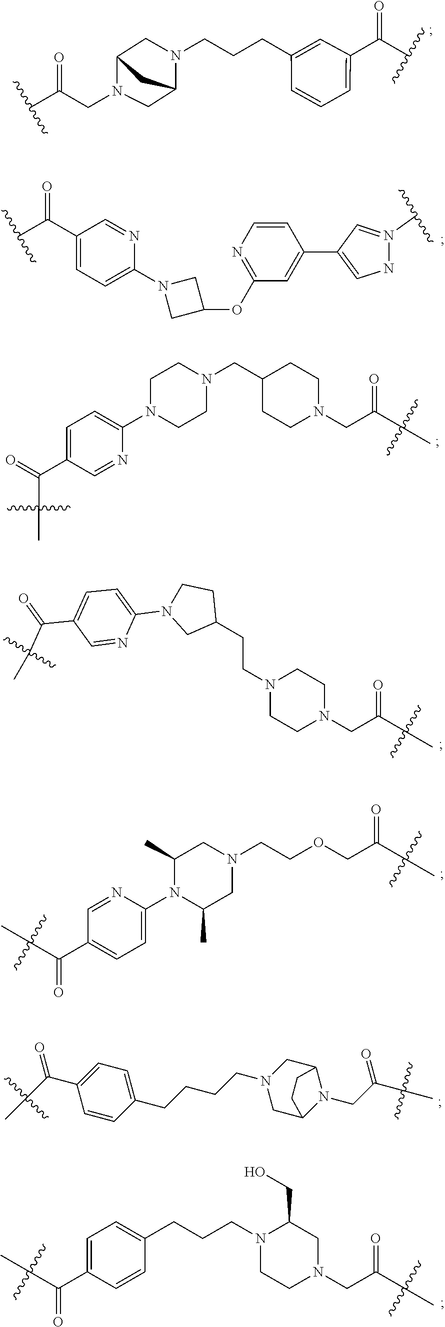

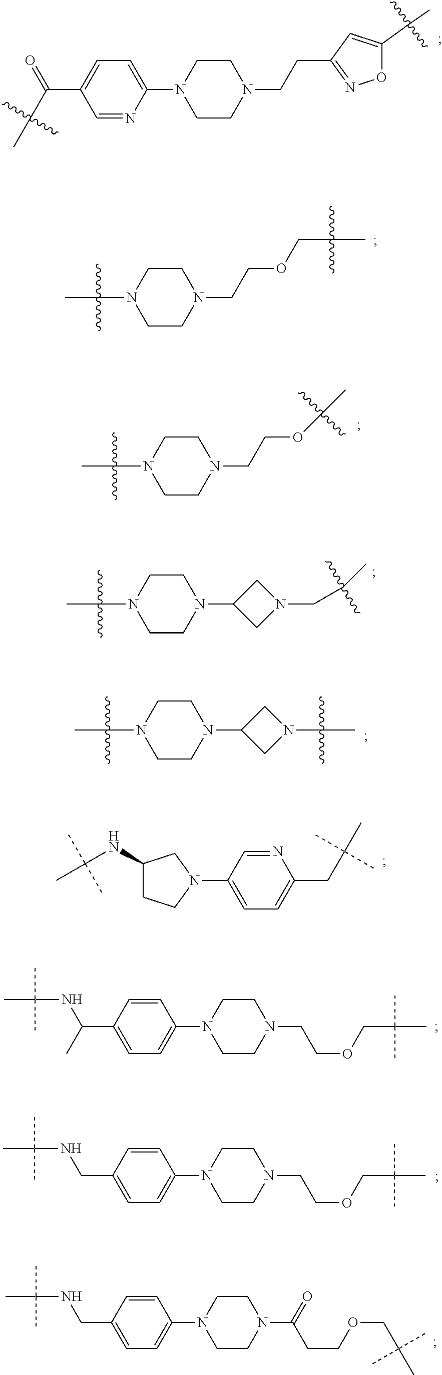

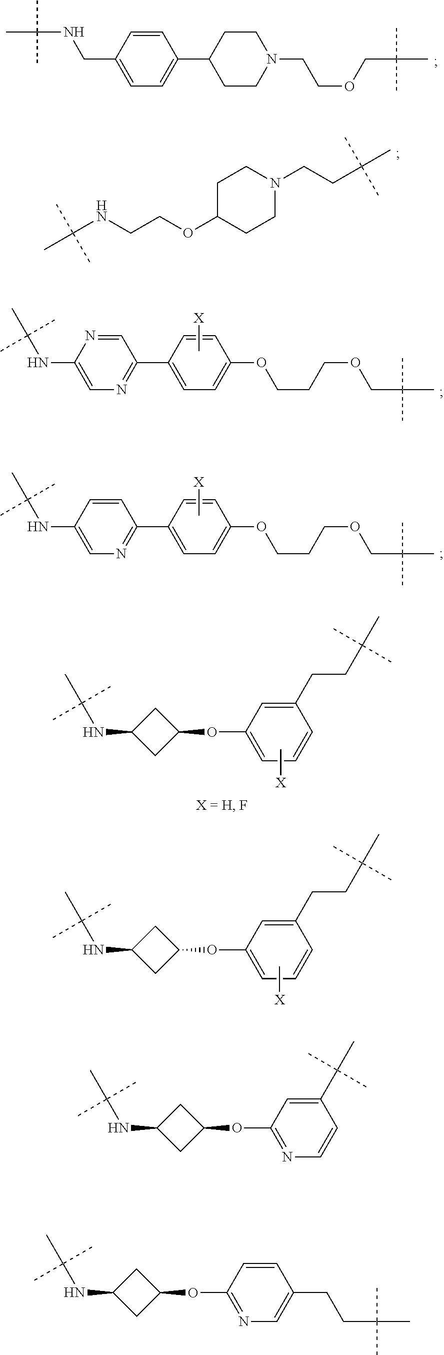

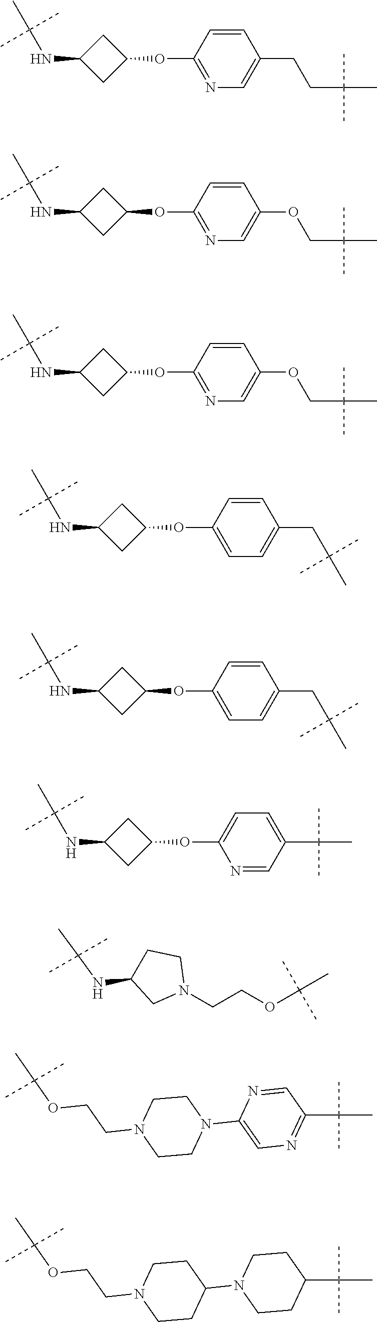

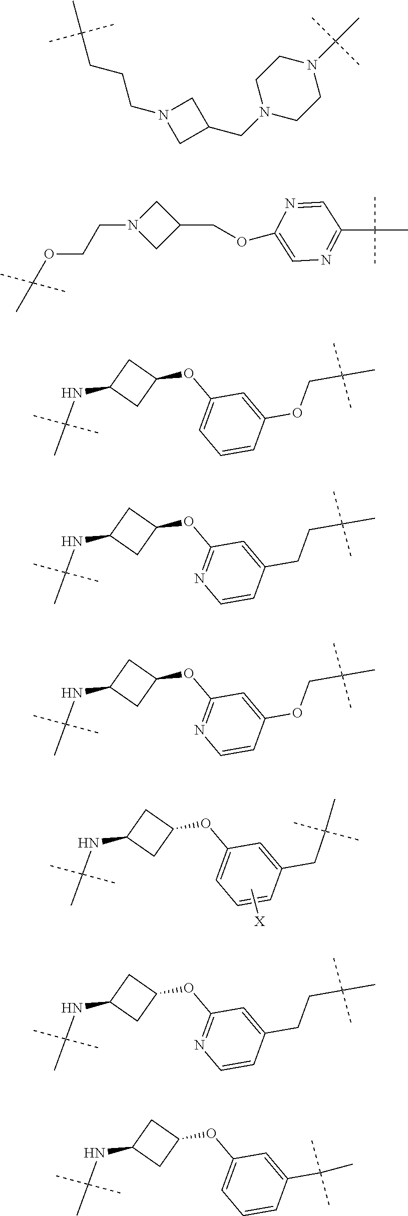

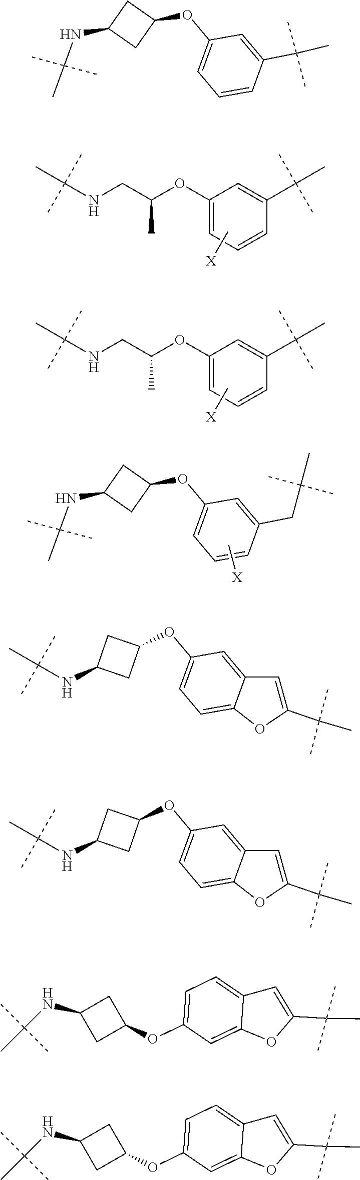

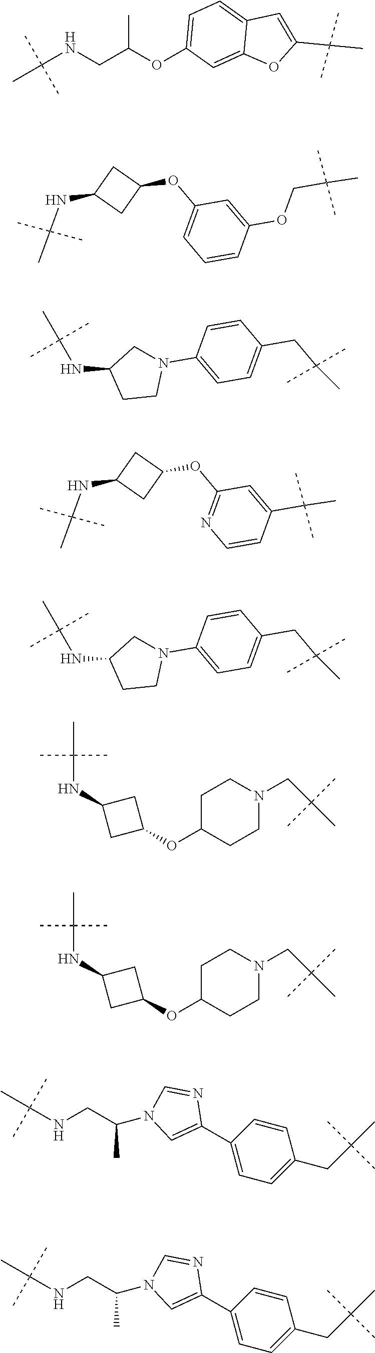

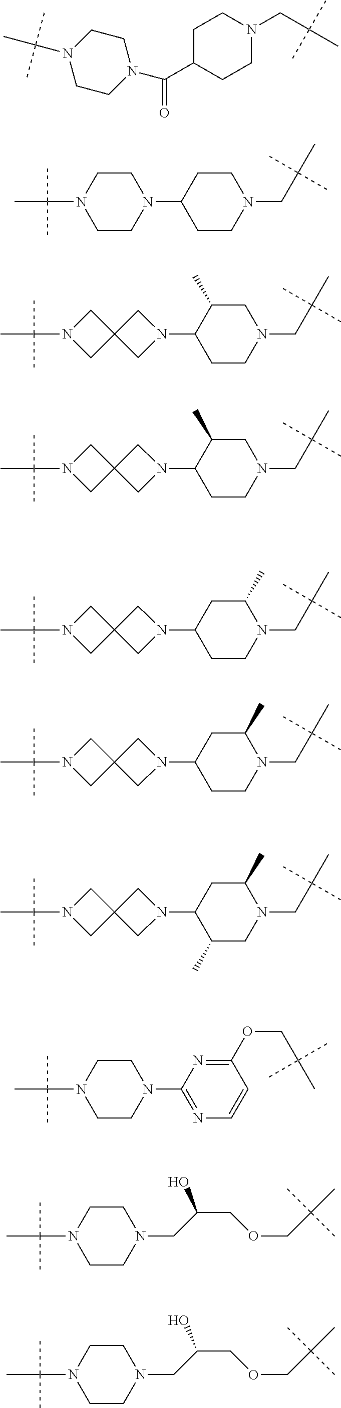

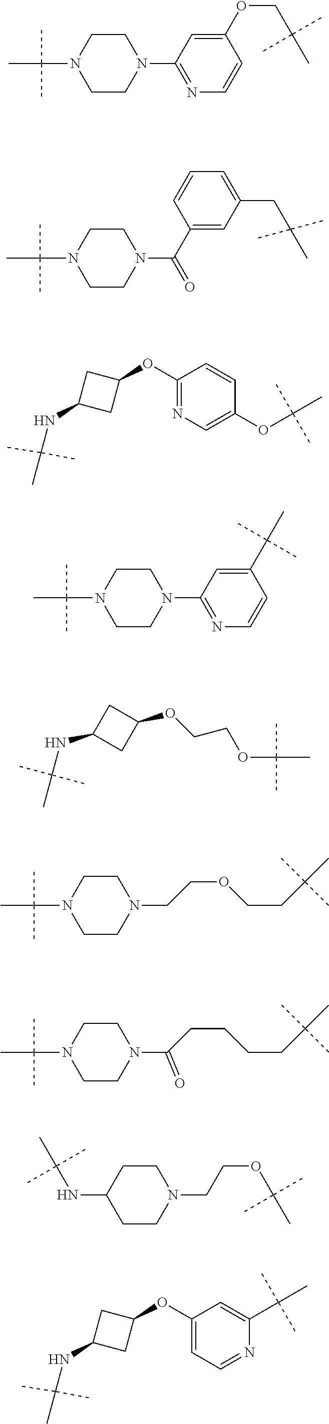

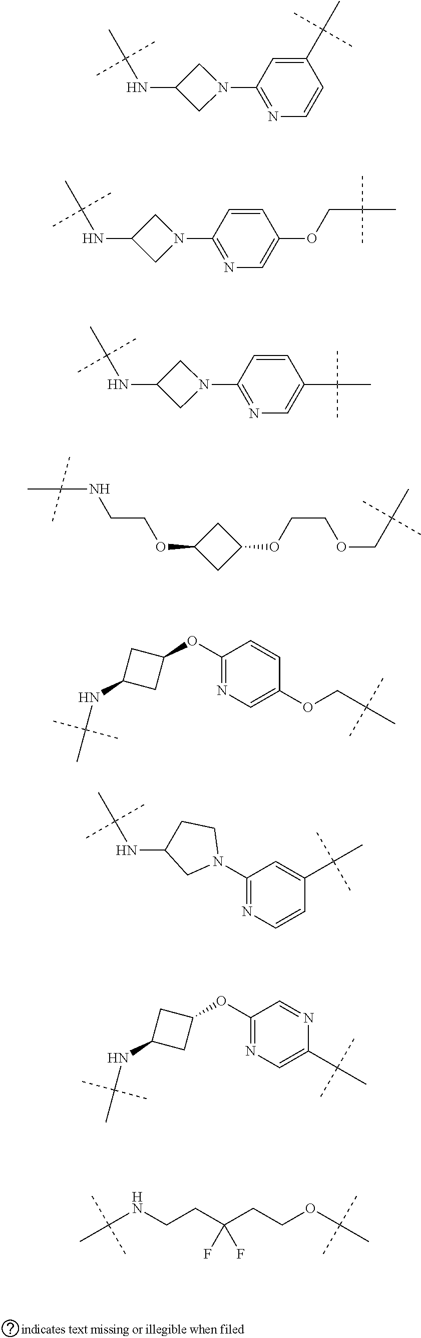

where each n and m of the linker can independently be 0, 1, 2, 3, 4, 5, or 6.

In one embodiment described herein, the unit A^{L} of linker (L) is selected from the group consisting of:

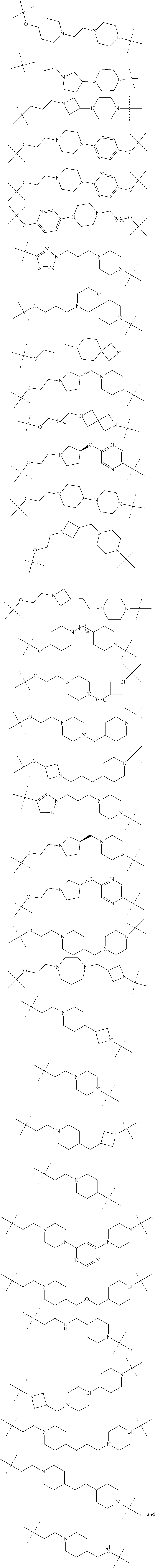

wherein each m and n is independently selected from 0, 1, 2, 3, 4, 5, or 6.

In one embodiment described herein, the unit A^{L} of linker (L) is selected from the group consisting of:

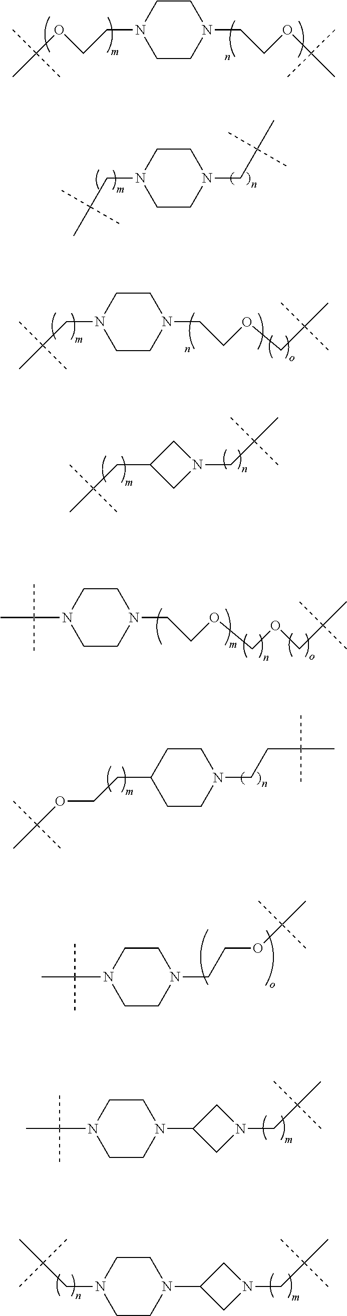

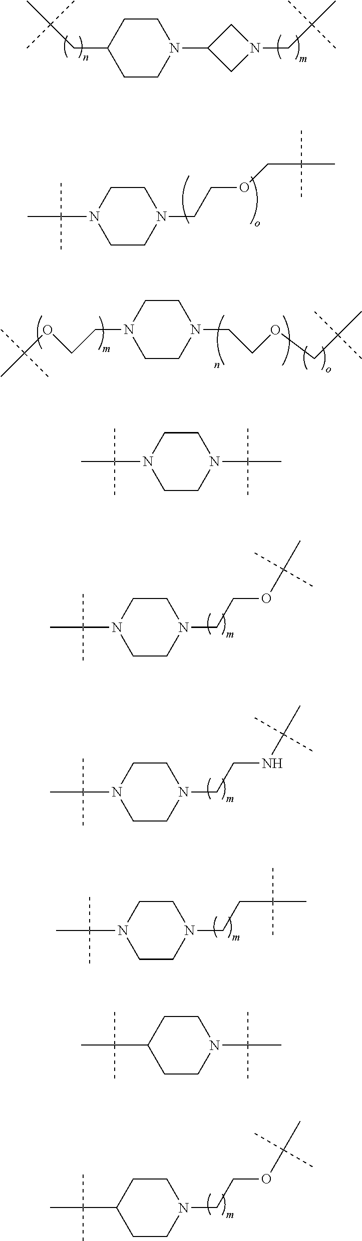

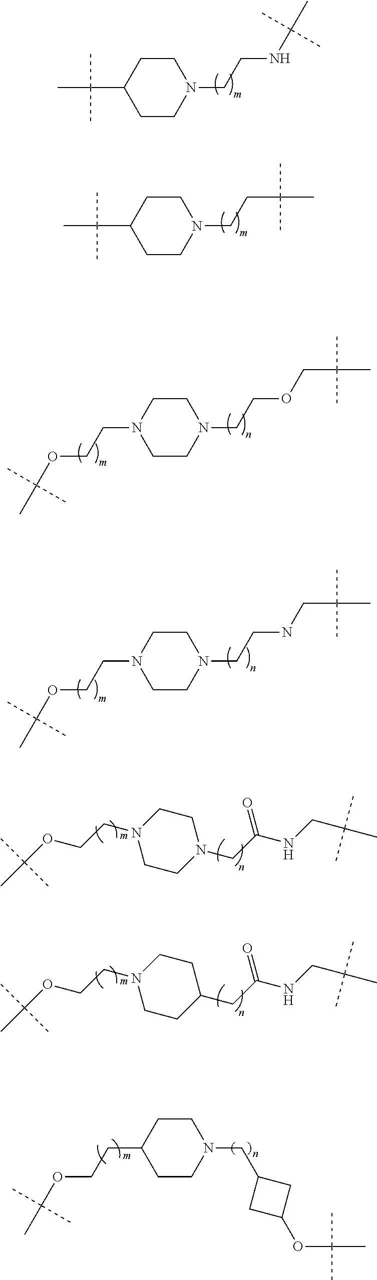

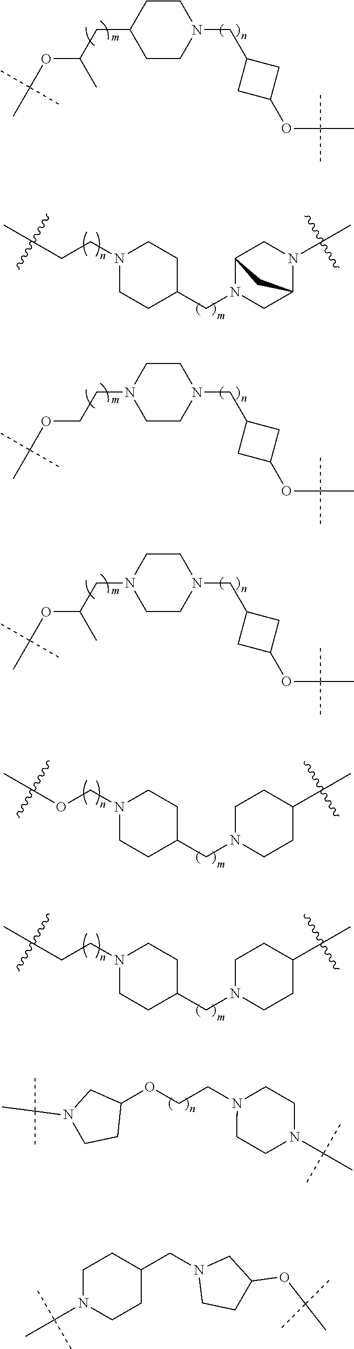

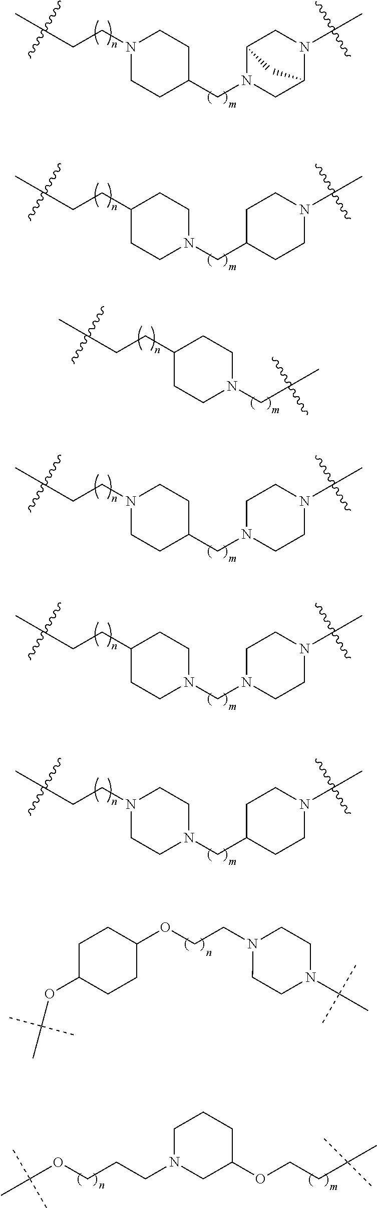

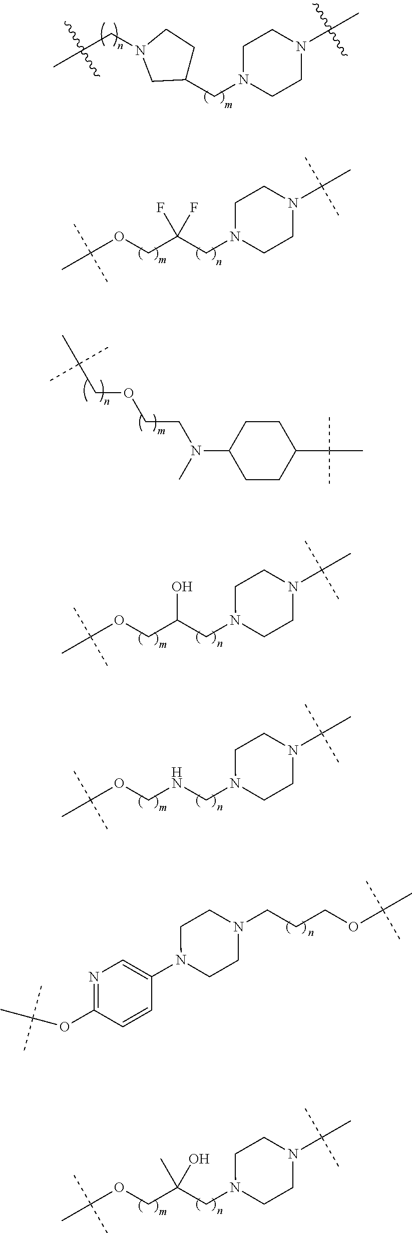

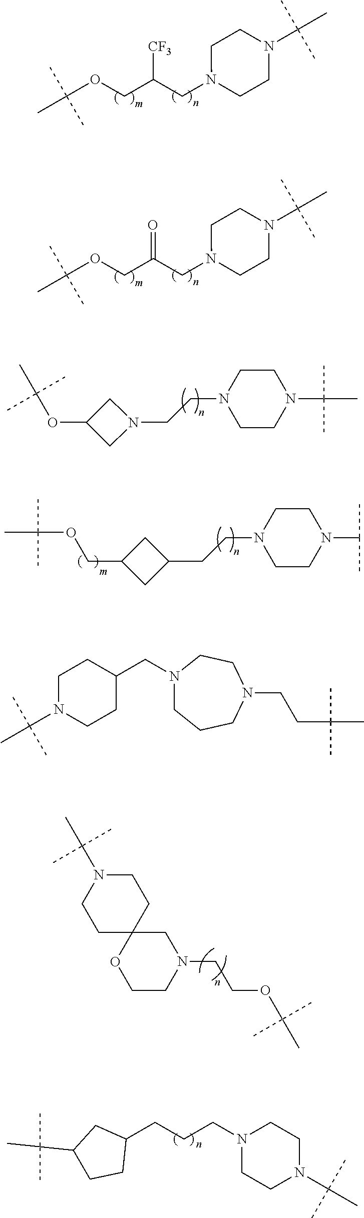

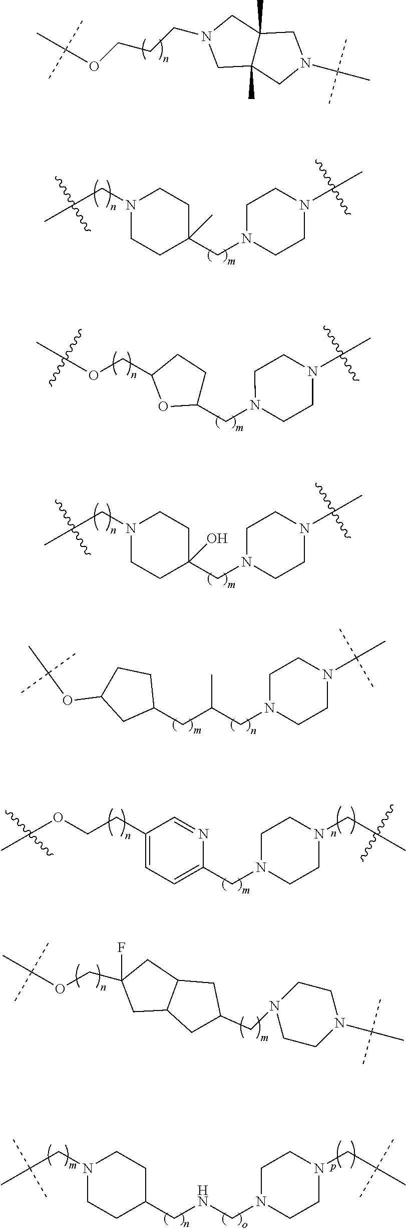

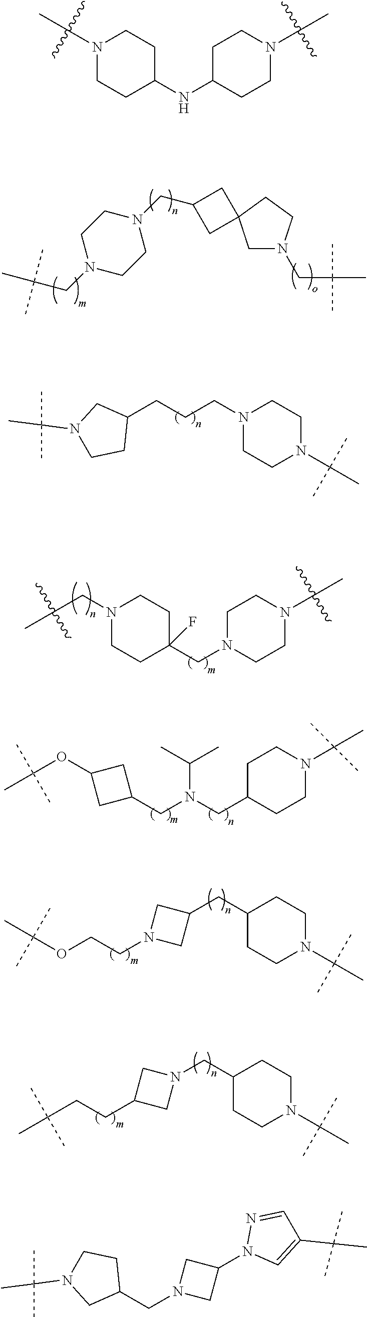

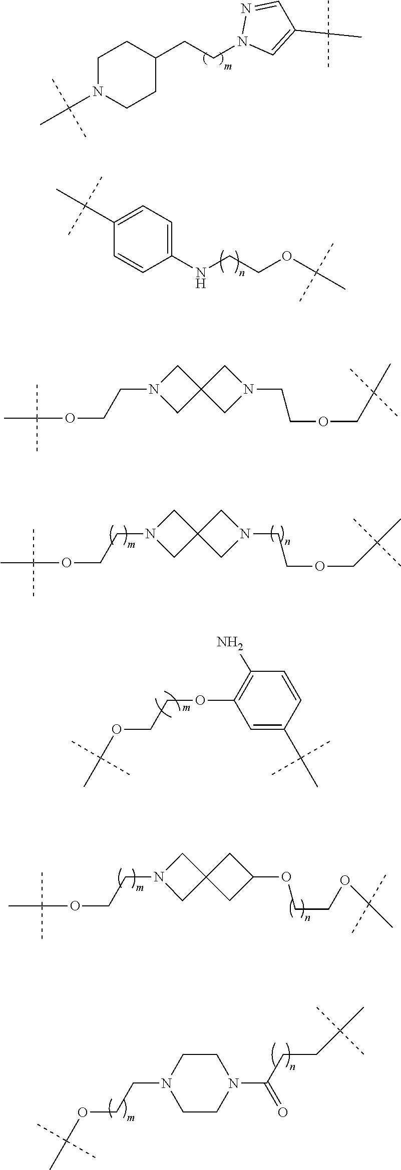

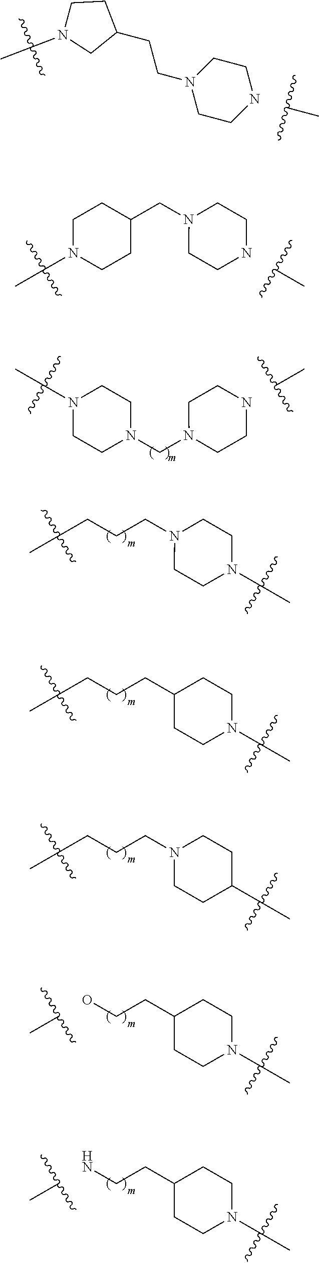

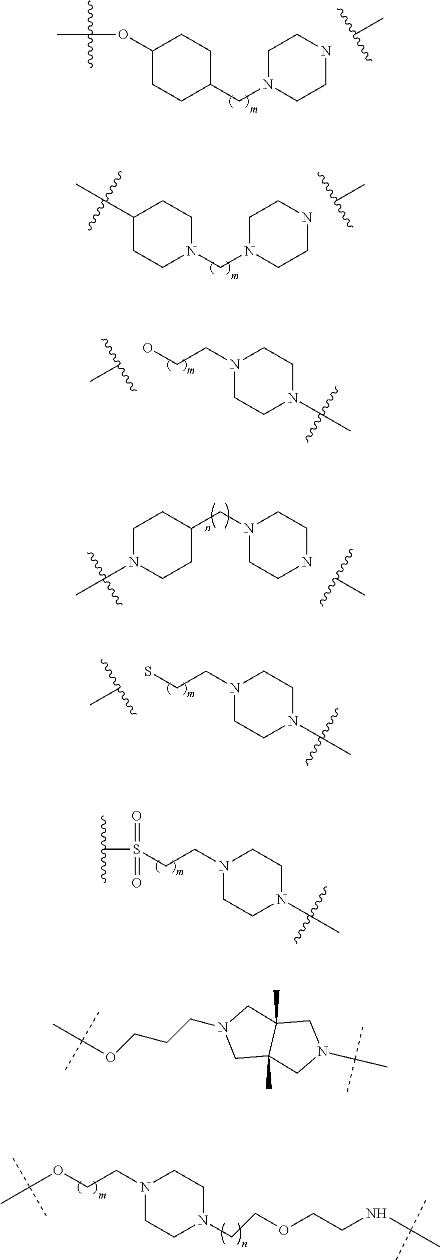

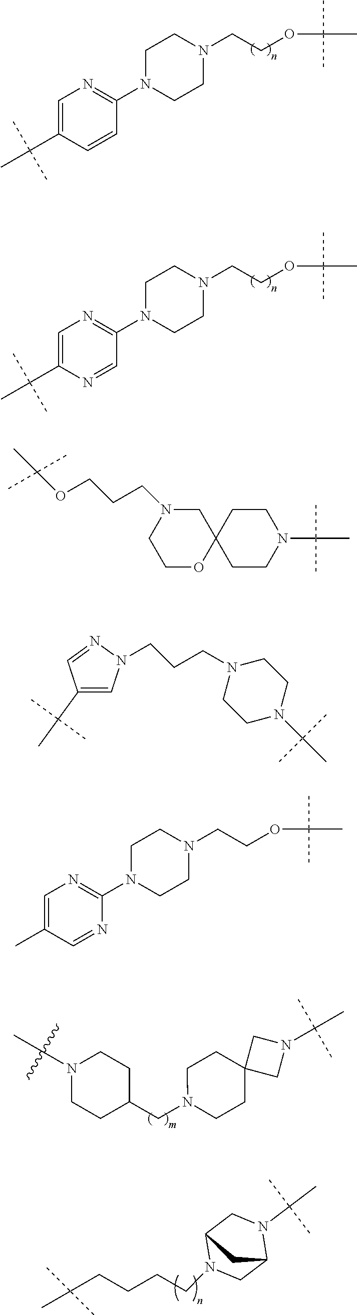

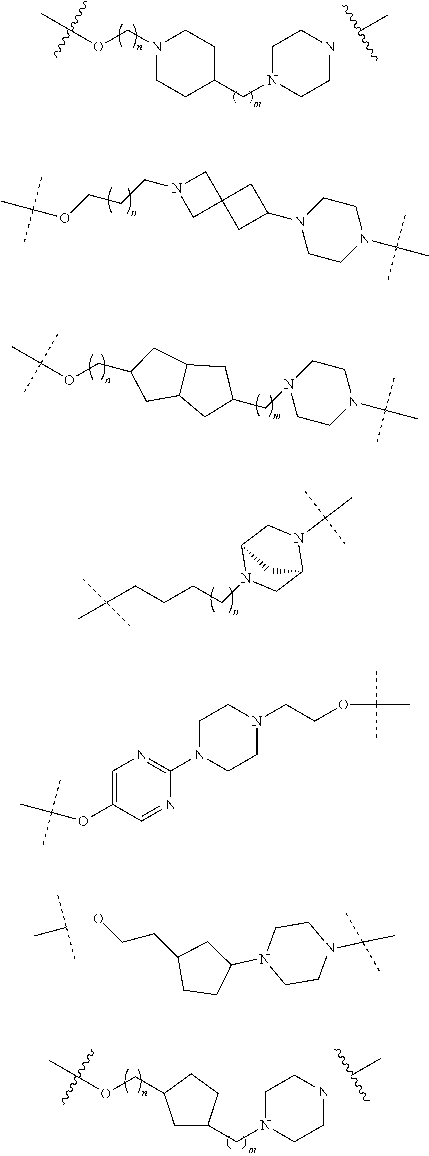

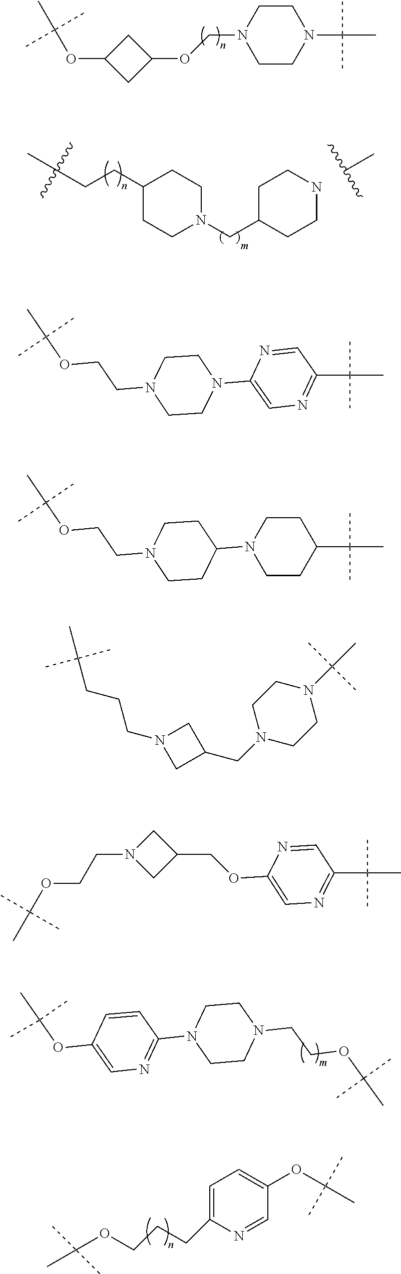

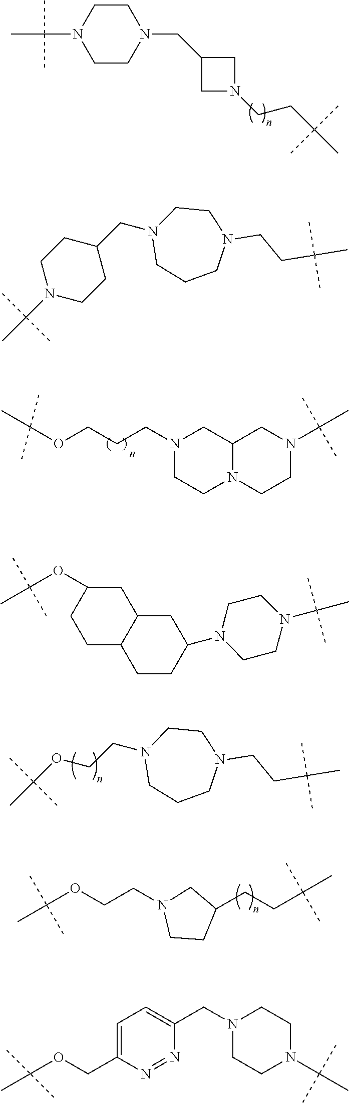

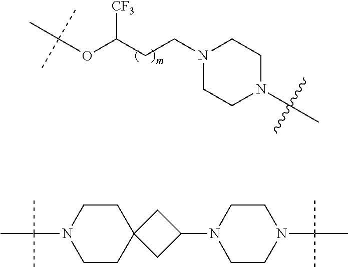

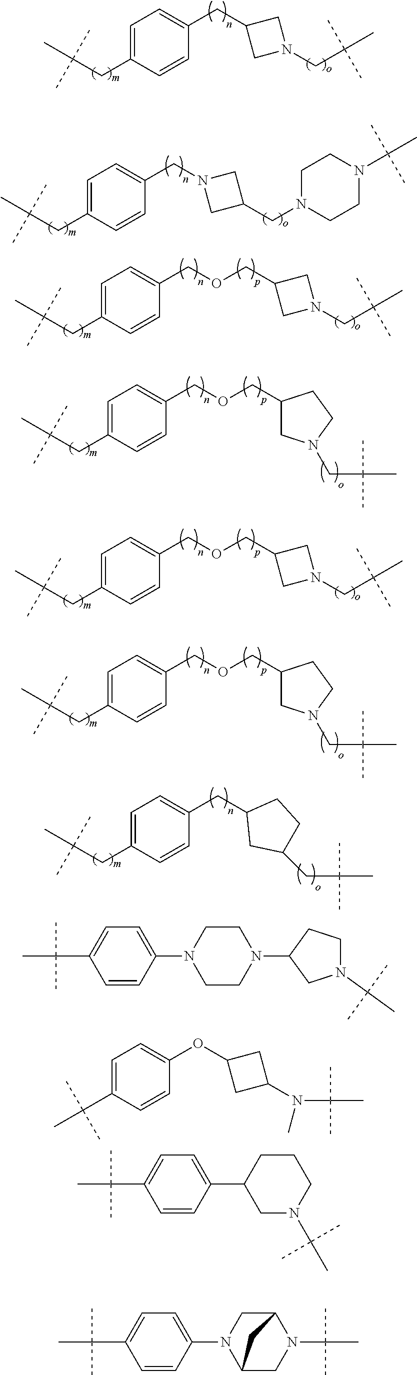

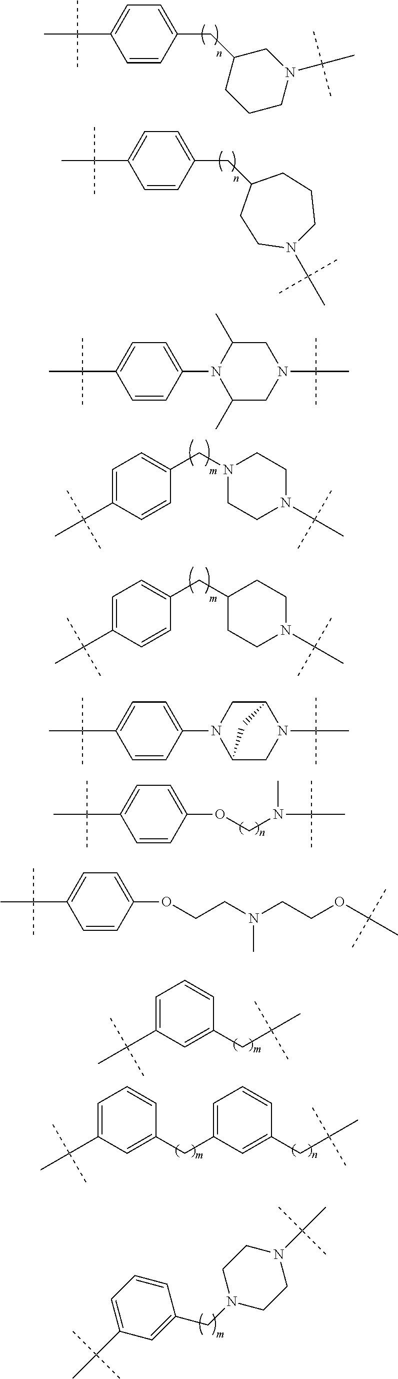

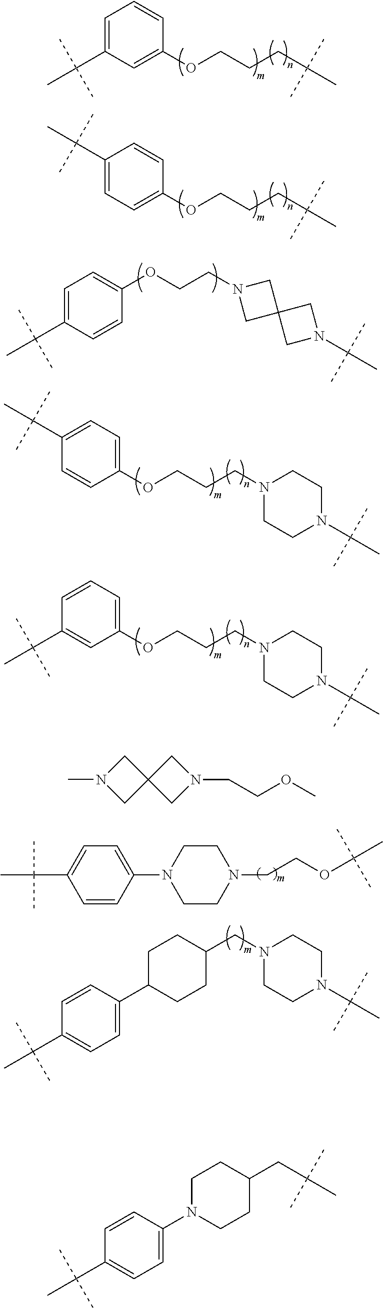

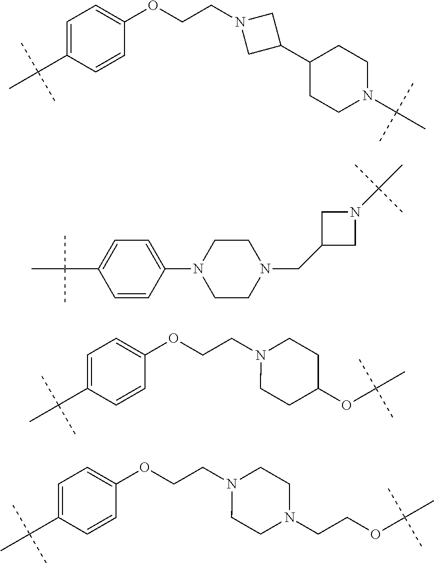

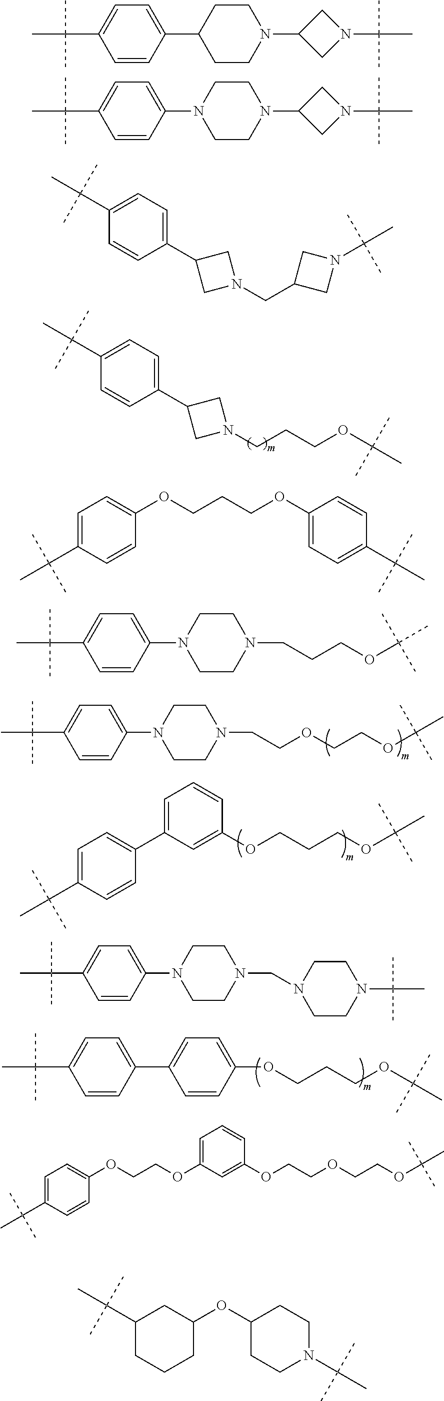

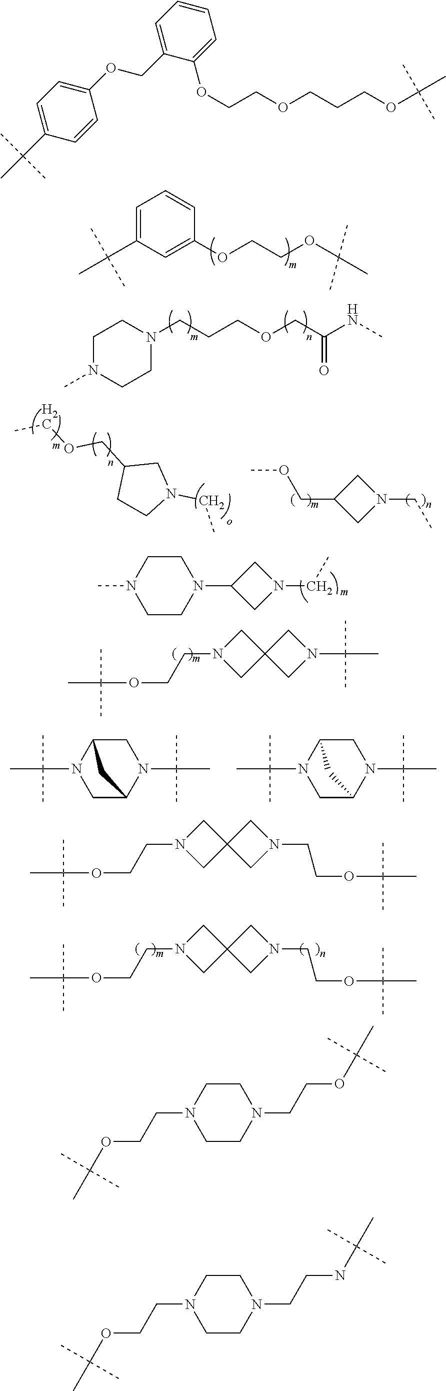

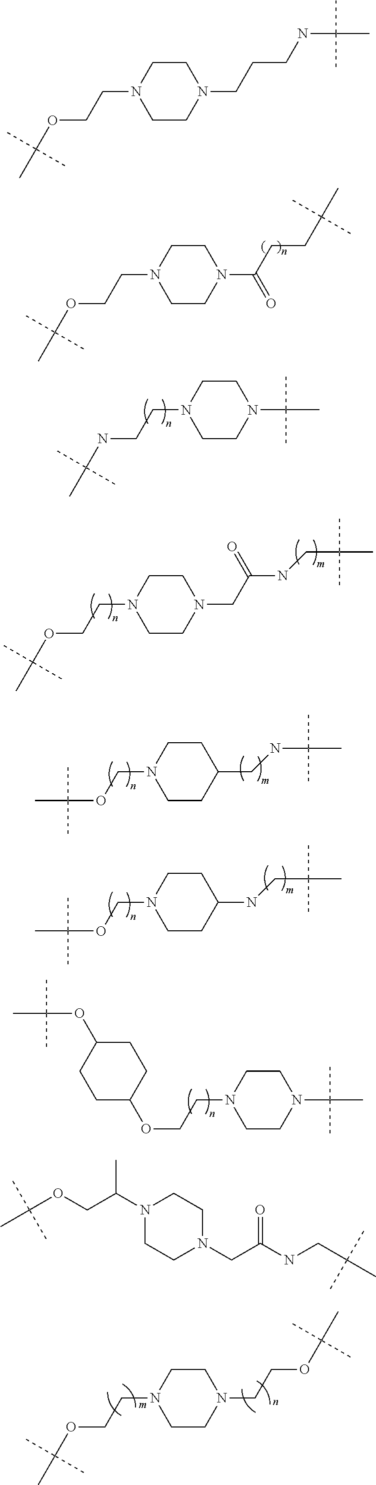

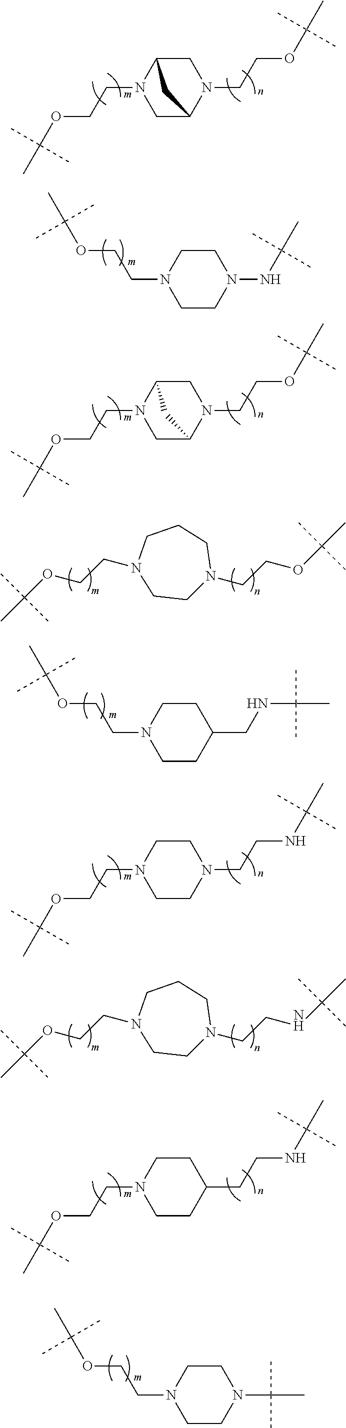

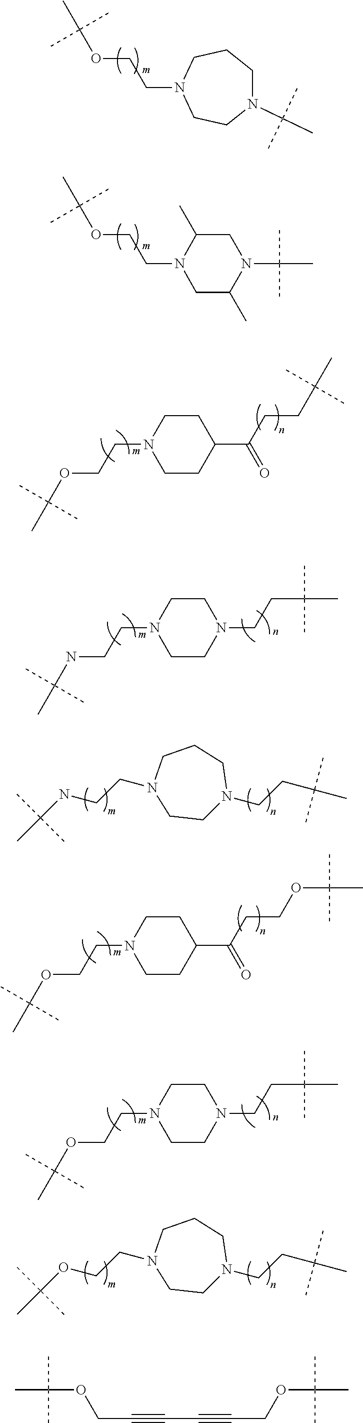

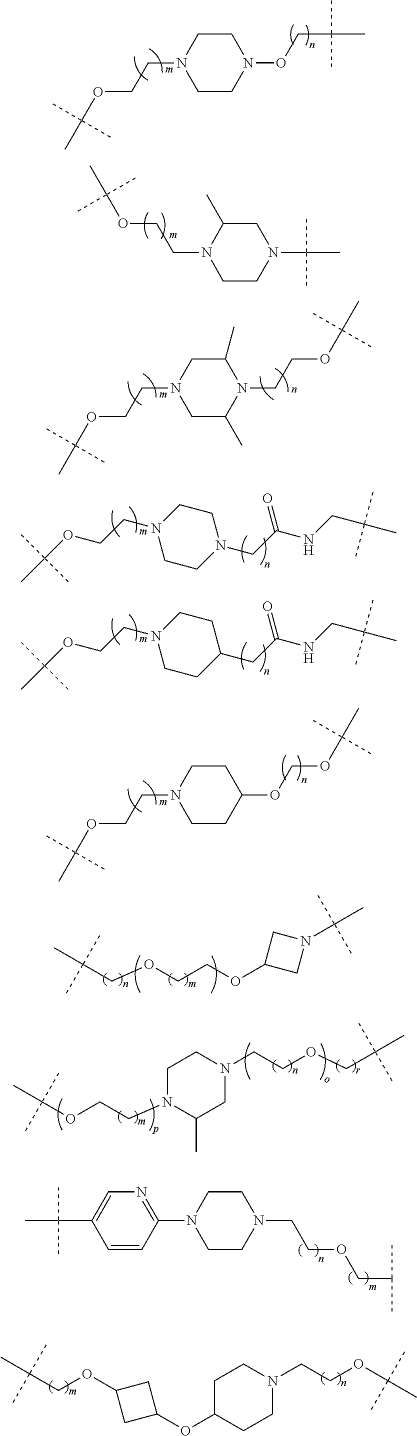

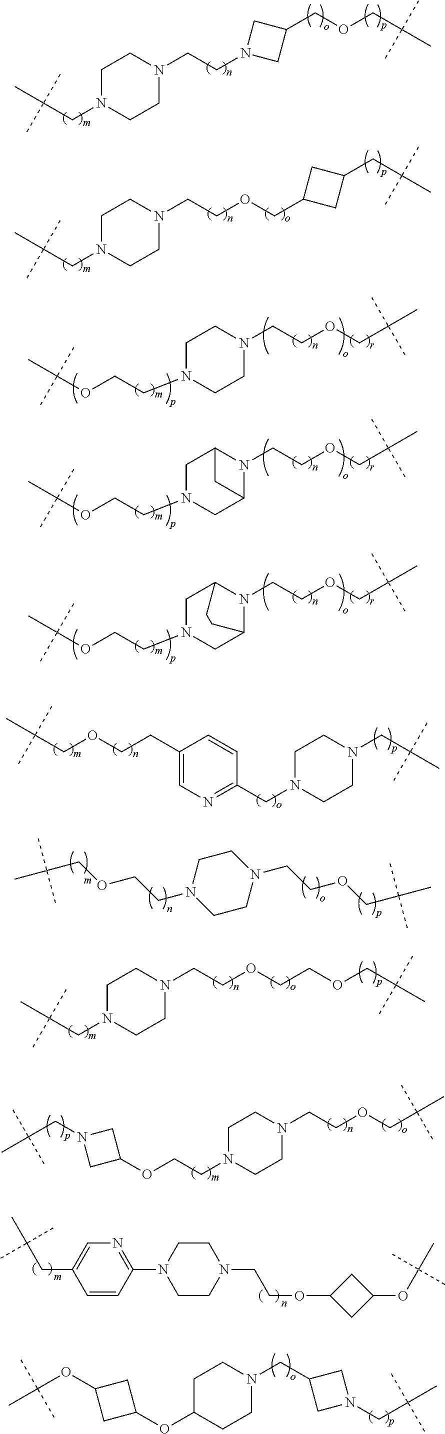

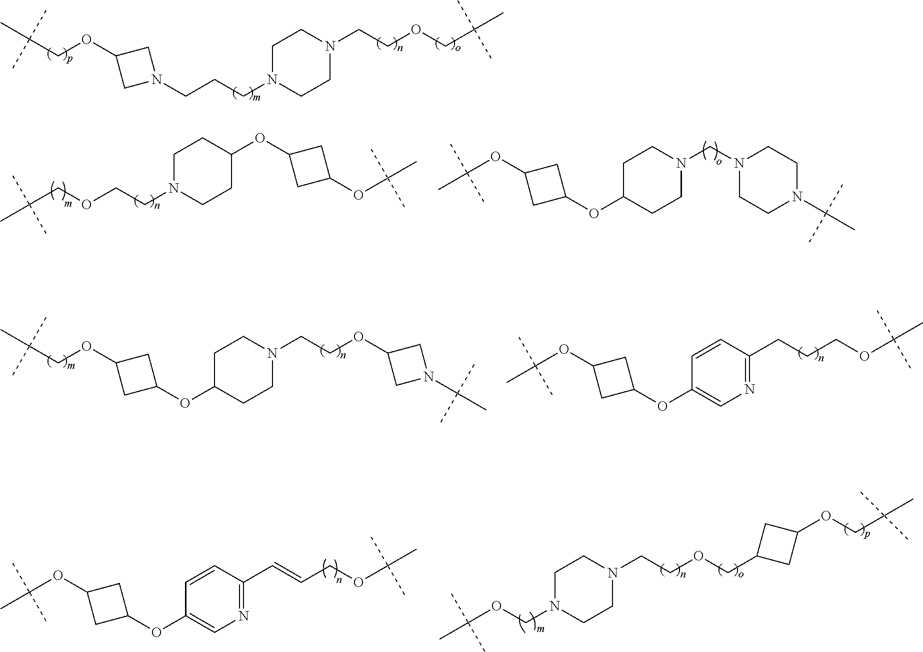

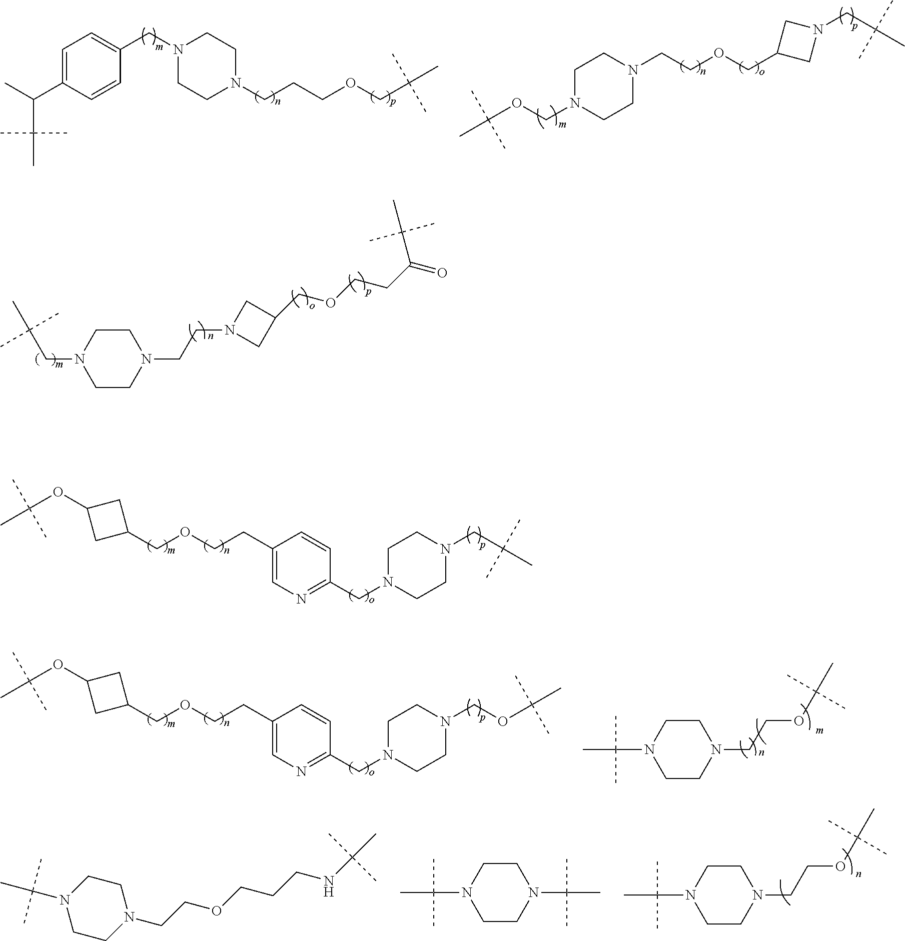

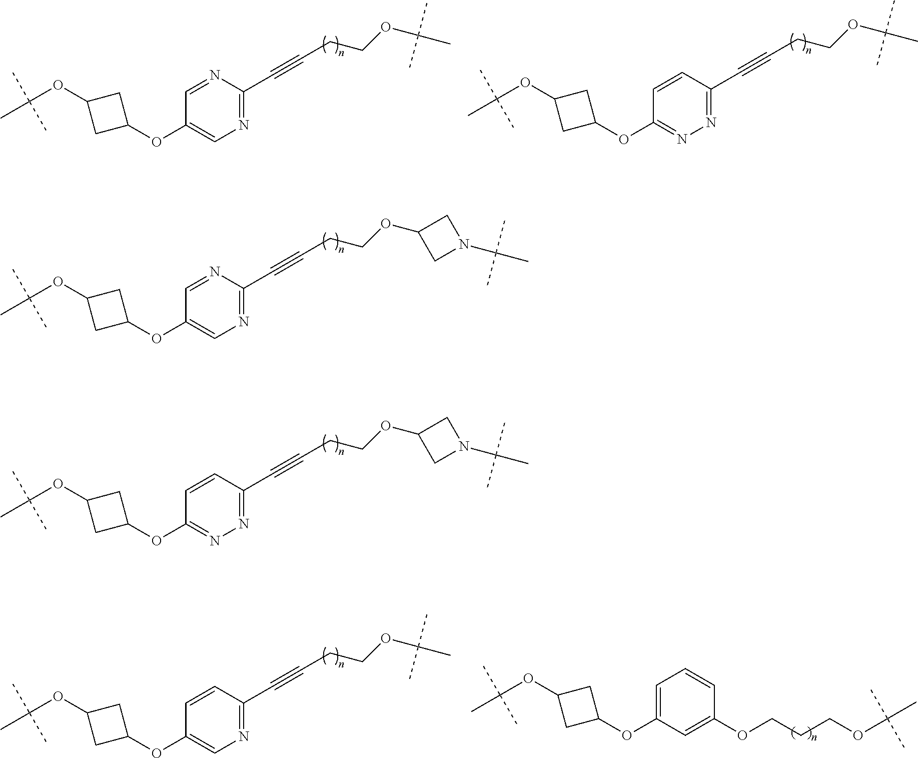

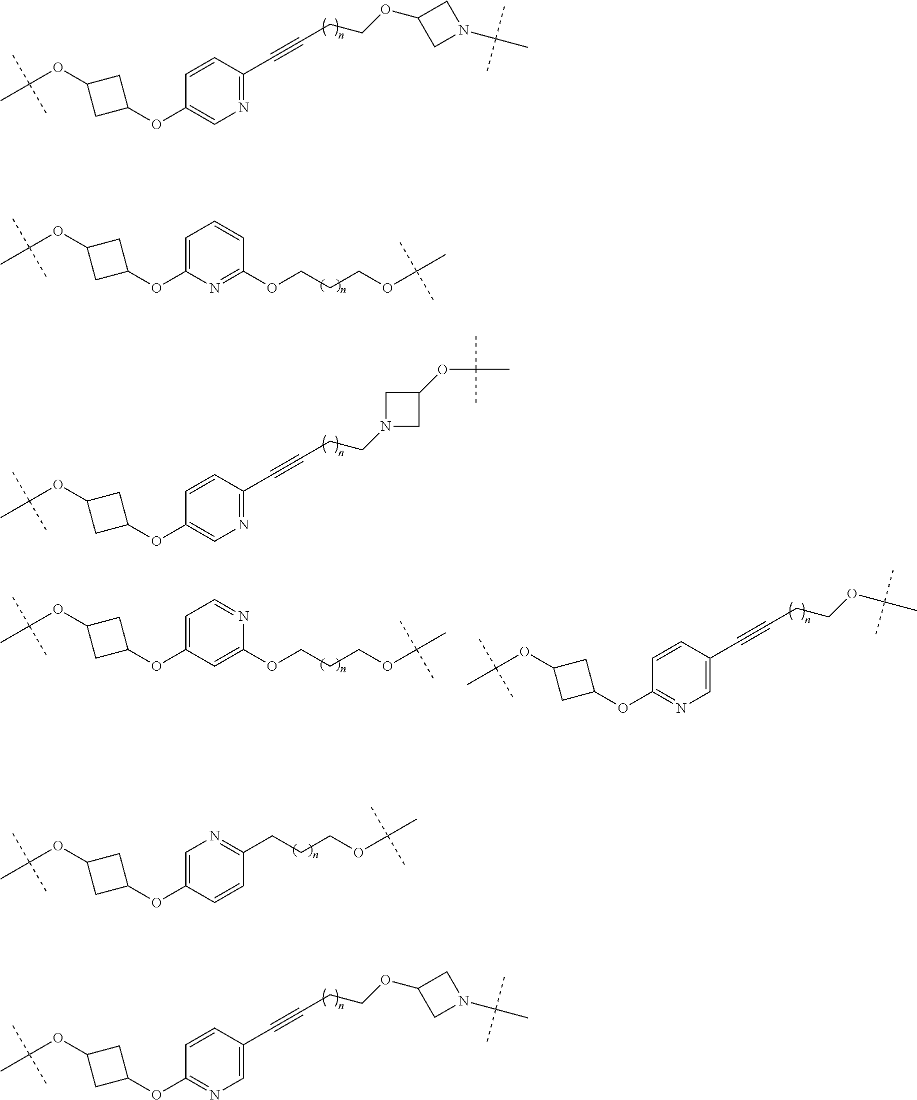

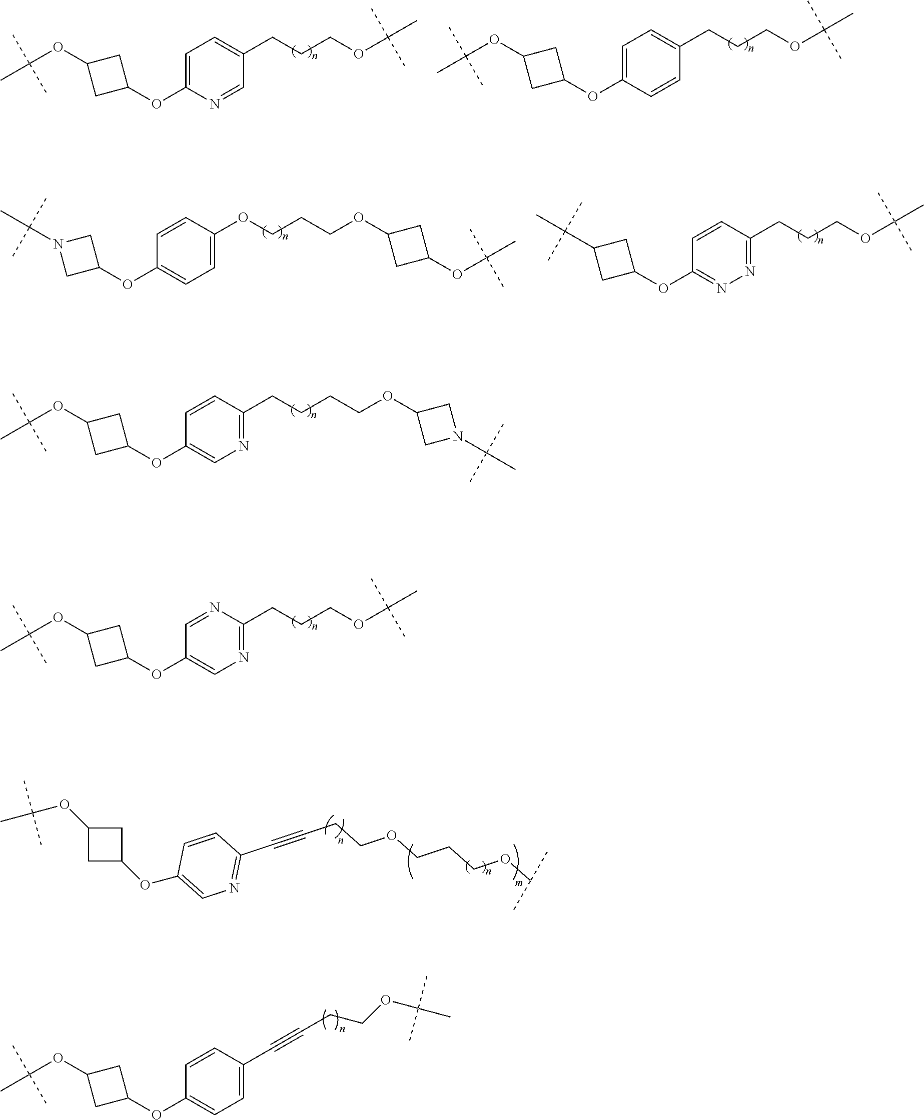

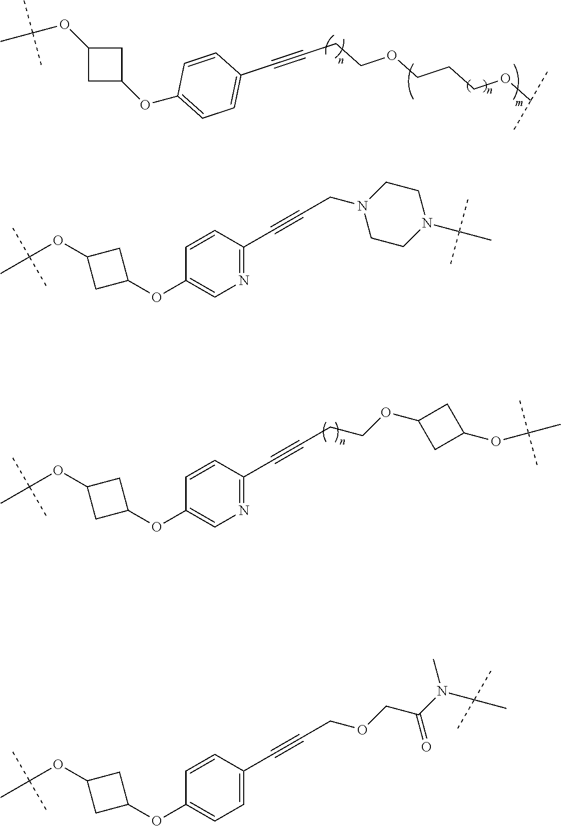

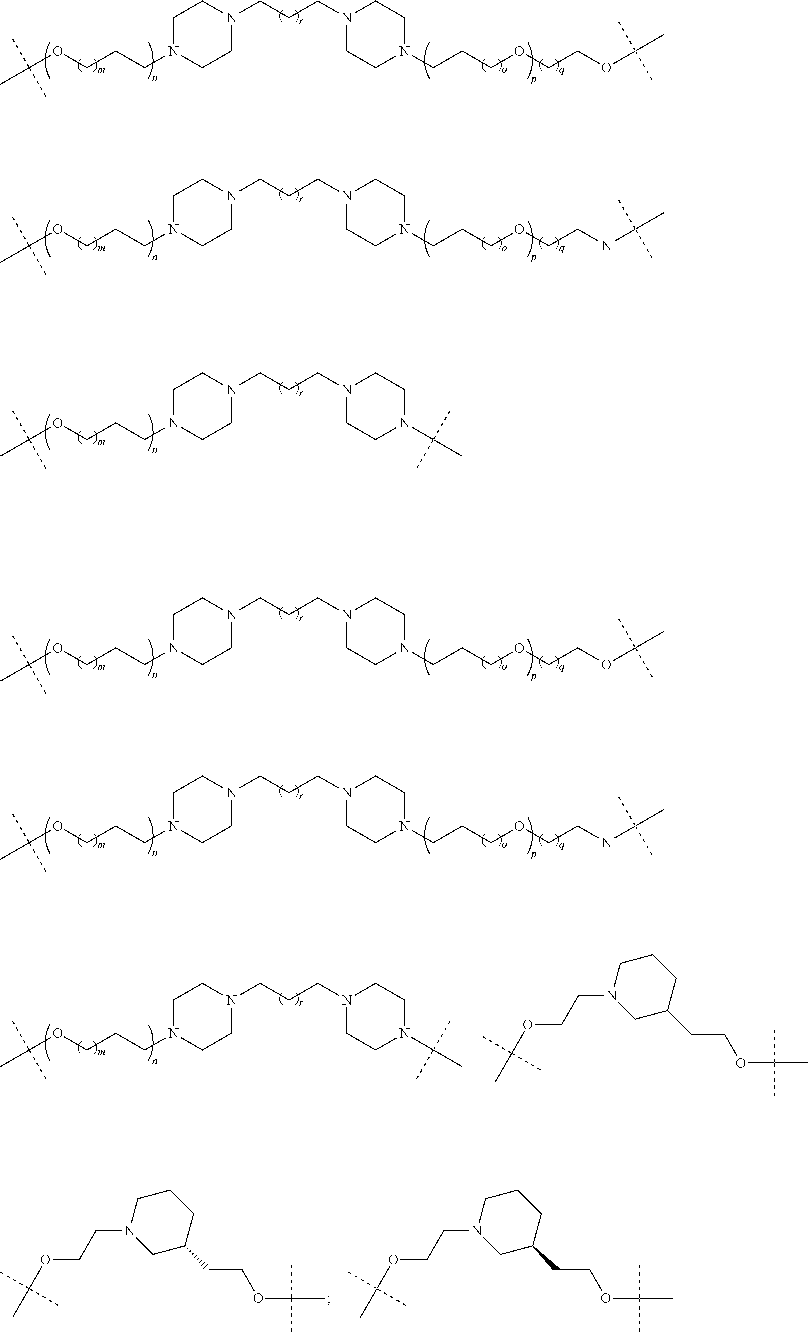

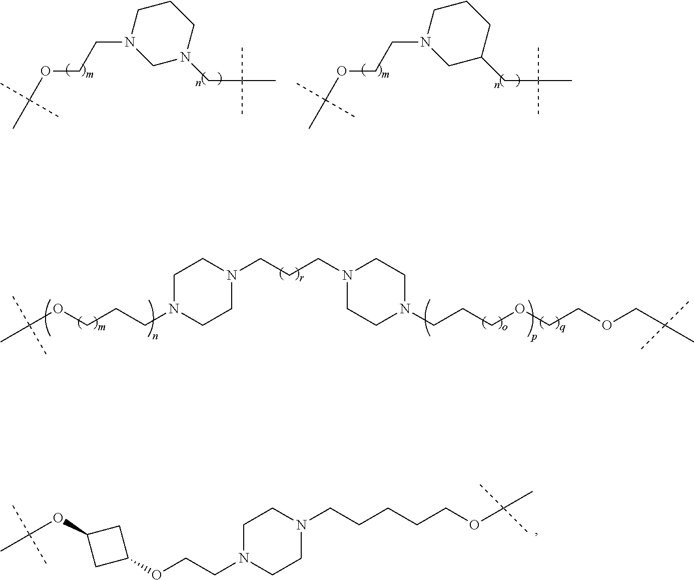

wherein each m, n, o, p, q, r, and s is independently 0, 1, 2, 3, 4, 5, 6, 7, 8, 9, 10, 11, 12, 13, 14, 15, 16, 17, 18, 19, or 20.

In one embodiment described herein, the unit A^{L} of linker (L) is selected from the group consisting of:

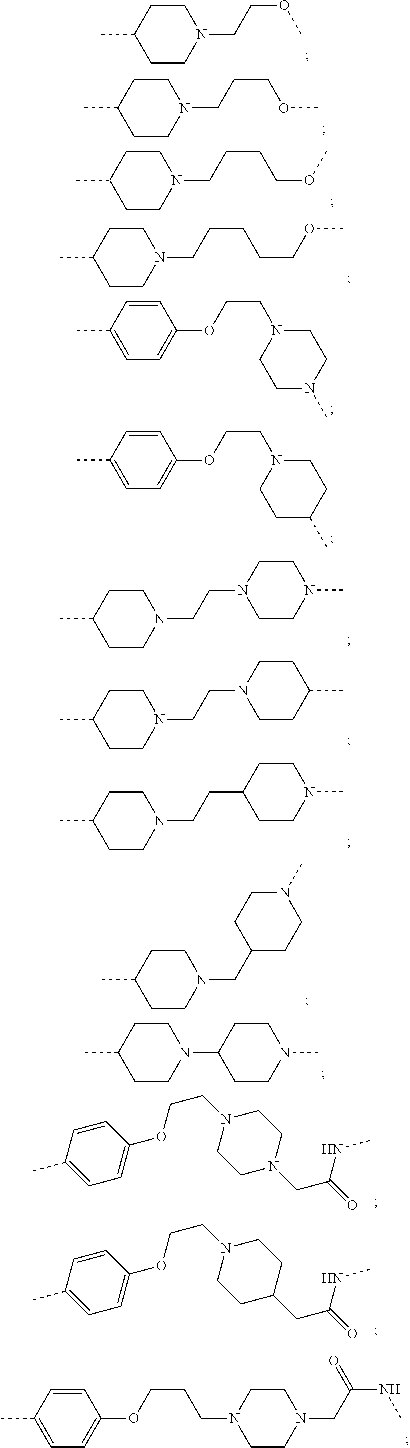

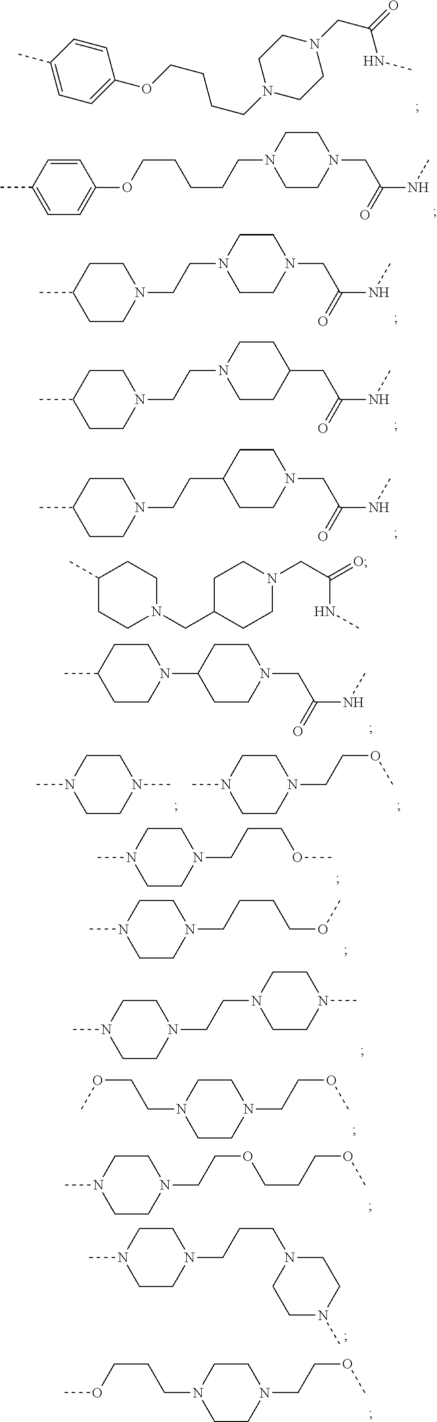

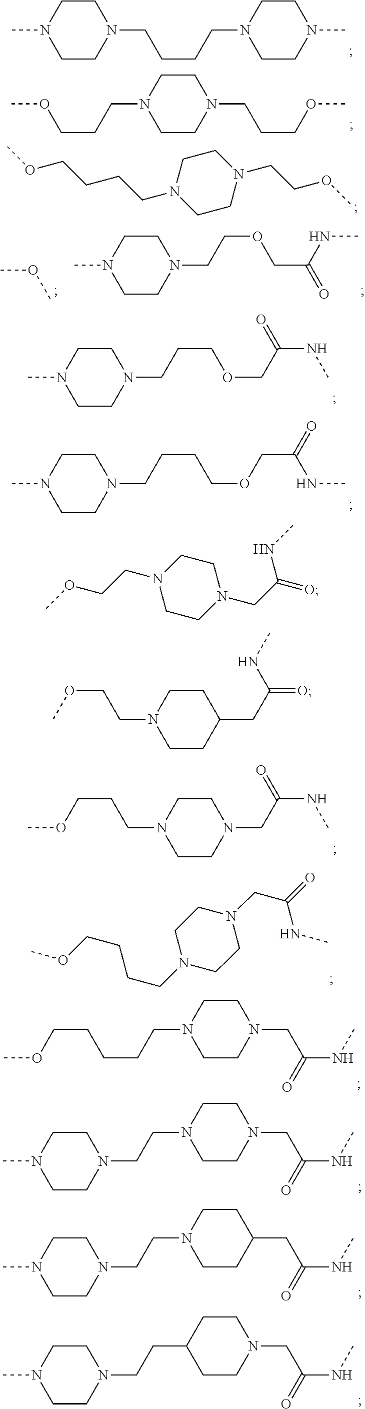

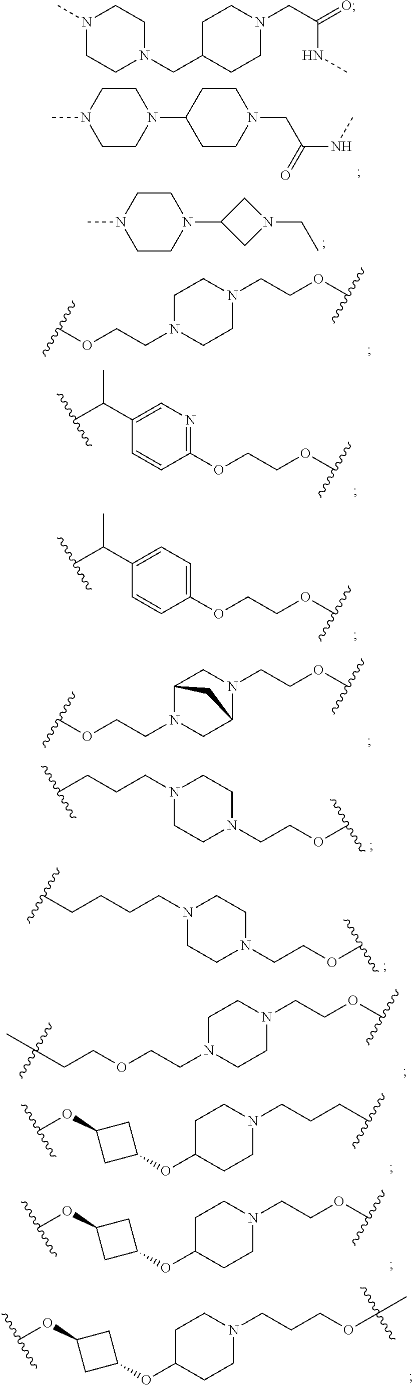

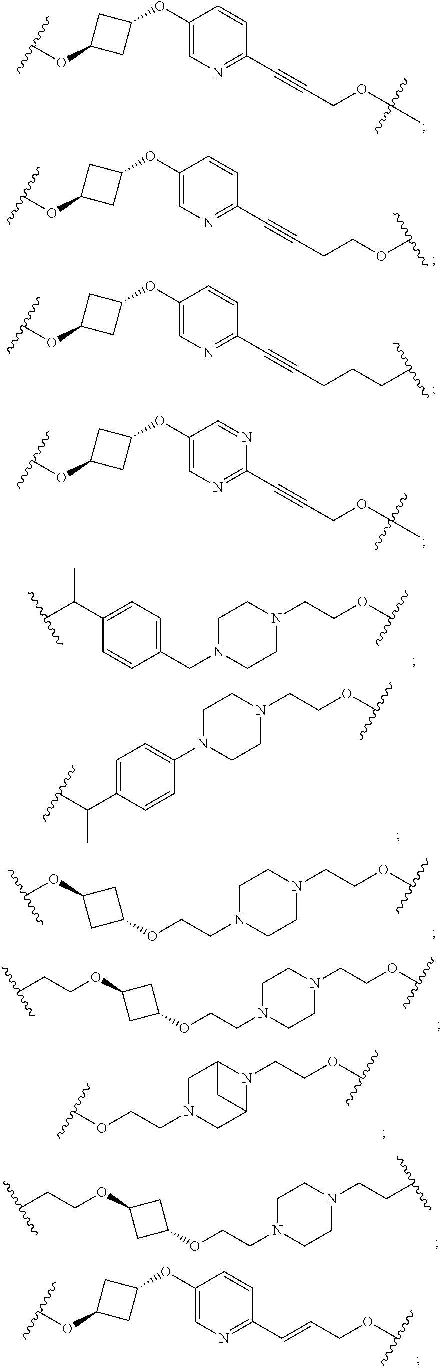

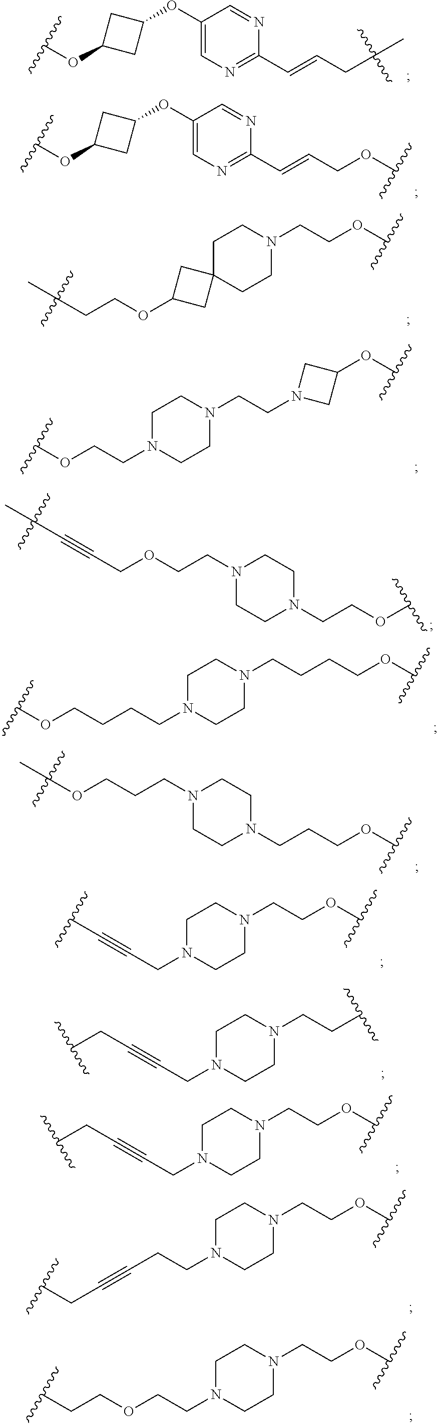

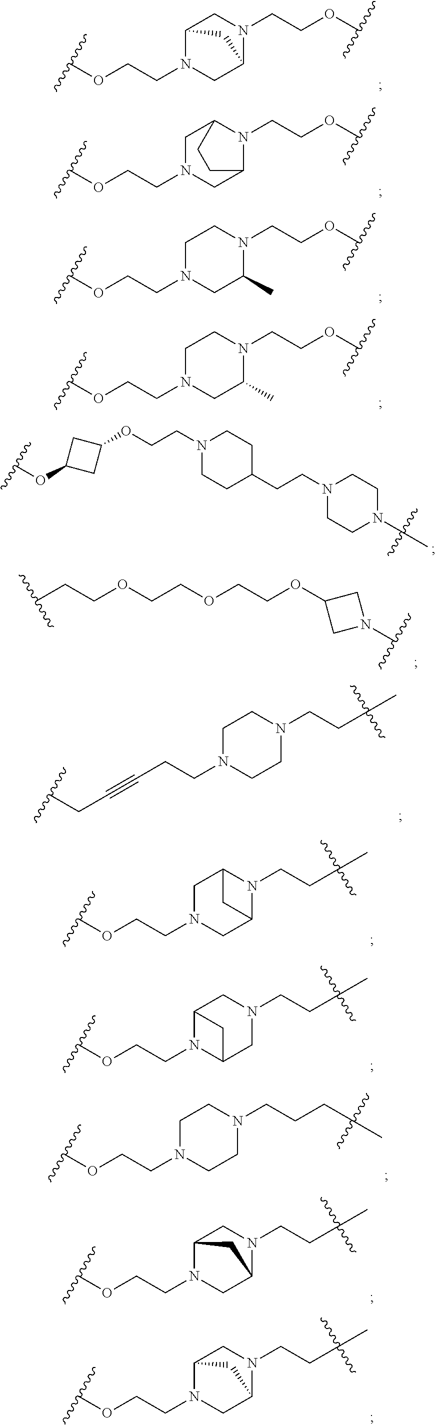

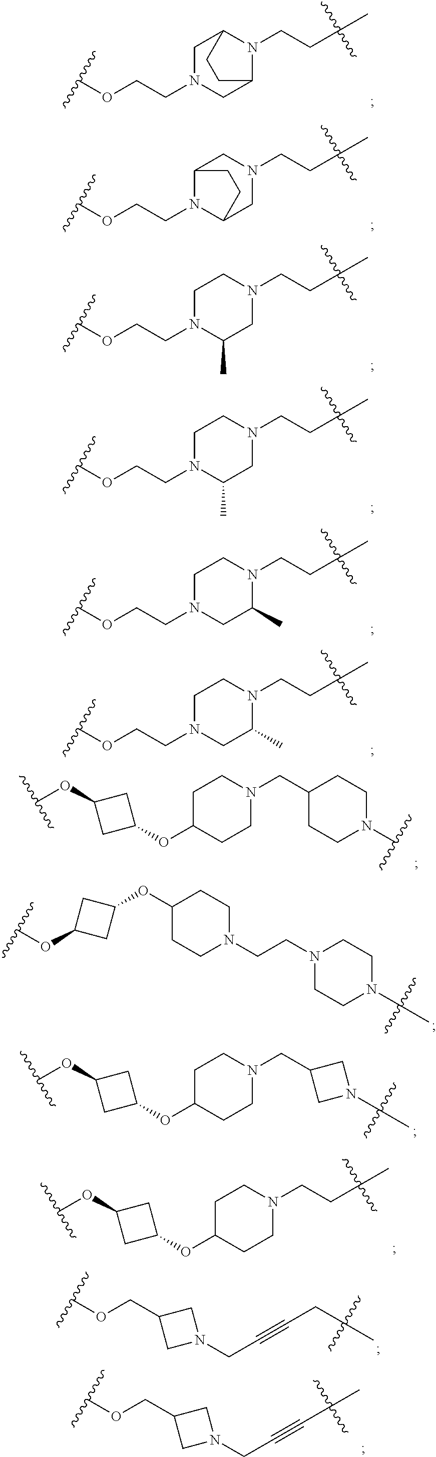

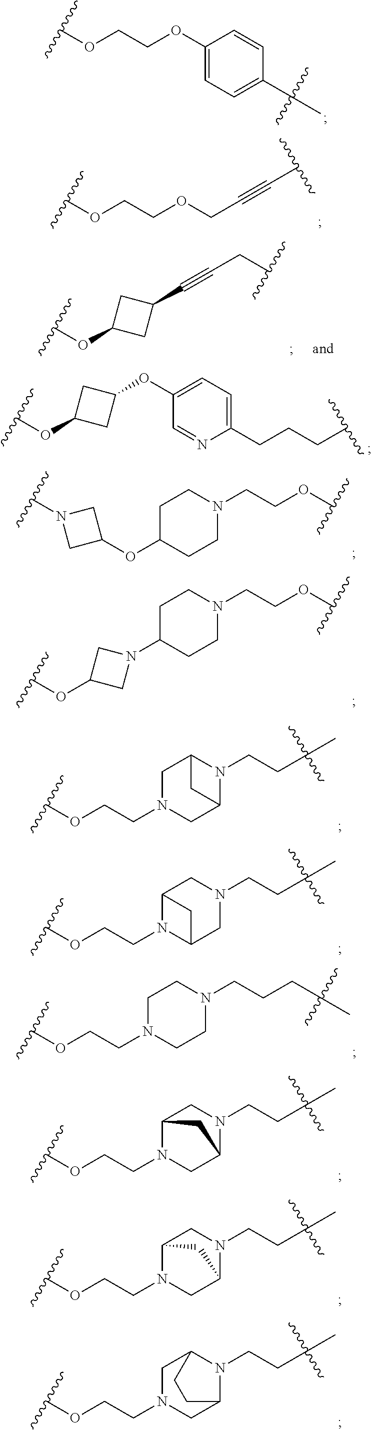

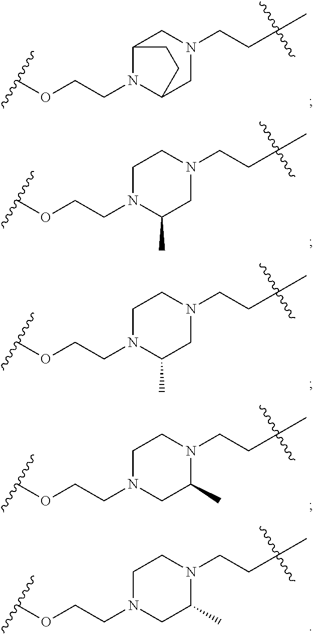

In one embodiment described herein, the linker (L) comprises a structure selected from the structure shown below:

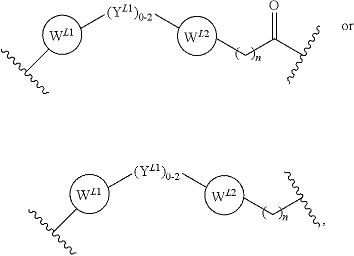

wherein:
W^{L1} and W^{L2} are each independently absent, a 4-8 membered ring with 0-4 heteroatoms, optionally substituted with R^{Q}, each R^{Q} is independently a H, halo, OH, CN, CF₃, optionally substituted linear or branched C₁-C₆ alkyl, optionally substituted linear or branched C₁-C₆ alkoxy, or 2 R^{Q} groups taken together with the atom they are attached to, form a 4-8 membered ring system containing 0-4 heteroatoms;
Y^{L1} is each independently a bond, optionally substituted linear or branched C₁-C₆ alkyl and optionally one or more C atoms are replaced with O; or optionally substituted linear or branched C₁-C₆ alkoxy;
n is 0-10; and

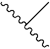

and [symbol] indicates the attachment point to the PTM or ULM moieties.

In one embodiment described herein, the linker (L) comprises a structure selected from the structure shown below:

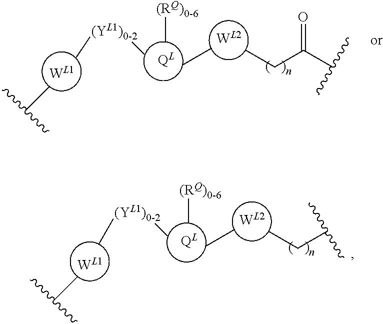

wherein:
W^{L1} and W^{L2} are each independently absent, aryl, heteroaryl, cyclic, heterocyclic, C₁₋₆ alkyl and optionally one or more C atoms are replaced with O, C₁₋₆ alkene and optionally one or more C atoms are replaced with O, C₁₋₆ alkyne and optionally one or more C atoms are replaced with O, bicyclic, biaryl, biheteroaryl, or biheterocyclic, each optionally substituted with R^{Q}, each R^{Q} is independently a H, halo, OH, CN, CF₃, hydroxyl, nitro, C≡CH, C₂₋₆ alkenyl, C₂₋₆ alkynyl, optionally substituted linear or branched C₁-C₆ alkyl, optionally substituted linear or branched C₁-C₆ alkoxy, optionally substituted OC₁₋₃alkyl (e.g., optionally substituted by 1 or more —F), OH, NH₂, NR^{Y1}R^{Y2}, CN, or 2 R^{Q} groups taken together with the atom they are attached to, form a 4-8 membered ring system containing 0-4 heteroatoms;
Y^{L1} is each independently a bond, NR^{YL1}, O, S, NR^{YL2}, CR^{YL1}R^{YL2}, C═O, C═S, SO, SO₂, optionally substituted linear or branched C₁-C₆ alkyl and optionally one or more C atoms are replaced with O; optionally substituted linear or branched C₁-C₆ alkoxy;
Q^{L} is a 3-6 membered alicyclic or aromatic ring with 0-4 heteroatoms, optionally bridged, optionally substituted with 0-6 R^{Q}, each R^{Q} is independently H, optionally substitute linear or branched C₁₋₆ alkyl (e.g., optionally substituted by 1 or more halo, C₁₋₆ alkoxyl), or 2 R^{Q} groups taken together with the atom they are attached to, form a 3-8 membered ring system containing 0-2 heteroatoms;
R^{YL1}, R^{YL2} are each independently H, OH, optionally substituted linear or branched C₁₋₆ alkyl (e.g., optionally substituted by 1 or more halo, C₁₋₆ alkoxyl), or R¹, R² together with the atom they are attached to, form a 3-8 membered ring system containing 0-2 heteroatoms;
n is 0-10; and

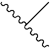

and [symbol] indicates the attachment point to the PTM or ULM moieties.

### Exemplary PTMs

In a preferred aspect of the disclosure, the PTM group is a group wherein the PTM group is also referred as the TTM group that binds to the target protein, TYK2.

The compositions described below exemplify members of TYK2 binding moieties that can be used according to the present invention. These binding moieties are linked to the ubiquitin ligase binding moiety preferably through a chemical linking group in order to present the TYK2 protein (to which TTM is bound) in proximity to the ubiquitin ligase for ubiquitination and subsequent degradation.

In certain contexts, the term “target protein” is used to refer to the TYK2 protein, a JAK family member that functions downstream of multiple cytokine receptors, which is a target protein to be ubiquitinated and degraded.

The term “protein target moiety” or PTM is used to describe a small molecule which binds to TYK2, and can be used to target the PTM for ubiquitination and degradation.

The compositions described herein exemplify the use of some of the members of these types of small molecule target protein binding moieties.

In one embodiment described herein, the PTM is a small molecule that binds TYK2. For example, in one embodiment described herein, the PTM is represented by the chemical structure PTMI or PTMII:

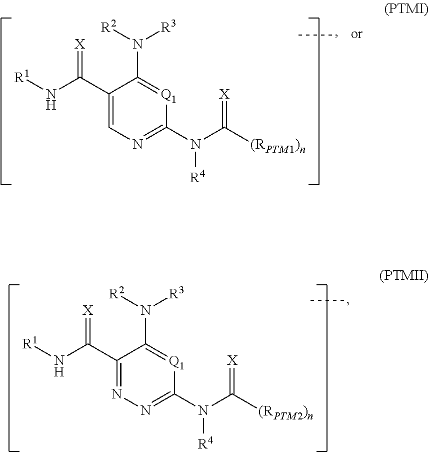

wherein:
R¹ is hydrogen, C1-C3 alkyl, C1-C3 alkyl substituted by 0-7 deuterium atoms, or C3-C6 cycloalkyl, each optionally substituted by 0-7 R^{1a};
R^{1a} at each occurrence is independently hydrogen, deuterium, CN or halo, e.g., F, Cl, Br;
R² is hydrogen, an alkyl, e.g., methyl, ethyl, propyl; furyl, pyranyl, cyclopropyl, cyclobutyl or cyclohexyl, cyclopentyl, pyridyl, pyrimidinyl, pyridazinyl, pyrazinyl, quinolinyl, pyrrolopyridinyl, each group substituted as valency allows by 0-4 groups selected from R^{2a}, —C(O)R^{2a}, C1-C6 alkyl, —(CH₂)ᵣ-3-14 membered carbocycle substituted with 0-1 R^{2a} or a 5-14 membered heterocycle containing 1-4 heteroatoms selected from N, O, and S, substituted with 0-4 R^{2a};
R^{2a} at each occurrence is independently hydrogen, ═O, halo, OCF₃, CN, NO₂, —(CH₂)ᵣOR^{b}, —(CH₂)ᵣSR^{b}, —(CH₂)ᵣC(O)R^{b}, —(CH₂)ᵣC(O)OR^{b}, —(CH₂)ᵣOC(O)R^{b}, (CH₂)ᵣNR^{u}Rⁿ, —(CH₂)ᵣC(O)NR¹¹R¹¹, —(CH₂)ᵣNR^{b}C(O)R^{c}, —(CH₂)ᵣNR^{b}C(O)OR^{c}, —NR^{b}C(O)NR¹¹R¹¹, —S(O)ₚNR¹¹R¹¹, NR^{b}S(O)ₚR^{c}, —S(O)ₚR^{c}, C1-C6 alkyl substituted with 0-3 R^{a}, C1-C6 haloalkyl, C2-C6 alkenyl substituted with 0-3 R^{a}, C2-C6 alkynyl substituted with 0-3 R^{a}, —(CH₂)ᵣ-3-14 membered carbocycle substituted with 0-1 R^{a}, a-(CH₂)ᵣ-5-7 membered heterocycle comprising carbon atoms or 1-4 heteroatoms selected from N, O, and S(0)ₚ substituted with 0-2 R^{a}, or a (CH₂)ᵣ-5-7 membered heterocycle containing 1-4 heteroatoms selected from N, O, and S(O)ₚ substituted with 0-2 R^{a};
R³ is hydrogen, C3-C10 cycloalkyl, C6-C10 aryl (e.g., phenyl) or a 5-10 membered heterocycle containing 1-4 heteroatoms selected from N, O, and S, each group substituted with 0-4 R^{3a};
wherein at least one R² or R³ is hydrogen;
R^{3a} at each occurrence is independently hydrogen, ═O, halo, OCF₃, CF₃, CHF₂, CN, NO₂, —(CH₂)ᵣOR^{b}, —(CH₂)ᵣSR^{b}, —(CH₂)ᵣC(O)R^{b}, —(CH₂)ᵣC(O)OR^{b}, —(CH₂)ᵣOC(O)R^{b}, —(CH₂)ᵣNR¹¹R¹¹ (CH₂)ᵣC(O)NR¹¹R¹¹ (CH₂)ᵣNR^{b}C(O)R^{c}, —(CH₂)ᵣNR^{b}C(O)OR^{c}, —NR^{b}C(O)NR¹¹R¹¹, —S(O)ₚNR¹¹R¹¹, NR^{b}S(O)ₚR^{c}, —S(O)ₚR′, C1-C6 alkyl substituted with 0-3 R^{a}, C₂₋₆ alkenyl substituted with 0-3 R^{a}, C₂₋₆ alkynyl substituted with 0-3 R^{a}, C₁-C₆ haloalkyl, —(CH₂)ᵣ-3-14 membered carbocycle substituted with 0-3 R^{a} or a —(CH₂)ᵣ-5-10 membered heterocycle containing 1-4 heteroatoms selected from N, O, and S(O)ₚ substituted with 0-3 R^{a};
or two R^{3a}, together with the atoms to which they are attached, combine to form a fused ring wherein said ring is selected from phenyl and a heterocycle comprising carbon atoms and 1-4 heteroatoms selected from N, O, and S(O)ₚ, each fused ring substituted with 0-3 R^{a1};
R⁴ is hydrogen, C₁-C₄ alkyl substituted with 0-1 R^{f}, (CH₂)ᵣ-phenyl substituted with 0-3 R^{d} or a —(CH₂)-5-7 membered heterocycle containing 1-4 heteroatoms selected from N, O, or S(O)ₚ;
R⁶ is hydrogen, halo, C₁-C₄alkyl, C₁-C₄haloalkyl, OC₁-C₄haloalkyl, OC₁-C₄alkyl, CN, NO₂ or OH;
R¹¹ at each occurrence is independently hydrogen, C₁₋₄ alkyl substituted with 0-3 R^{f}, CF₃, C₃₋₁₀ cycloalkyl substituted with 0-1 R^{f}, (CH)ᵣ-phenyl substituted with 0-3 R^{d} or —(CH₂)ᵣ-5-7 membered heterocycle containing 1-4 heteroatoms selected from N, O, and S(O)ₚ substituted with 0-3 R^{d};
R^{a} and R^{a1} at each occurrence are independently hydrogen, halogen, OH, OCF₃, CF₃, CHF₂, CN, NO₂, —(CH₂)ᵣOR^{b}, —(CH₂)ᵣSR^{b}, —(CH₂)ᵣC(O)R^{b}, —(CH₂)ᵣC(O)OR^{b}, —(CH₂)ᵣOC(O)R^{b}, —(CH₂)ᵣNR¹¹R¹¹, —(CH₂)ᵣC(O)NR¹¹R¹¹, —(CH₂)ᵣNR^{b}C(O)R^{c}, —(CH₂)ᵣNR^{b}C(O)OR^{c}, —NR^{b}C(O)NR¹¹R¹¹, —S(O)ₚNR¹¹R¹¹, —NR^{b}S(O)ₚR^{c}, —S(O)R^{c}, —S(O)₂R^{c}, C1-C6 alkyl substituted with 0-3 R^{f}, C1-C6 haloalkyl, C2-C6 alkenyl substituted with 0-3 R^{d}, C2-C6 alkynyl substituted with 0-3 R^{d}, —(CH₂)ᵣ-3-14 membered carbocycle or —(CH₂)ᵣ-5-7 membered heterocycle containing 1-4 heteroatoms selected from N, O, and S(O)ₚ substituted with 0-3 R^{f};
R^{b} is hydrogen, C1-C6 alkyl substituted with 0-3 R^{d}, C1-C6 haloalkyl, C3-C6 cycloalkyl substituted with 0-2 R^{d}, or —(CH₂)ᵣ-5-7 membered heterocycle containing 1-4 heteroatoms selected from N, O, and S(O)ₚ substituted with 0-3 R^{f} or (CH₂)ᵣ-phenyl substituted with 0-3 R^{d};
R^{c} is C1-C6 alkyl substituted with 0-3 R^{f}, (CH₂)ᵣ-C3-C6 cycloalkyl substituted with 0-3 R^{f} or (CH₂)ᵣ-phenyl substituted with 0-3 R^{f};
R^{d} at each occurrence is independently hydrogen, F, Cl, Br, OCF₃, CF₃, CN, NO₂, —OR^{e}, —(CH₂)ᵣC(O)R^{c}, —NR^{e}R^{e}, —NR^{e}C(O)OR^{c}, C1-C6 alkyl or (CH₂)ᵣ-phenyl substituted with 0-3 R^{f};
R^{e} at each occurrence is independently hydrogen, C1-C6 alkyl, C3-C6 cycloalkyl or (CH₂)ᵣ-phenyl substituted with 0-3 R^{f};
R^{f} independently at each occurrence is hydrogen, halo, CN, NH₂, OH, C₃₋₆ cycloalkyl, CF₃, O(C1-C6alkyl) or a —(CH₂)ᵣ-5-7 membered heterocycle containing 1-4 heteroatoms selected from N, O, and S(O)ₚ;
R_{PTM1} and R_{PTM2} are independently: H; halogen (e.g., Cl or F); —CN; —OH; —NO₂; —NH₂; optionally substituted linear or branched alkyl (e.g., optionally substituted linear or branched C1-C6 alkyl or optionally substituted linear or branched C1-C4 alkyl or C1-C8 alkyl optionally substituted with OH); optionally substituted cycloalkyl (e.g., cyclopropyl, cyclobutyl, cyclopentyl, cyclohexyl); O-optionally substituted linear or branched C1-C4 alkyl; an optionally substituted C1-C4 alkynyl; an optionally substituted C1-C4 alkyne; optionally substituted linear or branched hydroxyalkyl (e.g., optionally substituted linear or branched C1-C7 hydroxyalkyl); optionally substituted alkylcycloalkyl (e.g., includes optionally substituted C1-C6 alkyl, optionally substituted C3-C10 cycloalkyl; or both); optionally substituted alkyl-aryl (e.g., includes an optionally substituted linear or branched C1-C6 alkyl, an optionally substituted 5-10 member heteroaryl, or both); optionally substituted alkyl-heteroaryl (e.g., includes an optionally substituted linear or branched C1-C6 alkyl, an optionally substituted 5-10 member heteroaryl, or both); optionally substituted alkyl-heteroaryl (e.g., includes a C1-C6 alkyl, an optionally substituted 5 or 6 member heteroaryl, optionally substituted with a C1-C4 alkyl; the heteroaryl is selected from oxazol-4-yl, 1,3,4-triazol-2-yl, and imidazole-1-yl; or a combination thereof); optionally substituted —NH-alkyl-heteroaryl (e.g., an optionally substituted linear or branched C1-C5 alkyl, an optionally substituted 5-8 member heteroaryl, optionally substituted with a C1-C4 alkyl, N—CH₂-pyrazol-4-yl, or a combination thereof); optionally substituted alkoxy (e.g., an optionally substituted linear or branched C1-C6 alkyl or —OCH₃); optionally substituted O-heterocyclyl (e.g., includes an optionally substituted 3-12 or 4-7 member heterocyclyl; an optionally substituted heterocycloalkyl; an optionally substituted C₃₋₁₂ monocyclic or bicyclic heterocycloakly; optionally substituted with at least one OH, C1-C5 alkyl (such as a methyl), ═O, NH₂, or a combination thereof; or a combination thereof); optionally substituted S-heterocyclyl (e.g., includes an optionally substituted 4-7 member heterocyclyl; an optionally substituted heterocycloalkyl; optionally substituted with at least one C1-C4 alkyl (such as a methyl), ═O, or a combination thereof; or a combination thereof); optionally substituted

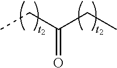

(e.g., optionally substituted with a linear or branched C1-C4 alkyl; —(CH₂)ₙCO(CH₂)ᵥCH₃, —COCH₃, or —CH₂CH₂COCH₃, wherein each u and v is independently selected from 1, 2, 3, 4 or 5);
optionally substituted

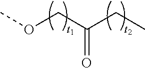

(e.g., optionally substituted with a linear or branched C1-C4 alkyl; —O(CH₂)ₙCO(CH₂)ᵥCH₃, —O(CH₂)ᵤCH((CH₂)ₓCH₃)(CH₂)_{w}CO(CH₂)ᵥCH₃, —O—CH₂COCH₃, —O—CH₂COCH₂CH₃, —O—CH(CH₃)COCH₃, —OCH₂COCH₃, or —OCH₂(CH₃)COCH₃, wherein each u, v, w, and x is independently selected from 1, 2, 3, 4 or 5); optionally substituted

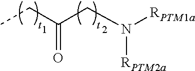

(e.g., optionally substituted with a linear or branched C1-C4 alkyl; —(CH₂)ᵤCO(CH₂)ᵥNR_{PTM1a}R_{PTM2a}, —CONR_{PTM1a}R_{PTM2a}, —CH₂CONR_{PTM1a}R_{PTM2a}, —CH₂CH₂CONR_{PTM1a}R_{PTM2a}, —CONHCH₃, or —CH₂CONHCH₃, wherein each u and v is independently selected from 1, 2, 3, 4 or 5); optionally substituted

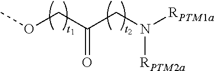

(e.g., optionally substituted with a linear or branched C1-C4 alkyl; —O(CH₂)ᵤCO(CH₂)ᵥNR_{PTM1a}R_{PTM2a}, —O(CH₂)ₙCH((CH₂)ₓCH₃)(CH₂)_{w}CO(CH₂)ᵥ NR_{PTM1a}R_{PTM2a}, —O—CH(CH₃)CONR_{PTM1a}R_{PTM2a}, —O—CH₂CONR_{PTM1a}R_{PTM2a}, or —OCH₂C(O)NHOCH₃, wherein each u, v, w, and x is independently selected from 1, 2, 3, 4 or 5); optionally substituted

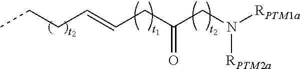

(e.g., optionally substituted with a linear or branched C1-C4 alkyl; —(CH₂)ₙCHCH(CH₂)_{w}CO(CH₂)ᵥ NR_{PTM1a}R_{PTM2a} or —CHCHCONR_{PTM1a}R_{PTM2a}, wherein each u, v, and w is independently selected from 1, 2, 3, 4 or 5); optionally substituted

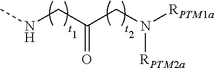

(e.g., optionally substituted with a linear or branched C1-C4 alkyl; —NH—(CH₂)ᵤCO(CH₂)ᵥNR_{PTM1a}R_{PTM2a} or —NH—CH₂CONR_{PTM1a}R_{PTM2a}, wherein each u and v is independently selected from 1, 2, 3, 4 or 5); fluoroalkoxy (e.g., a mono-, bi- and/or tri-fluoroalkoxy); optionally substituted monocylic or bicyclic cyclocalkyl (e.g., an optionally substituted 3-12 member cycloalkyl; optionally substituted with at least one of OH, ═O, linear or branched C1-C6 alkyl (such as a methyl, ethyl, or butyl), or NH₂; or a combination thereof); optionally substituted hydroxycycloalkyl; optionally substituted aryl (e.g., an optionally substitute C5-C10 aryl, an optionally substituted 5-7 member aryl; optionally substituted with at least one halogen or C1-C3 alkyl (e.g., methyl or ethyl); or a combination thereof), optionally substituted heteroaryl (e.g., an optionally substituted 5-10 or member heteroaryl, an optionally substituted 5-7 member heteroaryl; an optionally substituted 5-member heteroaryl; optionally substituted with at least one halogen or C1-C3 alkyl (e.g., methyl or ethyl); or a combination thereof) optionally linked to Q₁ via a C or N-atom of the heteroaryl (e.g., at least one of optionally linked to Q₁, optionally linked via an optionally substituted —(CH₂)ᵤO(CH₂)ᵥO(CH₂)ₓ—, or a combination thereof); optionally substituted monocyclic or bicyclic heterocyclyl (e.g., an optionally substituted 3-12 member heterocyclyl; an C3-C12 monocylcic or bicyclic heterocycloalkyl, azetidine1-yl, pyrrolidin-1-yl, piperidin-1yl, piperazin-1-yl, or morpholin-4-yl, or homopiperazin-1-yl, each optionally substituted with OH, a linear or branched C1-C5 alkyl (a methyl, ethyl, or butyl group) or NH₂) optionally linked to Q₁ via a C or N atom of the heterocyclyl (e.g., at least one of optionally linked to Q₁, optionally linked via an optionally substituted —(CH₂)ᵤO(CH₂)ᵥO(CH₂)ₓ—, or both);
each t₁ is independently selected from 1, 2, 3, 4, or 5;
each t₂ is independently is independently selected from 0, 1, 2, 3, 4, or 5;
each R_{PTM1a} and R_{PTM2a} are independently H, optionally substituted C1-C4 alkyl (e.g., a CH₃ or CH₂CH₃), optionally substituted C1-C4 alkoxy (e.g., —OCH₂ or —CH₂CH₃), CH₂OCH₃ or R_{PTM1a} and R_{PTM2a} are joined together form a 3-10 member ring;
Q₁ is CH, N, O, or C, each optionally substituted with one or more independently selected R_{PTM1} or R_{PTM2} (e.g., 1, 2, or 3 independently selected R_{PTM1} or R_{PTM2}, depending upon valency);
p is 0, 1, or 2;
r is 0, 1, 2, 3, or 4;
X is O, S, or CH₂;
n is an integer from 0 to 10; and
[symbol] of the PTM indicates the point of attachment with a chemical linker group or a ULM.

In one embodiment described herein, R_{PMT1} of PTMI or R_{PMT2} of PTMII is modified to be covalently linked to a linker group (L) or a ULM (such as CLM). In one embodiment described herein, R_{PTM1} of PTMI or R^{PTM2} of PTMII is modified to be covalently linked to a chemical linker group (L) or a ULM.

In one embodiment described herein, the X of the PTM (e.g., PTMI and PTMII) is O.

In one embodiment described herein, the PTMI has the chemical structure:

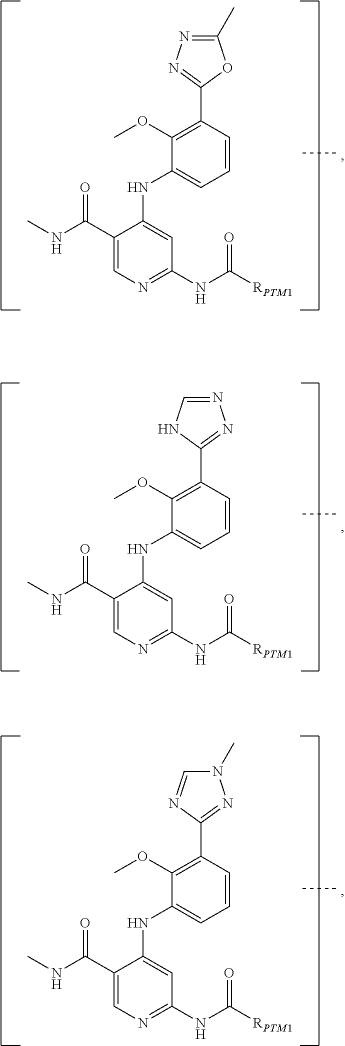

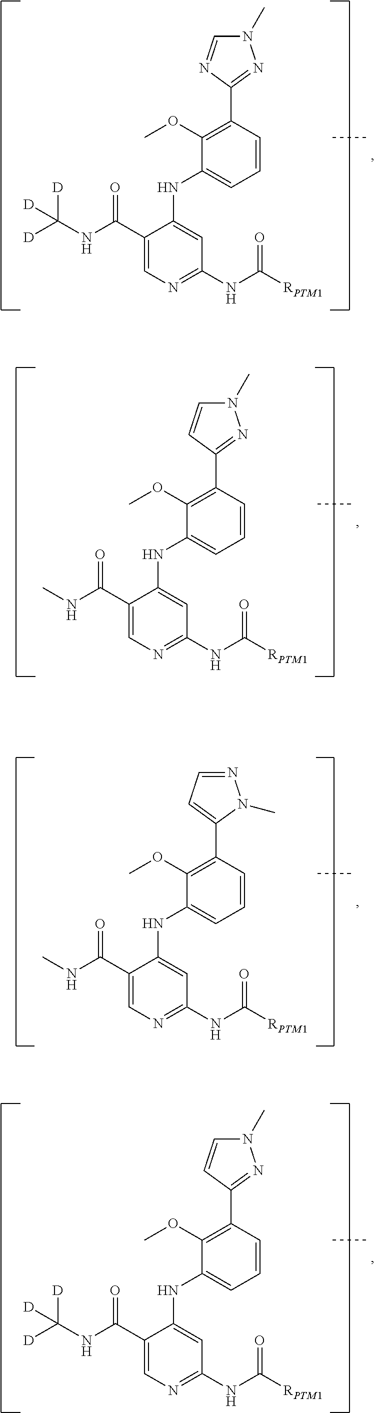

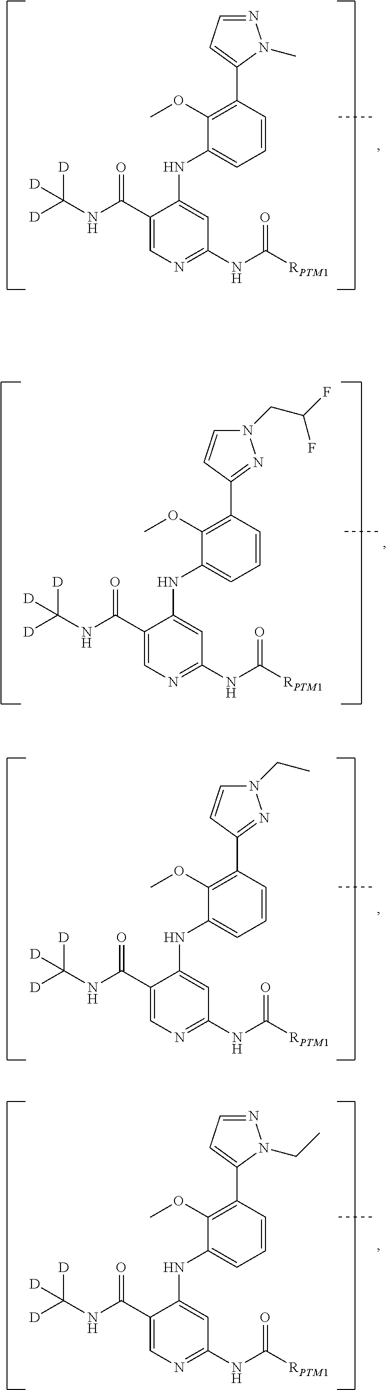

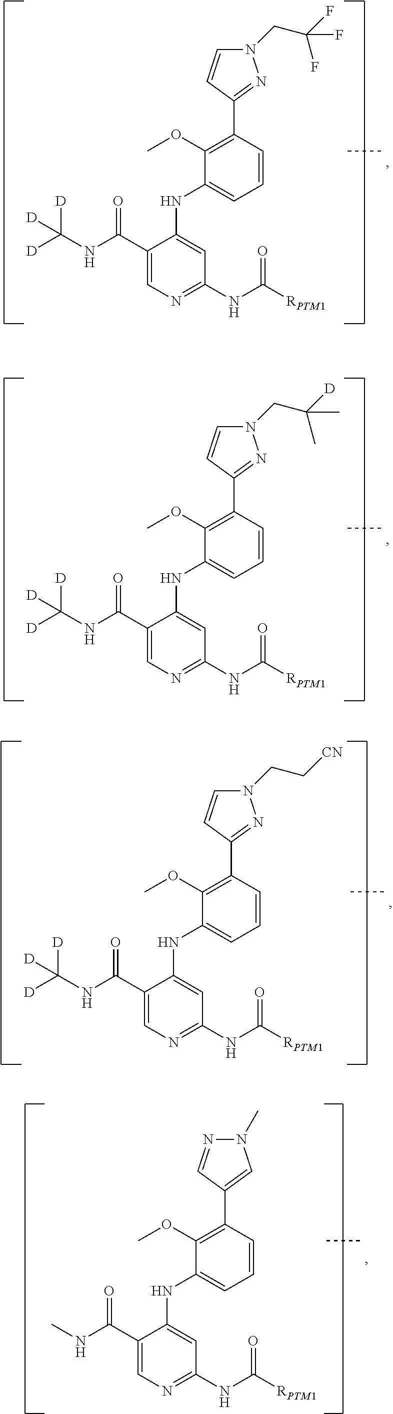

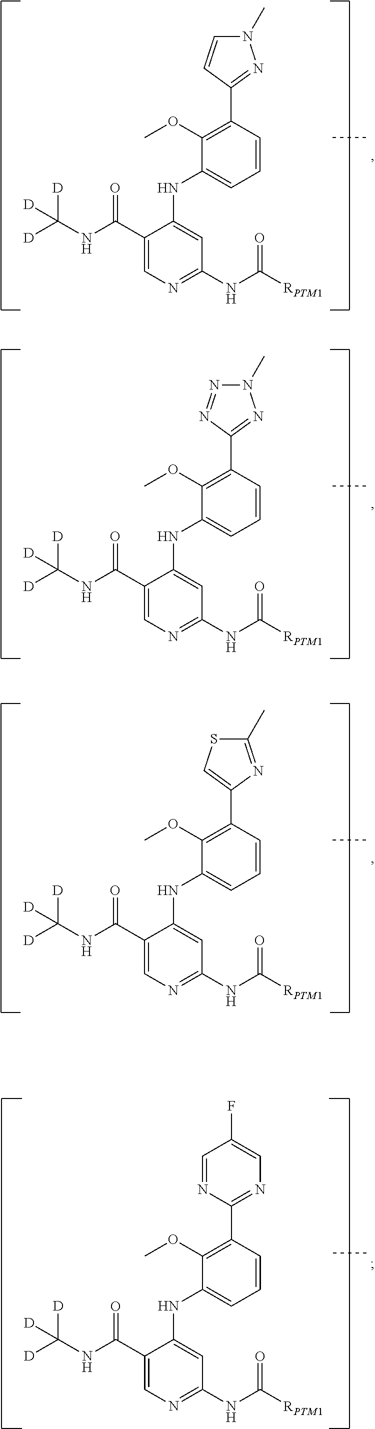

and wherein [symbol] the PTM indicates the point of attachment with a chemical linker group or a ULM.

In one embodiment described herein, the PTMII has the chemical structure:

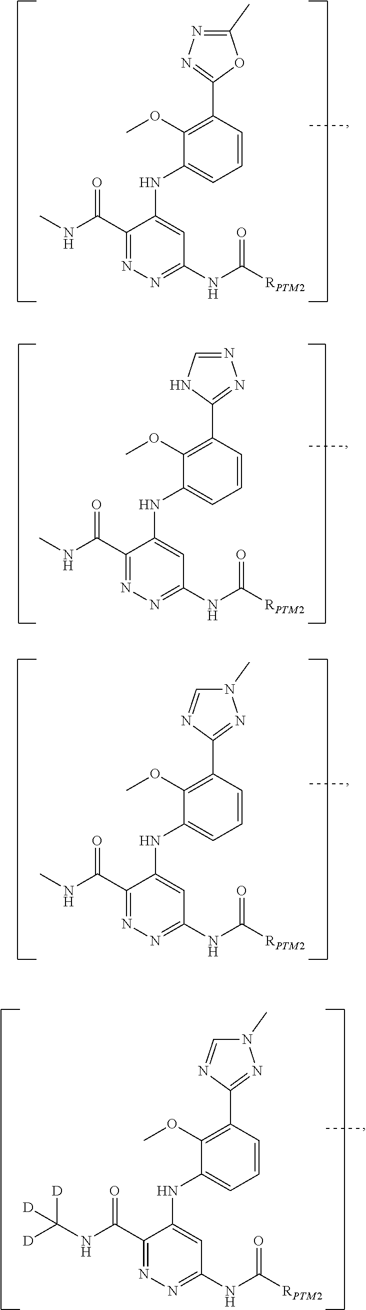

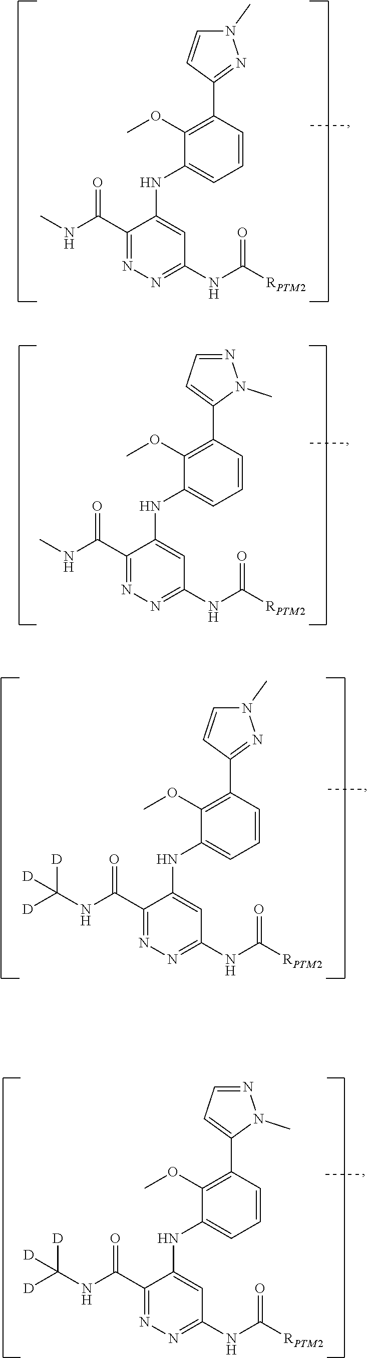

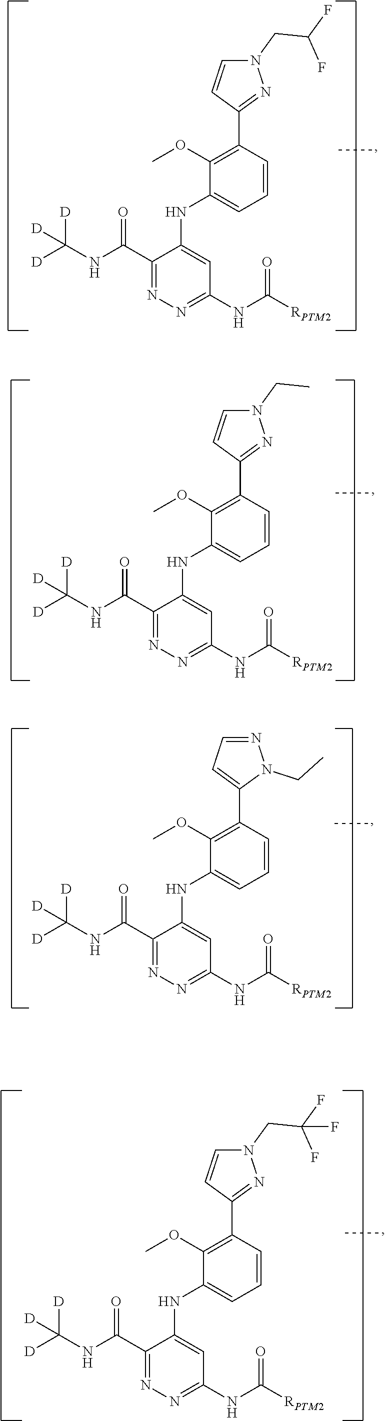

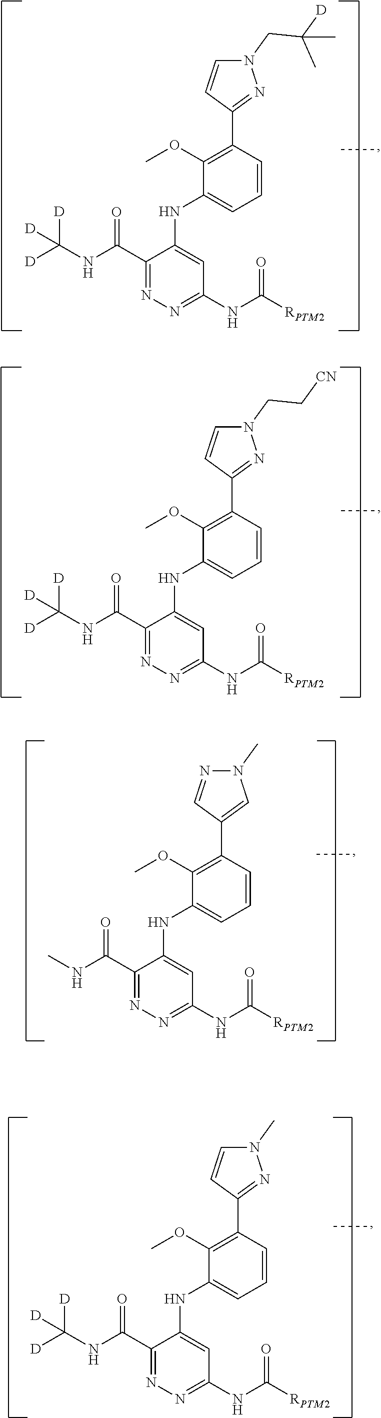

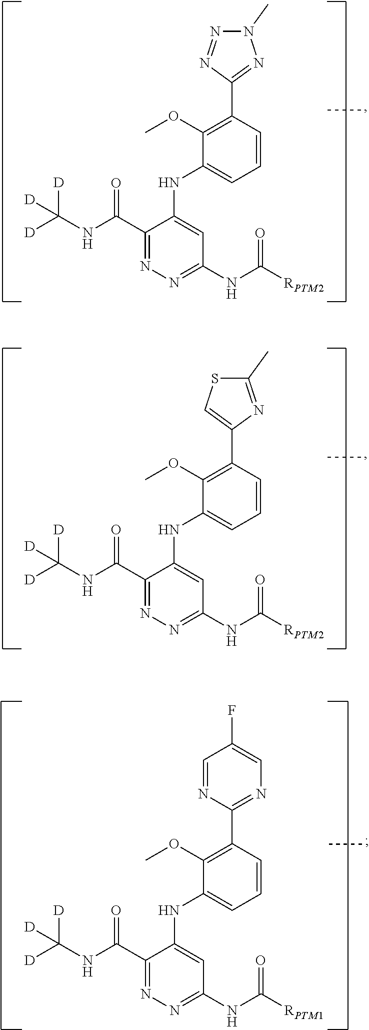

and wherein [symbol] of the PTM indicates the point of attachment with a chemical linker group or a ULM.

In one embodiment described herein, R_{PTM1} or R_{PTM2} is selected from

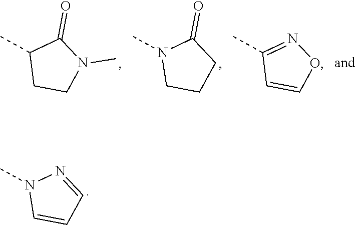

In one embodiment described herein, the PTM is selected from the group consisting of:

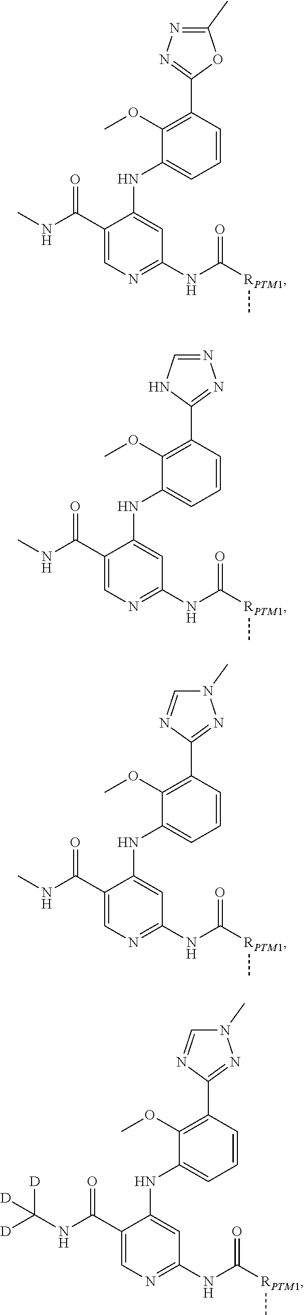

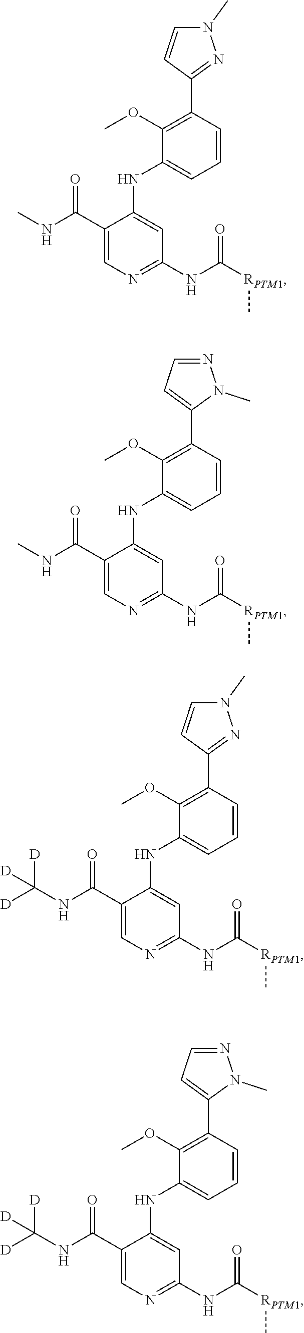

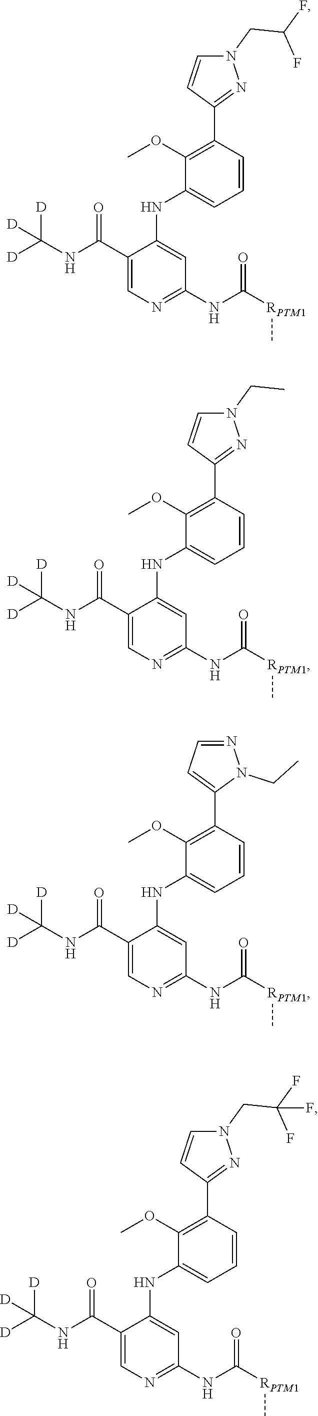

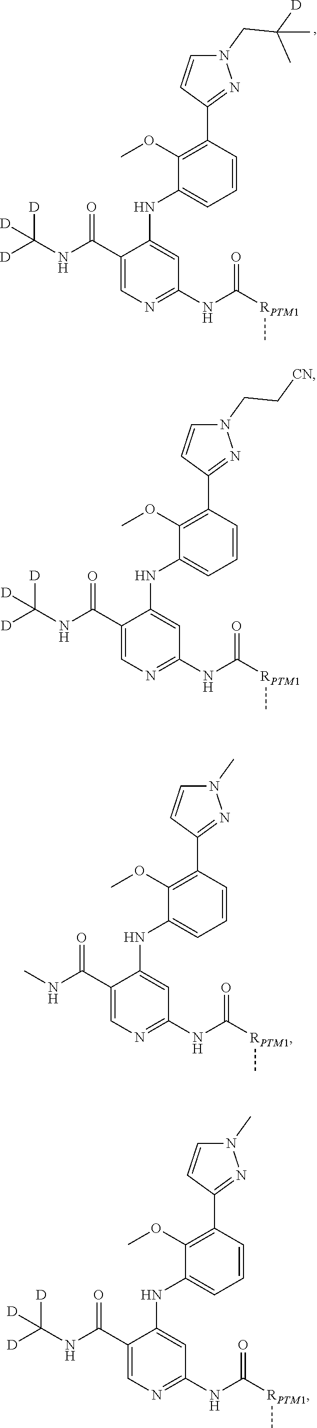

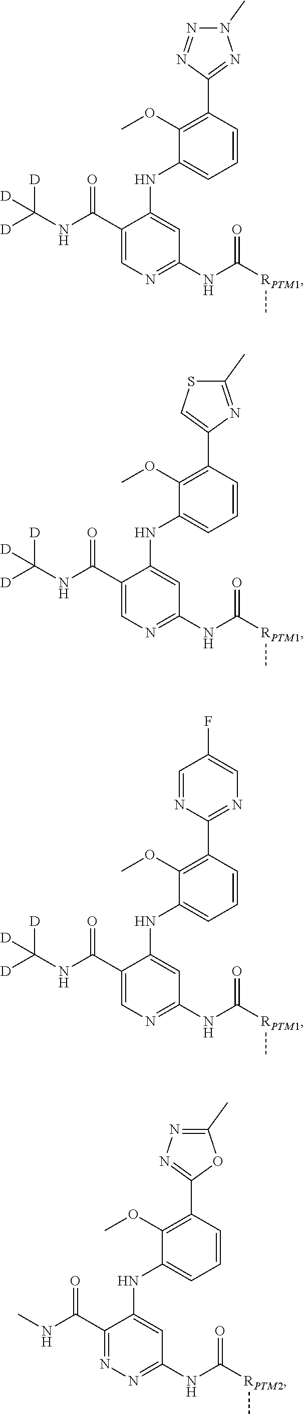

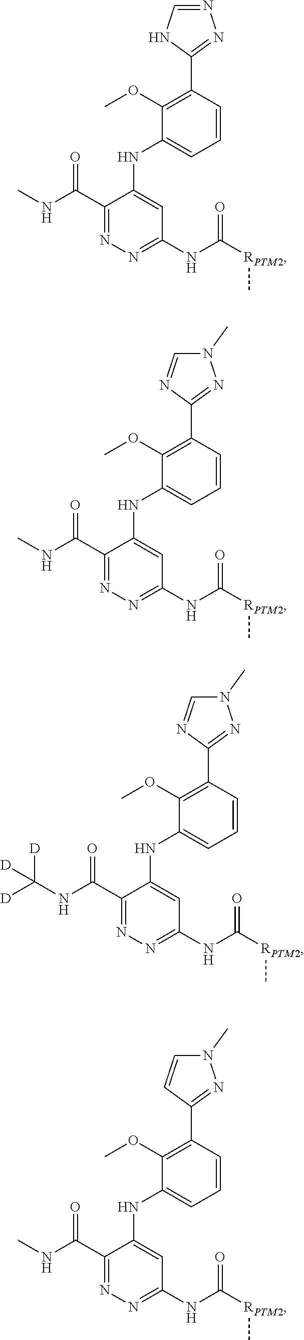

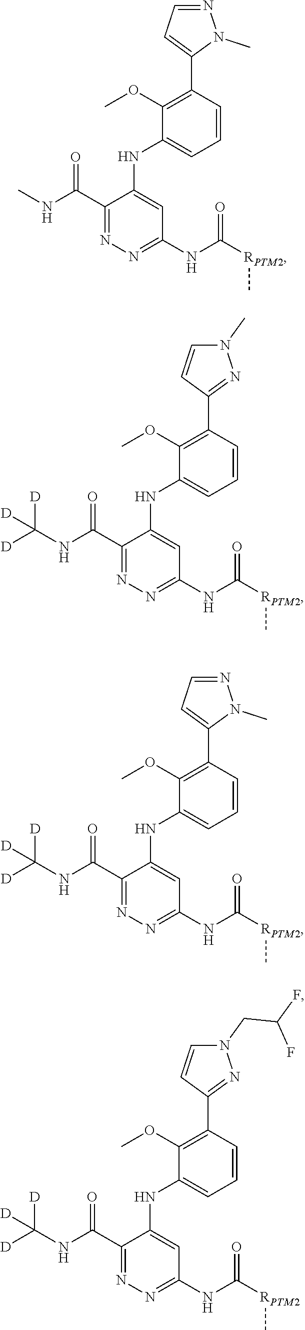

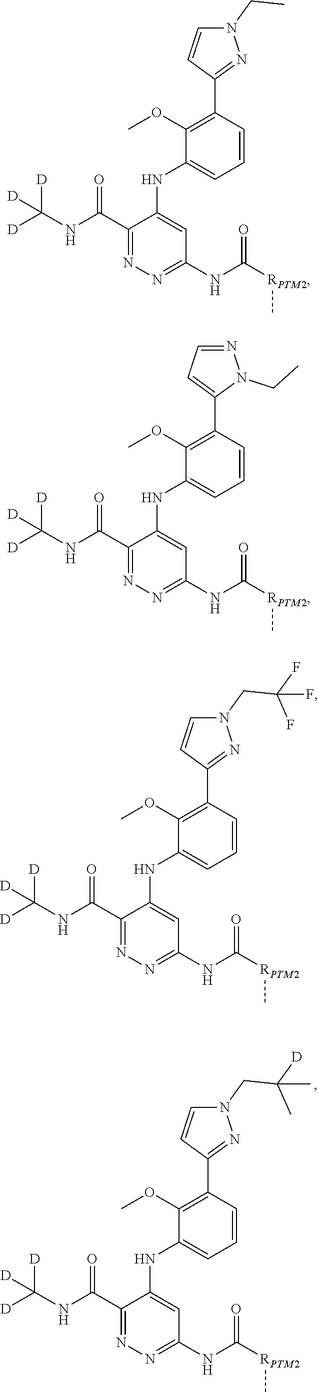

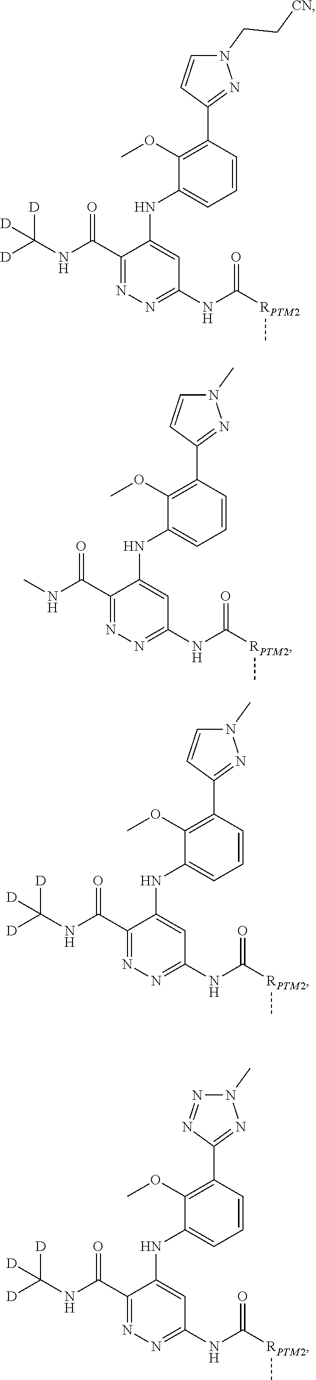

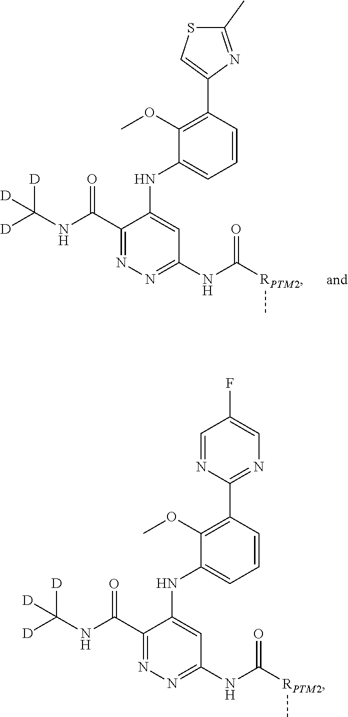

wherein R_{PTM1} and R_{PTM2} is a bond, an atom or chemical group as described herein coupled to a linker (L) or a ULM, and the [symbol] of the R_{PTM1} and R_{PTM2} indicates the point of attachment with L or a ULM.

In one embodiments described herein R_{PTM1} or R_{PTM2} is selected from: a bond, methyl, ethyl, propyl, butyl, pentyl, and hexyl, H, OH, ethyl,

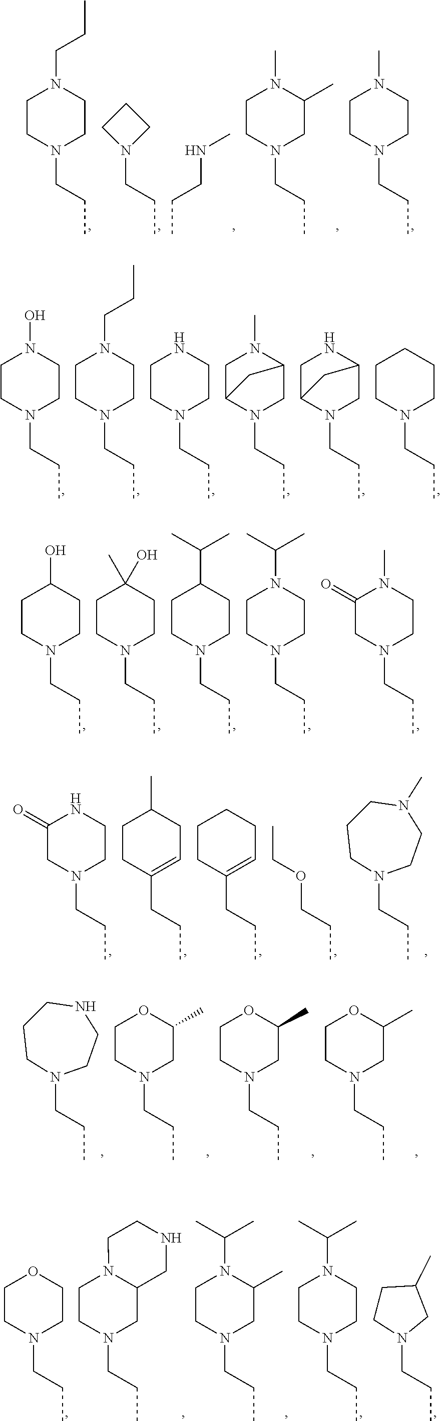

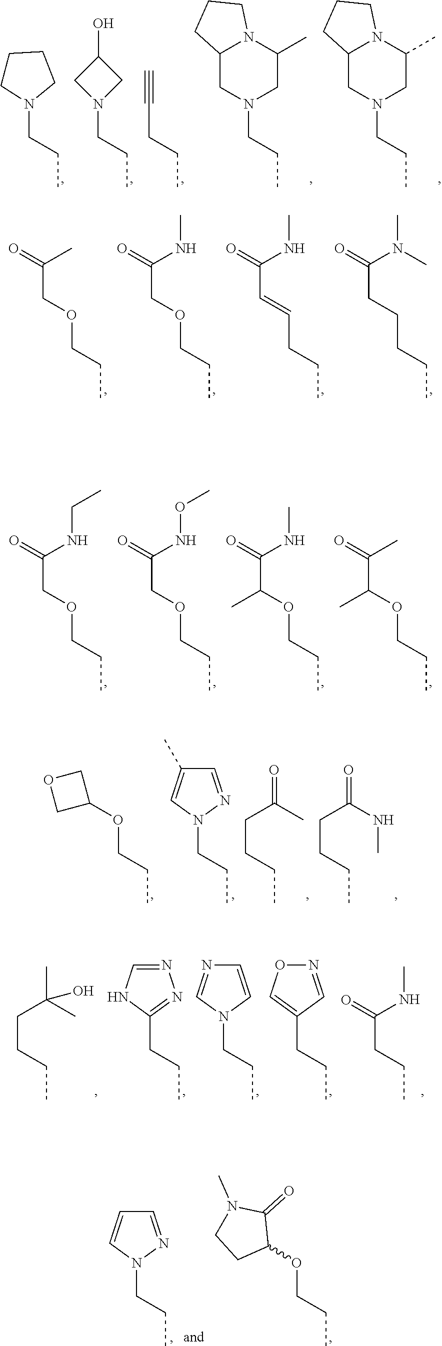

and wherein [symbol] represents a bond that may be stereospecific ((R) or (S)) or non-stereospecific; or a combination thereof.

In one embodiment described herein, the R_{PTM1} or R_{PTM2} or the corresponding location of any PTM described herein (e.g. PTMI or PTMII and derivatives thereof) is a linear or branched C1-C8 alkyl optionally substituted with OH.

In one embodiment described herein, the R_{PMT1} or R_{PTM2} or the corresponding location of any PTM described herein (e.g. PTMI or PTMII and derivatives thereof) is H, OH, CN, optionally substituted linear or branched C1-C4 alkyl, O-optionally substituted linear or branched C1-C4 alkyl, an optionally substituted C1-C4 alkynyl, an optionally substituted C1-C4 alkyne, an optionally substituted monocylic or bicyclic C3-C12 heterocyclyl (e.g., an optionally substituted C3-C12 monocyclic or bicyclic heterocycloalkyl, such as an C3-C12 monocylcic or bicyclic heterocycloalkyl, azetidine1-yl, pyrrolidin-1-yl, piperidin-1yl, piperazin-1-yl, or morpholin-4-yl, or homopiperazin-1-yl, each optionally substituted with one or more of OH, a linear or branched C1-C5 alkyl or NH₂), or an optionally substituted —O—C₃₋₁₂ monocylic or bicyclic heterocyclyl (e.g., an optionally substituted —O—C₃₋₁₂ monocyclic or bicyclic heterocycloalkyl, such as —O—C₃₋₁₂ monocylcic or bicyclic heterocycloalkyl optionally substituted with at least one OH, a linear or branched C1-C5 alkyl or NH₂), or an optionally substituted C3-C12 member ring (e.g., an optionally substituted C3-C12 non-aryl membered ring optionally substituted with one or more of OH, linear or branched C1-C5 alkyl, or NH₂), wherein when R_{PTM2} is a ring structure it is optionally covalently linked to Q₁₆ via a C or N of the R_{PTM2} ring.

In one embodiment described herein, the PTM is represented by a chemical structure selected from:

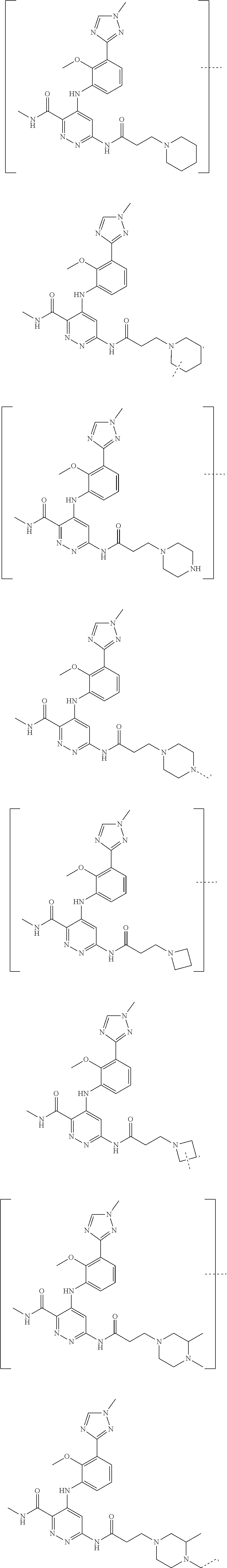

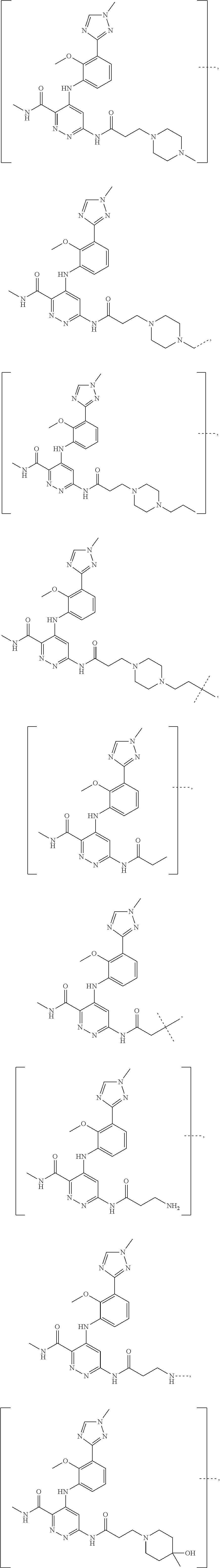

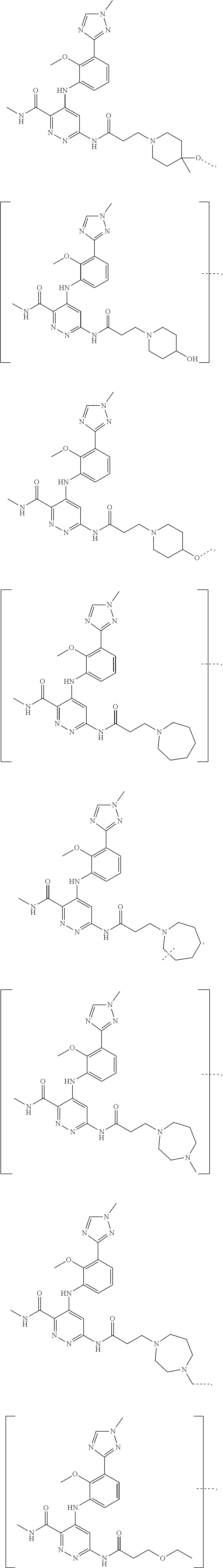

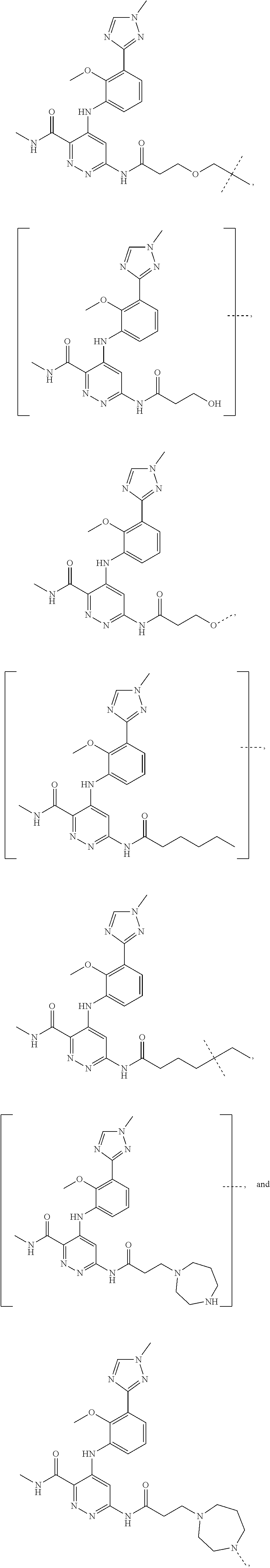

wherein [symbol] of the PTM indicates a point of attachment of a linker group (L) or a ULM, and wherein the PTM is coupled to at least one linker or ULM.

In one embodiment described herein, the PTM is selected from:

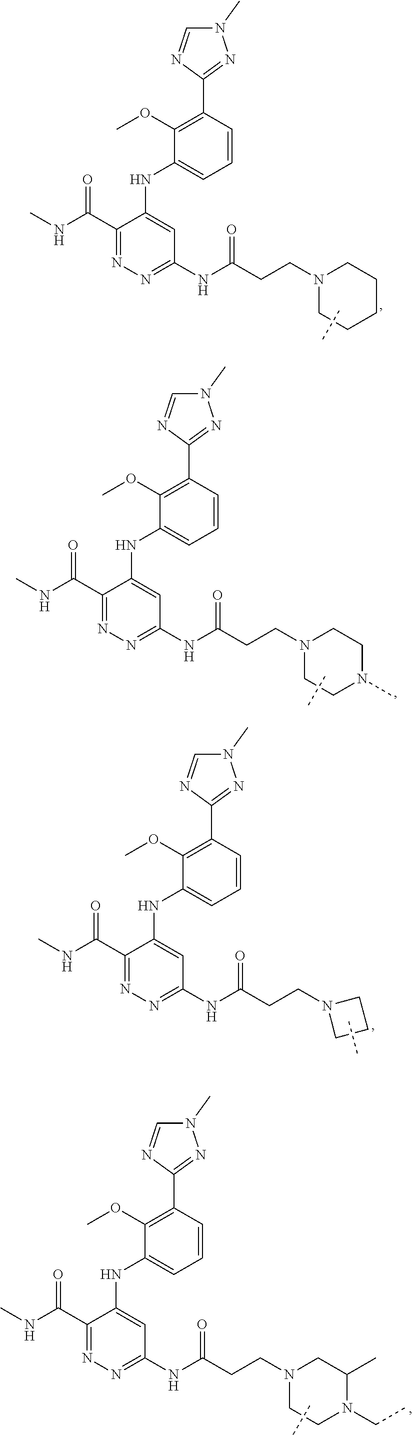

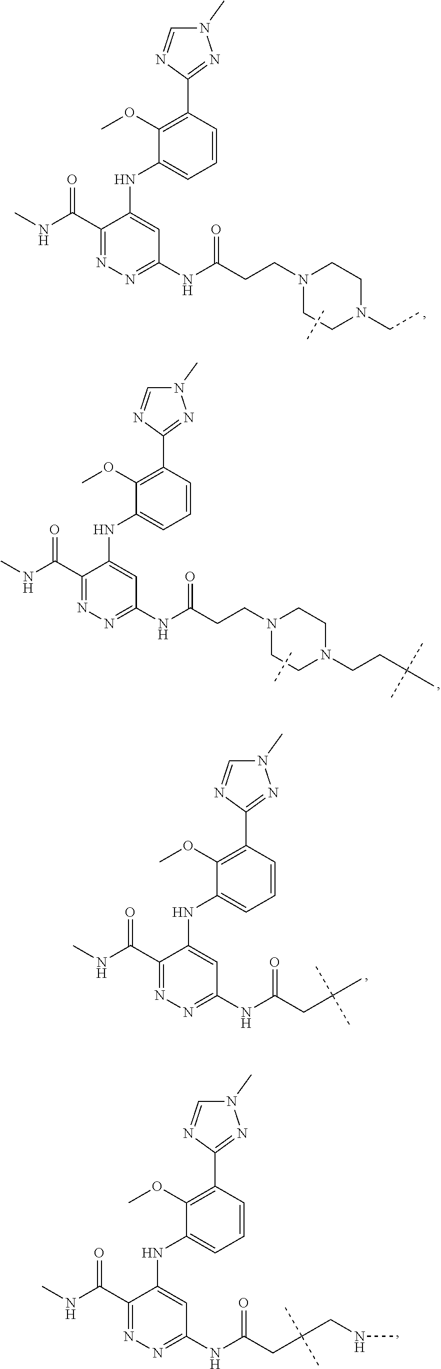

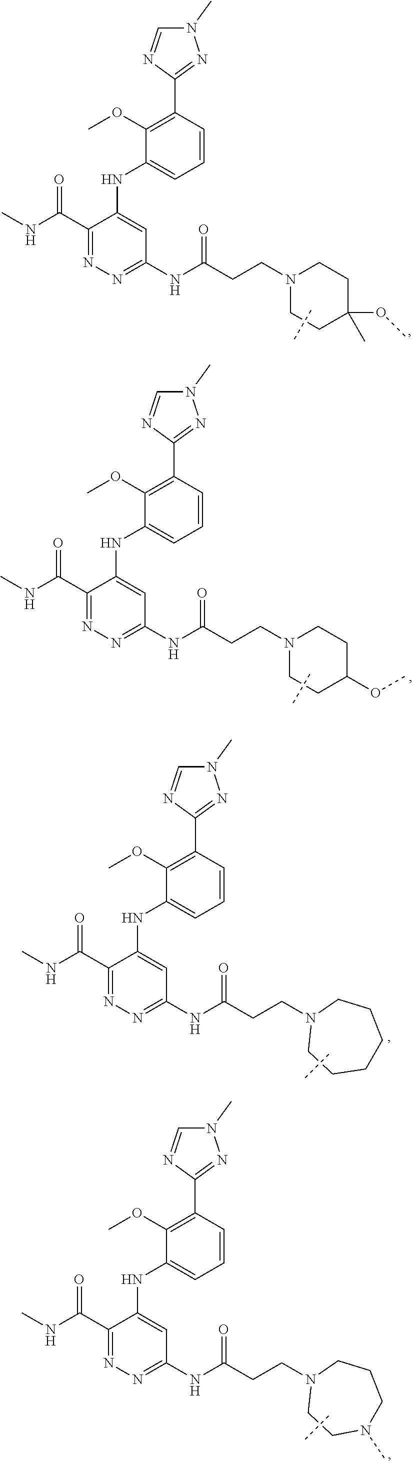

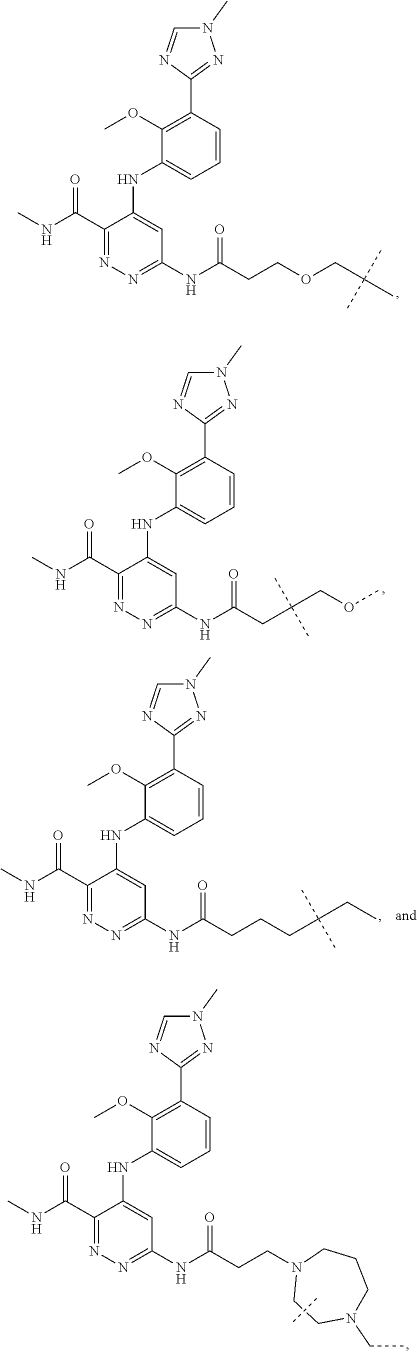

wherein [symbol] of the PTM indicates the point of attachment of a linker group (L) or a ULM, and wherein each PTM is coupled to at least one linker or ULM.

In one embodiment described herein, the PTM is selected from:

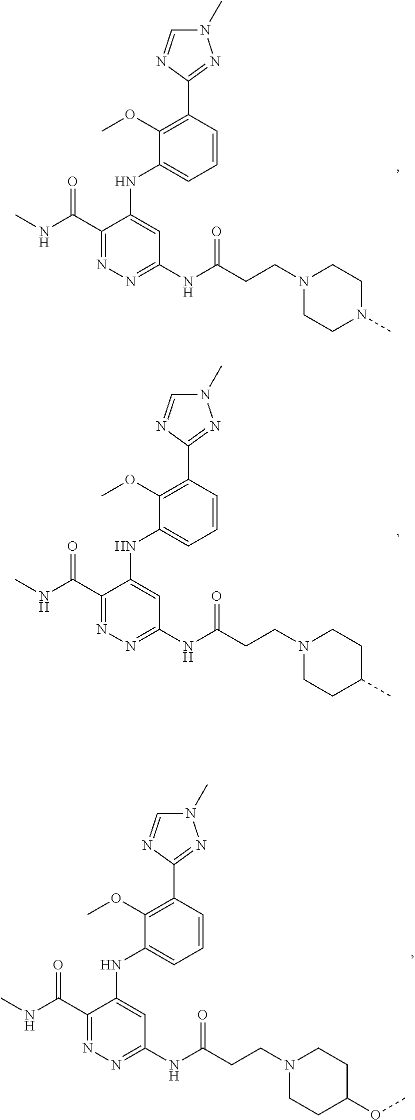

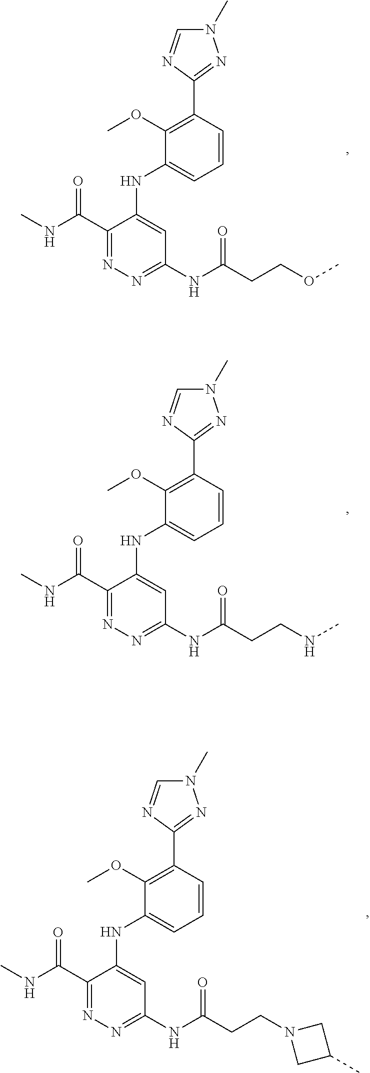

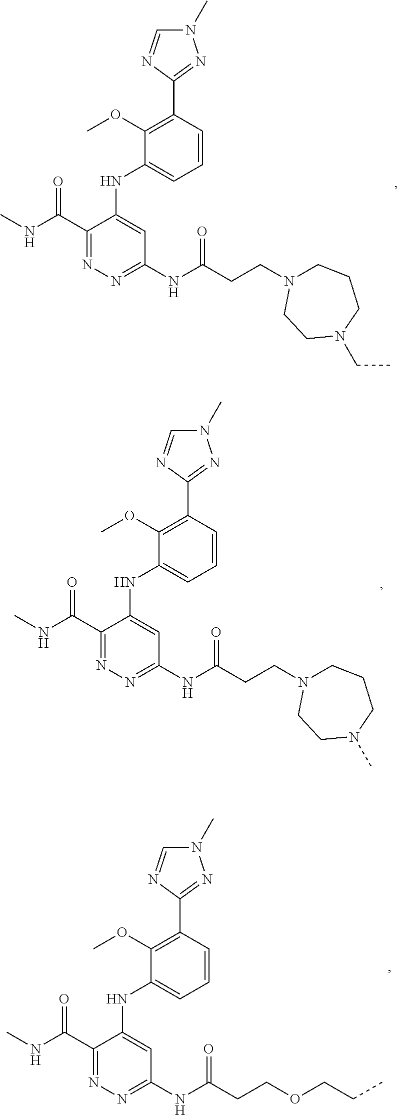

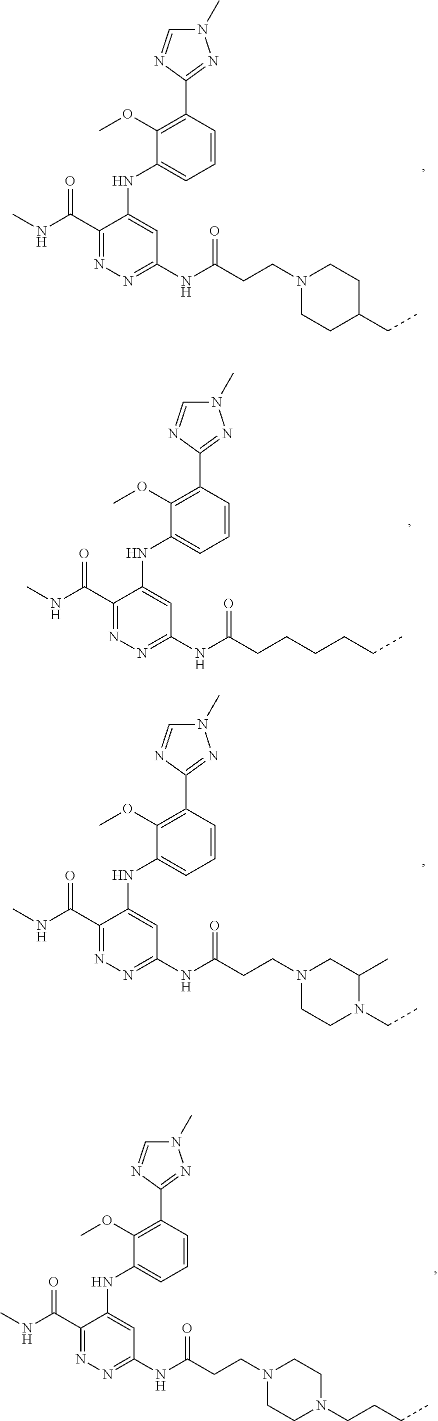

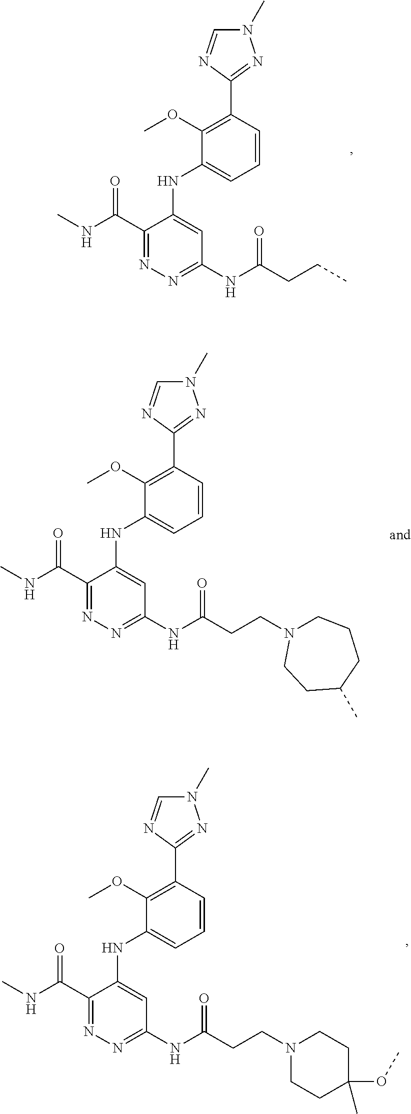

wherein [symbol] of the PTM indicates the point of attachment of a linker group (L) or a ULM.

In one embodiment described herein, the PTM is selected from:

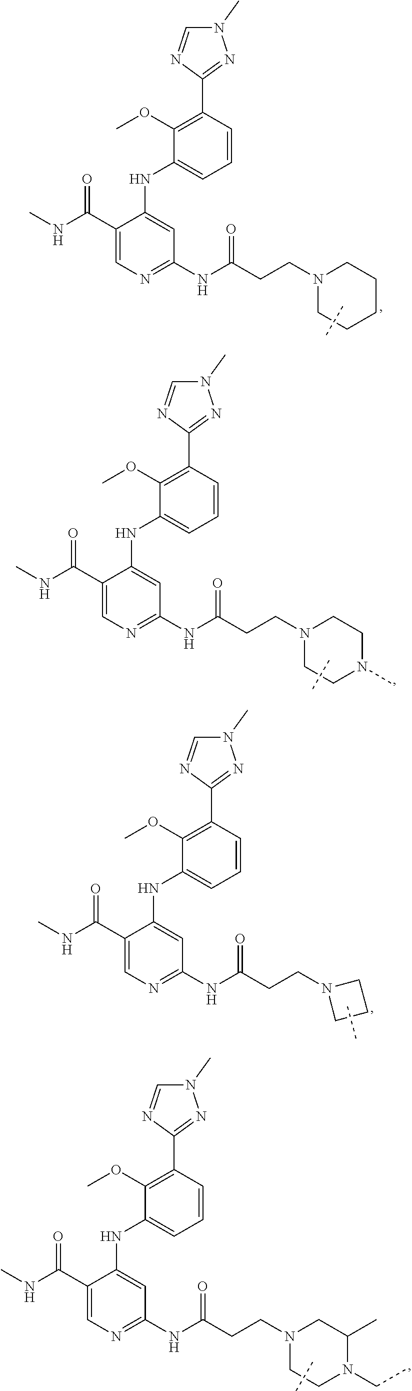

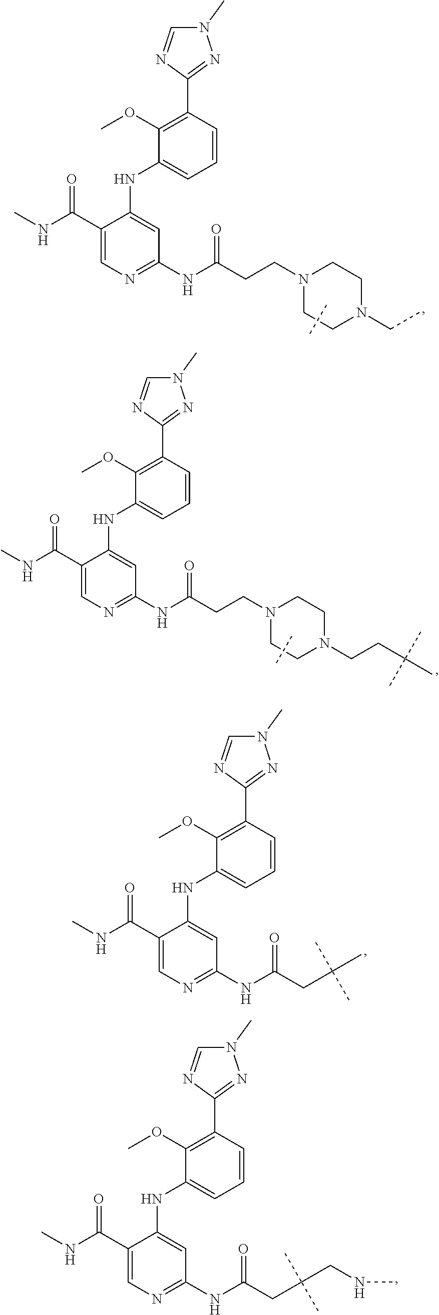

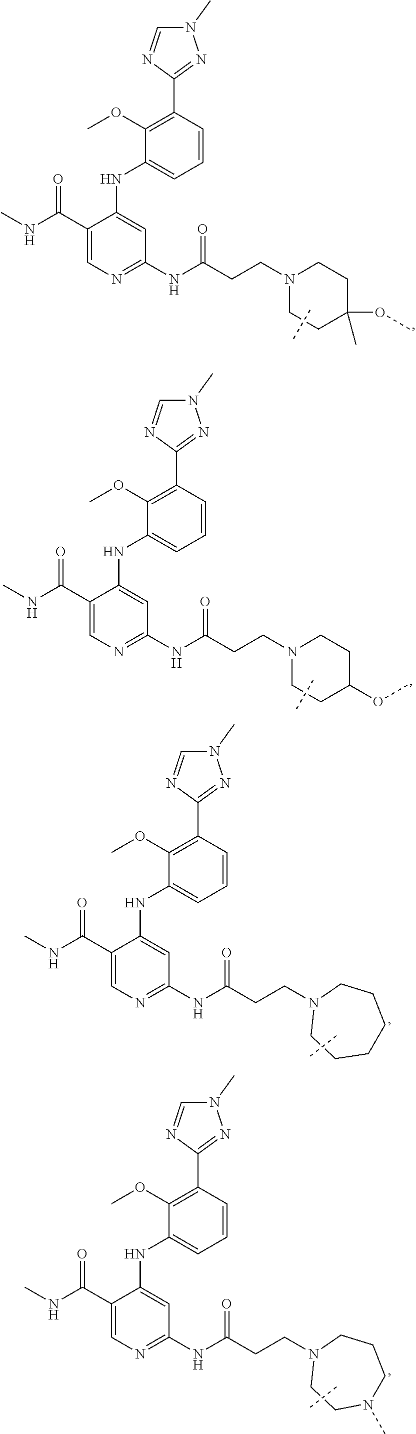

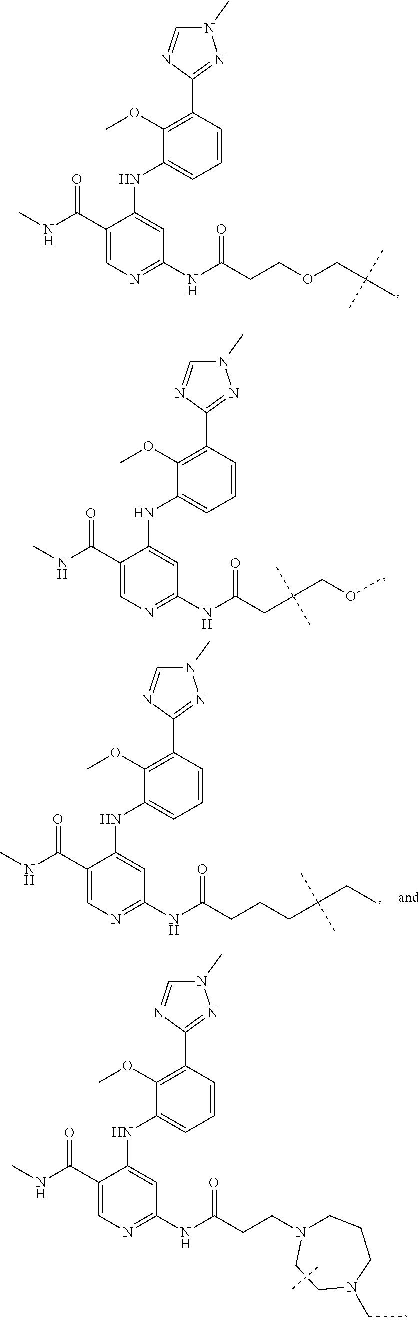

wherein [symbol] of the PTM indicates the point of attachment of a linker group (L) or a ULM, and wherein each PTM is coupled to at least one linker or ULM.

In one embodiment described herein, the PTM is selected from:

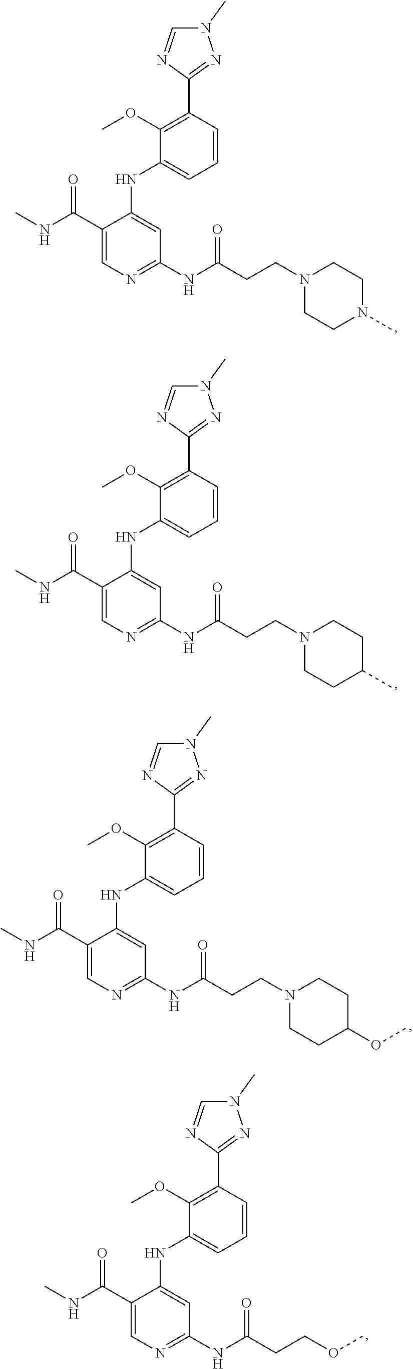

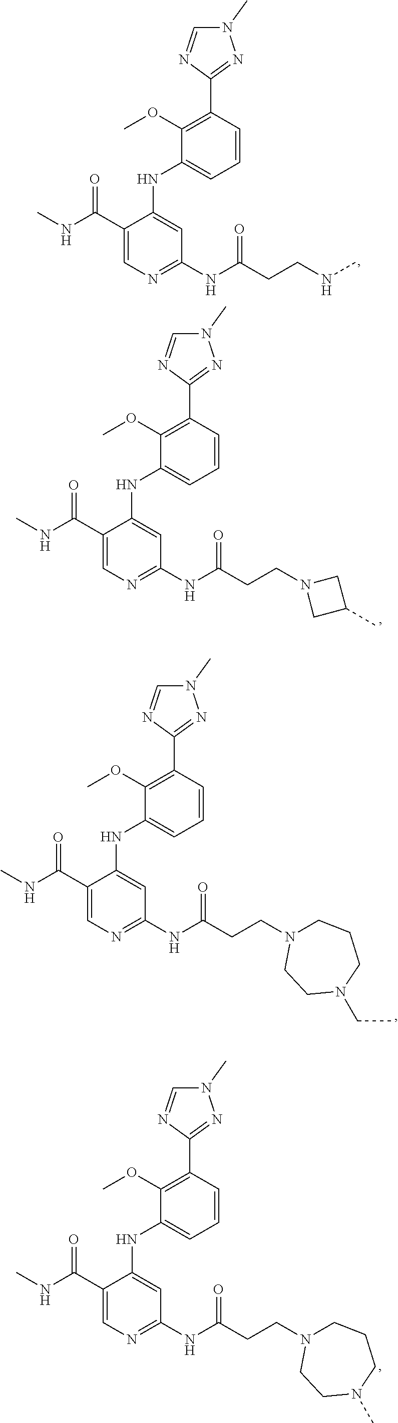

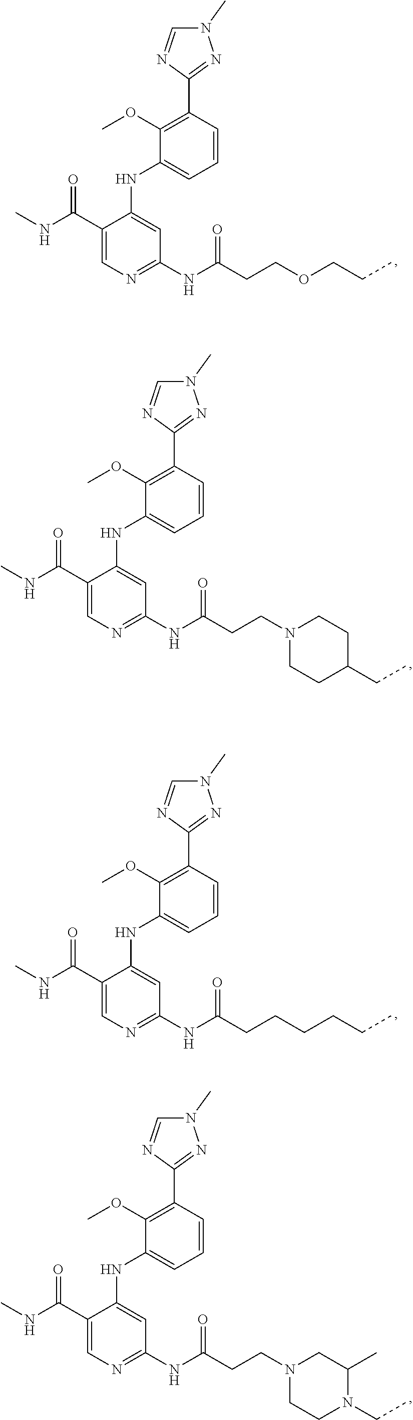

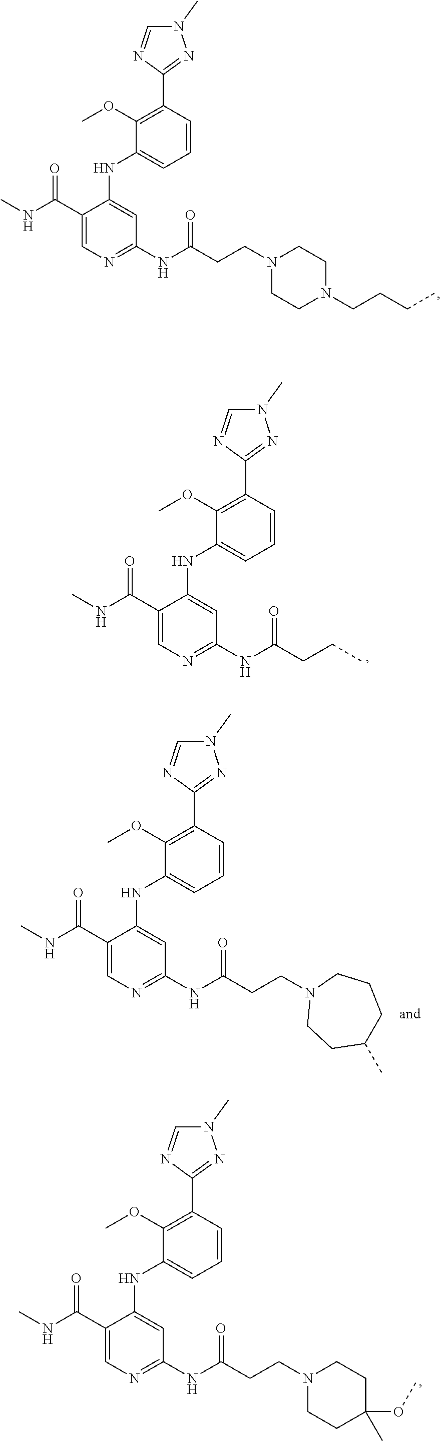

wherein [symbol] of the PTM indicates the point of attachment of a linker group (L) or a ULM.

### Therapeutic Compositions

The present invention further provides pharmaceutical compositions comprising therapeutically effective amounts of at least one bifunctional compound as described herein, in combination with a pharmaceutically acceptable carrier, additive or excipient.

In an additional aspect, the description provides therapeutic compositions comprising an effective amount of a compound as described herein or salt form thereof, and a pharmaceutically acceptable carrier, additive or excipient, and optionally an additional bioactive agent. The therapeutic compositions effect targeted protein degradation in a patient or subject, for example, an animal such as a human, and can be used for treating or ameliorating disease states or conditions which are modulated by degrading the target protein. In certain embodiments, the therapeutic compositions as described herein may be used to effectuate the degradation of protein for the treatment or amelioration of TYK2-mediated inflammatory diseases, autoimunune diseases or cancer. In certain additional embodiments, the disease is systemic lupus erythematosus (SLE), type 1 diabetes (ID), rheumatoid arthritis, psoriasis, Crohn's disease, inflammatory bowel disease, ulcerative colitis, or multiple sclerosis (MS).

In alternative aspects, the present disclosure relates to a method for treating a disease state or ameliorating one or more symptoms of a disease or condition in a subject in need thereof by degrading the TYK2 protein comprising administering to said patient or subject an effective amount, e.g., a therapeutically effective amount, of at least one compound as described herein, optionally in combination with a pharmaceutically acceptable carrier, additive or excipient, and optionally coadministered with an additional bioactive agent, wherein the composition is effective for treating or ameliorating the disease or disorder or one or more symptoms thereof in the subject. The method according to the present disclosure may be used to treat certain disease states or conditions including autoimmune diseases, by virtue of the administration of effective amounts of at least one compound described herein.

The present disclosure further includes pharmaceutical compositions comprising a pharmaceutically acceptable salt, in particular, acid or base addition salts of compounds as described herein. The acids which are used to prepare the pharmaceutically acceptable acid addition salts of the aforementioned compounds useful according to this aspect are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others.

Pharmaceutically acceptable base addition salts may also be used to produce pharmaceutically acceptable salt forms of the compounds according to the present disclosure. The chemical bases that may be used as reagents to prepare pharmaceutically acceptable base salts of the present compounds are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium, zinc and magnesium), ammonium or water-soluble amine addition salts such as N-methylglucamine-(meglumine), and the lower alkanolammonium and other base salts of pharmaceutically acceptable organic amines, among others.

The compounds as described herein may, in accordance with the disclosure, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual, intra nasal, intra ocular, intrathecal, and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present disclosure as sprays, mists, or aerosols for intra-nasal, intra-tracheal or pulmonary administration may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. Sustained or controlled release forms are preferably administered orally, but also in suppository and transdermal or other topical forms. Intramuscular injections in liposomal form or in depot formulation may also be used to control or sustain the release of compound at an injection site.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions as described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient may be combined with emulsifying and suspending agents. If desired, certain sweetening, flavoring or coloring agents may also be added.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The pharmaceutical compositions as described herein may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. In certain preferred aspects of the disclosure, the compounds may be coated onto a stent which is to be surgically implanted into a patient in order to inhibit or reduce the likelihood of occlusion occurring in the stent in the patient.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions as described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of active pharmaceutical ingredient in a pharmaceutical composition as described herein that may be combined with the carrier materials to produce a single dosage form will vary depending upon the condition of the subject and disease treated, as well as the particular mode of administration. Preferably, the compositions should be formulated to contain between about 0.05 milligram and about 750 milligrams or more, more preferably about 1 milligram to about 600 milligrams, and even more preferably about 10 milligrams to about 500 milligrams of active ingredient, alone or in combination with another compound according to the present disclosure.

It should also be understood that a specific dosage and treatment regimen for any particular patient will depend upon a variety of factors, including the activity and bioavailability of the specific compound employed, the age, body weight, general health, sex, diet, time of administration, rate of excretion, drug combination, and the judgment of the treating physician and the severity of the particular disease or condition being treated.

A patient or subject in need of therapy using compounds according to the methods described herein can be treated by administering to the patient (subject) an effective amount of the compound according to the present disclosure depending upon the pharmaceutically acceptable salt, solvate or polymorph, thereof optionally in a pharmaceutically acceptable carrier or diluent, either alone, or in combination with another known therapeutic agent.

The active compound is combined with the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 nanograms per kilograms (ng/kg) to 300 milligrams per kilograms (mg/kg), preferably 0.1 to 100 mg/kg per day, more generally 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to a dosage form containing less than 1 milligrams (mg), 1 mg to 3000 mg, or 5 mg to 500 mg of active ingredient per unit dosage form. An oral dosage of about 25 mg-250 mg is often convenient.

The active ingredient is preferably administered to achieve peak plasma concentrations of the active compound of about 0.00001-30 millimole (mM), preferably about 0.1-30 micromole (μM). This may be achieved, for example, by the intravenous injection of a solution or formulation of the active ingredient, optionally in saline, or an aqueous medium or administered as a bolus of the active ingredient. Oral administration may also be appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as anti-cancer agents, as described herein among others. In certain preferred aspects of the disclosure, one or more compounds according to the present disclosure are coadministered with another bioactive agent, such as an anti-cancer agent or a wound healing agent, including an antibiotic, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

### Therapeutic Methods

In an additional aspect, the description provides therapeutic methods comprising administration of an effective amount of a compound as described herein or salt form thereof, and a pharmaceutically acceptable carrier. The therapeutic methods are useful to effect protein degradation in a patient or subject in need thereof, for example, an animal such as a human, for treating or ameliorating a disease state, condition or related symptom that me be treated through targeted protein degradation.

The terms “treat”, “treating”, and “treatment”, etc., as used herein, refer to any action providing a benefit to a patient for which the present compounds may be administered, including the treatment of any disease state, condition, or symptom which is related to the protein to which the present compounds bind. Disease states or conditions, including cancer, which may be treated using compounds according to the present disclosure are set forth hereinabove.

The description provides therapeutic methods for effectuating the degradation of proteins of interest for the treatment or amelioration of a disease, e.g., cancer. In one embodiment, the disease is multiple myeloma. As such, in another aspect, the description provides a method of ubiquitinating/degrading a target protein in a cell. In certain embodiments, the method comprises administering a bifunctional compound of the invention. The control or reduction of specific protein levels in cells of a subject as afforded by the present disclosure provides treatment of a disease state, condition, or symptom. In one embodiment, the method comprises administering an effective amount of a compound as described herein, optionally including a pharmaceutically acceptable excipient, carrier, adjuvant, another bioactive agent or combination thereof.

In additional embodiments, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In another aspect, the description provides methods for identifying the effects of the degradation of proteins of interest in a biological system using compounds according to the present disclosure.

In another aspect, the description provides a process for making a molecule that can cause degradation of TYK2 in a cell, comprising the steps of: i. providing a small molecule that binds to the pseudokinase site of TYK2; ii. providing and E3 ubiquitin ligase binding moiety (ULM), preferably a CLM such as thalidomide, pomalidomide, lenalidomide or an analog thereof; and iii. covalently coupling the small molecule of step (i) to the ULM of step (ii) via a chemical linking group (L) to form a compound which binds to both a cereblon E3 ubiquitin ligase and TYK2 protein in the cell, such that the cereblon E3 ubiquitin ligase is in proximity to, and ubiquitinates TYK2 protein bound thereto, such that the ubiquitinated TYK2 is then degraded.

In another aspect, the description provides a method for detecting whether a molecule can trigger degradation of a TYK2 protein in a cell, the method comprising the steps of: (i) providing a molecule for which the ability to trigger degradation of TYK2 protein in a cell is to be detected, said molecule comprising the structure: CLM-L-PTM, wherein CLM is a cereblon E3 ubiquitin ligase binding moiety capable of binding a cereblon E3 ubiquitin ligase in a cell, which CLM is thalidomide, pomalidomide, lenalidomide, or an analog thereof; PTM is a protein targeting moiety, which is a small molecule that binds to TYK2, said TYK2 having at least one lysine residue available to be ubiquitinated by a cereblon E3 ubiquitin ligase bound to the CLM of the molecule; and L is a chemical linking group that covalently links the CLM to the PTM to form the molecule; (ii) incubating a TYK2 protein-expressing cell in the presence of the molecule of step (i); and (iii) detecting whether the TYK2 protein in the cell has been degraded.

In any of the aspects or embodiments described herein, the small molecule capable of binding TYK2, is a small molecule that binds the pseudokinase domain (JH2) of TYK2. In certain embodiments, the small molecule that binds the pseudokinase domain of TYK2 is as described herein.

In another aspect of said treatment, the present disclosure provides a method of treating a human patient in need of said treatment of a disease state, condition, or symptom causally related to TYK2 expression, over-expression, mutation, misfolding or dysregulation where the degradation of the TYK2 protein will produce a therapeutic effect in the patient, the method comprising administering to the patient an effective amount of a compound according to the present disclosure, optionally in combination with another bioactive agent. The disease state, condition, or symptom may be caused by a microbial agent or other exogenous agent such as a virus, bacteria, fungus, protozoa or other microbe, or may be a disease state, which is caused by expression, overexpression, mutation, misfolding, or dysregulation of the protein, which leads to a disease state, condition, or symptom.

In another aspect, the present disclosure provides a method of treating or ameliorating at least one symptom of a disease or condition in a subject, comprising the steps of: providing a subject identified as having a symptom of a disease or condition causally related to expression, overexpression, mutation, misfolding, or dysregulation of TYK2 protein in the subject, and the symptom of the disease or condition is treated or ameliorated by degrading TYK2 protein in cells of the subject; and administering to the subject therapeutically effective amount of a compound comprising a small molecule of the present invention such that the TYK2 protein is degraded, thereby treating or ameliorating at least one symptom of a disease or condition in the subject.

The term “disease state or condition” is used to describe any disease state or condition wherein protein expression overexpression, mutation, misfolding, or dysregulation (e.g., the amount of protein expressed in a patient is elevated) occurs and where degradation of the TYK2 protein to reduce or stabilize the level of TYK2 protein (whether mutated or not) in a patient provides beneficial therapy or relief of symptoms to a patient in need thereof. In certain instances, the disease state, condition, or symptom may be cured.

Disease state, condition, or symptom which may be treated using compounds according to the present disclosure include, for example, inflammatory conditions, and immunological conditions, asthma, autoimmune diseases such as multiple sclerosis, various cancers, ciliopathies, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKD1) or 4 (PKD2) Prader-Willi syndrome, Sickle-cell disease, Tay-Sachs disease, and Turner syndrome.

The term “autoimmune diseases” is used throughout the specification to refer to the pathological process that results in immune system over activity, where the body attacks and damages its own tissues, and which may result in a reduction of the body's ability to fight infections resulting in vulnerability to infections. Exemplary autoimmune diseases which may be treated by the present compounds either alone or in combination with at least one additional anti-autoimmune disease therapeutic agent include therapeutic agents to treat systemic lupus erythematosus (SLE), type 1 diabetes (TID), multiple sclerosis (MS), rheumatoid arthritis, psoriasis. Crohn's disease, inflammatory bowel disease, and ulcerative colitis. For example, infliximab, tofacitinib, adalimumab, etanercept, golimumab, and certolizumab pepol.

The term “bioactive agent” is used to describe an agent, other than a compound according to the present disclosure, which is used in combination with a present compound as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

The term “additional anti-autoimmune disease agent” is used to describe an anti-autoimmune disease therapeutic agent, which may be combined with a compound according to the present disclosure to treat autoimmune disease. These agents include, for example, infliximab, tofacitinib, baricitinib, secukinumab, adalimumab, etanercept, golimumab, certolizumab pepol, anti-proliferative drugs (for example, mycophenolate mofetil) and corticosteroids.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

### EXAMPLES

### Abbreviations

ACN Acetonitrile
AcOH Acetic acid
Boc tert-butoxycarbonyl
dba Dibenzylideneacetone
DBU 1,8-Diazabicyclo[5.4.0]undec-7-ene
DCM Dichloromethane
DMA Dimethylacetamide
DME Dimethoxyethane
DMF Dimethylformamide
DMSO Dimethyl Sulfoxide
DMAC/DMA Dimethylacetamide
DIEA N, N-Diisopropylethylamine
EDTA Ethylenediaminetetraacetic acid
EtOAc/EA Ethyl Acetate
EtOH Ethanol
FA Formic Acid
HPLC High pressure liquid chromatography
Hz Hertz
IBX 2-Iodoxybenzoic acid
LAH Lithium aluminium hydride
LCMS Liquid Chromatography/Mass Spectrometry
LiHMDS Lithium bis(trimethylsilyl)amide
MHz Megahertz
NBS N-Bromosuccinimide
NCS N-Chlorosuccinimide
NMR Nuclear Magnetic Resonance
NMP N-Methyl-2-pyrrolidone
MeOH Methanol
MPLC Medium pressure liquid chromatography
MTBE Methyl tert-butyl ether
PE Petroleum ether
Psi Pound-force per square inch
RT or r.t. Room temperature
SFC Supercritical fluid chromatography
TEA Triethylamine
THF Tetrahydrofuran
TFA Trifluoracetic acid
TLC Thin layer chromatography
TMS Trimethylsilyl

### General Synthetic Approach

The synthetic realization and optimization of the bifunctional molecules as described herein may be approached in a stepwise or modular fashion. For example, identification of compounds that bind to the target protein, i.e., TYK2 can involve high or medium throughput screening campaigns if no suitable ligands are immediately available. It is not unusual for initial ligands to require iterative design and optimization cycles to improve suboptimal aspects as identified by data from suitable in vitro and pharmacological and/or ADMET assays. Part of the optimization/SAR campaign would be to probe positions of the ligand that are tolerant of substitution and that might be suitable places on which to attach the chemical linking group previously referred to herein. Where crystallographic or NMR structural data are available, these can be used to focus such a synthetic effort.

In a very analogous way one can identify and optimize ligands for an E3 Ligase.

With PTMs and ULMs (e.g. CLMs) in hand, one skilled in the art can use known synthetic methods for their combination with or without a chemical linking group(s). Chemical linking group(s) can be synthesized with a range of compositions, lengths and flexibility and functionalized such that the PTM and ULM groups can be attached sequentially to distal ends of the linker. Thus, a library of bifunctional molecules can be realized and profiled in in vitro and in vivo pharmacological and ADMET/PK studies. As with the PTM and ULM groups, the final bifunctional molecules can be subject to iterative design and optimization cycles in order to identify molecules with desirable properties.

In some instances, protecting group strategies and/or functional group interconversions (FGIs) may be required to facilitate the preparation of the desired materials. Such chemical processes are well known to the synthetic organic chemist and many of these may be found in texts such as “Greene's Protective Groups in Organic Synthesis” Peter G. M. Wuts and Theodora W. Greene (Wiley), and “Organic Synthesis: The Disconnection Approach” Stuart Warren and Paul Wyatt (Wiley).

### Synthetic Procedures

### Synthesis of Intermediate 1: 6-acrylamido-4-((2-methoxy-3-(1-methyl-1H-1,2,4-triazol-3-yl)phenyl)amino)-N-methylpyridazine-3-carboxamide

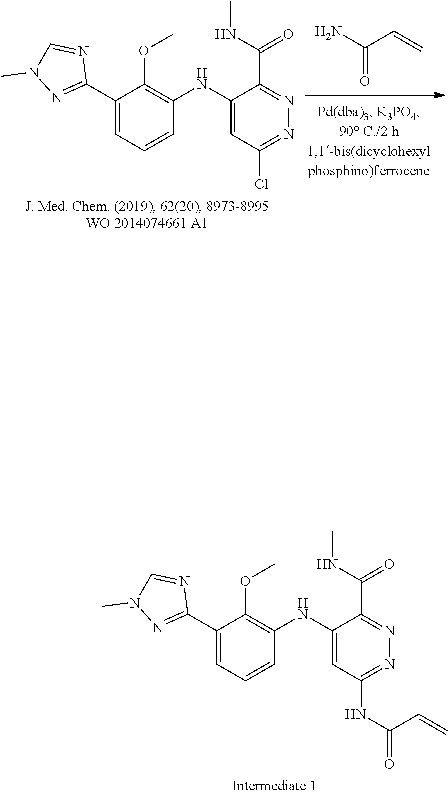

A mixture of 6-chloro-4-((2-methoxy-3-(1-methyl-1H-1,2,4-triazol-3-yl)phenyl)amino)-N-methylpyridazine-3-carboxamide (1 grams (g), 2.68 millimoles (mmol)) and acrylamide (0.23 g, 3.22 mmol) were taken up in 1,4-dioxane (5 milliliters (mL)) and nitrogen bubbled through the slurry for about 10-15 min. Pd₂(dba)₃ (0.24 g, 0.268 mmol) and 1,1′-bis(dicyclohexylphosphino)ferrocene (0.3 g, 0.54 mmol) were then added with a continued nitrogen flow for 5 min. An aqueous solution of potassium phosphate tribasic (2 molar (M), 2.7 mL, 5.36 mmol) was then added in one portion and the reaction heated to 95° C. for 2 hours (h). The reaction was allowed to cool to room temperature before diluting with DCM (50 mL) and water (20 mL). The organic layer was dried, concentrated and the crude product purified by column chromatography on silica (eluted with MeOH in DCM) to afford the desired product 6-acrylamido-4-((2-methoxy-3-(1-methyl-1H-1,2,4-triazol-3-yl)phenyl)amino)-N-methylpyridazine-3-carboxamide (270 mg, 25% yield) as a yellow solid. LCMS: (ES⁺): m/z 409.2 [M+1]⁺, retention time (t_{R})=3.20 min

Chemical Formula: C₁₉H₂₀N₈O₃; Molecular Weight: 408.41.

### Synthesis of Intermediate 2: Benzyl 4-(1-(2-(2,6-dioxopiperidin-3-yl)-1,3-dioxoisoindolin-5-yl)azetidin-3-yl)piperidine-1-carboxylate

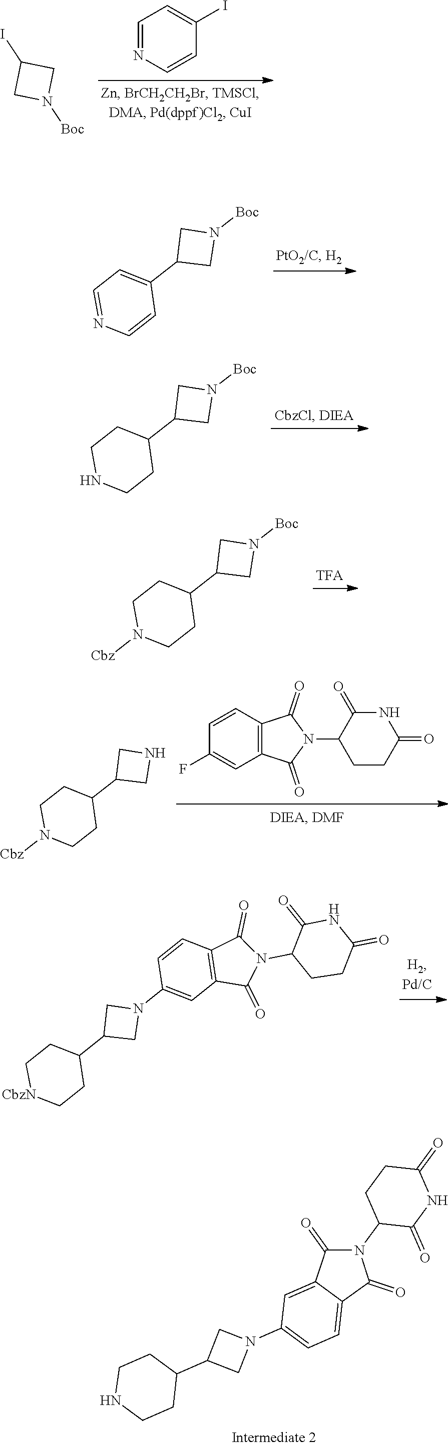

### Step 1: Tert-butyl 3-(pyridin-4-yl)azetidine-1-carboxylate

A 1-liter 3-neck round bottom flask equipped with a thermocouple, magnetic stirrer, condenser, heating mantle, and N₂ inlet adapter was charged with anhydrous dimethylacetamide (DMA, 50 mL) and zinc (2.8 g, 4.4 mmol). The mixture was stirred at 20° C. while a mixture of 1,2-dibromoethane (DBE, 653 milligrams (mg), 3 mmol) and trimethylsilyl chloride (TMS-C1, 381 mg, 3.5 mmol) was added at a rate to maintain the temperature below 65° C. over 30 min. The resulting slurry was aged for 15 min. A solution of tert-butyl 3-iodoazetidine-1-carboxylate (5 g, 17 mmol) in DMA (20 mL) was added dropwise over 1 h at a rate to maintain the temperature below 65° C. and the milky suspension was stirred for 30 min while slowly cooling to 200. Another 3-liter 4-neck round bottom flask equipped a thermocouple, mechanical stirrer, condenser, heating mantle, and N₂ inlet adapter was charged with [1,1′-bis(diphenylphosphino)ferrocene]dichloropalladium(II) dichloromethane complex (646 mg, 0.88 mmol), cuprous iodide (334 g, 0.44 mmol), and 4-iodopyridine 1b (3.6 g, 17 mmol) in DMA (25 mL) under N₂. The resulting mixture was degassed with alternate vacuum/N₂ purges. The above prepared zinc iodide reagent of compound 1a in DMA was added as a suspension. The mixture was degassed with vacuum/N₂ twice and then heated to 80° C. The progress of the reaction was monitored by HPLC and LC-MS and was complete after 2 h. The reaction mixture was cooled to 40° C. EtOAc (160 ml) was added and the mixture was stirred for 10 min. The insoluble material (excess Zn and Cu complexes/salts) was removed by passing through a diatomaceous earth pad, which was washed with EtOAc (20 mL×2). The combined filtrate was stirred with 1 N aqueous NH₄Cl (100 ml) at 20° C. for 30 min and the aqueous layer (pH=5-6) was adjusted to pH=9-10 using 3 N aqueous NaOH solution (˜48 mL) while a significant amount of brown precipitate was formed. The precipitate was removed by paper filtration and was washed with deionized water (10 mL). The separated aqueous phase was extracted with EtOAc (100 ml), and the combined organic phases were treated with saturated aqueous NH₄Cl (80 ml×2) and stirred for 15 min (repeated again), washed with 5 percent aqueous NaHSO₃ (50 mL) and brine (100 ml), and dried over MgSO₄. The crude product was chromatographed on silica gel (PE/EA 10:1→1:1) to give tert-butyl-3-(pyridin-4-yl)azetidine-1-carboxylate (200 mg, 5% yield) as a yellow oil.

Chemical Formula: C₁₃H₁₈N₂O₂; Molecular Weight: 234.30

LCMS: (ES+): m/z 235.3 [M+1]+, t_{R}=2.78 min.

### Step 2: Tert-butyl 3-(piperidin-4-yl)azetidine-1-carboxylate

A mixture of tert-butyl-3-(pyridin-4-yl)azetidine-1-carboxylate (200 mg, 0.85 mmol), PtO₂/C (50 mg) and CH₃COOH (0.1 ml) in EtOH (10 mL) was stirred at 50° C. for 3 h under H₂. The mixture was filtered through a Celite pad, and the filtrate was concentrated to give the crude product tert-butyl 3-(piperidin-4-yl)azetidine-1-carboxylate (150 mg, 74% yield) as a white solid.

Chemical Formula: C₁₃H₂₄N₂O₂; Molecular Weight: 240.35

LCMS: (ES+): m/z 241.3 [M+1]+, t_{R}=2.57 min.

### Step 3: benzyl 4-(1-(tert-butoxycarbonyl)azetidin-3-yl)piperidine-1-carboxylate

A solution of tert-butyl 3-(piperidin-4-yl)azetidine-1-carboxylate (300 mg, 1.2 mmol) and DIEA (500 mg, 3.75 mmol) in DCM (200 mL) was stirred at 0° C., then CbzCl (320 mg, 1.87 mmol) was added dropwise. The mixture was stirred at RT (room temperature) for 1 hour (h). H₂O (5 mL) was added and the mixture was extracted with ethyl acetate (300 mL). The organic phase was concentrated under vacuum to afford crude product. The crude product was be purified by preparative TLC to give benzyl 4-(1-(tert-butoxycarbonyl)azetidin-3-yl)piperidine-1-carboxylate (150 mg, 33% yield) as a colorless oil.

Chemical Formula: C₂₁H₃₀N₂O₄; Molecular Weight: 374.48

LCMS: (ES+): m/z 375.4 [M+1]+, t_{R}=4.41 min.

### Step 4: Benzyl 4-(azetidin-3-yl)piperidine-1-carboxylate

A solution of benzyl 4-(1-(tert-butoxycarbonyl)azetidin-3-yl)piperidine-1-carboxylate (200 mg, 0.53 mmol) in DCM (4 mL) and TFA (1 mL) was stirred at RT for 2 h. The organic phase was concentrated under vacuum to afford the desired product benzyl 4-(azetidin-3-yl)piperidine-1-carboxylate (160 mg, 58% yield) as a white solid.

### Step 5: Benzyl 4-(1-(2-(2,6-dioxopiperidin-3-yl)-1,3-dioxoisoindolin-5-yl)azetidin-3-yl)piperidine-1-carboxylate

A solution of benzyl 4-(azetidin-3-yl)piperidine-1-carboxylate (1 g, 3.63 mmol), 2-(2,6-dioxopiperidin-3-yl)-5-fluoroisoindoline-1,3-dione (1 g, 3.63 mmol) and DIEA (1.4 g 10.9 mmol) in DMF (20 mL) was stirred 130° C. for 1 hour. H₂O (50 mL) was added and the mixture was extracted with ethyl acetate (300 mL). The organic phase was concentrated under vacuum to afford crude product. The crude product was be purified by flash column chromatography on silica to give benzyl 4-(1-(2-(2,6-dioxopiperidin-3-yl)-1,3-dioxoisoindolin-5-yl)azetidin-3-yl)piperidine-1-carboxylate (1.2 g, 62% yield) as a yellow solid.

¹HNMR (400 MHz, MeOD-d₆): δ 11.06 (s, 1H), 7.64-7.62 (m, 1H), 7.39-7.29 (m, 5H), 6.75 (m, 1H), 6.63-6.61 (m, 1H), 5.06-5.02 (m, 3H), 4.09-4.02 (m, 4H), 3.82-3.73 (m, 2H), 3.58 (m, 2H), 2.91-2.79 (m, 3H), 2.67-2.55 (m, 1H), 2.07-1.98 (m, 1H), 1.67-1.65 (m, 3H), 1.03-0.95 (m, 2H).

Chemical Formula: C₂₉H₃₀N₄O₆; Molecular Weight: 530.58

LCMS: (ES+): m/z 531.2⁺, t_{R}=3.335.

### Step 6: 2-(2,6-Dioxopiperidin-3-yl)-5-(3-(piperidin-4-yl)azetidin-1-yl)isoindoline-1,3-dione

A solution of benzyl 4-(1-(2-(2,6-dioxopiperidin-3-yl)-1,3-dioxoisoindolin-5-yl)azetidin-3-yl)piperidine-1-carboxylate (100 mg, 0.188 mmol), Pd/C (100 mg, 10%) in THF (5 mL) was stirred at 25° C. under H₂ for 2 h. The reaction was filtered and the filtrate was concentrated to afford the desire product 2-(2,6-dioxopiperidin-3-yl)-5-(3-(piperidin-4-yl)azetidin-1-yl)isoindoline-1,3-dione (100 mg, crude) (=Intermediate 2) as a yellow oil.

Chemical Formula: C₂₁H₂₄N₄O₄; Molecular Weight: 396.45

LCMS: (ES⁺): m/z 397.1[M+1]⁺, t_{R}=2.738 min.

### Synthesis of Intermediate 3: 2-(2,6-Dioxopiperidin-3-yl)-5-(3-(piperidin-4-ylmethyl)azetidin-1-yl)isoindoline-1,3-dione

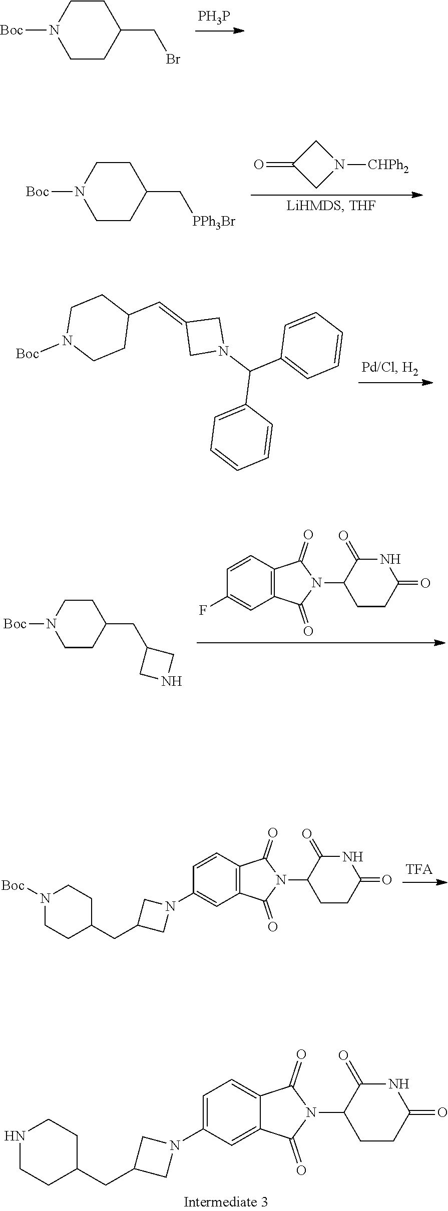

### Step 1: Tert-butyl 4-((bromotriphenyl-λ⁵-phosphaneyl)methyl)piperidine-1-carboxylate

A mixture of tert-butyl 4-(bromomethyl)piperidine-1-carboxylate (5.0 g, 18.0 mmol) and Ph₃P (7.1 g, 27.0 mmol) in toluene (5.0 mL) was heated to 130° C. for 4 h. After cooling to rt, the crude was slurried in tBuOCH₃ and the solid was filtered, dried at 45° C. under vacuum to afford a white solid tert-butyl 4-((bromotriphenyl-λ⁵-phosphaneyl)methyl)piperidine-1-carboxylate (4.4 g, 45% yield), which was used in the next reaction without further purification.

Chemical Formula: C₂₉H₃₅BrNO₂P; Molecular Weight: 540.48

¹HNMR (400 MHz, CDCl₃): 7.69-7.93 (m, 15H), 4.12-4.17 (br, 2H), 3.95-3.98 (m, 2H), 3.47-3.57 (m, 2H), 2.54 (br, 2H), 1.92-1.94 (br, 2H), 1.46 (s, 9H).

### Step 2: Tert-butyl 4-((1-benzhydrylazetidin-3-ylidene)methyl)piperidine-1-carboxylate

To a suspension of tert-butyl 4-((bromotriphenyl-λ⁵-phosphaneyl)methyl)piperidine-1-carboxylate (42 g, 77.7 mmol) in THF (300 mL) was added LiHMDS (1.0 M, 78 mL) at 0° C. for 30 minutes. After stirring for 1 h at rt, 1-benzhydrylazetidin-3-one (15.1 g, 63.6 mmol) in THF was added to the mixture at 0° C. After stirring for 1 h, the reaction was warmed to r.t. and stirred overnight. TLC showed reaction was completed. The reaction was quenched with AcOH/THF, taken up with EtOAc, washed with brine, dried, concentrated. The crude was purified by column chromatography on silica with PE/EA (30-15/1) to give the desired product tert-butyl 4-((1-benzhydrylazetidin-3-ylidene)methyl)piperidine-1-carboxylate (14.2 g, 53% yield) as a light yellow solid.

Chemical Formula: C₂₇H₃₄N₂O₂; Molecular Weight: 418.58

¹HNMR (400 MHz, CDCl₃): 7.70-7.75 (m, 4H), 7.25-7.44 (m, 4H), 7.16-7.20 (m, 2H), 5.05 (d, J=8.0 Hz, 1H), 4.48 (s, 1H), 4.09 (br, 2H), 3.80 (s, 2H), 3.74 (s, 2H), 2.66 (t, J=12.0 Hz, 2H), 1.99-2.04 (m, 1H), 1.54-1.62 (m, 2H), 1.46 (s, 9H), 1.21-1.24 (m, 2H).

### Step 3: Tert-butyl 4-(azetidin-3-ylmethyl)piperidine-1-carboxylate

A mixture of tert-butyl 4-((1-benzhydrylazetidin-3-ylidene)methyl)piperidine-1-carboxylate (14.2 g, 33.9 mmol) and Pd/C (wet, 10%, 8.0 g) in EtOH (100 mL) was stirred at r.t. overnight. The mixture was filtered through Celite, and the filtrate was concentrated, purified by column chromatography on silica with DCM/CH₃OH (20-5/1) to afford desired product of tert-butyl 4-(azetidin-3-ylmethyl)piperidine-1-carboxylate (3.7 g, ˜70% purity) as a light yellow oil.

Chemical Formula: C₁₄H₂₆N₂O₂; Molecular Weight: 254.37

¹HNMR (400 MHz, CDCl₃): 4.06 (br, 2H), 3.70 (t, J=8.0 Hz, 2H), 3.39 (t, J=8.8 Hz, 2H), 2.59-2.67 (m, 2H), 2.46-2.49 (m, 2H), 1.52-1.57 (m, 3H), 1.45 (s, 9H), 1.26 (br, 1H), 1.08-1.09 (m, 2H).

### Step 4: Tert-butyl 4-((1-(2-(2,6-dioxopiperidin-3-yl)-1,3-dioxoisoindolin-5-yl)azetidin-3-yl)methyl)piperidine-1-carboxylate

A mixture of tert-butyl 4-(azetidin-3-ylmethyl)piperidine-1-carboxylate (500 mg, 1.96 mmol) and 2-(2,6-dioxopiperidin-3-yl)-5-fluoroisoindoline-1,3-dione (480 mg, 1.73 mmol) in DIEA/DMF ( 1/10, 10 mL) was heated to 130° C. for 3 h. LCMS showed it was completed. After cooling to rt. The mixture was taken up with EtOAc, washed with aq. NH₄Cl, brine, dried, concentrated. The crude was purified by column chromatography on silica with DCM/EtOAc (20-5/1) to afford desired product of tert-butyl 4-((1-(2-(2,6-dioxopiperidin-3-yl)-1,3-dioxoisoindolin-5-yl)azetidin-3-yl)methyl)piperidine-1-carboxylate (217 mg, 25% yield) as a yellow solid.

Chemical Formula: C₂₇H₃₄N₄O₆; Molecular Weight: 510.59

¹HNMR (400 MHz, CDCl₃): 8.01 (s, 1H), 7.63 (d, J=8.0 Hz, 1H), 6.75 (s, 1H), 6.48 (dd, J=2.0, 8.4 Hz, 1H), 4.92-4.97 (m, 1H), 4.12-4.21 (m, 4H), 3.64-3.68 (m, 2H), 2.61-2.93 (m, 6H), 2.11-2.13 (m, 1H), 1.64-1.68 (m, 4H), 1.46 (s, 9H), 1.12-1.19 (m, 2H).

LCMS: (ES⁺): m/z 455.2[M-tBu+1]⁺, t_{R}=4.68 min.

### Step 5: 2-(2,6-Dioxopiperidin-3-yl)-5-(3-(piperidin-4-ylmethyl)azetidin-1-yl)isoindoline-1,3-dione

To a solution of tert-butyl 4-((1-(2-(2,6-dioxopiperidin-3-yl)-1,3-dioxoisoindolin-5-yl)azetidin-3-yl)methyl)piperidine-1-carboxylate (100 mg, 0.196 mmol) in DCM (5 mL) was added TFA (0.5 mL). The mixture was stirred at 25° C. for 1 h. The reaction was concentrated to afford the product 2-(2,6-dioxopiperidin-3-yl)-5-(3-(piperidin-4-ylmethyl)azetidin-1-yl)isoindoline-1,3-dione (100 mg, crude) (=Intermediate 3) as a yellow oil.

Chemical Formula: C₂₂H₂₆N₄O₄; Molecular Weight: 410.47

LCMS: (ES⁺): m/z 411.2[M+1]⁺, t_{R}=2.835 min.

### Synthesis of Intermediate 4: 2-(2,6-Dioxopiperidin-3-yl)-5-(4-(6-(piperazin-1-yl)pyrimidin-4-yl)piperazin-1-yl)isoindoline-1,3-dione

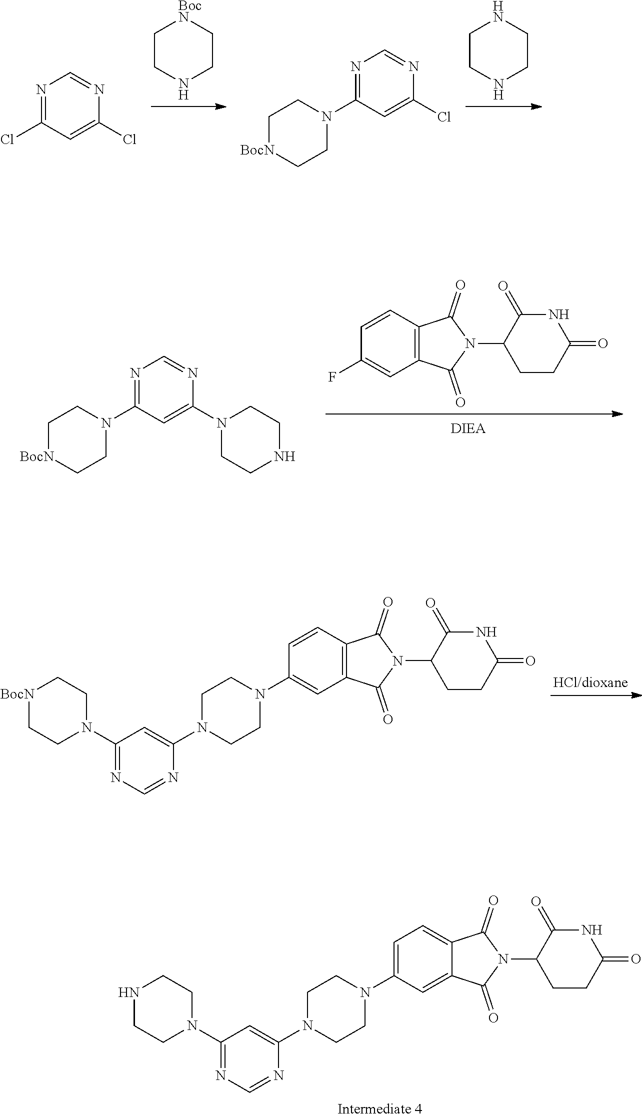

### Step 1: Tert-butyl 4-(6-chloropyrimidin-4-yl)piperazine-1-carboxylate

To a solution of 4,6-dichloropyrimidine (0.5 g, 3.36 mmol) in EtOH (10 mL) were added tert-butyl piperazine-1-carboxylate (0.626 g, 3.36 mmol) and TEA (0.51 g, 5.04 mmol) at room temperature (r.t.). Then stirred at heated to 70° C. for 15 min. The mixture was concentrated, quenched with water (20 mL), extracted with EA (20 mL), concentrated and purified by column chromatography with PE:EA=6:1-3:1 to give the desired product tert-butyl 4-(6-chloropyrimidin-4-yl)piperazine-1-carboxylate (0.9 g, 89% yield) as a white solid.

Chemical Formula: C₁₃H₁₉ClN₄O₂; Molecular Weight: 298.77

LC-MS: (ES+): m/z 299.2 [M+1]⁺, t_{R}=3.21 min.

### Step 2: Tert-butyl 4-(6-(piperazin-1-yl)pyrimidin-4-yl)piperazine-1-carboxylate

To a solution of tert-butyl 4-(6-chloropyrimidin-4-yl)piperazine-1-carboxylate (9.5 g, 0.032 mol) in EtOH (150 mL) were added piperazine (8.21 g, 0.095 mol) and TEA (10 g, 0.095 mol) at r.t. Then stirred at heated to 70° C. for 16 h. The mixture was concentrated, quenched with water (100 mL), extracted with EA (100 mL), concentrated and purified by flash to desired product (8 g, 73% yield) as a white solid.

Chemical Formula: C₁₁H₂₈N₆O₂; Molecular Weight: 348.44

¹H NMR (400 MHz, CD₃OD): δ 8.16 (s, 1H), 5.86 (s, 1H), 3.59-3.59 (m, 4H), 3.49-3.41 (m, 4H), 3.39-3.35 (m, 4H), 2.74-2.67 (m, 4H), 1.41 (s, 9H).

LC-MS: (ES+): m/z 349.2 [M+1]⁺, t_{R}=2.01 min.

### Step 3: Tert-butyl 4-(6-(4-(2-(2,6-dioxopiperidin-3-yl)-1,3-dioxoisoindolin-5-yl)piperazin-1-yl)pyrimidin-4-yl)piperazine-1-carboxylate

To a solution of tert-butyl 4-(6-(piperazin-1-yl)pyrimidin-4-yl)piperazine-1-carboxylate (3.02 g, 8.69 mmol) in DMF (20 mL) were added 2-(2,6-dioxopiperidin-3-yl)-5-fluoroisoindoline-1,3-dione (2 g, 7.24 mmol) and DIEA (2.34 g, 18.1 mmol) at r.t. The mixture was stirred at 130° C. for 3 h. The mixture was quenched with water (80 mL), extracted with EA (100 mL), concentrated and purified by flash to give the desired product Tert-butyl 4-(6-(4-(2-(2,6-dioxopiperidin-3-yl)-1,3-dioxoisoindolin-5-yl)piperazin-1-yl)pyrimidin-4-yl)piperazine-1-carboxylate (1.77 g, 40% yield) as a yellow solid.

Chemical Formula: C₃₀H₃₆N₈O₆; Molecular Weight: 604.66

¹H NMR (400 MHz, DMSO-d₆): δ 11.01 (s, 1H), 8.28 (s, 1H), 7.34 (d, J=8.4 Hz, 1H), 7.40 (s, 1H), 7.32-7.23 (m, 1H), 6.07 (s, 1H), 5.14-5.02 (m, 1H), 4.90-4.78 (m, 4H), 4.71-7.58 (m, 8H), 4.50-4.38 (m, 4H), 2.97-2.82 (m, 1H), 2.65-2.52 (m, 2H), 2.09-1.98 (m, 1H), 1.42 (s, 9H).

LC-MS: (ES⁺): m/z 605.3 [M+1]⁺, t_{R}=2.65 min.

### Step 4: 2-(2,6-Dioxopiperidin-3-yl)-5-(4-(6-(piperazin-1-yl)pyrimidin-4-yl)piperazin-1-yl)isoindoline-1,3-dione

A solution of tert-butyl 4-(6-(4-(2-(2,6-dioxopiperidin-3-yl)-1,3-dioxoisoindolin-5-yl)piperazin-1-yl)pyrimidin-4-yl)piperazine-1-carboxylate (100 mg, 0.165 mmol) in HCl/dioxane (5 mL) was stirred at 25° C. for 2 h. The reaction was concentrated to afford the desire product 2-(2,6-dioxopiperidin-3-yl)-5-(4-(6-(piperazin-1-yl)pyrimidin-4-yl)piperazin-1-yl)isoindoline-1,3-dione (100 mg, crude) (=Intermediate 4) as a white solid.

Chemical Formula: C₂₅H₂₈N₈O₄; Molecular Weight: 504.55

LCMS: (ES⁺): m/z 505.2[M+1]⁺, t_{R}=2.616 min.

### Synthesis of Intermediate 5: 2-(2,6-Dioxopiperidin-3-yl)-5-(4-((piperidin-4-ylmethoxy)methyl)piperidin-1-yl)isoindoline-1,3-dione

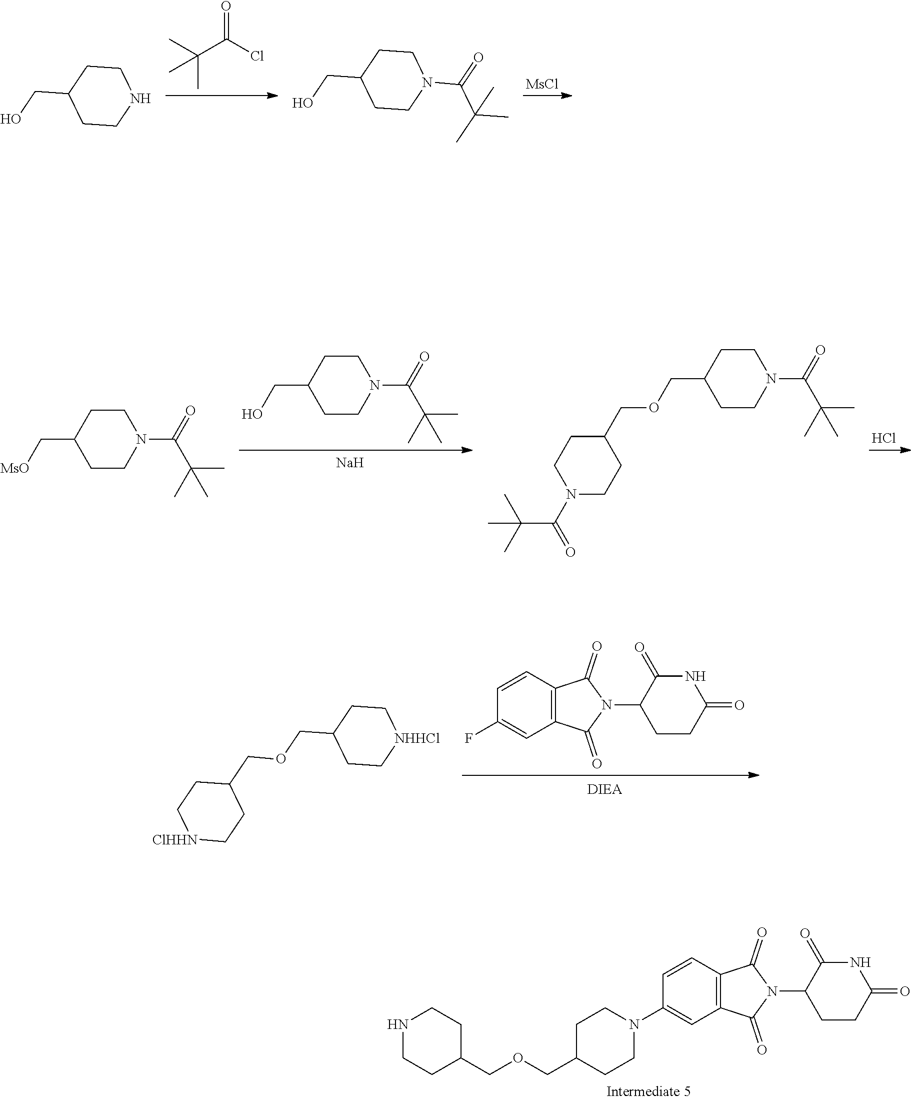

### Step 1: 1-(4-(Hydroxymethyl)piperidin-1-yl)-2,2-dimethylpropan-1-one

To a solution of piperidin-4-ylmethanol (30 g, 0.26 mol) in DCM (300 mL) was added DIEA (95 mL, 0.578 mol) and pivaloyl chloride (33.6 mL, 0.274 mol) at 0° C. Then the mixture was stirred at r.t. for 16 h. The mixture was quenched with H₂O (20 mL) and extracted with DCM (100 mL). The combined filtrate was concentrated to give the desired product 1-(4-(Hydroxymethyl)piperidin-1-yl)-2,2-dimethylpropan-1-one (28 g, 54% yield) as a yellow oil.

Chemical Formula: C₁₁H₂₁NO₂; Molecular Weight: 199.29

LC-MS: (ES⁺): m/z 200.2 [M+1]⁺, t_{R}=3.00 min.

### Step 2: (1-Pivaloylpiperidin-4-yl)methyl methanesulfonate

To a solution of 1-(4-(hydroxymethyl)piperidin-1-yl)-2,2-dimethylpropan-1-one (20 g, 0.117 mole (mol)) in DCM (200 mL) was added TEA (35.5 g, 0.351 mol) and MsCl (16 g, 0.14 mol) at 0° C. Then the mixture was stirred at r.t. for 2 h. The mixture was quenched with H₂O (200 mL). The combined filtrate was concentrated to give the desired product (1-Pivaloylpiperidin-4-yl)methyl methanesulfonate (20 g, 69% yield) as a yellow oil.

Chemical Formula: C₁₂H₂₃NO₄S; Molecular Weight: 277.38

LC-MS: (ES⁺): m/z 278.2 [M+1]⁺, t_{R}=3.52 min.

### Step 3: 1,1′-((Oxybis(methylene))bis(piperidine-4,1-diyl))bis(2,2-dimethylpropan-1-one)

To a solution of 1-(4-(hydroxymethyl)piperidin-1-yl)-2,2-dimethylpropan-1-one (5 g, 0.025 mol) in DMF (50 mL) was added NaH (1.2 g, 0.3 mol) at 0° C. under N₂. After stirred at 0° C. for 0.5 h. (1-Pivaloylpiperidin-4-yl)methyl methanesulfonate (8.35 g, 0.03 mol) was added to the mixture at 0° C., Then the mixture was stirred at 100° C. for 16 h. The mixture was quenched with NH₄Cl solution (20 mL), diluted with H₂O (100 mL) and extracted with EA (100 mL*2). The combined filtrate was concentrated to give the crude product, which was purified by chromatography column with PE:EA=3:1-2:1 to give the desired product 1,1′-((Oxybis(methylene))bis(piperidine-4,1-diyl))bis(2,2-dimethylpropan-1-one) (2 g, 20.9% yield) as a yellow oil.

Chemical Formula: C₂₂H₄₀N₂O₃; Molecular Weight: 380.57

LC-MS: (ES+): m/z 381.3 [M+1]⁺, t_{R}=4.71 min.

### Step 4: 4,4′-(Oxybis(methylene))dipiperidine dihydrochloride

A solution of 1,1′-((oxybis(methylene))bis(piperidine-4,1-diyl))bis(2,2-dimethylpropan-1-one) (1.1 g, 2.89 mmol) in HCl/dioxane (8 mL, 4 molar (M)) was stirred at 65° C. for 16 h. The mixture was cooled to 0° C., filtered to afford the product (550 mg, 67.2% yield) as a yellow oil.

Chemical Formula: C₁₂H₂₆Cl₂N₂₀; Molecular Weight: 285.25 (bis HCl salt); Molecular Weight: 212.34 (free base) ¹H NMR (400 MHz, D₂O): δ 3.48-3.33 (m, 8H), 3.03-2.89 (m, 4H), 1.99-1.85 (m, 6H), 1.48-1.30 (m, 4H).

LC-MS: (ES+): m/z 213.3 [M+H]⁺, t_{R}=0.5 min.

### Step 5: 2-(2,6-Dioxopiperidin-3-yl)-5-(4-((piperidin-4-ylmethoxy)methyl)piperidin-1-yl)isoindoline-1,3-dione

To a solution of 4,4′-(oxybis(methylene))dipiperidine dihydrochloride (500 mg, 1.77 mmol) in DMSO (5 mL) were added 2-(2,6-dioxopiperidin-3-yl)-5-fluoroisoindoline-1,3-dione (480 mg, 1.77 mmol) and DIEA (1.14 g, 8.85 mmol) at r.t. After the mixture was heated to 90° C. for 16 h. The mixture was purified by flash to give the desired product 2-(2,6-Dioxopiperidin-3-yl)-5-(4-((piperidin-4-ylmethoxy)methyl)piperidin-1-yl)isoindoline-1,3-dione (350 mg, 42.3% yield) (=Intermediate 5) as a yellow solid.

Chemical Formula: C₂₅H₃₂N₄O₅; Molecular Weight: 468.55

¹H NMR (400 MHz, DMSO-d₆): δ 11.07 (s, 1H), 8.69 (s, 1H), 7.66 (d, J=8.8 Hz, 1H), 7.31 (s, 1H), 7.24 (d, J=8.8 Hz, 1H), 5.12-5.00 (m, 1H), 4.12-3.99 (m, 2H), 3.28-3.17 (m, 5H), 3.02-2.79 (m, 5H), 2.65-2.52 (m, 2H), 2.09-1.95 (m, 1H), 1.90-1.67 (m, 6H), 1.43-1.16 (m, 5H).

LC-MS: (ES⁺): m/z 469.3 [M+1]⁺, t_{R}=2.2 min.

### Synthesis of Intermediate 6: 5-(4-(Aminomethyl)piperidin-1-yl)-2-(2,6-dioxopiperidin-3-yl)isoindoline-1,3-dione

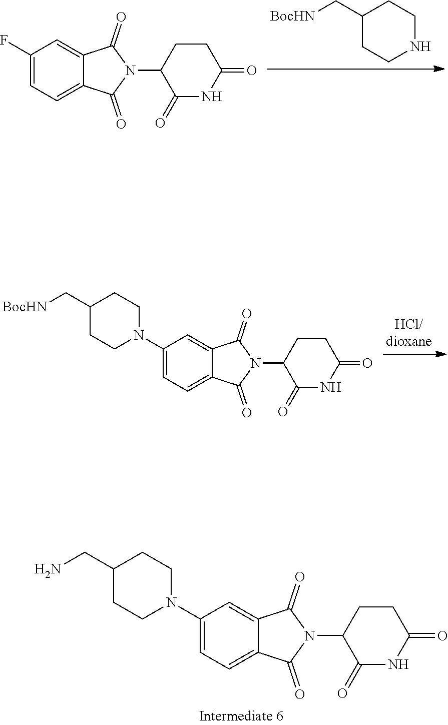 [SMILES: O=C1CCC(N2C(=O)c3ccc(F)cc3C2=O)C(=O)N1 ; CC(C)(C)OC(=O)NCC1CCNCC1 ; CC(C)(C)OC(=O)NCC1CCN(c2ccc3c(c2)C(=O)N(C2CCC(=O)NC2=O)C3=O)CC1 ; NCC1CCN(c2ccc3c(c2)C(=O)N(C2CCC(=O)NC2=O)C3=O)CC1]

### Step 1: Tert-butyl ((1-(2-(2,6-dioxopiperidin-3-yl)-1,3-dioxoisoindolin-5-yl)piperidin-4-yl)methyl)carbamate

To a solution of tert-butyl (piperidin-4-ylmethyl)carbamate (500 mg, 2.33 mmol) and 2-(2,6-dioxopiperidin-3-yl)-5-fluoroisoindoline-1,3-dione (644 mg, 2.33 mmol) in DMAC (8 mL) was added DIEA (904 mg, 6.99 mmol). The mixture was stirred at 120° C. for 5 h. The reaction was diluted with water (30 mL) and extracted with DCM (30 mL). The organic phase was concentrated and purified by silica gel column to afford the desired product tert-butyl ((1-(2-(2,6-dioxopiperidin-3-yl)-1,3-dioxoisoindolin-5-yl)piperidin-4-yl)methyl)carbamate (1 g, 91% yield) as a yellow oil.

Chemical Formula: C₂₄H₃₀N₄O₆; Molecular Weight: 470.53

LCMS: (ES⁺): m/z 415.2[M-55]⁺, t_{R}=4.082 min.

### Step 2: 5-(4-(Aminomethyl)piperidin-1-yl)-2-(2,6-dioxopiperidin-3-yl)isoindoline-1,3-dione

A solution of tert-butyl ((1-(2-(2,6-dioxopiperidin-3-yl)-1,3-dioxoisoindolin-5-yl)piperidin-4-yl)methyl)carbamate (600 mg, 1.276 mmol) in HCl/dioxane (10 mL, 4 M) was stirred at 25° C. for 2 h. The reaction was concentrated to afford the desired product 5-(4-(aminomethyl)piperidin-1-yl)-2-(2,6-dioxopiperidin-3-yl)isoindoline-1,3-dione (600 mg, crude) (=Intermediate 6) as a yellow solid.

Chemical Formula: C₁₉H₂₂N₄O₄; Molecular Weight: 370.41

LCMS: (ES⁺): m/z 371.2[M+1]⁺, t_{R}=2.552 min.

### Synthesis of Intermediate 7: 5-(4-(4-(Azetidin-3-ylmethyl)piperazin-1-yl)piperidin-1-yl)-2-(2,6-dioxopiperidin-3-yl)isoindoline-1,3-dione

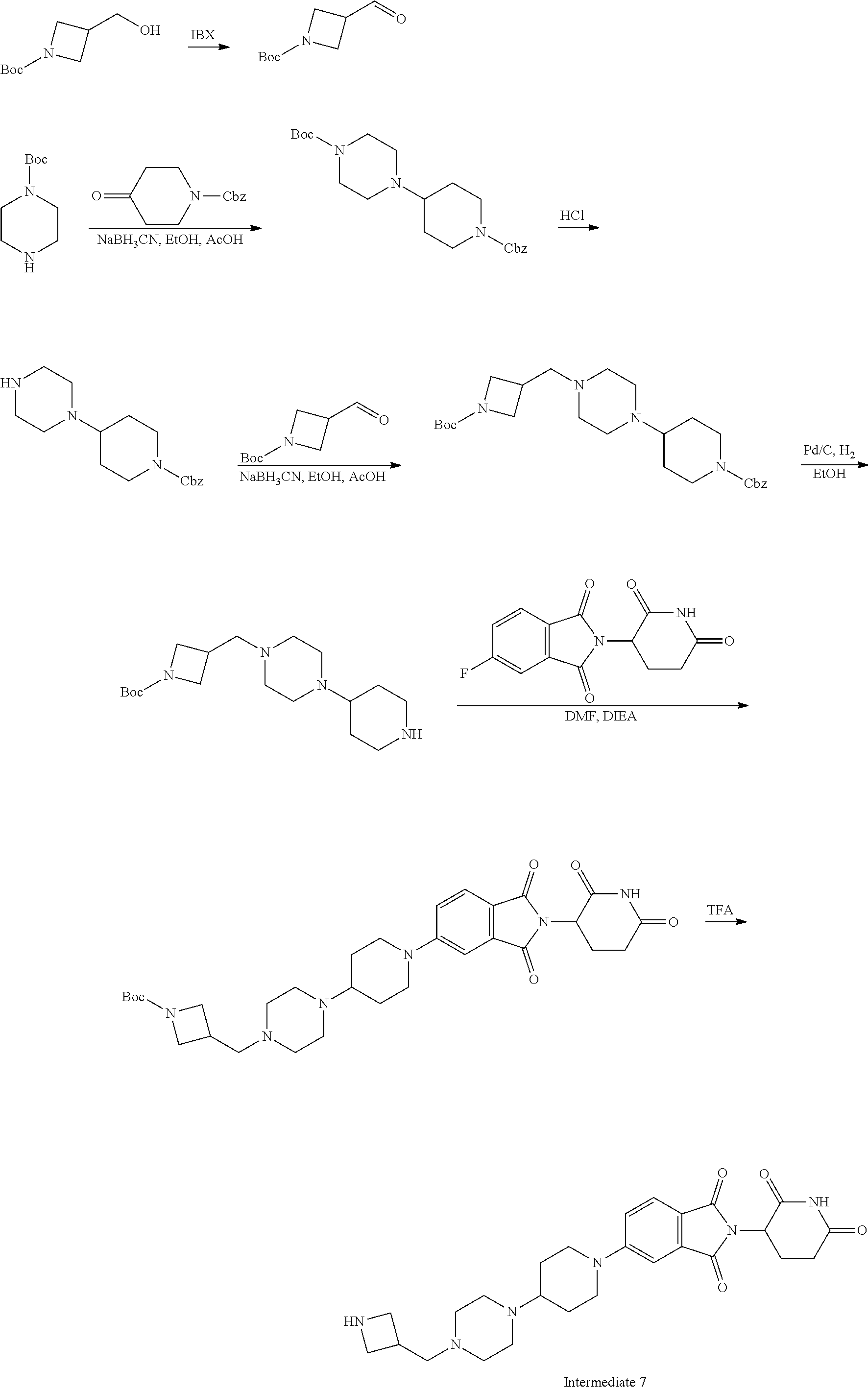

### Step 1: Tert-butyl 3-formylazetidine-1-carboxylate

A mixture of tert-butyl 3-(hydroxymethyl)azetidine-1-carboxylate (1.0 g, 5.3 mmol) and IBX (2.2 g, 8.0 mmol) in CH₃CN (20 mL) was heated to 80° C. for 3 h. TLC showed reaction was completed. After cooling to RT, the mixture was filtered through Celite, and the filtrate was concentrated to give the crude product as light-yellow oil, which was used into next reaction without further purification.

### Step 2: tert-butyl 4-(1-((benzyloxy)carbonyl)piperidin-4-yl)piperazine-1-carboxylate

A mixture of tert-butyl piperazine-1-carboxylate (5.0 g, 26.8 mmol) and benzyl 4-oxopiperidine-1-carboxylate (6.2 g, 26.8 mmol) in AcOH/EtOH ( 1/10, 50 mL) was stirred for 1 h at RT. Then NaBH₃CN (1.7 g, 26.8 mmol) was added in portions at 0° C. The reaction was stirred overnight. TLC showed reaction was completed. The solvent was removed under vacuum, the residue was taken up with EtOAc, washed with aqueous (aq.) NaHCO₃ solution and concentrated to give crude desired product as a light-brown oil, which was used into next reaction without further purification.

### Step 3: Benzyl 4-(piperazin-1-yl)piperidine-1-carboxylate

A mixture of tert-butyl piperazine-1-carboxylate (5.0 g, 26.8 mmol) and benzyl 4-oxopiperidine-1-carboxylate (6.2 g, 26.8 mmol) in AcOH/EtOH ( 1/10, 50 mL) was stirred for 1 h at RT. Then NaBH₃CN (1.7 g, 26.8 mmol) was added in portions at 0° C. The reaction was stirred overnight. TLC showed reaction was completed. The solvent was removed under vacuum, the residue was taken up with EtOAc, washed with aq. NaHCO₃ solution and concentrated to give crude desired product as a light-brown oil, which was used into next reaction without further purification.

To a solution of tert-butyl 4-(1-((benzyloxy)carbonyl)piperidin-4-yl)piperazine-1-carboxylate in CH₃OH (50 mL) was added concentrated (conc.) HCl (20 mL) at RT. After stirring for 2 hours, LCMS showed complete conversion. The solvent was removed under vacuum. The residue was taken up in water. The pH of the aqueous solution was adjusted ˜9 with solid NaOH. The mixture was extracted with EtOAc (50 mL×3), the combined organic layers were concentrated to afford crude desired product as light-yellow oil (3.2 g), which was used into next reaction without further purification.

Chemical Formula: C₁₇H₂₅N₃O₂; Molecular Weight: 303.41

¹HNMR (400 MHz, CDCl₃):7 27-7.36 (m, 5H), 5.12 (s, 2H), 4.23 (br, 2H), 2.89-2.91 (m, 4H), 2.78 (br, 2H), 2.53-2.54 (m, 4H), 2.34-2.41 (m, 1H), 1.80-1.83 (m, 2H), 1.42-1.46 (m, 2H).

### Step 4: Benzyl 4-(4-((1-(tert-butoxycarbonyl)azetidin-3-yl)methyl)piperazin-1-yl)piperidine-1-carboxylate

A solution of benzyl 4-(piperazin-1-yl)piperidine-1-carboxylate (2.0 g, 6.6 mmol) and tert-butyl 3-formylazetidine-1-carboxylate (1.1 g, crude) in AcOH/EtOH ( 1/10, 30 mL) was stirred for 1 h at rt. Then NaBH₃CN (0.41 g, 6.6 mmol) was added. And the reaction stirred at r.t. The solvent was removed under vacuum. The residue was partitioned between ethyl acetate and aqueous NaHCO₃ solution. The aqueous layer was extracted with ethyl acetate (50 mL×3). The combined organic layers were washed with brine, dried over MgSO₄ and concentrated. The crude product was purified by column chromatography on silica with DCM/EtOAc (10-0/1) to afford desired product benzyl 4-(4-((1-(tert-butoxycarbonyl)azetidin-3-yl)methyl)piperazin-1-yl)piperidine-1-carboxylate as a light-yellow oil (1.4 g).

Chemical Formula: C₂₆H₄₀N₄O₄; Molecular Weight: 472.63

¹HNMR (400 MHz, CDCl₃): 7 27-7.36 (m, 5H), 5.12 (s, 2H), 4.23 (br, 2H), 4.00 (t, J=8.4 Hz, 2H), 3.57-3.60 (m, 2H), 2.68-2.79 (m, 4H), 2.59-2.62 (m, 7H), 2.45-2.49 (m, 5H), 1.82-1.84 (m, 2H), 1.43 (m, 9H).

### Step 5: Tert-butyl 3-((4-(piperidin-4-yl)piperazin-1-yl)methyl)azetidine-1-carboxylate

A mixture of benzyl 4-(4-((1-(tert-butoxycarbonyl)azetidin-3-yl)methyl)piperazin-1-yl)piperidine-1-carboxylate (1.4 g, 3.0 mmol) and Pd/C (wet, 10%, 500 mg) in EtOH (20 mL) was stirred overnight at RT under hydrogen. TLC showed a small amount of starting material remaining. The mixture was filtered through Celite, and the filtrate was concentrated. The residue was chromatographed on silica gel (CH₂Cl₂/MeOH 10:1-3:1) to afford desired product of tert-butyl 3-((4-(piperidin-4-yl)piperazin-1-yl)methyl)azetidine-1-carboxylate (675 mg, 66% yield) as white solid.

Chemical Formula: C₁₈H₃₄N₄O₂; Molecular Weight: 338.50

¹HNMR (400 MHz, DMSO-d₆+D₂O): 3.65 (br, 2H), 3.38 (br, 2H), 2.75-3.23 (m, 16H), 2.01-2.04 (m, 2H), 1.63-1.66 (m, 2H), 1.38 (m, 9H).

LCMS: (ES⁺): m/z 339.3 [M+1]⁺, t_{R}=2.31 min.

### Step 6: Tert-butyl 3-((4-(1-(2-(2,6-dioxopiperidin-3-yl)-1,3-dioxoisoindolin-5-yl)piperidin-4-yl)piperazin-1-yl)methyl)azetidine-1-carboxylate

A mixture of tert-butyl 3-((4-(piperidin-4-yl)piperazin-1-yl)methyl)azetidine-1-carboxylate (1.4 g, 3.0 mmol) and 2-(2,6-dioxopiperidin-3-yl)-5-fluoroisoindoline-1,3-dione (995 mg, 3.6 mmol) in DIEA/DMF ( 1/10, 20 mL) was heated to 130° C. for 4 h. LCMS showed it was completed. The mixture was taken up with EtOAc, washed with brine, dried, concentrated. The residue was chromatographed on silica gel (CH₂Cl₂/EtOAc 10:1-1:1) to afford desired product of tert-butyl 3-((4-(1-(2-(2,6-dioxopiperidin-3-yl)-1,3-dioxoisoindolin-5-yl)piperidin-4-yl)piperazin-1-yl)methyl)azetidine-1-carboxylate (1.1 g, 62% yield) as yellow solid.

Chemical Formula: C₃₁H₄₂N₆O₆; Molecular Weight: 594.71

¹HNMR (400 MHz, CDCl₃): 8.37 (s, 1H), 7.71 (d, J=7.6 Hz, 1H), 7.07 (dd, J=2.0, 8.4 Hz, 1H), 4.92-4.97 (m, 1H), 4.03-4.12 (m, 4H), 3.64-3.68 (m, 3H), 3.26-3.45 (m, 9H), 3.13 (d, J=7.2 Hz, 2H), 2.76-3.01 (m, 6H), 2.12-2.15 (m, 3H), 1.81-1.85 (m, 2H), 1.43 (m, 9H).

LCMS: (ES⁺): m/z 595.3 [M+1]⁺, t_{R}=3.04 min.

### Step 7: 5-(4-(4-(Azetidin-3-ylmethyl)piperazin-1-yl)piperidin-1-yl)-2-(2,6-dioxopiperidin-3-yl)isoindoline-1,3-dione

A solution of tert-butyl 3-((4-(1-(2-(2,6-dioxopiperidin-3-yl)-1,3-dioxoisoindolin-5-yl)piperidin-4-yl)piperazin-1-yl)methyl)azetidine-1-carboxylate (100 mg, 0.168 mmol), TFA (0.5 mL) in DCM (5 mL) was stirred at 25° C. for 2 h. The reaction was concentrated to afford the product 5-(4-(4-(azetidin-3-ylmethyl)piperazin-1-yl)piperidin-1-yl)-2-(2,6-dioxopiperidin-3-yl)isoindoline-1,3-dione (100 mg, crude) (=Intermediate 7) as a yellow oil.

Chemical Formula: C₂₆H₃₄N₆O₄; Molecular Weight: 494.60

LCMS: (ES⁺): m/z 495.2[M+1]⁺, t_{R}=2.352 min.

### Synthesis of Intermediate 8: 2-(2,6-Dioxopiperidin-3-yl)-5-(4-(3-(piperidin-4-yl)propyl)piperazin-1-yl)isoindoline-1,3-dione

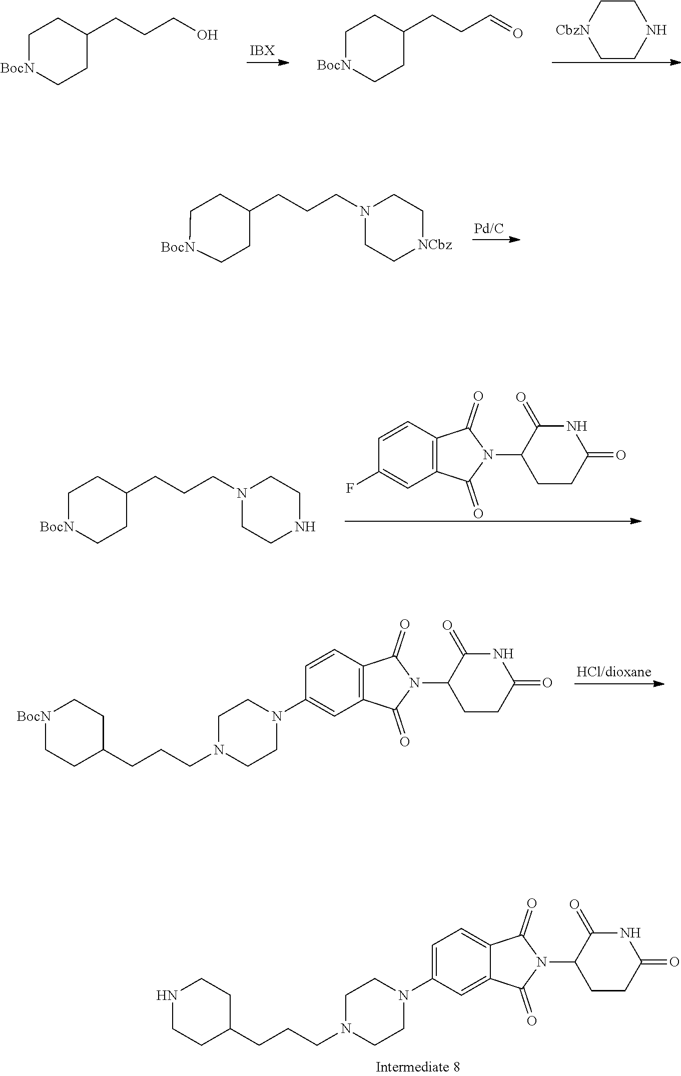

### Step 1: Tert-butyl 4-(3-oxopropyl)piperidine-1-carboxylate

To a solution of tert-butyl 4-(3-hydroxypropyl)piperidine-1-carboxylate (8 g, 0.0329 mol) in MeCN (150 mL) was added IBX (11 g, 0.0395 mol) at r.t. The mixture was heated to 80° C. for 3 h. The mixture was filtered and concentrated to afford the product (8 g, crude) as a yellow oil.

Chemical Formula: C₁₃H₂₃NO₃; Molecular Weight: 241.33

LC-MS: (ES+): m/z 186.1 [M-tBu+H]⁺, t_{R}=3.21 min.

### Step 2: Benzyl 4-(3-(1-(tert-butoxycarbonyl)piperidin-4-yl)propyl)piperazine-1-carboxylate

To a solution of tert-butyl 4-(3-oxopropyl)piperidine-1-carboxylate (8 g, 0.0264 mol) in DCM:EtOH=1:1 (80 mL) was added benzyl piperazine-1-carboxylate (10.87 g, 0.0493 mol) and NaBH₃CN (6.2 g, 0.0987 mol) at r.t. The mixture was stirred at r.t for 2 h. The mixture was concentrated to give the crude product, which was purified by chromatography column with PE:EtOAc=10:1-3:1 to give the product (6.4 g, 43% yield) as a yellow oil.

Chemical Formula: C₂₅H₃₉N₃O₄; Molecular Weight: 445.59

LC-MS: (ES+): m/z 446.3 [M+1]⁺, t_{R}=2.66 min.

### Step 3: Tert-butyl 4-(3-(piperazin-1-yl)propyl)piperidine-1-carboxylate

To a solution of benzyl 4-(3-(1-(tert-butoxycarbonyl)piperidin-4-yl)propyl)piperazine-1-carboxylate (6.4 g, 0.0144 mol) in ethanol (150 mL) was added 10% Pd/C (0.5 g) at r.t. The mixture was stirred at r.t under H₂ for 3 d. The mixture was filtered through a celite pad and concentrated to give the crude product, which was purified by chromatography column with DCM:MeOH=10:1-5:1 to afford the desired product (2.6 g, 58.2% yield) as a yellow oil.

Chemical Formula: C₁₇H₃₃N₃O₂; Molecular Weight: 311.46

¹H NMR (400 MHz, CD₃OD): δ 4.12-4.01 (m, 2H), 3.28-3.16 (m, 4H), 2.84-2.68 (m, 6H), 2.55-2.48 (m, 2H), 1.74-1.65 (m, 2H), 1.62-1.52 (m, 2H), 1.51-1.40 (m, 11H), 1.33-1.25 (m, 2H), 1.12-1.00 (m, 2H).

### Step 4: Tert-butyl 4-(3-(4-(2-(2,6-dioxopiperidin-3-yl)-1,3-dioxoisoindolin-5-yl)piperazin-1-yl)propyl)piperidine-1-carboxylate

To a solution of tert-butyl 4-(3-(piperazin-1-yl)propyl)piperidine-1-carboxylate (1.6 g, 5.14 mmol) in DMF (20 mL) were added 2-(2,6-dioxopiperidin-3-yl)-5-fluoroisoindoline-1,3-dione (1.49 g, 5.39 mmol) and DIEA (1.66 g, 12.85 mmol) at r.t. After the mixture was stirred at 130° C. for 3 h. The mixture was quenched with water (80 mL), extracted with EtOAc (100 mL), concentrated, purified by flash to desired product (0.945 g, 32.5% yield) as a yellow solid.

Chemical Formula: C₃₀H₄₁N₅O₆; Molecular Weight: 567.69

¹H NMR (400 MHz, DMSO-d₆): δ 11.06 (s, 1H), 7.68 (d, J=7.2 Hz, 1H), 7.33 (s, 1H), 7.40 (d, J=8.8 Hz, 1H), 5.13-4.99 (m, 1H), 4.00-3.82 (m, 2H), 3.48-3.39 (m, 4H), 2.95-2.82 (m, 1H), 2.72-2.52 (m, 4H), 2.50-2.47 (m, 3H), 2.35-2.25 (m, 2H), 2.06-1.96 (m, 1H), 1.68-1.57 (m, 2H), 1.52-1.39 (m, 3H), 1.39-1.33 (m, 10H), 1.23-1.15 (m, 2H), 1.00-0.90 (m, 2H).

LC-MS: (ES+): m/z 468.2 [M-Boc+H]⁺, t_{R}=3.15 min.

### Step 5: 2-(2,6-Dioxopiperidin-3-yl)-5-(4-(3-(piperidin-4-yl)propyl)piperazin-1-yl)isoindoline-1,3-dione

A solution of tert-butyl 4-(3-(4-(2-(2,6-dioxopiperidin-3-yl)-1,3-dioxoisoindolin-5-yl)piperazin-1-yl)propyl)piperidine-1-carboxylate (55 mg, 0.097 mmol) in HCl/dioxane (5 mL, 4 M) was stirred at 25° C. for 2 h. The reaction was concentrated to afford the product 2-(2,6-dioxopiperidin-3-yl)-5-(4-(3-(piperidin-4-yl)propyl)piperazin-1-yl)isoindoline-1,3-dione (55 mg, crude) (=Intermediate 8) as a white solid.

Chemical Formula: C₂₅H₃₃N₅O₄; Molecular Weight: 467.57

LCMS: (ES⁺): m/z 468.2[M+1]⁺, t_{R}=2.256 min.

### Synthesis of Intermediate 9: 2-(2,6-dioxopiperidin-3-yl)-5-(4-(4-(2-(piperidin-4-yl)ethyl)piperidin-1-yl)phenoxy)isoindoline-1,3-dione

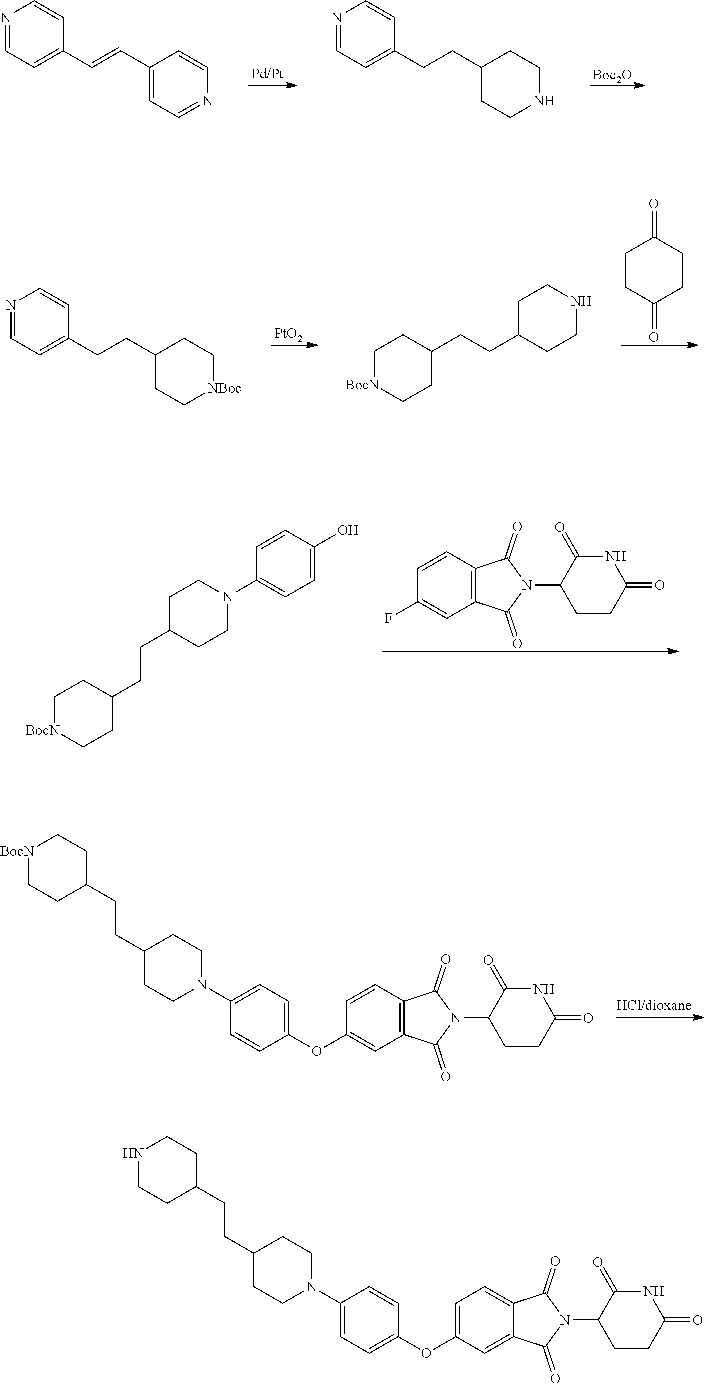

### Step 1: 4-(2-(Piperidin-4-yl)ethyl)pyridine

A mixture of (E)-1,2-di(pyridin-4-yl)ethene (1 g, 5.5 mmol), 10% Pd/C (300 mg) and 10% Pt/C (300 mg) in ethyl acetate (EA) (20 mL) was heated at 70° C. for 4 h under H₂ (2 megapascals (MPa)). After cooling to rt, the mixture was filtered and the filtrate was evaporated to afford a crude product which was purified by column chromatography (MeOH:DCM=1:50-1:10) to afford 4-(2-(Piperidin-4-yl)ethyl)pyridine (350 mg, 33% yield) as a white solid.

Chemical Formula: C₁₂H₁₈N₂ Exact Mass: 190.15

LCMS: (ES+): 191.2 [M+1]⁺, t_{R}=0.53 min.

### Step 2: Tert-butyl 4-(2-(pyridin-4-yl)ethyl)piperidine-1-carboxylate

A mixture of 4-(2-(Piperidin-4-yl)ethyl)pyridine (330 mg, 1.7 mmol), Et₃N (376 mg, 0.56 mmol) and Boc₂O (489 mg, 0.34 mmol) in DCM (10 mL) was stirred at r.t. for 2 h. After quenched with H₂O (3 mL), the mixture was extracted with DCM (20 mL). The organic phase was concentrated under vacuum and purified by silica gel column to get the desired compound Tert-butyl 4-(2-(pyridin-4-yl)ethyl)piperidine-1-carboxylate (410 mg, 83% yield) as a white solid.

Chemical Formula: C₁₇H₂₆N₂O₂ Exact Mass: 290.20.

### Step 3: Tert-butyl 4-(2-(piperidin-4-yl)ethyl)piperidine-1-carboxylate

A mixture of Tert-butyl 4-(2-(pyridin-4-yl)ethyl)piperidine-1-carboxylate (330 mg, 1.14 mmol), 10% PtO₂/C (60 mg) in EtOH and CH₃COOH was stirred at r.t. for 16 h under H₂. The mixture was filtered and the filtrate was evaporated to afford a crude product which was purified by column chromatography (MeOH:DCM=1:50-1:10) to afford Tert-butyl 4-(2-(piperidin-4-yl)ethyl)piperidine-1-carboxylate (310 mg, 92% yield) as a white solid.

¹H NMR (400 MHz, CD₃OD): δ 4.02-4.05 (m, 2H), 3.29-3.31 (m, 1H), 2.85-2.88 (m, 2H), 2.72-2.81 (m, 2H), 1.85-1.89 (m, 4H), 1.59-1.67 (m, 2H), 1.45-1.46 (m, 1H), 1.44 (s, 9H), 1.28-1.33 (m, 6H), 1.02-1.04 (m, 2H).

Chemical Formula: C₁₇H₃₂N₂O₂ Exact Mass: 296.25.

### Step 4: Tert-butyl 4-(2-(1-(4-hydroxyphenyl)piperidin-4-yl)ethyl)piperidine-1-carboxylate

To a solution of tert-butyl 4-(2-(piperidin-4-yl)ethyl)piperidine-1-carboxylate (500 mg, 1.686 mmol) in ethanol (5 mL) was added cyclohexane-1,4-dione (378 mg, 3.37 mmol) at r.t, the mixture was heated to 60° C. for 16 h. The mixture was concentrated, diluted with EtOAc (30 mL), washed with water (20 mL), dried and concentrated to give the crude product, which was purified by chromatography column with PE:EtOAc=5:1-3:1 to afford the desired product (180 mg, 27.5% yield) as a yellow oil.

Chemical Formula: C₂₃H₃₆N₂O₃; Molecular Weight: 388.54

¹H NMR (400 MHz, CD₃OD): δ 6.90 (d, J=8.8 Hz, 2H), 6.71 (d, J=8.8 Hz, 2H), 4.10-3.99 (m, 2H), 3.44-3.35 (m, 2H), 2.82-2.68 (m, 2H), 2.62-2.51 (m, 2H), 1.88-1.77 (m, 2H), 1.77-1.64 (m, 2H), 1.45 (m, 9H), 1.42-1.28 (m, 2H), 1.11-0.98 (m, 2H).

LC-MS: (ES+): m/z 389.3 [M+1]⁺, t_{R}=2.55 min.

### Step 5: Tert-butyl 4-(2-(1-(4-((2-(2,6-dioxopiperidin-3-yl)-1,3-dioxoisoindolin-5-yl)oxy)phenyl)piperidin-4-yl)ethyl)piperidine-1-carboxylate

To a solution of tert-butyl 4-(2-(1-(4-hydroxyphenyl)piperidin-4-yl)ethyl)piperidine-1-carboxylate (600 mg, 2.6 mmol) in DMA (5 mL) were added 2-(2,6-dioxopiperidin-3-yl)-5-fluoroisoindoline-1,3-dione (865 mg, 3.13 mmol) and K₂CO₃ (717 mg, 5.2 mmol) at r.t. After the mixture was heated to 80° C. for 24 h. The mixture was quenched with water (15 mL), extracted with EtOAc (20 mL), concentrated, purified by flash to desired product (136 mg, 8.1% yield) as a yellow solid.

Chemical Formula: C₃₆H₄₄N₄O₇; Molecular Weight: 644.76

¹H NMR (400 MHz, DMSO-d₆): δ 11.09 (s, 1H), 7.94-7.87 (m, 1H), 7.38-7.30 (m, 1H), 7.22 (s, 1H), 7.10-6.96 (m, 4H), 5.18-5.02 (m, 1H), 3.99-3.82 (m, 2H), 3.73-3.62 (m, 2H), 2.96-2.82 (m, 1H), 2.79-2.51 (m, 6H), 2.11-1.99 (m, 1H), 1.84-1.71 (m, 2H), 1.70-1.61 (m, 2H), 1.38 (s, 9H), 1.36-1.16 (m, 8H), 1.02-0.92 (m, 2H).

LC-MS: (ES+): m/z 545.3 [M-99]⁺, t_{R}=3.84 min.

### Step 6: 2-(2,6-Dioxopiperidin-3-yl)-5-(4-(4-(2-(piperidin-4-yl)ethyl)piperidin-1-yl)phenoxy)isoindoline-1,3-dione

A solution of tert-butyl 4-(2-(1-(4-((2-(2,6-dioxopiperidin-3-yl)-1,3-dioxoisoindolin-5-yl)oxy)phenyl)piperidin-4-yl)ethyl)piperidine-1-carboxylate (100 mg, 0.155 mmol) in HCl/dioxane (5 mL) was stirred at 25° C. for 2 h. The reaction was concentrated to afford the desire product 2-(2,6-dioxopiperidin-3-yl)-5-(4-(4-(2-(piperidin-4-yl)ethyl)piperidin-1-yl)phenoxy)isoindoline-1,3-dione (100 mg, crude) (=Intermediate 9) as a white solid.

Chemical Formula: C₃₁H₃₆N₄O₅; Molecular Weight: 544.65

LCMS−: (ES⁺): m/z 545.2[M+1]⁺, t_{R}=2.972 min.

### Synthesis of Intermediate 10: 2-(2,6-Dioxopiperidin-3-yl)-5-((piperidin-4-ylmethyl)amino)isoindoline-1,3-dione

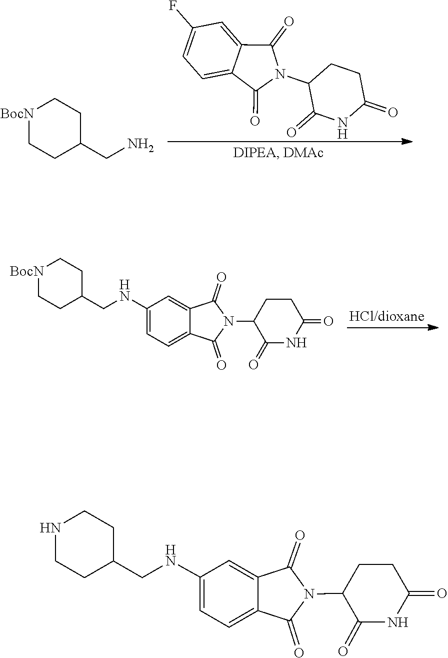 [SMILES: CC(C)(C)OC(=O)N1CCC(CN)CC1 ; O=C1CCC(N2C(=O)c3ccc(F)cc3C2=O)C(=O)N1 ; CC(C)(C)OC(=O)N1CCC(CNc2ccc3c(c2)C(=O)N(C2CCC(=O)NC2=O)C3=O)CC1 ; O=C1CCC(N2C(=O)c3ccc(NCC4CCNCC4)cc3C2=O)C(=O)N1]

### Step 1: Tert-butyl 4-(((2-(2,6-dioxopiperidin-3-yl)-1,3-dioxoisoindolin-5-yl)amino)methyl)piperidine-1-carboxylate

To a solution of tert-butyl 4-(aminomethyl)piperidine-1-carboxylate (1 g, 4.68 mmol) and 2-(2,6-dioxopiperidin-3-yl)-5-fluoroisoindoline-1,3-dione (1.3 g, 4.68 mmol) in NMP (10 mL) was added DIEA (1.81 g, 14.04 mmol). The mixture was stirred at 100° C. for 16 h. The reaction was diluted with water (30 mL) and extracted with DCM (30 mL). The organic phase was concentrated and purified by silica gel column to afford the desired product tert-butyl 4-(((2-(2,6-dioxopiperidin-3-yl)-1,3-dioxoisoindolin-5-yl)amino)methyl)piperidine-1-carboxylate (0.52 g, 24% yield) as a yellow solid.

Chemical Formula: C₂₄H₃₀N₄O₆; Molecular Weight: 470.53.

### Step 2: 2-(2,6-Dioxopiperidin-3-yl)-5-((piperidin-4-ylmethyl)amino)isoindoline-1,3-dione

A solution of tert-butyl 4-(((2-(2,6-dioxopiperidin-3-yl)-1,3-dioxoisoindolin-5-yl)amino)methyl)piperidine-1-carboxylate (250 mg, 0.494 mmol) in HCl/dioxane (8 mL, 4 M) was stirred at 25° C. for 3 h. The reaction was concentrated to afford the desired product 2-(2,6-Dioxopiperidin-3-yl)-5-((piperidin-4-ylmethyl)amino)isoindoline-1,3-dione (230 mg, crude) (=Intermediate 10) as a yellow solid.

Chemical Formula: C₁₉H₂₂N₄O₄; Molecular Weight: 370.41

LCMS: (ES⁺): m/z 371.1[M+1]⁺, t_{R}=2.50 min.

### Synthesis of PROTAC® Compounds 1-10:

### General Scheme:

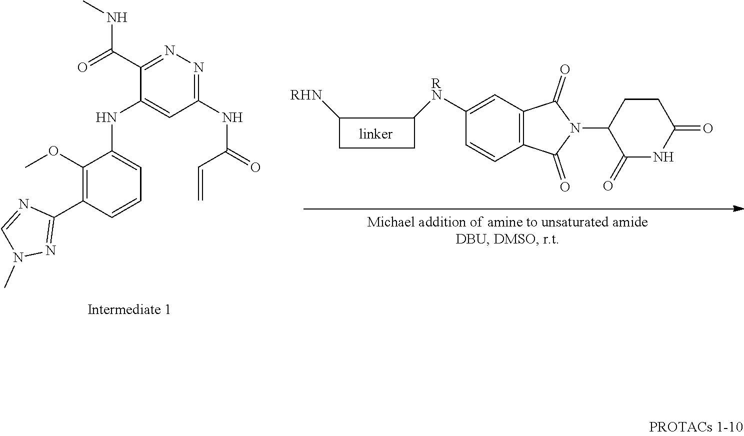

### Synthesis of PROTAC 1: 6-(3-(4-(1-(2-(2,6-Dioxopiperidin-3-yl)-1,3-dioxoisoindolin-5-yl)azetidin-3-yl)piperidin-1-yl)propanamido)-4-((2-methoxy-3-(1-methyl-1H-1,2,4-triazol-3-yl)phenyl)amino)-N-methylpyridazine-3-carboxamide

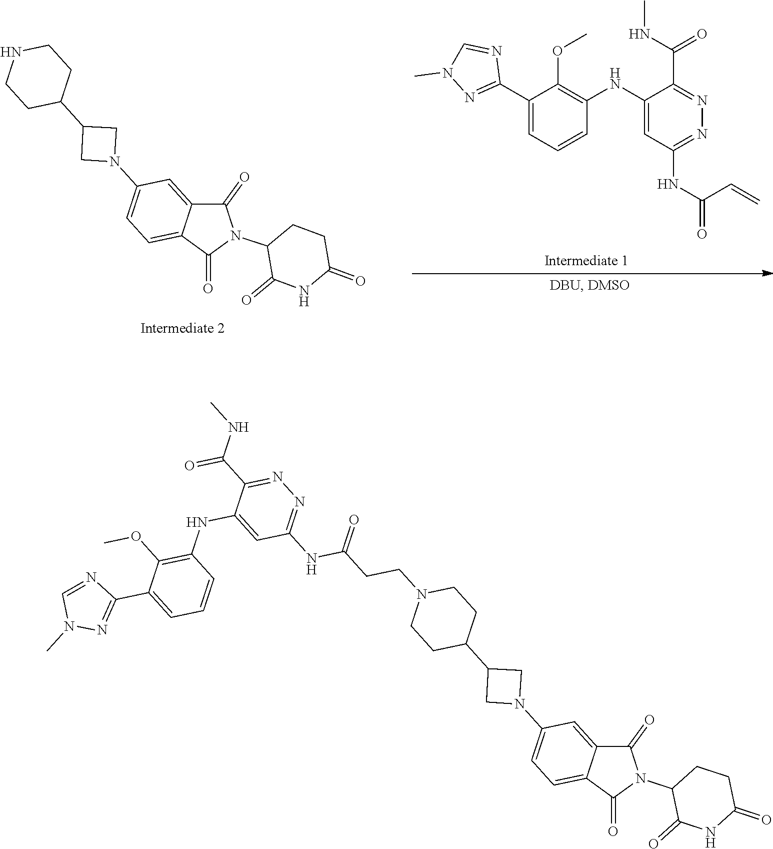 [SMILES: O=C1CCC(N2C(=O)c3ccc(N4CC(C5CCNCC5)C4)cc3C2=O)C(=O)N1 ; C=CC(=O)Nc1cc(Nc2cccc(-c3ncn(C)n3)c2OC)c(C(=O)NC)nn1 ; CNC(=O)c1nnc(NC(=O)CCN2CCC(C3CN(c4ccc5c(c4)C(=O)N(C4CCC(=O)NC4=O)C5=O)C3)CC2)cc1Nc1cccc(-c2ncn(C)n2)c1OC]

To a solution of 2-(2,6-dioxopiperidin-3-yl)-5-(3-(piperidin-4-yl)azetidin-1-yl)isoindoline-1,3-dione (Intermediate 2, 100 mg, 0.252 mmol) and 6-acrylamido-4-((2-methoxy-3-(1-methyl-1H-1,2,4-triazol-3-yl)phenyl)amino)-N-methylpyridazine-3-carboxamide (Intermediate 1, 50 mg, 0.126 mmol) in DMSO (4 mL) was added DBU (149 mg, 0.980 mmol) at 0° C. The mixture was stirred at 25° C. for 30 min. The reaction was quenched with saturated NH₄Cl (20 mL) and extracted with DCM (30 mL) and CH₃OH (3 mL). The organic phase was washed with saturated NaCl (20 mL). The organic was concentrated and purified by prep-HPLC with the following conditions (Welch Ultimate XB-C18, 21.2*250 mm Sum), eluted with H₂O in CH₃CN containing 0.05% TFA). The product containing prep-HPLC fraction was lyophilized to dryness to afford the desired product 6-(3-(4-(1-(2-(2,6-Dioxopiperidin-3-yl)-1,3-dioxoisoindolin-5-yl)azetidin-3-yl)piperidin-1-yl)propanamido)-4-((2-methoxy-3-(1-methyl-1H-1,2,4-triazol-3-yl)phenyl)amino)-N-methylpyridazine-3-carboxamide (40 mg, 20% yield) as a yellow solid.

Chemical Formula: C₄₀H₄₄N₁₂O₇; Molecular Weight: 804.87

¹H NMR (400 MHz, CD₃OD, ppm): δ 8.55 (s, 1H), 7.78-7.75 (m, 2H), 7.62-7.56 (m, 2H), 7.33-7.29 (m, 1H), 6.78-6.77 (m, 1H), 6.62-6.60 (m, 1H), 5.07-5.02 (m, 1H), 4.13-4.09 (m, 2H), 4.02 (s, 3H), 3.79-3.76 (m, 2H), 3.73 (s, 3H), 3.67-3.65 (m, 2H), 3.49 (s, 2H), 3.07-2.98 (m, 7H), 2.89-2.80 (m, 1H), 2.74-2.60 (m, 3H), 2.11-1.87 (m, 4H), 1.48-1.45 (m, 2H).

LCMS: (ES⁺): m/z 805.3[M+1]⁺, t_{R}=3.087 min.

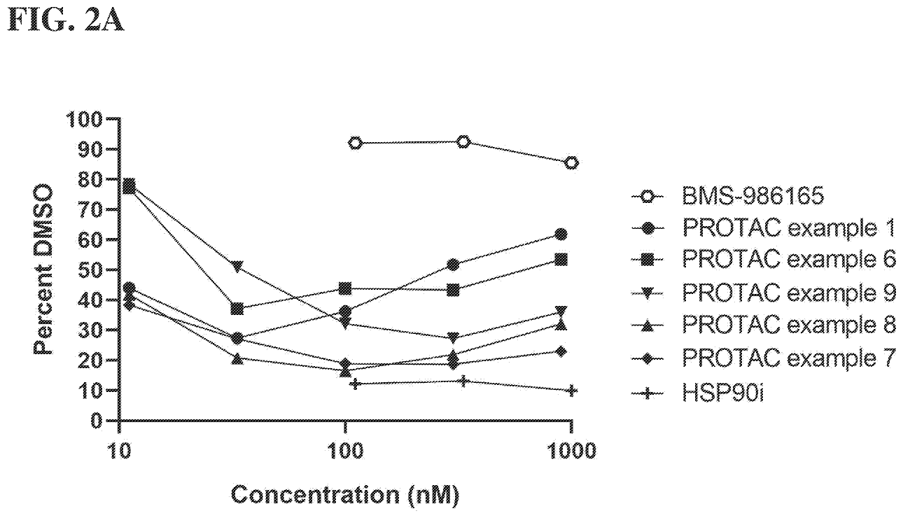

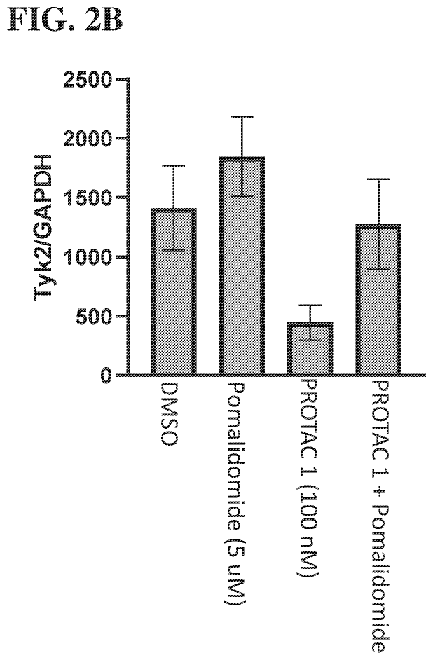

FIG. 2B shows the amount of TYK2 protein, normalized to amount of GADPH under various competition conditions. DMSO is used as a negative control, Pomalidomide alone, PROTAC 1 shows degradation, PROTAC+Pomalidomide (competing for CRBN binding) significantly reduces extent of degradation.

### Synthesis of PROTAC 2: 6-(3-(4-(2-(2,6-Dioxopiperidin-3-yl)-1,3-dioxoisoindolin-5-yl)piperazin-1-yl)propanamido)-4-((2-methoxy-3-(1-methyl-1H-1,2,4-triazol-3-yl)phenyl)amino)-N-methylpyridazine-3-carboxamide

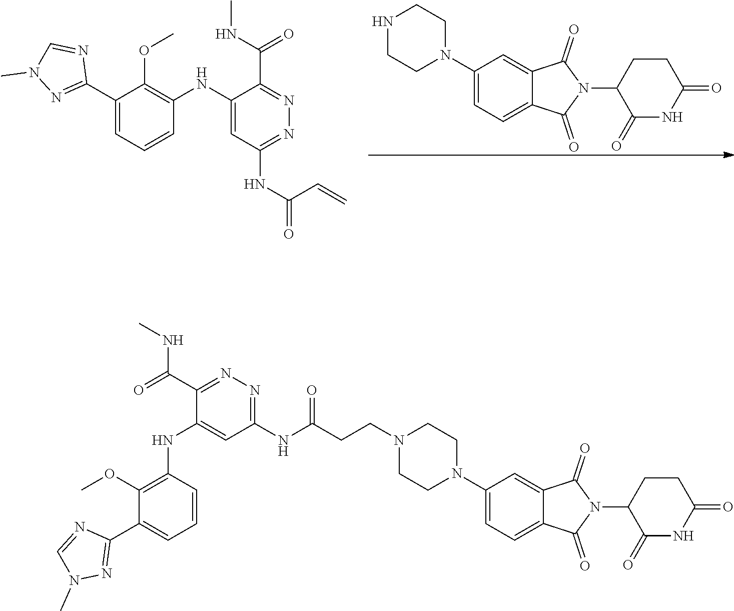 [SMILES: C=CC(=O)Nc1cc(Nc2cccc(-c3ncn(C)n3)c2OC)c(C(=O)NC)nn1 ; O=C1CCC(N2C(=O)c3ccc(N4CCNCC4)cc3C2=O)C(=O)N1 ; CNC(=O)c1nnc(NC(=O)CCN2CCN(c3ccc4c(c3)C(=O)N(C3CCC(=O)NC3=O)C4=O)CC2)cc1Nc1cccc(-c2ncn(C)n2)c1OC]

A mixture of 6-acrylamido-4-((2-methoxy-3-(1-methyl-1H-1,2,4-triazol-3-yl)phenyl)amino)-N-methylpyridazine-3-carboxamide (Intermediate 1, 9 mg, 0.022 mmol) and 2-(2,6-dioxopiperidin-3-yl)-5-(piperazin-1-yl)isoindoline-1,3-dione (U.S. Pat. Appl. Publ., 20180125821, 8.32 mg, 0.022 mmol) and DBU in DMSO (3 mL) was stirred at 25° C. for 1 h. The reaction was quenched with saturated NH₄Cl (2 mL) and extracted with DCM (10 mL) and CH₃OH (1 mL). The organic phase was washed with saturated NaCl (2 mL). The organic layer was concentrated and purified by prep-HPLC with the following conditions (Welch Ultimate XB-C18, 21.2*250 mm Sum), eluted with H₂O in CH₃CN containing 0.05% TFA). The product containing prep-HPLC fraction was lyophilized to dryness to afford the desired product 6-(3-(4-(2-(2,6-Dioxopiperidin-3-yl)-1,3-dioxoisoindolin-5-yl)piperazin-1-yl)propanamido)-4-((2-methoxy-3-(1-methyl-1H-1,2,4-triazol-3-yl)phenyl)amino)-N-methylpyridazine-3-carboxamide (5 mg, 30% yield) as a yellow solid.

Chemical Formula: C₃₆H₃₈N₁₂O₇; Molecular Weight: 750.78

1H NMR (400 MHz, CD₃OD, ppm): δ 8.52 (s, 1H), 7.97 (s, 1H), 7.77-7.72 (m, 2H), 7.58 (d, J=8.0 Hz, 1H), 7.47 (s, 1H), 7.35-7.30 (m, 2H), 5.11-5.07 (m, 1H), 4.01 (d, J=2.4 Hz, 3H), 3.74-3.70 (m, 6H), 3.61-3.52 (m, 7H), 3.11-3.10 (m, 2H), 2.99 (s, 3H), 2.86-2.70 (m, 3H), 2.16-2.12 (m, 1H).

LCMS: (ES⁺): m/z 751.2[M+1]⁺, t_{R}=2.94 min.

### Synthesis of PROTAC 3: 6-(3-(4-((1-(2-(2,6-Dioxopiperidin-3-yl)-1,3-dioxoisoindolin-5-yl)azetidin-3-yl)methyl)piperidin-1-yl)propanamido)-4-((2-methoxy-3-(1-methyl-1H-1,2,4-triazol-3-yl)phenyl)amino)-N-methylpyridazine-3-carboxamide

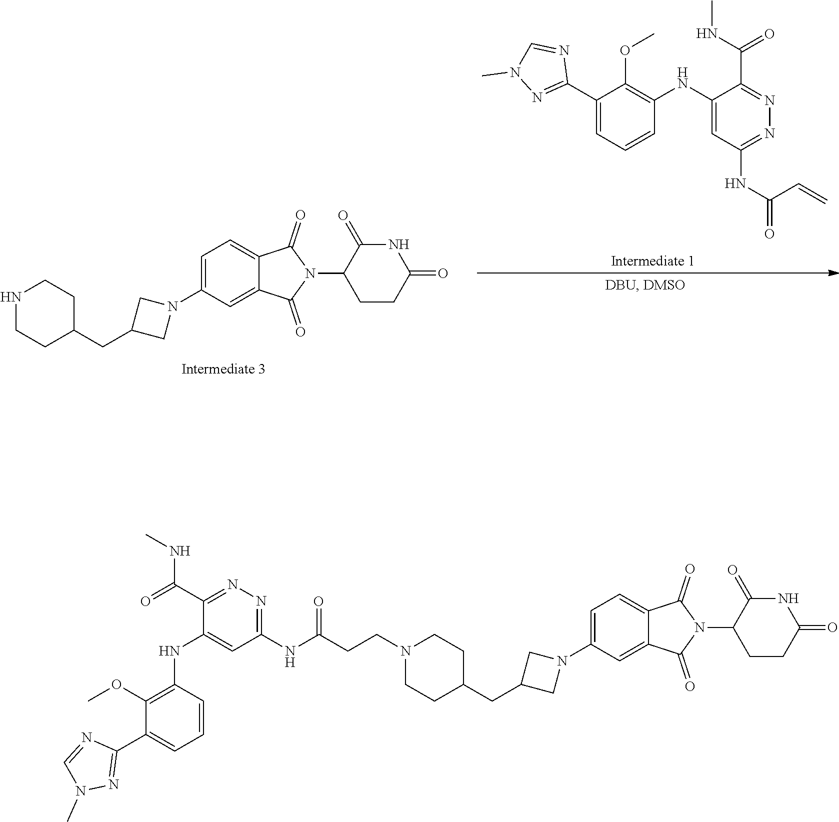 [SMILES: O=C1CCC(N2C(=O)c3ccc(N4CC(CC5CCNCC5)C4)cc3C2=O)C(=O)N1 ; C=CC(=O)Nc1cc(Nc2cccc(-c3ncn(C)n3)c2OC)c(C(=O)NC)nn1 ; CNC(=O)c1nnc(NC(=O)CCN2CCC(CC3CN(c4ccc5c(c4)C(=O)N(C4CCC(=O)NC4=O)C5=O)C3)CC2)cc1Nc1cccc(-c2ncn(C)n2)c1OC]

To a solution of 2-(2,6-dioxopiperidin-3-yl)-5-(3-(piperidin-4-ylmethyl)azetidin-1-yl)isoindoline-1,3-dione (Intermediate 3, 100 mg, 0.244 mmol) and 6-acrylamido-4-((2-methoxy-3-(1-methyl-1H-1,2,4-triazol-3-yl)phenyl)amino)-N-methylpyridazine-3-carboxamide (Intermediate 1, 50 mg, 0.122 mmol) in DMSO (4 mL) was added DBU (149 mg, 0.980 mmol) at 0° C. The mixture was stirred at 25° C. for 30 min. The reaction was quenched with saturated NH₄Cl (20 mL) and extracted with DCM (30 mL) and CH₃OH (3 mL). The organic phase was washed with saturated NaCl (20 mL). The organic was concentrated and purified by prep-HPLC with the following conditions (Welch Ultimate XB-C18, 21.2*250 mm 5 um), eluted with H₂O in CH₃CN containing 0.05% TFA). The product containing prep-HPLC fraction was lyophilized to dryness to afford the desired product 6-(3-(4-((1-(2-(2,6-Dioxopiperidin-3-yl)-1,3-dioxoisoindolin-5-yl)azetidin-3-yl)methyl)piperidin-1-yl)propanamido)-4-((2-methoxy-3-(1-methyl-1H-1,2,4-triazol-3-yl)phenyl)amino)-N-methylpyridazine-3-carboxamide (33 mg, 33% yield) as a yellow solid.

Chemical Formula: C₄₁H₄₆N₁₂O₇; Molecular Weight: 818.90

¹H NMR (400 MHz, CD₃OD, ppm): δ 8.55 (s, 1H), 7.80-7.75 (m, 2H), 7.61-7.57 (m, 2H), 7.33-7.29 (m, 1H), 6.76-6.75 (m, 1H), 6.61-6.58 (m, 1H), 5.06-5.02 (m, 1H), 4.18-4.14 (m, 2H), 4.02 (s, 3H), 3.73 (s, 3H), 3.67-3.60 (m, 4H), 3.47 (s, 2H), 3.06-2.64 (m, 11H), 2.11-2.07 (m, 1H), 1.98-1.95 (m, 2H), 1.69 (s, 3H), 1.57-1.47 (m, 2H).

LCMS: (ES⁺): m/z 818.9[M+1]⁺, t_{R}=3.180 min.

### Synthesis of PROTAC 4: 6-(3-(4-(6-(4-(2-(2,6-Dioxopiperidin-3-yl)-1,3-dioxoisoindolin-5-yl)piperazin-1-yl)pyrimidin-4-yl)piperazin-1-yl)propanamido)-4-((2-methoxy-3-(1-methyl-1H-1,2,4-triazol-3-yl)phenyl)amino)-N-methylpyridazine-3-carboxamide

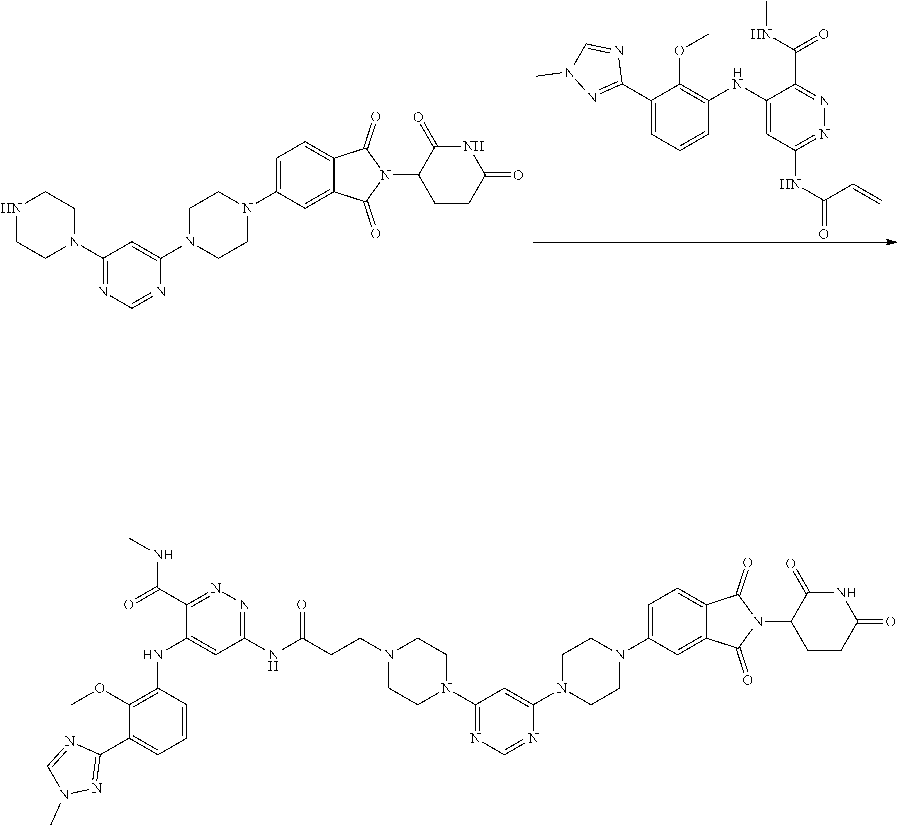 [SMILES: O=C1CCC(N2C(=O)c3ccc(N4CCN(c5cc(N6CCNCC6)ncn5)CC4)cc3C2=O)C(=O)N1 ; C=CC(=O)Nc1cc(Nc2cccc(-c3ncn(C)n3)c2OC)c(C(=O)NC)nn1 ; CNC(=O)c1nnc(NC(=O)CCN2CCN(c3cc(N4CCN(c5ccc6c(c5)C(=O)N(C5CCC(=O)NC5=O)C6=O)CC4)ncn3)CC2)cc1Nc1cccc(-c2ncn(C)n2)c1OC]

To a solution of 2-(2,6-dioxopiperidin-3-yl)-5-(4-(6-(piperazin-1-yl)pyrimidin-4-yl)piperazin-1-yl)isoindoline-1,3-dione (Intermediate 4, 100 mg, 0.198 mmol) and 6-acrylamido-4-((2-methoxy-3-(1-methyl-1H-1,2,4-triazol-3-yl)phenyl)amino)-N-methylpyridazine-3-carboxamide (Intermediate 1, 50 mg, 0.126 mmol) in DMSO (4 mL) was added DBU (149 mg, 0.980 mmol) at 0 TC. The mixture was stirred at 25° C. for 1.5 h. The reaction was quenched with saturated NH₄Cl (20 mL) and extracted with DCM (30 mL) and CH₃OH (3 mL). The organic phase was washed with saturated NaCl (20 mL). The organic was concentrated and purified by prep-HPLC with the following conditions (Welch Ultimate XB-C18, 21.2*250 mm 5 um), eluted with H₂ in CH₃CN containing 0.05% TFA). The product containing prep-HPLC fraction was lyophilized to dryness to afford the desired product 6-(3-(4-(6-(4-(2-(2,6-Dioxopiperidin-3-yl)-1,3-dioxoisoindolin-5-yl)piperazin-1-yl)pyrimidin-4-yl)piperazin-1-yl)propanamido)-4-((2-methoxy-3-(1-methyl-1H-1,2,4-triazol-3-yl)phenyl)amino)-N-methylpyridazine-3-carboxamide (37 mg, 32% yield) as a yellow solid.

Chemical Formula: C₄₄H₄₈N₁₆O₇; Molecular Weight: 912.97

¹H NMR (400 MHz, CD₃OD, ppm): δ 8.55 (s, 1H), 8.30 (s, 1H), 7.78-7.69 (m, 3H), 7.59-7.57 (m, 1H), 7.33-7.29 (m, 2H), 7.21-7.19 (m, 1H), 6.13 (s, 1H), 5.09-5.05 (m, 1H), 4.06-4.02 (m, 7H), 3.90-3.87 (m, 4H), 3.72 (s, 3H), 3.69-3.67 (m, 4H), 3.60-3.57 (m, 2H), 3.49 (s, 4H), 3.14-3.12 (m, 2H), 2.98 (s, 3H), 2.90-2.81 (m, 1H), 2.76-2.65 (m, 2H), 2.12-2.09 (m, 1H).

LCMS: (ES⁺): m/z 913.3[M+1]⁺, t_{R}=2.997 min.

### Synthesis of PROTAC 5: 6-(3-(4-(((1-(2-(2,6-Dioxopiperidin-3-yl)-1,3-dioxoisoindolin-5-yl)piperidin-4-yl)methoxy)methyl)piperidin-1-yl)propanamido)-4-((2-methoxy-3-(1-methyl-1H-1,2,4-triazol-3-yl)phenyl)amino)-N-methylpyridazine-3-carboxamide

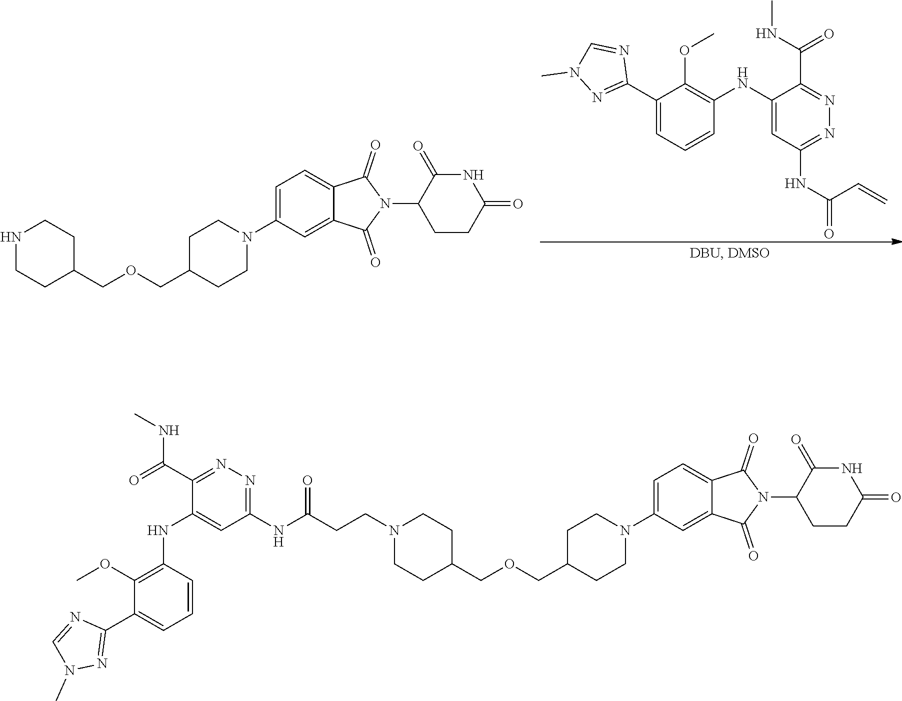 [SMILES: O=C1CCC(N2C(=O)c3ccc(N4CCC(COCC5CCNCC5)CC4)cc3C2=O)C(=O)N1 ; C=CC(=O)Nc1cc(Nc2cccc(-c3ncn(C)n3)c2OC)c(C(=O)NC)nn1 ; CNC(=O)c1nnc(NC(=O)CCN2CCC(COCC3CCN(c4ccc5c(c4)C(=O)N(C4CCC(=O)NC4=O)C5=O)CC3)CC2)cc1Nc1cccc(-c2ncn(C)n2)c1OC]

To a solution of 2-(2,6-dioxopiperidin-3-yl)-5-(4-((piperidin-4-ylmethoxy)methyl)piperidin-1-yl)isoindoline-1,3-dione (Intermediate 5, 100 mg, 0.213 mmol) and 6-acrylamido-4-((2-methoxy-3-(1-methyl-1H-1,2,4-triazol-3-yl)phenyl)amino)-N-methylpyridazine-3-carboxamide (Intermediate 1, 50 mg, 0.126 mmol) in DMSO (4 mL) was added DBU (149 mg, 0.980 mmol) at 0° C. The mixture was stirred at 25° C. for 30 min. The reaction was quenched with saturated NH₄Cl (20 mL) and extracted with DCM (30 mL) and CH₃OH (3 mL). The organic phase was washed with saturated NaCl (20 mL). The organic was concentrated and purified by prep-HPLC with the following conditions (Welch Ultimate XB-C18, 21.2*250 mm 5 um), eluted with H₂O in CH₃CN containing 0.05% TFA). The product containing prep-HPLC fraction was lyophilized to dryness to afford the desired product 6-(3-(4-(((1-(2-(2,6-Dioxopiperidin-3-yl)-1,3-dioxoisoindolin-5-yl)piperidin-4-yl)methoxy)methyl)piperidin-1-yl)propanamido)-4-((2-methoxy-3-(1-methyl-1H-1,2,4-triazol-3-yl)phenyl)amino)-N-methylpyridazine-3-carboxamide (42 mg, 38% yield) as a yellow solid.

Chemical Formula: C₄₄H₅₂N₁₂O₈; Molecular Weight: 876.98

¹H NMR (400 MHz, CD₃OD, ppm): δ 8.56 (s, 1H), 7.78-7.73 (m, 2H), 7.64-7.56 (m, 2H), 7.33-7.29 (m, 2H), 7.18-7.16 (m, 1H), 5.07-5.03 (m, 1H), 4.04-4.02 (m, 5H), 3.72 (s, 3H), 3.64-3.60 (m, 2H), 3.48-3.44 (m, 2H), 3.33-3.32 (m, 4H), 3.05-2.95 (m, 9H), 2.90-2.80 (m, 1H), 2.76-2.64 (m, 2H), 2.11-2.05 (m, 1H), 1.98-1.79 (m, 6H), 1.61-1.55 (m, 2H), 1.41-1.32 (m, 2H).

LCMS: (ES⁺): m/z 877.3[M+1]⁺, t_{R}=3.301 min.

### Synthesis of PROTAC 6: 6-(3-(((1-(2-(2,6-Dioxopiperidin-3-yl)-1,3-dioxoisoindolin-5-yl)piperidin-4-yl)methyl)amino)propanamido)-4-((2-methoxy-3-(1-methyl-1H-1,2,4-triazol-3-yl)phenyl)amino)-N-methylpyridazine-3-carboxamide

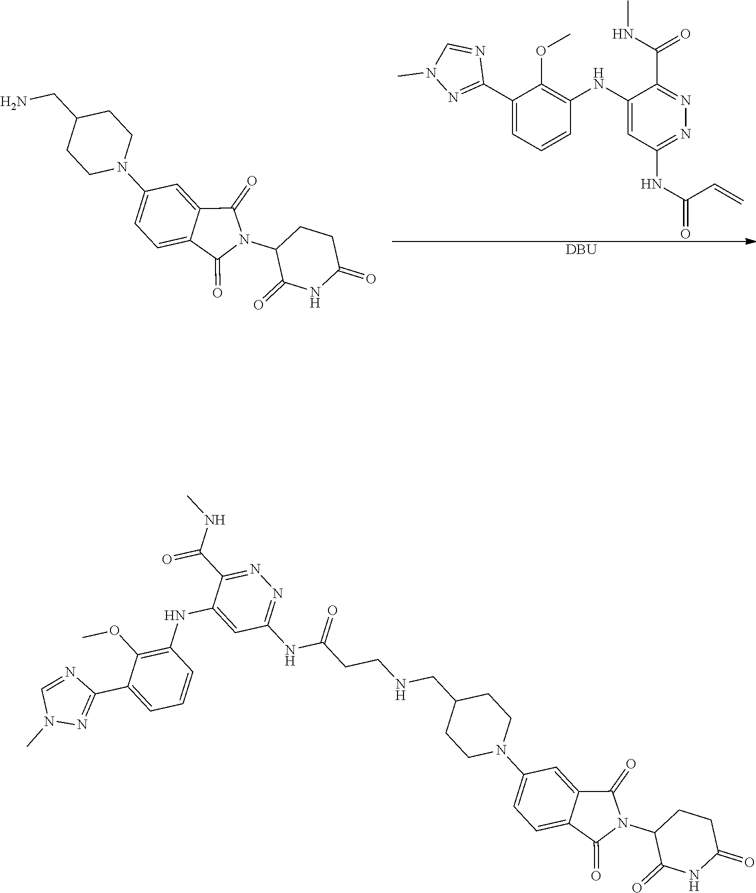 [SMILES: NCC1CCN(c2ccc3c(c2)C(=O)N(C2CCC(=O)NC2=O)C3=O)CC1 ; C=CC(=O)Nc1cc(Nc2cccc(-c3ncn(C)n3)c2OC)c(C(=O)NC)nn1 ; "DBU" ; CNC(=O)c1nnc(NC(=O)CCNCC2CCN(c3ccc4c(c3)C(=O)N(C3CCC(=O)NC3=O)C4=O)CC2)cc1Nc1cccc(-c2ncn(C)n2)c1OC]

A solution of 5-(4-(Aminomethyl)piperidin-1-yl)-2-(2,6-dioxopiperidin-3-yl)isoindoline-1,3-dione (Intermediate 6, 97.5 mg, 0.24 mmol) and 6-acrylamido-4-((2-methoxy-3-(1-methyl-1H-1,2,4-triazol-3-yl)phenyl)amino)-N-methylpyridazine-3-carboxamide (Intermediate 1, 50 mg, 0.12 mmol) and DBU (146 mg, 0.96 mmol) in DMSO (5 mL) was stirred at 25° C. for 6 h. The reaction was quenched with saturated NH₄Cl (2 mL) and extracted with DCM (10 mL) and CH₃OH (1 mL). The organic phase was washed with saturated NaCl (2 mL). The organic was concentrated and purified by prep-HPLC with the following conditions (Welch Ultimate XB-C18, 21.2*250 mm Sum), eluted with H₂O in CH₃CN containing 0.05% TFA). The product containing prep-HPLC fraction was lyophilized to dryness to afford the desired product 6-(3-(((1-(2-(2,6-Dioxopiperidin-3-yl)-1,3-dioxoisoindolin-5-yl)piperidin-4-yl)methyl)amino)propanamido)-4-((2-methoxy-3-(1-methyl-1H-1,2,4-triazol-3-yl)phenyl)amino)-N-methylpyridazine-3-carboxamide (25 mg, 27% yield) as a yellow solid.

Chemical Formula: C₃₈H₄₂N₁₂O₇; Molecular Weight: 778.83

¹H NMR (400 MHz, CD₃OD, ppm): δ 8.52 (s, 1H), 7.99 (s, 1H), 7.74-7.73 (m, 1H), 7.66 (d, J=8.4 Hz, 1H), 7.58-7.57 (m, 1H), 7.34-7.28 (m, 2H), 7.22-7.20 (m, 1H), 5.09-5.04 (m, 1H), 4.07-4.01 (m, 5H), 3.71 (s, 3H), 3.38-3.37 (m, 2H), 3.01-2.98 (m, 9H), 2.85-2.72 (m, 3H), 2.03-1.93 (m, 2H), 1.92-1.89 (m, 2H), 3.39-3.37 (m, 2H).

LCMS: (ES⁺): m/z 779.3[M+1]⁺, t_{R}=3.07 min.

### Synthesis of PROTAC 7: 6-(3-(3-((4-(1-(2-(2,6-Dioxopiperidin-3-yl)-1,3-dioxoisoindolin-5-yl)piperidin-4-yl)piperazin-1-yl)methyl)azetidin-1-yl)propanamido)-4-((2-methoxy-3-(1-methyl-1H-1,2,4-triazol-3-yl)phenyl)amino)-N-methylpyridazine-3-carboxamide

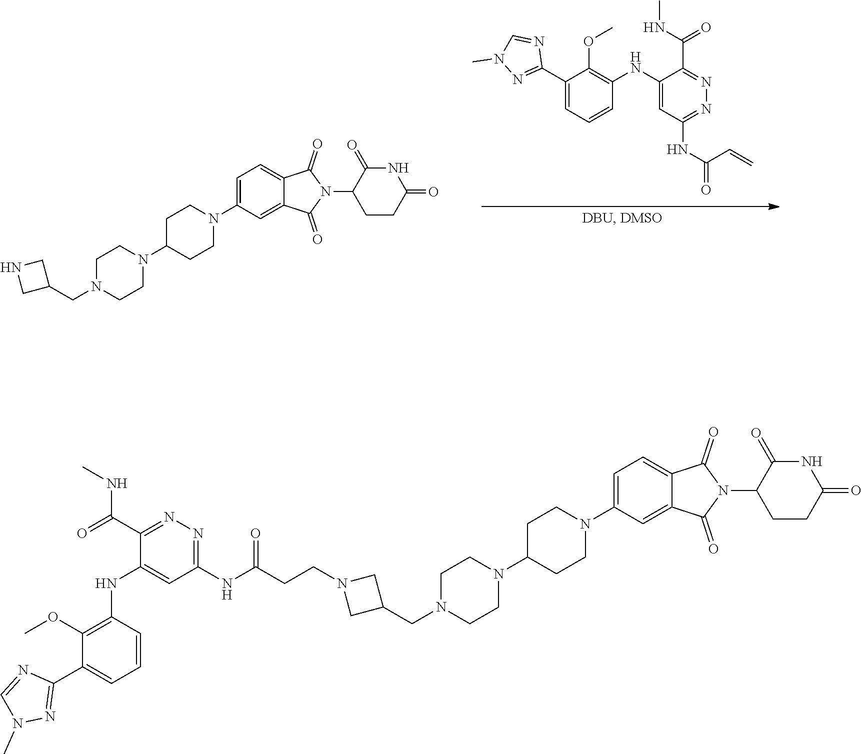 [SMILES: O=C1CCC(N2C(=O)c3ccc(N4CCC(N5CCN(CC6CNC6)CC5)CC4)cc3C2=O)C(=O)N1 ; C=CC(=O)Nc1cc(Nc2cccc(-c3ncn(C)n3)c2OC)c(C(=O)NC)nn1 ; CNC(=O)c1nnc(NC(=O)CCN2CC(CN3CCN(C4CCN(c5ccc6c(c5)C(=O)N(C5CCC(=O)NC5=O)C6=O)CC4)CC3)C2)cc1Nc1cccc(-c2ncn(C)n2)c1OC]

To a solution of 5-(4-(4-(azetidin-3-ylmethyl)piperazin-1-yl)piperidin-1-yl)-2-(2,6-dioxopiperidin-3-yl)isoindoline-1,3-dione (Intermediate 7, 100 mg, 0.202 mmol) and 6-acrylamido-4-((2-methoxy-3-(1-methyl-1H-1,2,4-triazol-3-yl)phenyl)amino)-N-methylpyridazine-3-carboxamide (Intermediate 1, 50 mg, 0.126 mmol) in DMSO (4 mL) was added DBU (149 mg, 0.980 mmol) at 0° C. The mixture was stirred at 25° C. for 30 mi. The reaction was quenched with saturated NH₄Cl (20 mL) and extracted with DCM (30 mL) and CH₃OH (3 mL). The organic phase was washed with saturated NaCl (20 mL). The organic was concentrated and purified by prep-HPLC with the following conditions (Welch Ultimate XB-C18, 21.2*250 mm 5 um), eluted with H₂O in CH₃CN containing 0.05% TFA). The product containing prep-HPLC fraction was lyophilized to dryness to afford the desired product 6-(3-(3-((4-(1-(2-(2,6-Dioxopiperidin-3-yl)-1,3-dioxoisoindolin-5-yl)piperidin-4-yl)piperazin-1-yl)methyl)azetidin-1-yl)propanamido)-4-((2-methoxy-3-(1-methyl-1H-1,2,4-triazol-3-yl)phenyl)amino)-N-methylpyridazine-3-carboxamide (28 mg, 25% yield) as a yellow solid.

Chemical Formula: C₄₅H₅₄N₁₄O₇; Molecular Weight: 903.02

¹H NMR (400 MHZ, CD₃OD, ppm): δ 8.54 (s, 1H), 7.85 (s, 1H), 7.75-7.74 (m, 1H), 7.70-7.68 (m, 1H), 7.59-7.57 (m, 1H), 7.39-7.38 (m, 1H), 7.32-7.25 (m, 2H), 5.09-5.05 (m, 1H), 4.27-4.18 (m, 4H), 4.02-3.90 (m, 5H), 3.72 (s, 3H), 3.55 (s, 2H), 3.45-3.42 (m, 5H), 3.14-2.98 (m, 7H), 2.91-2.66 (m, 10H), 2.23-2.20 (m, 2H), 2.12-2.08 (m, 1H), 1.81-1.73 (m, 2H).

LCMS: (ES⁺): m/z 903.4[M+1]⁺, t_{R}=2.772 min.

### Synthesis of PROTAC 8: 6-(3-(4-(3-(4-(2-(2,6-Dioxopiperidin-3-yl)-1,3-dioxoisoindolin-5-yl)piperazin-1-yl)propyl)piperidin-1-yl)propanamido)-4-((2-methoxy-3-(1-methyl-1H-1,2,4-triazol-3-yl)phenyl)amino)-N-methylpyridazine-3-carboxamide

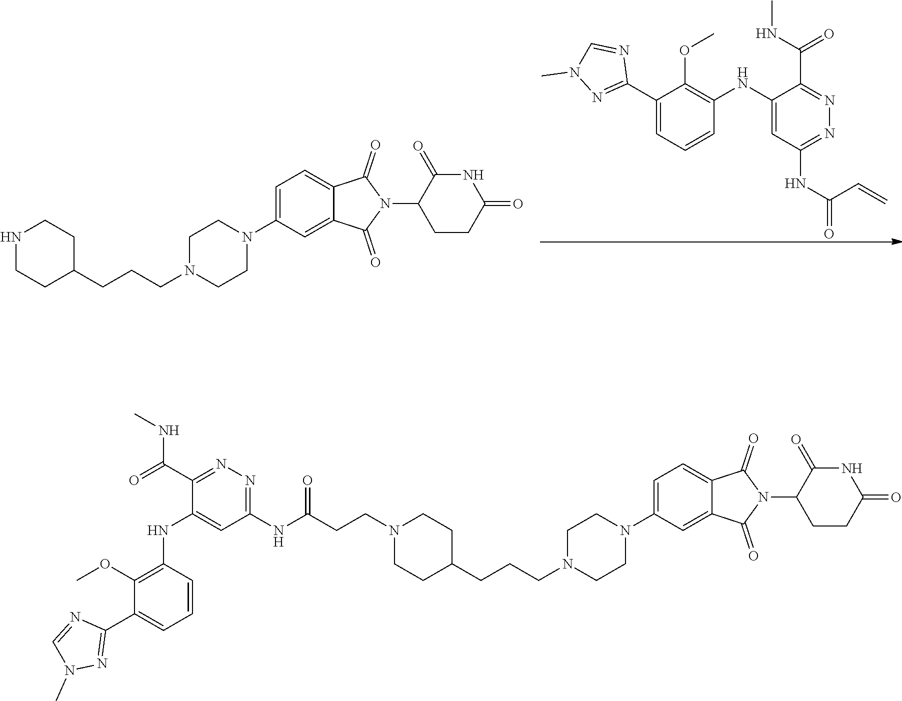 [SMILES: O=C1CCC(N2C(=O)c3ccc(N4CCN(CCCC5CCNCC5)CC4)cc3C2=O)C(=O)N1 ; C=CC(=O)Nc1cc(Nc2cccc(-c3ncn(C)n3)c2OC)c(C(=O)NC)nn1 ; CNC(=O)c1nnc(NC(=O)CCN2CCC(CCCN3CCN(c4ccc5c(c4)C(=O)N(C4CCC(=O)NC4=O)C5=O)CC3)CC2)cc1Nc1cccc(-c2ncn(C)n2)c1OC]

To a solution of 2-(2,6-dioxopiperidin-3-yl)-5-(4-(3-(piperidin-4-yl)propyl)piperazin-1-yl)isoindoline-1,3-dione (Intermediate 8, 55 mg, 0.117 mmol) and 6-acrylamido-4-((2-methoxy-3-(1-methyl-1H-1,2,4-triazol-3-yl)phenyl)amino)-N-methylpyridazine-3-carboxamide (Intermediate 1, 40 mg, 0.098 mmol) in DMSO (4 mL) was added DBU (119 mg, 0.784 mmol) at 0° C. The mixture was stirred at 25° C. for 30 m. The reaction was quenched with saturated NH₄Cl (20 mL) and extracted with DCM (30 mL) and CH₃OH (3 mL). The organic phase was washed with saturated NaCl (20 mL). The organic was concentrated and purified by prep-HPLC with the following conditions (Welch Ultimate XB-C18, 21.2*250 mm 1 um), eluted with H₂ in CH₃CN containing 0.05% TFA). The product containing prep-HPLC fraction was lyophilized to dryness to afford the desired product 6-(3-(4-(3-(4-(2-(2,6-Dioxopiperidin-3-yl)-1,3-dioxoisoindolin-5-yl)piperazin-1-yl)propyl)piperidin-1-yl)propanamido)-4-((2-methoxy-3-(1-methyl-1H-1,2,4-triazol-3-yl)phenyl)amino)-N-methylpyridazine-3-carboxamide (25 mg, 29% yield) as a yellow solid.

Chemical Formula: C₄₄H₅₃N₁₃O₇; Molecular Weight: 875.99

¹H NMR (400 MHz, CD₃OD, ppm): δ 8.52 (s, 1H), 7.91 (s, 1H), 7.75-7.72 (m, 2H), 7.59-7.57 (m, 1H), 7.46-7.45 (m, 1H), 7.34-7.28 (m, 2H), 5.11-5.06 (m, 1H), 4.19-4.12 (m, 1H), 4.02 (s, 3H), 3.72-3.61 (m, 7H), 3.53-3.47 (m, 3H), 3.31-3.30 (m, 2H), 3.22-3.18 (m, 4H), 3.06-2.98 (m, 7H), 2.91-2.65 (m, 3H), 2.13-2.09 (m, 1H), 2.02-1.99 (m, 2H), 1.86-1.78 (m, 2H), 1.66-1.65 (m, 1H), 1.50-1.29 (m, 4H).

LCMS: (ES⁺): m/z 876.4[M+1]⁺, t_{R}=2.772 min.

### Synthesis of PROTAC 9: 6-(3-(4-(2-(1-(4-((2-(2,6-Dioxopiperidin-3-yl)-1,3-dioxoisoindolin-5-yl)oxy)phenyl)piperidin-4-yl)ethyl)piperidin-1-yl)propanamido)-4-((2-methoxy-3-(1-methyl-1H-1,2,4-triazol-3-yl)phenyl)amino)-N-methylpyridazine-3-carboxamide

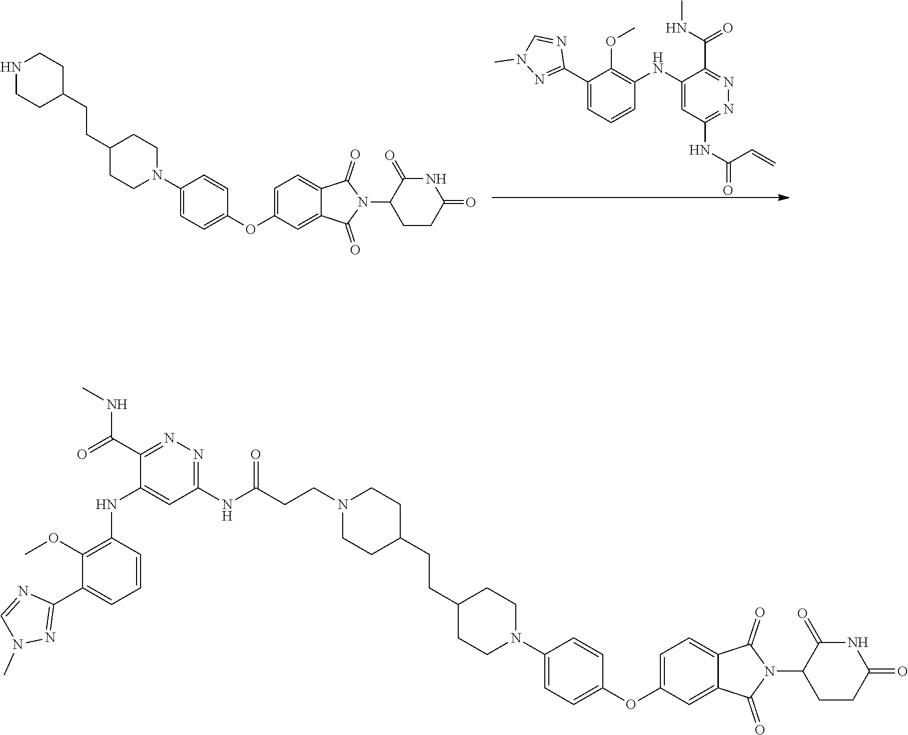 [SMILES: O=C1CCC(N2C(=O)c3ccc(Oc4ccc(N5CCC(CCC6CCNCC6)CC5)cc4)cc3C2=O)C(=O)N1 ; C=CC(=O)Nc1cc(Nc2cccc(-c3ncn(C)n3)c2OC)c(C(=O)NC)nn1 ; CNC(=O)c1nnc(NC(=O)CCN2CCC(CCC3CCN(c4ccc(Oc5ccc6c(c5)C(=O)N(C5CCC(=O)NC5=O)C6=O)cc4)CC3)CC2)cc1Nc1cccc(-c2ncn(C)n2)c1OC]

To a solution of 2-(2,6-dioxopiperidin-3-yl)-5-(4-(4-(2-(piperidin-4-yl)ethyl)piperidin-1-yl)phenoxy)isoindoline-1,3-dione (Intermediate 9, 100 mg, 0.183 mmol) and 6-acrylamido-4-((2-methoxy-3-(1-methyl-1H-1,2,4-triazol-3-yl)phenyl)amino)-N-methylpyridazine-3-carboxamide (Intermediate 1, 50 mg, 0.126 mmol) in DMSO (4 mL) was added DBU (149 mg, 0.980 mmol) at 0° C. The mixture was stirred at 25° C. for 1.5 h. The reaction was quenched with saturated NH₄Cl (20 mL) and extracted with DCM (30 mL) and CH₃OH (3 mL). The organic phase was washed with saturated NaCl (20 mL). The organic was concentrated and purified by prep-HPLC with the following conditions (Welch Ultimate XB-C18, 21.2*250 mm 5 um), eluted with H₂O in CH₃CN containing 0.05% TFA). The product containing prep-HPLC fraction was lyophilized to dryness to afford the desired product 6-(3-(4-(2-(1-(4-((2-(2,6-Dioxopiperidin-3-yl)-1,3-dioxoisoindolin-5-yl)oxy)phenyl)piperidin-4-yl)ethyl)piperidin-1-yl)propanamido)-4-((2-methoxy-3-(1-methyl-1H-1,2,4-triazol-3-yl)phenyl)amino)-N-methylpyridazine-3-carboxamide (35 mg, 29% yield) as a yellow solid.

Chemical Formula: C₅₀H₅₆N₁₂O₈; Molecular Weight: 953.07

¹H NMR (400 MHz, CD₃OD, ppm): δ 8.53 (s, 1H), 7.91-7.85 (m, 2H), 7.75-7.68 (m, 3H), 7.59-7.57 (m, 1H), 7.45-7.42 (m, 1H), 7.39-7.38 (m, 1H), 7.33-7.29 (m, 3H), 5.14-5.10 (m, 1H), 4.02 (s, 3H), 3.73-3.70 (m, 5H), 3.63-3.47 (m, 6H), 3.06-2.98 (m, 7H), 2.91-2.65 (m, 3H), 2.15-1.99 (m, 5H), 1.70-1.61 (m, 4H), 1.51-1.40 (m, 6H).

LCMS: (ES⁺): m/z 953.4[M+1]⁺, t_{R}=3.294 min.

FIG. 2A shows the amount of protein detected in cell lysate after 24 hour treatment with PROTAC, relative to DMSO-treated control, versus concentration of PROTAC, illustrating the “inverse dose-response” (less degradation at higher doses, a known phenomenon in PROTAC induced protein degradation, also called “hook effect”). The TYK2 inhibitors alone (PROTAC examples 1, 6-9) does not reduce the amount of TYK2 protein.

### Synthesis of PROTAC 10: 6-(3-(4-(((2-(2,6-Dioxopiperidin-3-yl)-1,3-dioxoisoindolin-5-yl)amino)methyl)piperidin-1-yl)propanamido)-4-((2-methoxy-3-(1-methyl-1H-1,2,4-triazol-3-yl)phenyl)amino)-N-methylpyridazine-3-carboxamide

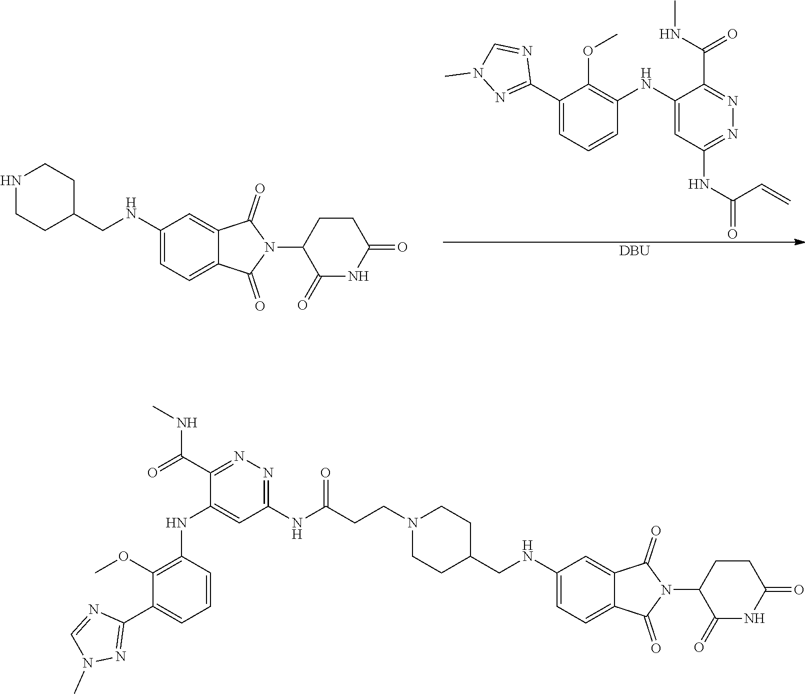 [SMILES: O=C1CCC(N2C(=O)c3ccc(NCC4CCNCC4)cc3C2=O)C(=O)N1 ; C=CC(=O)Nc1cc(Nc2cccc(-c3ncn(C)n3)c2OC)c(C(=O)NC)nn1 ; "DBU" ; CNC(=O)c1nnc(NC(=O)CCN2CCC(CNc3ccc4c(c3)C(=O)N(C3CCC(=O)NC3=O)C4=O)CC2)cc1Nc1cccc(-c2ncn(C)n2)c1OC]

A mixture of 6-acrylamido-4-((2-methoxy-3-(1-methyl-1H-1,2,4-triazol-3-yl)phenyl)amino)-N-methylpyridazine-3-carboxamide (Intermediate 1, 50 mg, 0.12 mmol) and 2-(2,6-Dioxopiperidin-3-yl)-5-((piperidin-4-ylmethyl)amino)isoindoline-1,3-dione (Intermediate 10, 106 mg, 0.24 mmol) and DBU (146 mg, 0.96 mmol) in DMSO (3 mL) was stirred at 25° C. for 6 h. The reaction was quenched with saturated NH₄Cl (2 mL) and extracted with DCM (20 mL) and CH₃OH (2 mL). The organic phase was washed with saturated NaCl (2 mL). The organic was concentrated and purified by prep-HPLC with the following conditions (Welch Ultimate XB-C18, 21.2*250 mm 5 um), eluted with H₂O in CH₃CN containing 0.05% TFA). The product containing prep-HPLC fraction was lyophilized to dryness to afford the desired product 6-(3-(4-(((2-(2,6-Dioxopiperidin-3-yl)-1,3-dioxoisoindolin-5-yl)amino)methyl)piperidin-1-yl)propanamido)-4-((2-methoxy-3-(1-methyl-1H-1,2,4-triazol-3-yl)phenyl)amino)-N-methylpyridazine-3-carboxamide (45 mg, 48% yield) as a white solid.

Chemical Formula: C₃₈H₄₂N₁₂O₇; Molecular Weight: 778.83

¹H NMR (400 MHz, CD₃OD, ppm): δ 8.55 (s, 1H), 7.77-7.75 (m, 2H), 7.58-7.54 (m, 2H), 7.31 (t, J=8.0 Hz, 1H), 6.99 (d, J=2.0 Hz, 1H), 6.87-6.84 (m, 1H), 5.06-5.01 (m, 1H), 4.02 (s, 3H), 3.72 (s, 3H), 3.70-3.62 (m, 2H), 3.50-3.46 (m, 2H), 3.18-3.17 (m, 2H), 3.07-2.98 (m, 7H), 2.95-2.65 (m, 3H), 2.15-1.96 (m, 4H), 1.65-1.51 (m, 2H).

LCMS: (ES⁺): m/z 779.3 [M+1]⁺, t_{R}=3.01 min.

### Protein Level Control

This description also provides methods for the control of protein levels within a cell. The method is based on the use of compounds as described herein such that degradation of the target protein TYK2 in vivo will result in the reducing the amount of the target protein in a biological system, preferably to provide a particular therapeutic benefit.

The following examples are used to assist in describing the present disclosure, but should not be seen as limiting the present disclosure in any way.

In certain embodiments, the description provides the following exemplary TYK2-degrading bifunctional molecules (compounds of Table 1 or PROTACs 1-10), including salts, polymorphs, analogs, derivatives, and deuterated forms thereof.

### Assay for Testing TYK2 Degradation Driven by Compounds Including PROTACs Designed to Target TYK2

With reference to FIGS. 2A-2B. MOLT-4 cells (ATCC CRL-1582) were acquired from ATCC and maintained in RPMI-1640 medium supplemented with 10% fetal bovine serum (ATCC 30-2020), the combination henceforth referred to as “media”. Cells were maintained according to ATCC, (see product sheet), including incubation at 37° C. with 5% C₀₂ in air other than during brief manipulations in a cell culture hood or during centrifugation.

18-24 hours before compound treatment, cells growing for 2-3 days in a flask were centrifuged at 200×g for 8 min, the media was aspirated, and fresh media was used to resuspend the cells. Cells were then counted, diluted with fresh media, and seeded at 650,000-750,000 cells/ml in 6-well plates, with 2 ml of volume per well.

Compounds to be tested were solubilized and stored in dimethyl sulfoxide (DMSO) at 10 mM. Compounds were first diluted in DMSO and then media so that the DMSO concentration was identical across treatments and the final concentration of DMSO was below 0.1% (typically 0.04%). Cells in each well of a 6-well plate were treated by adding 0.5 ml of fresh media containing varying concentrations of compounds to be tested—or only DMSO as a “no treatment” control.

Cells were harvested 24 hours after treatment by collecting all volume from each well into labeled microfuge tubes followed by centrifugation at 200×g for 8 min. Cells were washed in 1 ml of phosphate buffered saline (DPBS; Gibco 141190144) and then 0.5 ml of DPBS by centrifugation (as above) and aspiration. Cell pellets were resuspended by pipetting up-and-down in 100 microliters of ice cold RIPA buffer (Thermo, 89900) supplemented with 1× protease inhibitor cocktail and 1×EDTA (Pierce, 87786) and 1× phosphatase inhibitor cocktail (Pierce, 78420). Cell extracts were incubated on ice for 10 min and then centrifuged at 21,130×g for 20 min in a microfuge. Supernatants were collected and protein concentration for each sample was determined using a BCA kit (Thermo, 23225). Each sample was then denatured by adding 4×LDS buffer (Invitrogen, NP0008) to 1× followed by heating at 95° C. for 5 minutes.

Room temperature protein extracts in 1×LDS buffer were loaded on NuPAGE 4-12% Bis-Tris gels (Invitrogen, WG1402BOX) so that each lane had an equal amount of protein for a given experiment (typically 10-15 micrograms protein per lane). Proteins were separated by electrophoresis using a XCell4 Surelock Midi-Cell apparatus (Invitrogen) and then transferred to PVDF membranes within iBlot 2 Transfer Stacks (Invitrogen, IB24001) using an iBlot 2 system (Invitrogen). Membranes were cut horizontally at a 50 kDa molecular weight marker and each piece of membrane was blocked in TBS-T+5% non-fat dry milk (NFDM) for 1-24 hours. Membranes were then incubated with antibody to TYK2 (Cell Signaling Technologies, 14193) or GAPDH (Cell Signaling Technologies, 5174; as loading control) in TBS-T+1% NFDM for 1 hour. Membranes were washed four times in TBS-T for 10 min each time, and then incubated with a HRP-conjugated secondary antibody (Cell Signaling Technologies, 7074) in TBS-T+1% NFDM for one hour. Membranes were washed four times for 10 min in TBS-T. Membranes were then incubated with reagents according to the SuperSignal West Femto Kit (Thermo, 34095) for 1 min at room temperature. Membranes were then imaged using a Gel Doc XR+ system (BioRad).

Captured images were processed using Image Lab software (BioRad). Using the volume tool, band intensities within rectangles of equal area were determined with the local subtraction method. The effects of compounds on TYK2 levels were related to the level of TYK2 from cells treated with only DMSO (no compound).

TABLE 1
Degradation and characterization of exemplary bifunctional compounds of the present disclosure

| | | | | Tyk2 | |
|---|---|---|---|---|---|
| | | | | De- | |
| | | | | grada- | |
| | | Mol | | tion | E3 |
| PROTAC | Structure | Weight | NMR | Score | Ligase |
| 1 | 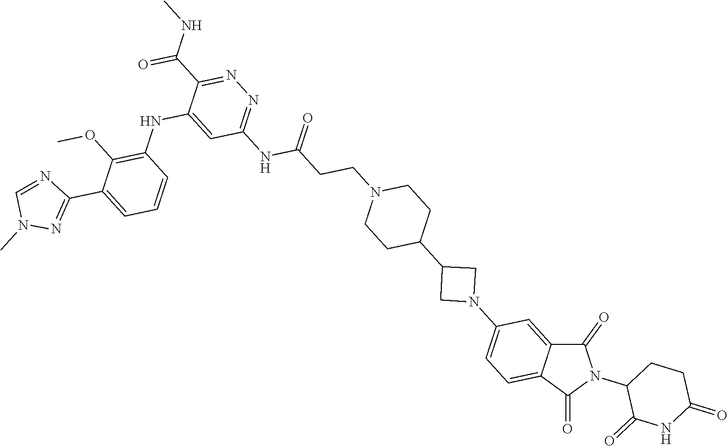 [SMILES: CNC(=O)c1nnc(NC(=O)CCN2CCC(C3CN(c4ccc5c(c4)C(=O)N(C4CCC(=O)NC4=O)C5=O)C3)CC2)cc1Nc1cccc(-c2ncn(C)n2)c1OC] | 804.87 | ¹H NMR (400 MHz, CD₃OD, ppm): δ 8.55 (s, 1H), 7.78-7.75 (m, 2H), 7.62-7.56 (m, 2H), 7.33-7.29 (m, 1H), 6.78-6.77 (m, 1H), 6.62-6.60 (m, 1H), 5.07-5.02 (m, 1H), 4.13-4.09 (m, 2H), 4.02 (s, 3H), 3.79-3.76 (m, 2H), 3.73 (s, 3H), 3.67-3.65 (m, 2H), 3.49 (s, 2H), 3.07-2.98 (m, 7H), 2.89-2.80 (m, 1H), 2.74-2.60 (m, 3H), 2.11-1.87 (m, 4H), 1.48- 1.45 (m, 2H). | + | CRBN |
| 2 | 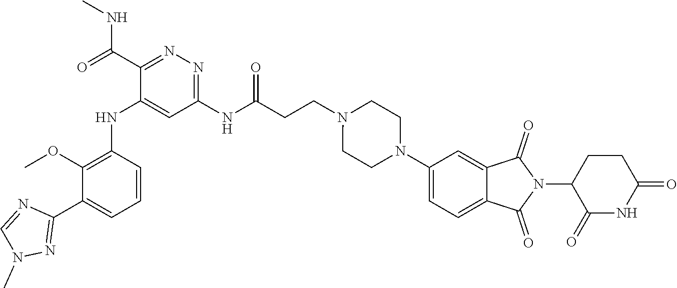 [SMILES: CNC(=O)c1nnc(NC(=O)CCN2CCN(c3ccc4c(c3)C(=O)N(C3CCC(=O)NC3=O)C4=O)CC2)cc1Nc1cccc(-c2ncn(C)n2)c1OC] | 750.78 | 1H NMR (400 MHz, CD₃OD, ppm): δ 8.52 (s, 1H), 7.97 (s, 1H), 7.77-7.72 (m, 2H), 7.58 (d, J = 8.0 Hz, 1H), 7.47 (s, 1H), 7.35- 7.30 (m, 2H), 5.11-5.07 (m, 1H), 4.01 (d, J = 2.4 Hz, 3H), 3.74-3.70 (m, 6H), 3.61-3.52 (m, 7H), 3.11-3.10 (m, 2H), 2.99 (s, 3H), 2.86-2.70 (m, 3H), 2.16-2.12 (m, 1H). | — | CRBN |
| 3 | 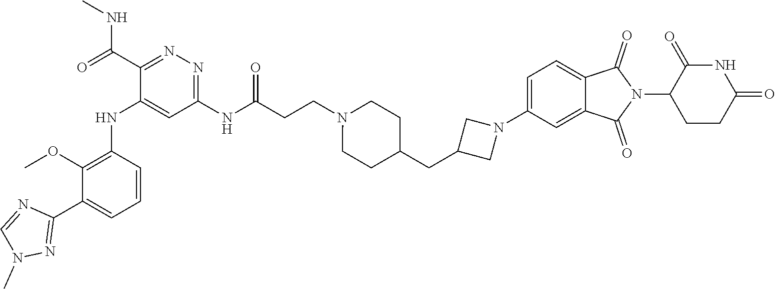 [SMILES: CNC(=O)c1nnc(NC(=O)CCN2CCC(CC3CN(c4ccc5c(c4)C(=O)N(C4CCC(=O)NC4=O)C5=O)C3)CC2)cc1Nc1cccc(-c2ncn(C)n2)c1OC] | 818.90 | ¹H NMR (400 MHz, CD₃OD, ppm): δ 8.55 (s, 1H), 7.80-7.75 (m, 2H), 7.61-7.57 (m, 2H), 7.33- 7.29 (m, 1H), 6.76-6.75 (m, 1H), 6.61-6.58 (m, 1H), 5.06-5.02 (m, 1H), 4.18-4.14 (m, 2H), 4.02 (s, 3H), 3.73 (s, 3H), 3.67-3.60 (m, 4H), 3.47 (s, 2H), 3.06-2.64 (m, 11H), 2.11-2.07 (m, 1H), 1.98-1.95 (m, 2H), 1.69 (s, 3H), 1.57-1.47 (m, 2H). | ++ | CRBN |
| 4 | 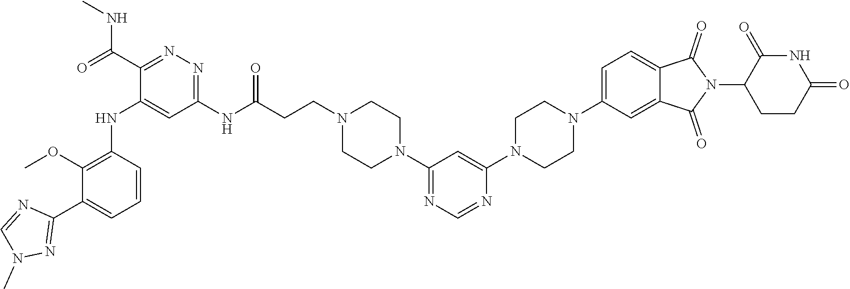 [SMILES: CNC(=O)c1nnc(NC(=O)CCN2CCN(c3cc(N4CCN(c5ccc6c(c5)C(=O)N(C5CCC(=O)NC5=O)C6=O)CC4)ncn3)CC2)cc1Nc1cccc(-c2ncn(C)n2)c1OC] | 912.97 | ¹H NMR (400 MHz, CD₃OD, ppm): δ 8.55 (s, 1H), 8.30 (s, 1H), 7.78-7.69 (m, 3H), 7.59-7.57 (m, 1H), 7.33-7.29 (m, 2H), 7.21-7.19 (m, 1H), 6.13 (s, 1H), 5.09-5.05 (m, 1H), 4.06-4.02 (m, 7H), 3.90-3.87 (m, 4H), 3.72 (s, 3H), 3.69-3.67 (m, 4H), 3.60-3.57 (m, 2H), 3.49 (s, 4H), 3.14- 3.12 (m, 2H), 2.98 (s, 3H), 2.90-2.81 (m, 1H), 2.76-2.65 (m, 2H), 2.12-2.09 (m, 1H). | + | CRBN |
| 5 | 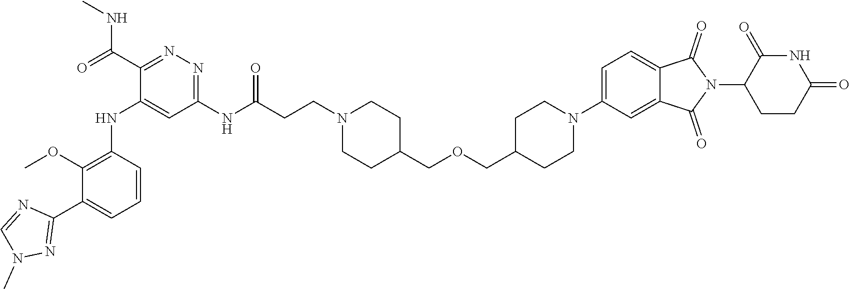 [SMILES: CNC(=O)c1nnc(NC(=O)CCN2CCC(COCC3CCN(c4ccc5c(c4)C(=O)N(C4CCC(=O)NC4=O)C5=O)CC3)CC2)cc1Nc1cccc(-c2ncn(C)n2)c1OC] | 876.98 | ¹H NMR (400 MHz, CD₃OD, ppm): δ 8.56 (s, 1H), 7.78-7.73 (m, 2H), 7.64-7.56 (m, 2H), 7.33- 7.29 (m, 2H), 7.18-7.16 (m, 1H), 5.07-5.03 (m, 1H), 4.04-4.02 (m, 5H), 3.72 (s, 3H), 3.64-3.60 (m, 2H), 3.48-3.44 (m, 2H), 3.33-3.32 (m, 4H), 3.05-2.95 (m, 9H), 2.90- 2.80 (m, 1H), 2.76-2.64 (m, 2H), 2.11-2.05 (m, 1H), 1.98-1.79 (m, 6H), 1.61-1.55 (m, 2H), 1.41-1.32 (m, 2H). | + | CRBN |
| 6 | 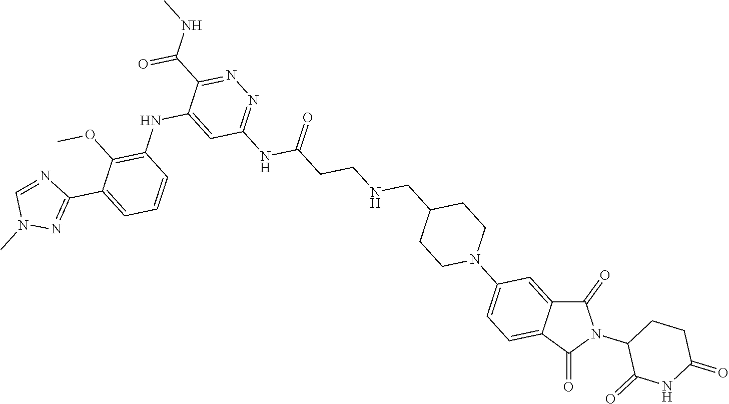 [SMILES: CNC(=O)c1nnc(NC(=O)CCNCC2CCN(c3ccc4c(c3)C(=O)N(C3CCC(=O)NC3=O)C4=O)CC2)cc1Nc1cccc(-c2ncn(C)n2)c1OC] | 778.83 | ¹H NMR (400 MHz, CD₃OD, ppm): δ 8.52 (s, 1H), 7.99 (s, 1H), 7.74- 7.73 (m, 1H), 7.66 (d, J = 8.4 Hz, 1H), 7.58- 7.57 (m, 1H), 7.34-7.28 (m, 2H), 7.22-7.20 (m, 1H), 5.09-5.04 (m, 1H), 4.07-4.01 (m, 5H), 3.71 (s, 3H), 3.38-3.37 (m, 2H), 3.01-2.98 (m, 9H), 2.85-2.72 (m, 3H), 2.03-1.93 (m, 2H), 1.92- 1.89 (m, 2H), 3.39-3.37 (m, 2H). | ++ | CRBN |
| 7 | 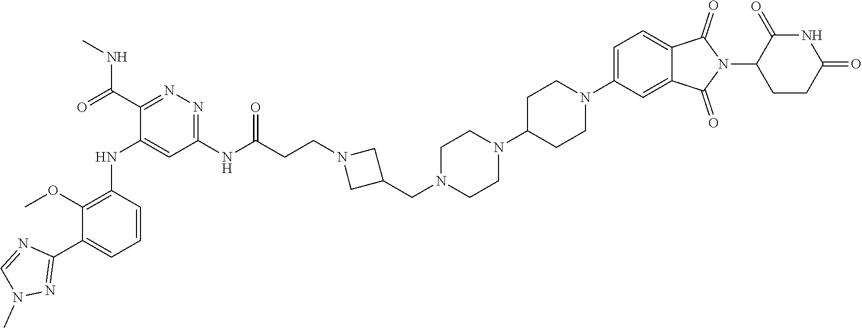 [SMILES: CNC(=O)c1nnc(NC(=O)CCN2CC(CN3CCN(C4CCN(c5ccc6c(c5)C(=O)N(C5CCC(=O)NC5=O)C6=O)CC4)CC3)C2)cc1Nc1cccc(-c2ncn(C)n2)c1OC] | 903.02 | ¹H NMR (400 MHz, CD₃OD, ppm): δ 8.54 (s, 1H), 7.85 (s, 1H), 7.75- 7.74 (m, 1H), 7.70-7.68 (m, 1H), 7.59-7.57 (m, 1H), 7.39-7.38 (m, 1H), 7.32-7.25 (m, 2H), 5.09- 5.05 (m, 1H), 4.27-4.18 (m, 4H), 4.02-3.90 (m, 5H), 3.72 (s, 3H), 3.55 (s, 2H), 3.45-3.42 (m, 5H), 3.14-2.98 (m, 7H), 2.91-2.66 (m, 10H), 2.23-2.20 (m, 2H), 2.12- 2.08 (m, 1H), 1.81-1.73 (m, 2H). | +++ | CRBN |
| 8 | 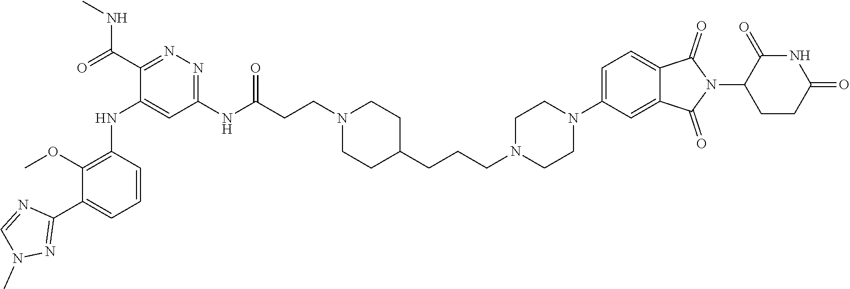 [SMILES: CNC(=O)c1nnc(NC(=O)CCN2CCC(CCCN3CCN(c4ccc5c(c4)C(=O)N(C4CCC(=O)NC4=O)C5=O)CC3)CC2)cc1Nc1cccc(-c2ncn(C)n2)c1OC] | 875.99 | ¹H NMR (400 MHz, CD₃OD, ppm): δ 8.52 (s, 1H), 7.91 (s, 1H), 7.75- 7.72 (m, 2H), 7.59-7.57 (m, 1H), 7.46-7.45 (m, 1H), 7.34-7.28 (m, 2H), 5.11-5.06 (m, 1H), 4.19- 4.12 (m, 1H), 4.02 (s, 3H), 3.72-3.61 (m, 7H), 3.53-3.47 (m, 3H), 3.31-3.30 (m, 2H), 3.22- 3.18 (m, 4H), 3.06-2.98 (m, 7H), 2.91-2.65 (m, 3H), 2.13-2.09 (m, 1H), 2.02-1.99 (m, 2H), 1.86- 1.78 (m, 2H), 1.66-1.65 (m, 1H), 1.50-1.29 (m, 4H). | +++ | CRBN |
| 9 | 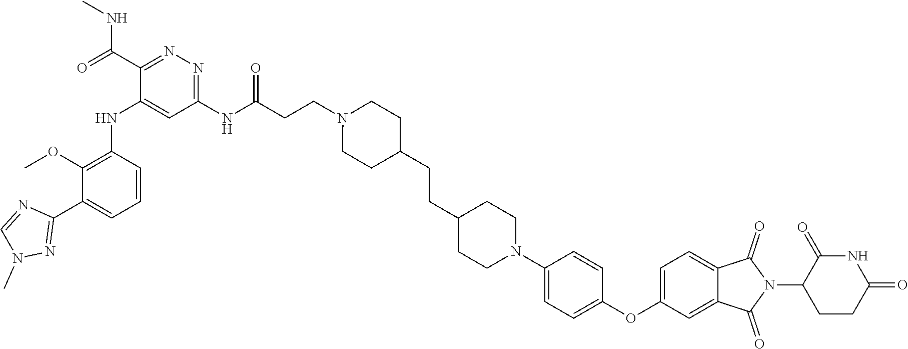 [SMILES: CNC(=O)c1nnc(NC(=O)CCN2CCC(CCC3CCN(c4ccc(Oc5ccc6c(c5)C(=O)N(C5CCC(=O)NC5=O)C6=O)cc4)CC3)CC2)cc1Nc1cccc(-c2ncn(C)n2)c1OC] | 953.07 | ¹H NMR (400 MHz, CD₃OD, ppm): δ 8.53 (s, 1H), 7.91-7.85 (m, 2H), 7.75-7.68 (m, 3H), 7.59- 7.57 (m, 1H), 7.45-7.42 (m, 1H), 7.39-7.38 (m, 1H), 7.33-7.29 (m, 3H), 5.14-5.10 (m, 1H), 4.02 (s, 3H), 3.73-3.70 (m, 5H), 3.63-3.47 (m, 6H), 3.06-2.98 (m, 7H), 2.91- 2.65 (m, 3H), 2.15-1.99 (m, 5H), 1.70-1.61 (m, 4H), 1.51-1.40 (m, 6H). | ++ | CRBN |
| 10 | 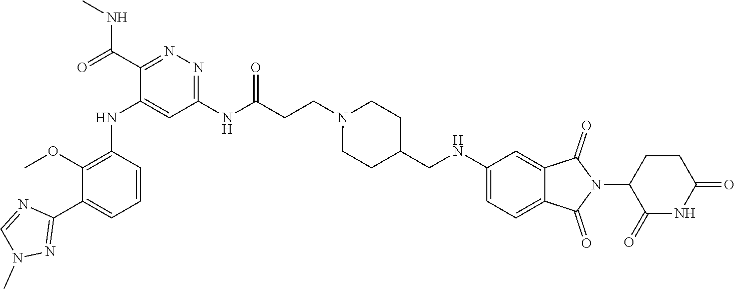 [SMILES: CNC(=O)c1nnc(NC(=O)CCN2CCC(CNc3ccc4c(c3)C(=O)N(C3CCC(=O)NC3=O)C4=O)CC2)cc1Nc1cccc(-c2ncn(C)n2)c1OC] | 778.83 | ¹H NMR (400 MHz, CD₃OD, ppm): δ 8.55 (s, 1H), 7.77-7.75 (m, 2H), 7.58-7.54 (m, 2H), 7.31 (t, J = 8.0 Hz, 1H), 6.99 (d, J = 2.0 Hz, 1H), 6.87-6.84 (m, 1H), 5.06- 5.01 (m ,1H), 4.02 (s, 3H), 3.72 (s, 3H), 3.70-3.62 (m, 2H), 3.50- 3.46 (m, 2H), 3.18-3.17 (m, 2H), 3.07-2.98 (m, 7H), 2.95-2.65 (m, 3H), 2.15-1.96 (m, 4H), 1.65- 1.51 (m, 2H). | ++ | CRBN |

— < 25% degradation
+ 25-50% degradation
++ 51-75% degradation
+++ >75% degradation

A novel bifunctional molecule, which contains a TYK2 recruiting moiety and an E3 ubiquitin ligase recruiting moiety is described. The bifunctional molecules of the present disclosure actively degrades TYK2, leading to robust cellular proliferation suppression and apoptosis induction. Protein degradation mediated by the bifunctional compounds of the present disclosure provides a promising strategy in targeting the “undruggable” pathological proteins by traditional approaches.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A hetero-bifunctional compound having the chemical structure: 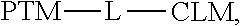
or a pharmaceutically acceptable salt or solvate thereof,
wherein:
the CLM is a small molecule E3 ubiquitin ligase binding moiety that binds a cereblon E3 ubiquitin ligase;
the PTM is a small molecule Tyrosine kinase 2 (TYK2) targeting moiety that binds to the pseudokinase domain of human TYK2; and
the L is a bond or a chemical linking moiety that covalently couples the CLM to the PTM.

2. The compound according to claim 1, wherein PTM is represented by: 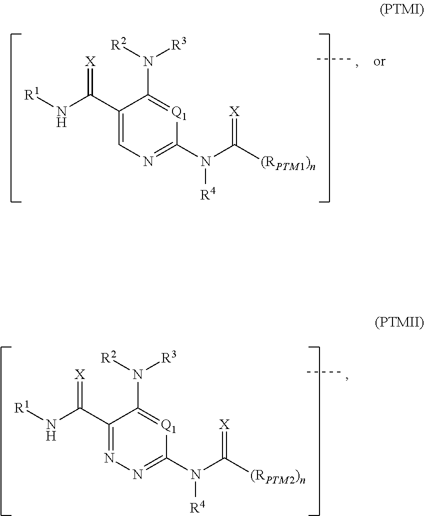
wherein:
R¹ is hydrogen, C1-C3 alkyl, C1-C3 alkyl substituted by 0-7 deuterium atoms, or C3-C6 cycloalkyl, each optionally substituted by 0-7 R^{1a};
R^{1a} at each occurrence is independently hydrogen, deuterium, CN, or halogen, e.g., F, Cl, Br;
R² is hydrogen, an alkyl, e.g., methyl, ethyl, propyl; furyl, pyranyl, cyclopropyl, cyclobutyl or cyclohexyl, cyclopentyl, pyridyl, pyrimidinyl, pyridazinyl, pyrazinyl, quinolinyl, pyrrolopyridinyl, each group substituted as valency allows by 0-4 groups selected from R^{2a}, —C(O)R^{2a}, C1-C6 alkyl, —(CH₂)ᵣ-3-14 membered carbocycle substituted with 0-1 R^{2a} or a 5-14 membered heterocycle containing 1-4 heteroatoms selected from N, O, and S, substituted with 0-4 R^{2a};
R^{2a} at each occurrence is independently hydrogen, ═O, halo, OCF₃, CN, NO₂, —(CH₂)ᵣOR^{b}, —(CH₂)ᵣSR^{b}, —(CH₂)ᵣC(O)R^{b}, —(CH₂)ᵣC(O)OR^{b}, —(CH₂)ᵣOC(O)R^{b}, (CH₂)ᵣNR^{u}Rⁿ, —(CH₂)ᵣC(O)NR¹¹R¹¹, —(CH₂)ᵣNR^{b}C(O)R^{c}, —(CH₂)ᵣNR^{b}C(O)OR^{c}, —NR^{b}C(O)NR¹¹R¹¹, —S(O)ₚNR¹¹R¹¹, —NR^{b}S(O)ₚR^{c}, —S(O)ₚR^{c}, C1-C6 alkyl substituted with 0-3 R^{a}, C1-C6 haloalkyl, C2-C6 alkenyl substituted with 0-3 R^{a}, C2-C6 alkynyl substituted with 0-3 R^{a}, —(CH₂)ᵣ-3-14 membered carbocycle substituted with 0-1 R^{a}, a-(CH₂)ᵣ-5-7 membered heterocycle comprising carbon atoms or 1-4 heteroatoms selected from N, O, and S(0)ₚ substituted with 0-2 R^{a}, or a (CH₂)ᵣ-5-7 membered heterocycle containing 1-4 heteroatoms selected from N, O, and S(O)ₚ substituted with 0-2 R^{a};
R³ is hydrogen, C3-C10 cycloalkyl, C6-C10 aryl, or a 5-10 membered heterocycle containing 1-4 heteroatoms selected from N, O, and S, each group substituted with 0-4 R^{3a};
wherein at least one R² or R³ is hydrogen;
R^{3a} at each occurrence is independently hydrogen, ═O, halo, OCF₃, CF₃, CHF₂, CN, NO₂, —(CH₂)ᵣOR^{b}, —(CH₂)ᵣSR^{b}, —(CH₂)ᵣC(O)R^{b}, —(CH₂)ᵣC(O)OR^{b}, —(CH₂)ᵣOC(O)R^{b}, —(CH₂)ᵣNR¹¹R¹¹, —(CH₂)ᵣC(O)NR¹¹R¹¹, —(CH₂)ᵣNR^{b}C(O)R^{c}, —(CH₂)ᵣNR^{b}C(O)OR^{c}, —NR^{b}C(O)NR¹¹R¹¹, —S(O)ₚNR¹¹R¹¹, —NR^{b}S(O)ₚR^{c}, —S(O)ₚR^{c}, C1-C6 alkyl substituted with 0-3 R^{a}, C₂₋₆ alkenyl substituted with 0-3 R^{a}, C₂₋₆ alkynyl substituted with 0-3 R^{a}, C1-C6 haloalkyl, —(CH₂)ᵣ-3-14 membered carbocycle substituted with 0-3 R^{a} or a —(CH₂)ᵣ-5-10 membered heterocycle containing 1-4 heteroatoms selected from N, O, and S(O)ₚ substituted with 0-3 R^{a};
or two R^{3a}, together with the atoms to which they are attached, combine to form a fused ring wherein said ring is selected from phenyl and a heterocycle comprising carbon atoms and 1-4 heteroatoms selected from N, O, and S(O)ₚ, each fused ring substituted with 0-3 R^{a1};
R⁴ is hydrogen, C1-C4 alkyl substituted with 0-1 R^{f}, (CH₂)ᵣ-phenyl substituted with 0-3 R^{d} or a —(CH₂)-5-7 membered heterocycle containing 1-4 heteroatoms selected from N, O, or S(O)ₚ;
R⁶ is hydrogen, halo, C1-C4alkyl, C1-C4haloalkyl, OC1-C4haloalkyl, OC1-C4alkyl, CN, NO₂ or OH;
R¹¹ at each occurrence is independently hydrogen, C₁₋₄ alkyl substituted with 0-3 R^{f}, CF₃, C₃₋₁₀ cycloalkyl substituted with 0-1 R^{f}, (CH)ᵣ-phenyl substituted with 0-3 R^{d} or —(CH₂)ᵣ-5-7 membered heterocycle containing 1-4 heteroatoms selected from N, O, and S(O)ₚ substituted with 0-3 R^{d};
R^{a} and R^{a1} at each occurrence are independently hydrogen, halogen, OH, OCF₃, CF₃, CHF₂, CN, NO₂, —(CH₂)ᵣOR^{b}, —(CH₂)ᵣSR^{b}, —(CH₂)ᵣC(O)R^{b}, —(CH₂)ᵣC(O)OR^{b}, —(CH₂)ᵣOC(O)R^{b}, —(CH₂)ᵣNR¹¹R¹¹, —(CH₂)ᵣC(O)NR¹¹R¹¹, —(CH₂)ᵣNR^{b}C(O)R, —(CH₂)ᵣNR^{b}C(O)OR, —NR^{b}C(O)NR¹¹R¹¹, —S(O)ₚNR¹¹R¹¹, —NR^{b}S(O)ₚR^{c}, —S(O)R^{e}, —S(O)₂R^{e}, C1-C6 alkyl substituted with 0-3 R^{f}, C1-C6 haloalkyl, C2-C6 alkenyl substituted with 0-3 R^{a}, C2-C6 alkynyl substituted with 0-3 R^{a}, —(CH₂)ᵣ-3-14 membered carbocycle or —(CH₂)ᵣ-5-7 membered heterocycle containing 1-4 heteroatoms selected from N, O, and S(O)ₚ substituted with 0-3 R^{f};
R^{b} is hydrogen, C1-C6 alkyl substituted with 0-3 R^{d}, C1-C6haloalkyl, C3-C6 cycloalkyl substituted with 0-2 R^{d}, or —(CH₂)ᵣ-5-7 membered heterocycle containing 1-4 heteroatoms selected from N, O, and S(O)ₚ substituted with 0-3 R^{f} or (CH₂)ᵣ-phenyl substituted with 0-3 R^{d};
R^{c} is C1-C6 alkyl substituted with 0-3 R^{f}, (CH₂)ᵣ-C3-C6 cycloalkyl substituted with 0-3 R^{f} or (CH₂)ᵣ-phenyl substituted with 0-3 R^{f};
R^{d} at each occurrence is independently hydrogen, F, Cl, Br, OCF₃, CF₃, CN, NO₂, —OR^{e}, —(CH₂)ᵣC(O)R^{c}, —NR^{e}R^{e}, —NR^{e}C(O)OR^{e}, C1-C6 alkyl or (CH₂)ᵣ-phenyl substituted with 0-3 R^{f};
R^{e} at each occurrence is independently hydrogen, C1-C6 alkyl, C3-C6 cycloalkyl or (CH₂)ᵣ-phenyl substituted with 0-3 R^{f};
R^{f} independently at each occurrence is hydrogen, halo, CN, NH₂, OH, C₃₋₆ cycloalkyl, CF₃, O(C1-C6alkyl) or a —(CH₂)ᵣ-5-7 membered heterocycle containing 1-4 heteroatoms selected from N, O, and S(O)ₚ;
R_{PTM1} and R_{PTM2} are independently: H; halogen (e.g., Cl or F); —CN; —OH; —NO₂; —NH₂; optionally substituted linear or branched alkyl (e.g., optionally substituted linear or branched C1-C6 alkyl or optionally substituted linear or branched C1-C4 alkyl or C1—C8 alkyl optionally substituted with OH); optionally substituted cycloalkyl (e.g., cyclopropyl, cyclobutyl, cyclopentyl, cyclohexyl); O-optionally substituted linear or branched C1-C4 alkyl; an optionally substituted C1-C4 alkynyl; an optionally substituted C1-C4 alkyne; optionally substituted linear or branched hydroxyalkyl (e.g., optionally substituted linear or branched C1-C7 hydroxyalkyl); optionally substituted alkylcycloalkyl (e.g., includes optionally substituted C1-C6 alkyl, optionally substituted C3-C10 cycloalkyl; or both); optionally substituted alkyl-aryl (e.g., includes an optionally substituted linear or branched C1-C6 alkyl, an optionally substituted 5-10 member heteroaryl, or both); optionally substituted alkyl-heteroaryl (e.g., includes an optionally substituted linear or branched C1-C6 alkyl, an optionally substituted 5-10 member heteroaryl, or both); optionally substituted alkyl-heteroaryl (e.g., includes a C1-C6 alkyl, an optionally substituted 5 or 6 member heteroaryl, optionally substituted with a C1-C4 alkyl; the heteroaryl is selected from oxazol-4-yl, 1,3,4-triazol-2-yl, and imidazole-1-yl; or combination thereof); optionally substituted —NH-alkyl-heteroaryl (e.g., an optionally substituted linear or branched C1-C5 alkyl, an optionally substituted 5-8 member heteroaryl, optionally substituted with a C1-C4 alkyl, N—CH₂-pyrazol-4-yl, or a combination thereof); optionally substituted alkoxy (e.g., an optionally substituted linear or branched C1-C6 alkyl or —OCH₃); optionally substituted O-heterocyclyl (e.g., includes an optionally substituted 3-12 or 4-7 member heterocyclyl; an optionally substituted heterocycloalkyl; an optionally substituted C₃₋₁₂ monocyclic or bicyclic heterocycloakly; optionally substituted with at least one OH, C1-C5 alkyl (such as a methyl), ═O, NH₂, or a combination thereof; or a combination thereof); optionally substituted S-heterocyclyl (e.g., includes an optionally substituted 4-7 member heterocyclyl; an optionally substituted heterocycloalkyl; optionally substituted with at least one C1-C4 alkyl (such as a methyl), ═O, or a combination thereof; or a combination thereof); optionally substituted 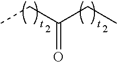
(e.g., optionally substituted with a linear or branched C1-C4 alkyl; —(CH₂)ₙCO(CH₂)ᵥCH₃, —COCH₃, or —CH₂CH₂COCH₃, wherein each u and v is independently selected from 1, 2, 3, 4 or 5);
optionally substituted 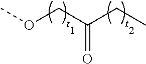
(e.g., optionally substituted with a linear or branched C1-C4 alkyl; —O(CH₂)ₙCO(CH₂)ᵥCH₃, —O(CH₂)ᵤCH((CH₂)ₓCH₃)(CH₂)_{w}CO(CH₂)ᵥCH₃, —O—CH₂COCH₃, —O—CH₂COCH₂CH₃, —O—CH(CH₃)COCH₃, —OCH₂COCH₃, or —OCH₂(CH₃)COCH₃, wherein each u, v, w, and x is independently selected from 1, 2, 3, 4 or 5); optionally substituted 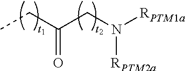
(e.g., optionally substituted with a linear or branched C1-C4 alkyl; —(CH₂)ᵤCO(CH₂)ᵥNR_{PTM1a}R_{PTM2a}, —CONR_{PTM1a}R_{PTM2a}, —CH₂CONR_{PTM1a}R_{PTM2a}, —CH₂CH₂CONR_{PTM1a}R_{PTM2a}, —CONHCH₃, or —CH₂CONHCH₃, wherein each u and v is independently selected from 1, 2, 3, 4 or 5); optionally substituted 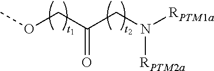
(e.g., optionally substituted with a linear or branched C1-C4 alkyl; —O(CH₂)ₙCO(CH₂)ᵥNR_{PTM1a}R_{PTM2a}, —O(CH₂)ₙCH((CH₂)ₓCH₃)(CH₂)_{w}CO(CH₂)ᵥ NR_{PTM1a}R_{PTM2a}, —O—CH(CH₃)CONR_{PTM1a}R_{PTM2a}, —O—CH₂CONR_{PTM1a}R_{PTM2a}, or —OCH₂C(O)NHOCH₃, wherein each u, v, w, and x is independently selected from 1, 2, 3, 4 or 5); optionally substituted 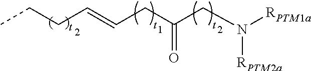
(e.g., optionally substituted with a linear or branched C1-C4 alkyl; —(CH₂)ₙCHCH(CH₂)_{w}CO(CH₂)ᵥ NR_{PTM1a}R_{PTM2a} or —CHCHCONR_{PTM1a}R_{PTM2a}, wherein each u, v, and w is independently selected from 1, 2, 3, 4 or 5); optionally substituted 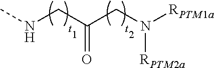
(e.g., optionally substituted with a linear or branched C1-C4 alkyl; —NH—(CH₂)ₙCO(CH₂)ᵥNR_{PTM1a}R_{PTM2a} or —NH—CH₂CONR_{PTM1a}R_{PTM2a}, wherein each u and v is independently selected from 1, 2, 3, 4 or 5); fluoroalkoxy (e.g., a mono-, bi- and/or tri-fluoroalkoxy); optionally substituted monocylic or bicyclic cyclocalkyl (e.g., an optionally substituted 3-12 member cycloalkyl; optionally substituted with at least one of OH, ═O, linear or branched C1-C6 alkyl (such as a methyl, ethyl, or butyl), or NH₂; or a combination thereof); optionally substituted hydroxycycloalkyl; optionally substituted aryl (e.g., an optionally substitute C5-C10 aryl, an optionally substituted 5-7 member aryl; optionally substituted with at least one halogen or C1-C3 alkyl (e.g., methyl or ethyl); or a combination thereof), optionally substituted heteroaryl (e.g., an optionally substituted 5-10 or member heteroaryl, an optionally substituted 5-7 member heteroaryl; an optionally substituted 5-member heteroaryl; optionally substituted with at least one halogen or C1-C3 alkyl (e.g., methyl or ethyl); or a combination thereof) optionally linked to Q₁ via a C or N-atom of the heteroaryl (e.g., at least one of optionally linked to Q₁, optionally linked via an optionally substituted —(CH₂)ᵤO(CH₂)ᵥO(CH₂)ₓ—, or a combination thereof); optionally substituted monocyclic or bicyclic heterocyclyl (e.g., an optionally substituted 3-12 member heterocyclyl; an C3-C12 monocylcic or bicyclic heterocycloalkyl, azetidine1-yl, pyrrolidin-1-yl, piperidin-1yl, piperazin-1-yl, or morpholin-4-yl, or homopiperazin-1-yl, each optionally substituted with OH, a linear or branched C1-C5 alkyl (a methyl, ethyl, or butyl group) or NH₂) optionally linked to Q₁ via a C or N atom of the heterocyclyl (e.g., at least one of optionally linked to Q₁, optionally linked via an optionally substituted —(CH₂)ᵤO(CH₂)ᵥO(CH₂)ₓ—, or both);
each t₁ is independently selected from 1, 2, 3, 4, or 5;
each t₂ is independently is independently selected from 0, 1, 2, 3, 4, or 5;
each R_{PTM1a} and R_{PTM2a} are independently H, optionally substituted C1-C4 alkyl (e.g., a CH₃ or CH₂CH₃), optionally substituted C1-C4 alkoxy (e.g., —OCH₂ or —CH₂CH₃), CH₂OCH₃ or R_{PTM1a} and R_{PTM2a} are joined together form a 3-10 member ring;
Q₁ is CH, N, O, or C, each optionally substituted with one or more independently selected R_{PTM1} or R_{PTM2} (e.g., 1, 2, or 3 independently selected R_{PTM1} or R_{PTM2}, depending upon valency);
p is 0, 1, or 2;
r is 0, 1, 2, 3, or 4;
X is O, S, or CH₂;
n is an integer from 0 to 10; and [symbol]
of the PTM indicates the point of attachment with a chemical linker group or a ULM.

3. The compound according to claim 2, or a stereoisomer or pharmaceutically acceptable salt thereof, wherein R¹ is CH₃, C2H₅, CD₃ or CD₂CD₃.

4. The compound according to any of claims 1-3, wherein the PTM has a chemical structure selected from: 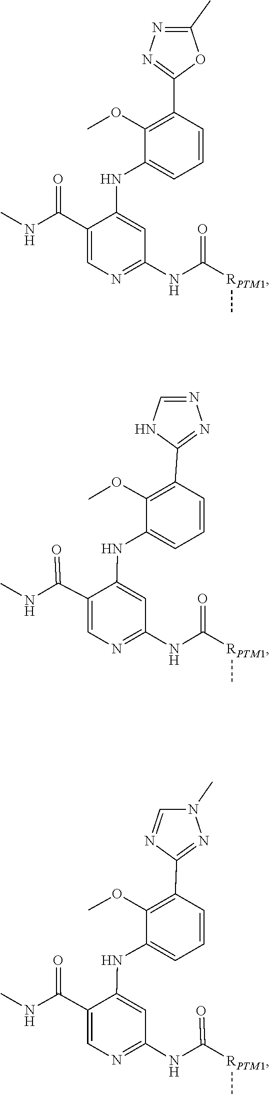 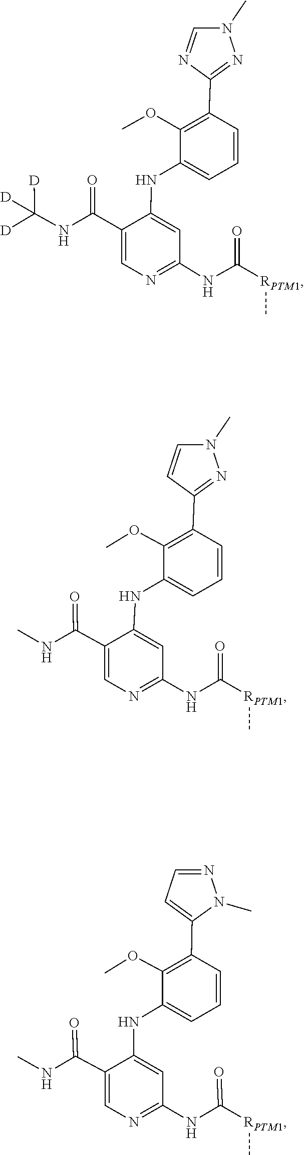 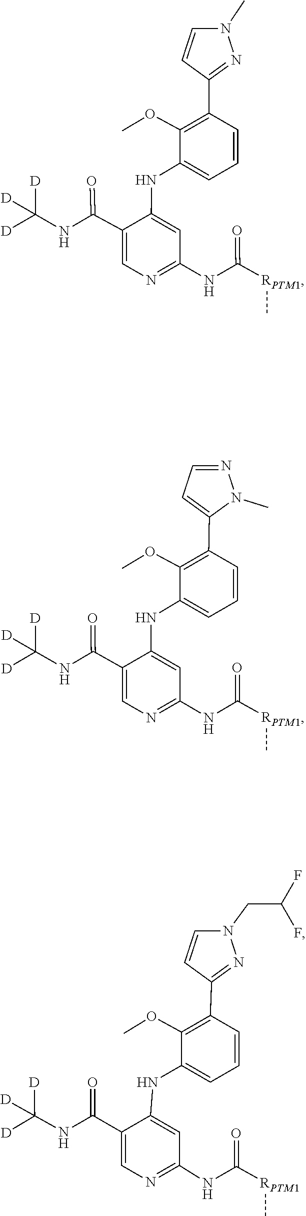 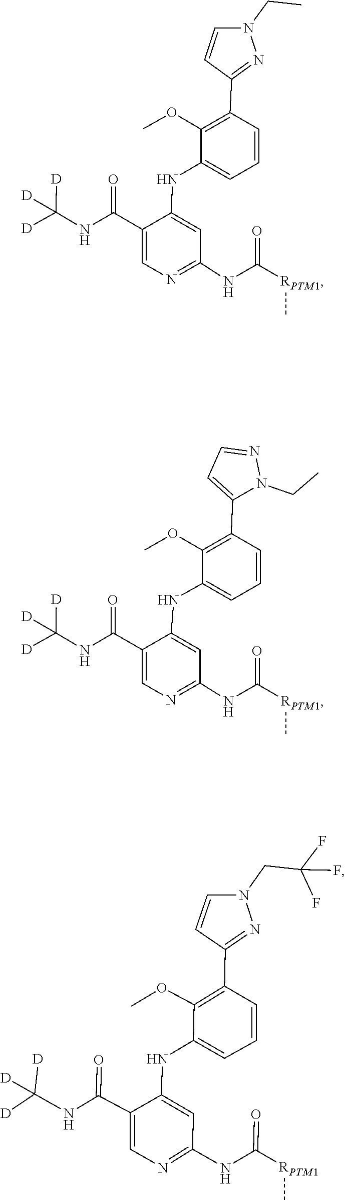 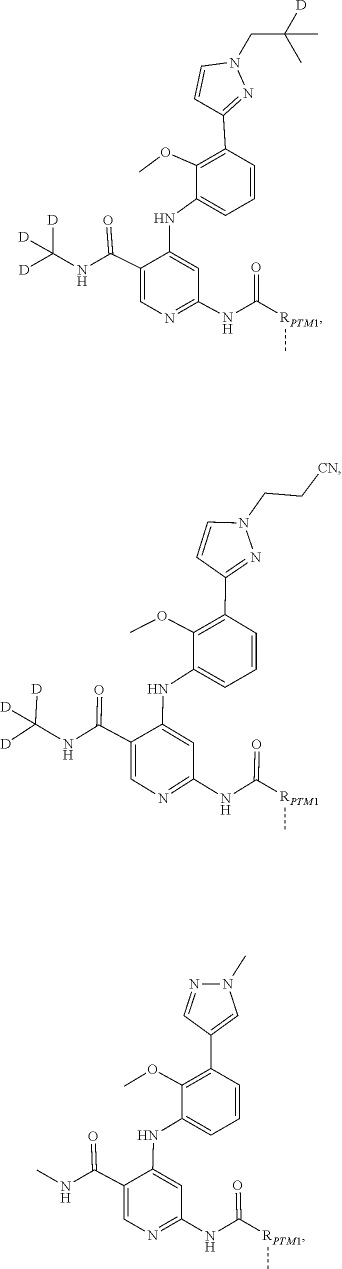 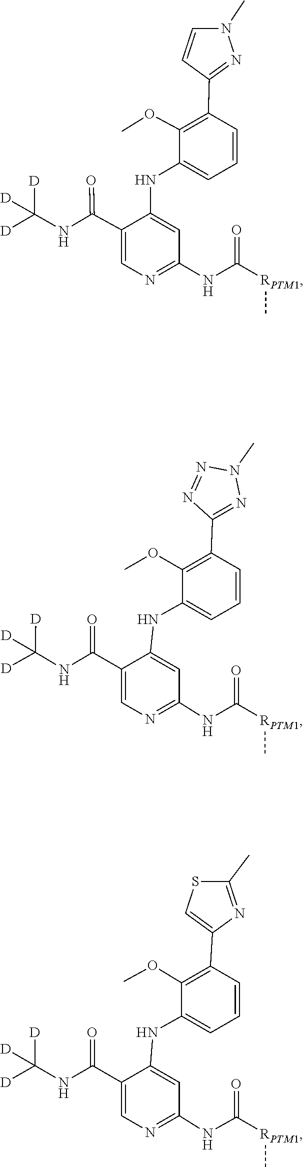 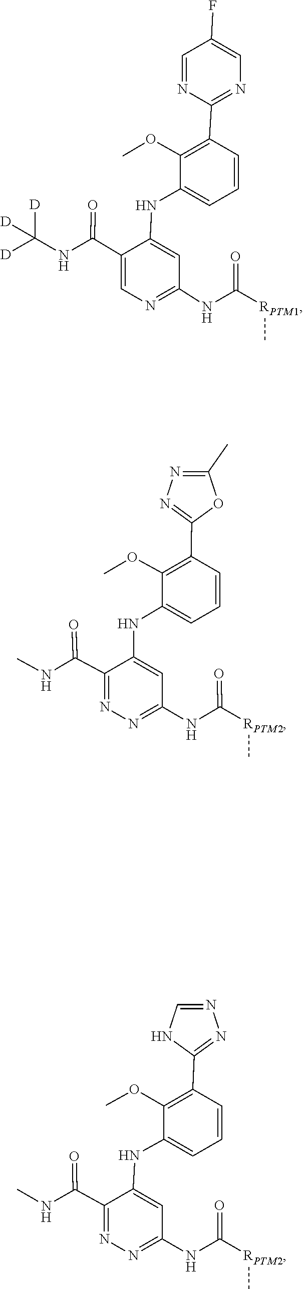 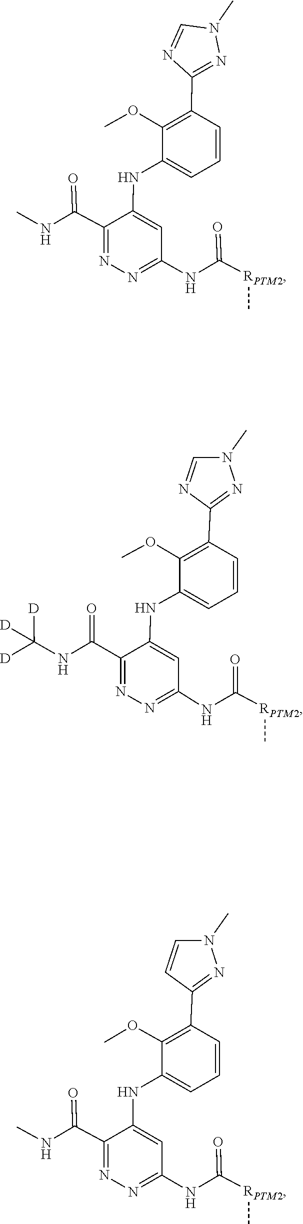 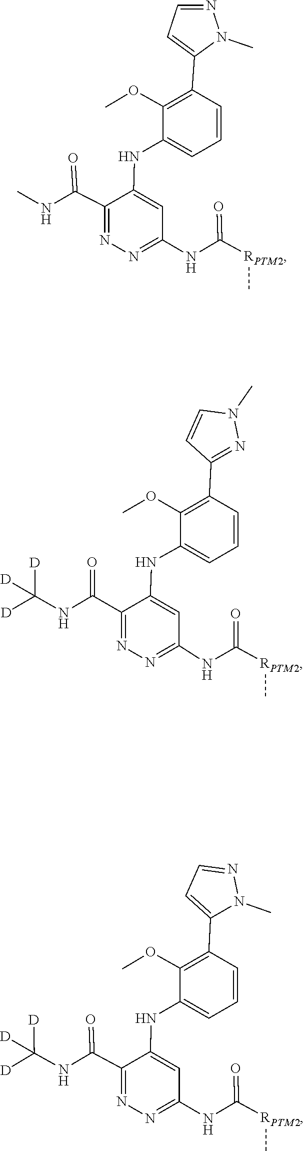 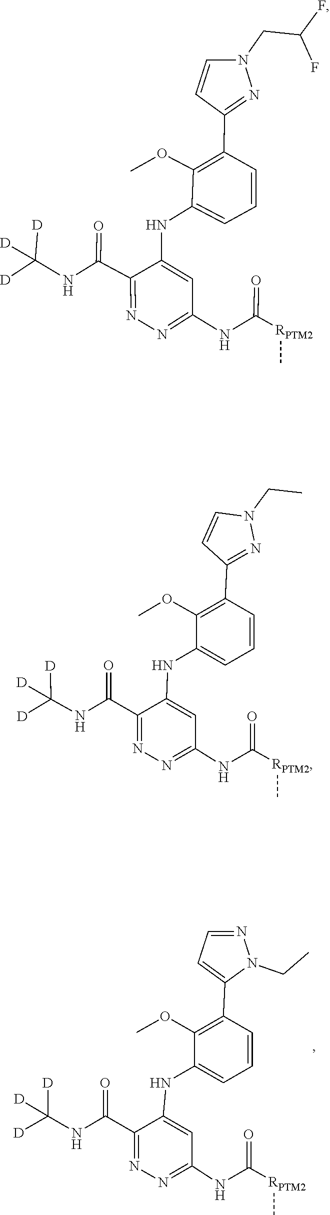 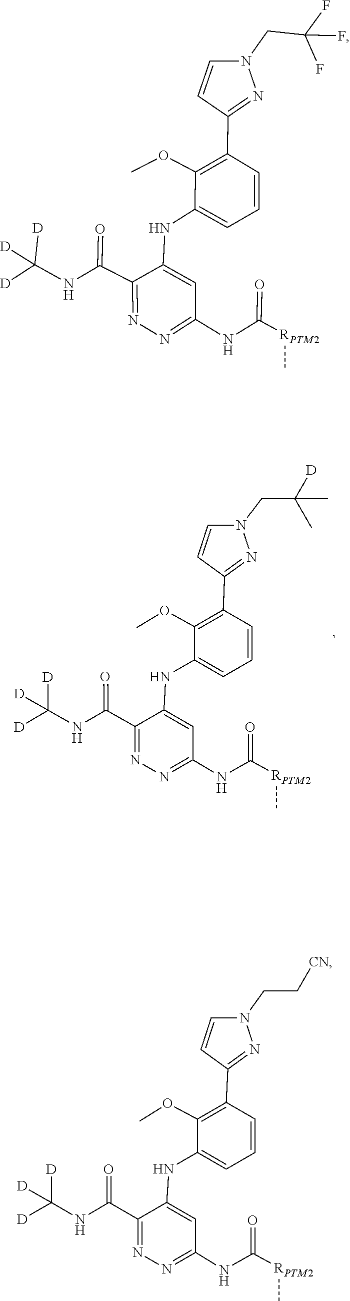 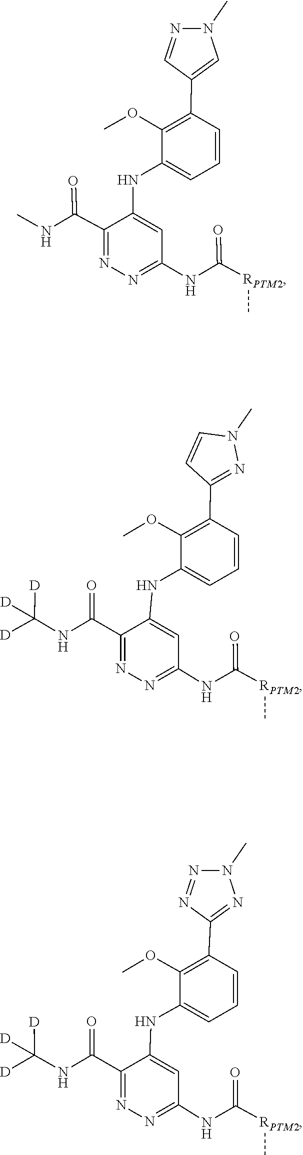 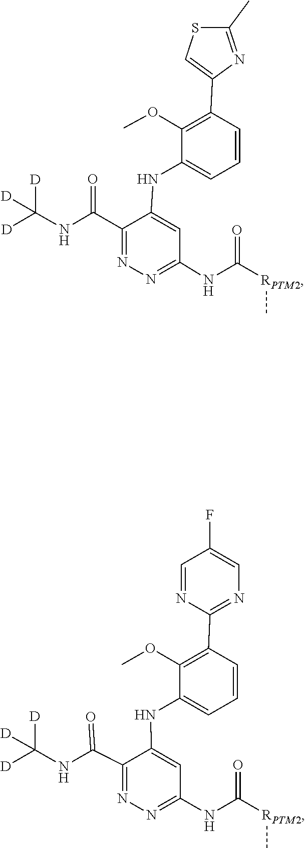
wherein R_{PTM1} and R_{PTM2} are independently selected from a bond, atom or chemical group coupling the PTM to a linker group (L) or ULM, and the [symbol] of the R_{PTM1} and R_{PTM2} indicate the point of attachment with L or a ULM.

5. The compound according to any of claims 1-4, wherein R_{PTM1} and R_{PTM2} are independently selected from: methyl, ethyl, propyl, butyl, pentyl, hexyl, H, OH, ethyl, 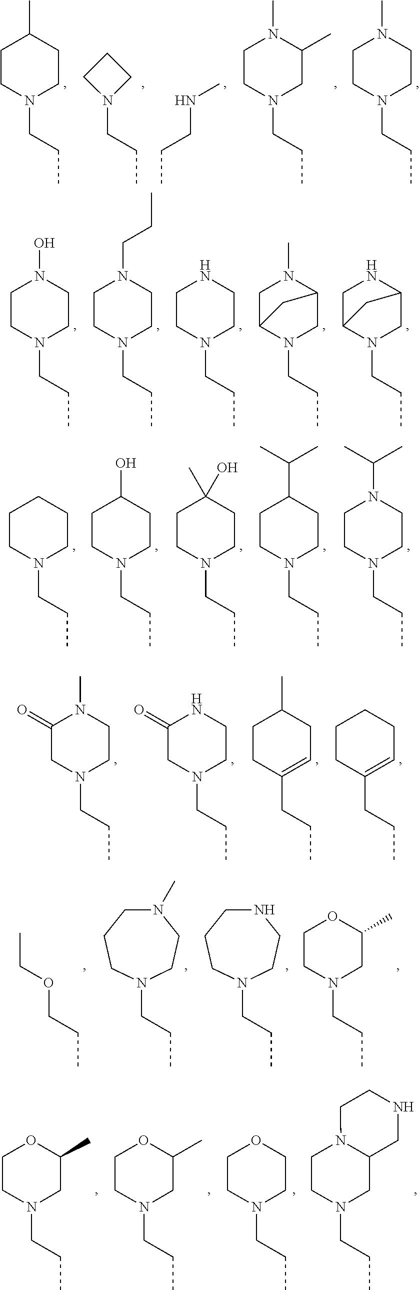 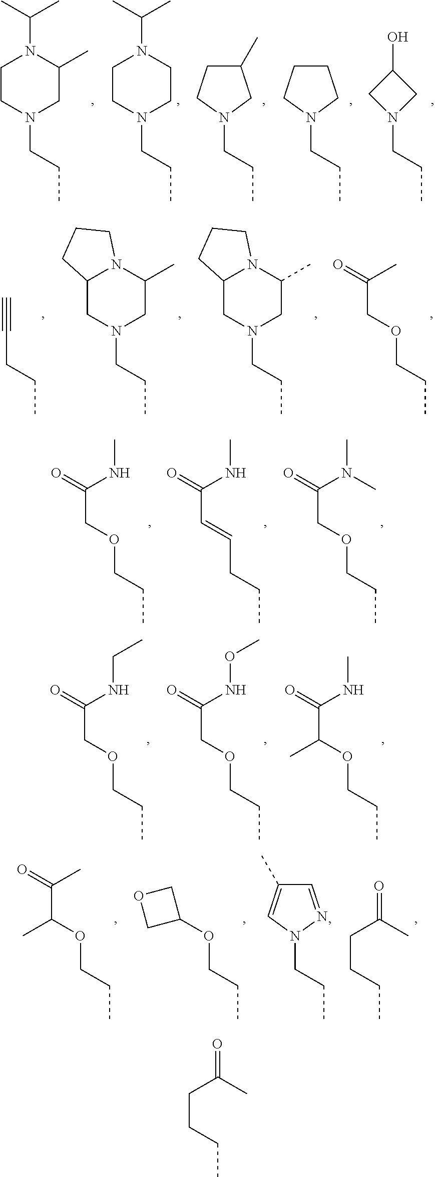
wherein [symbol] represents a bond that may be stereospecific ((R) or (S)) or non-stereospecific; or a combination thereof.

6. The compound according to any of claims 1-5, wherein PTM is represented by chemical structure: 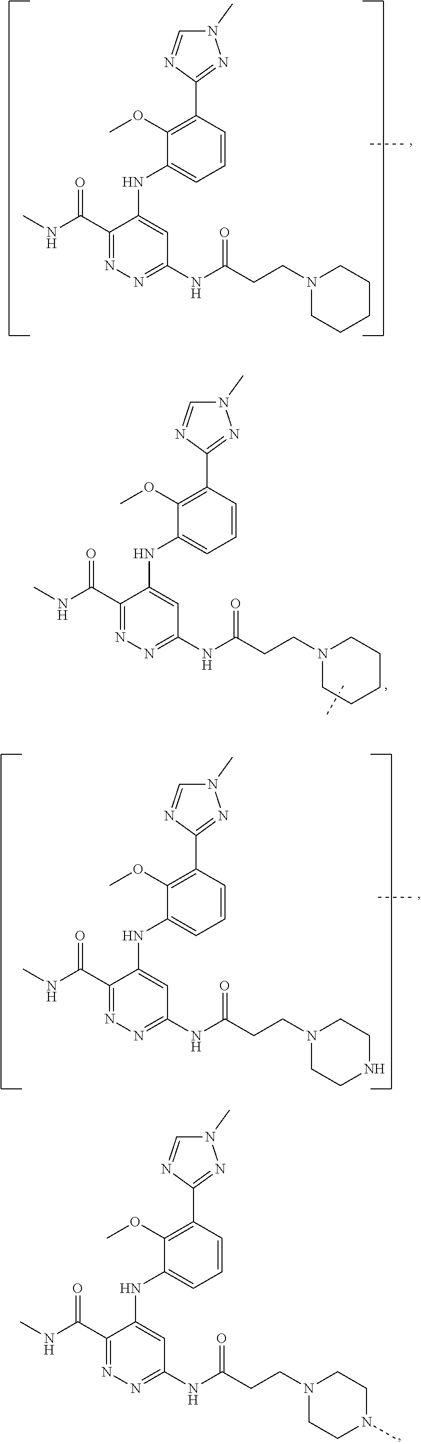 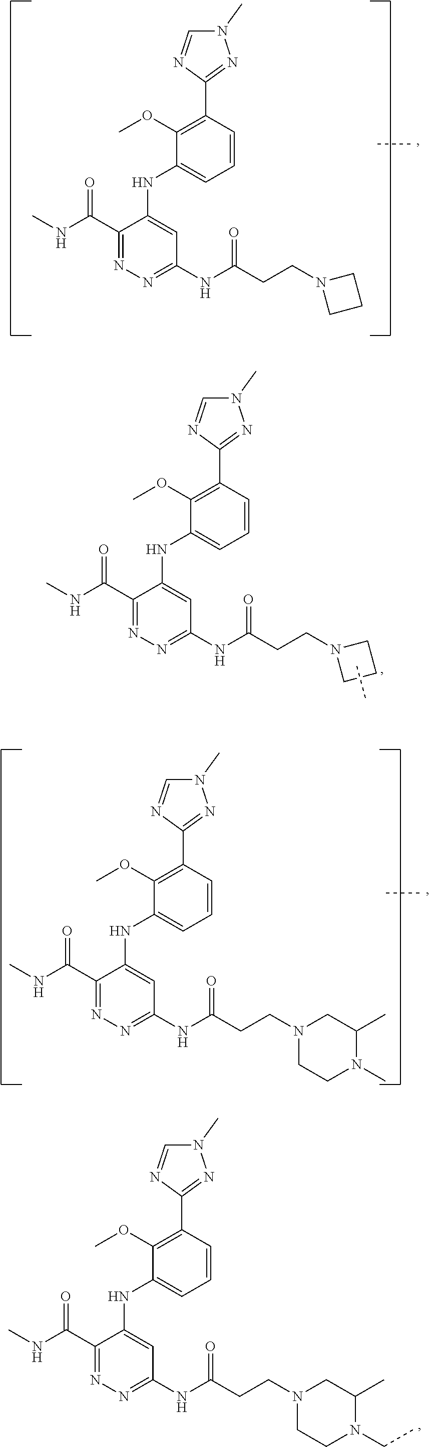 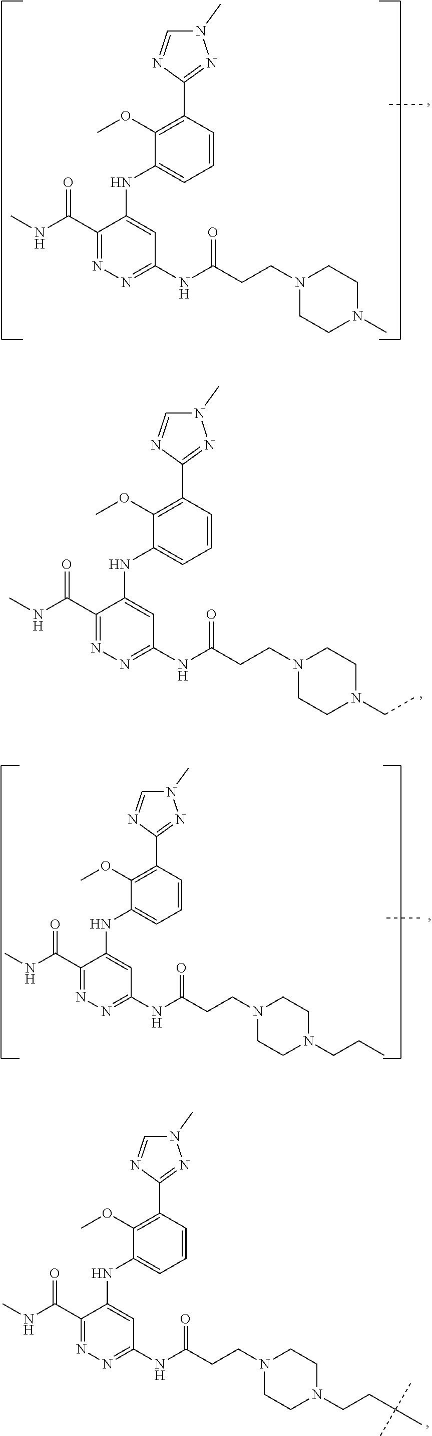 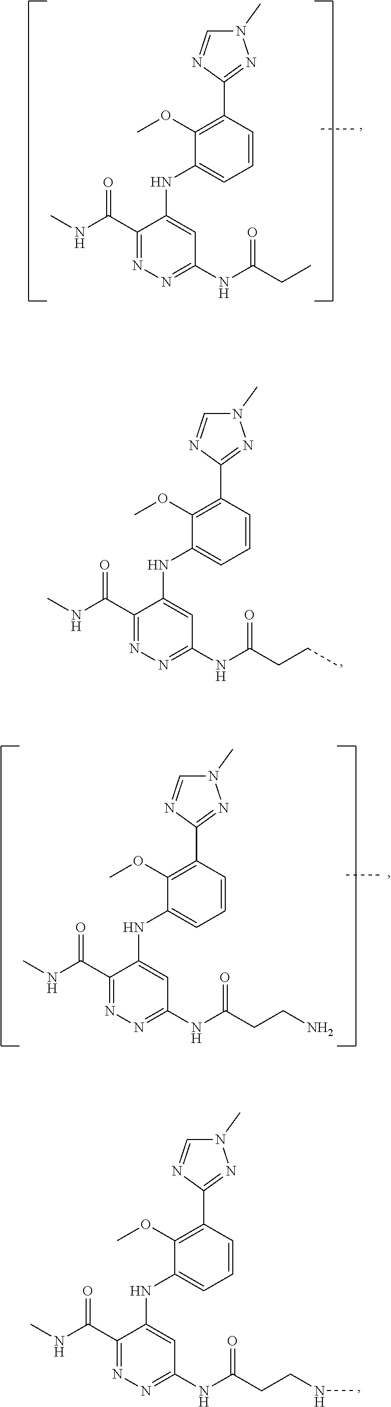 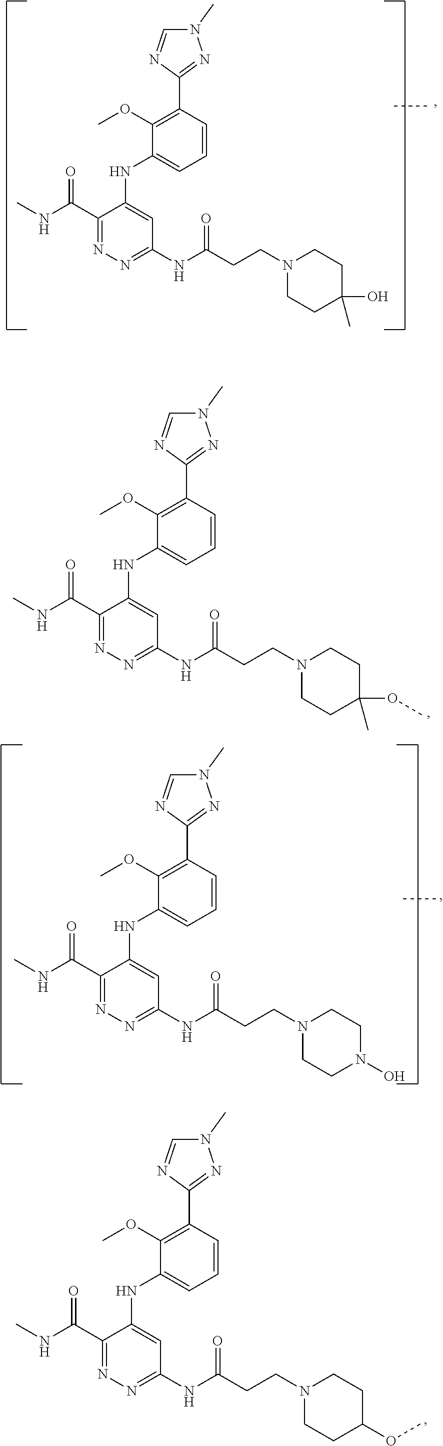 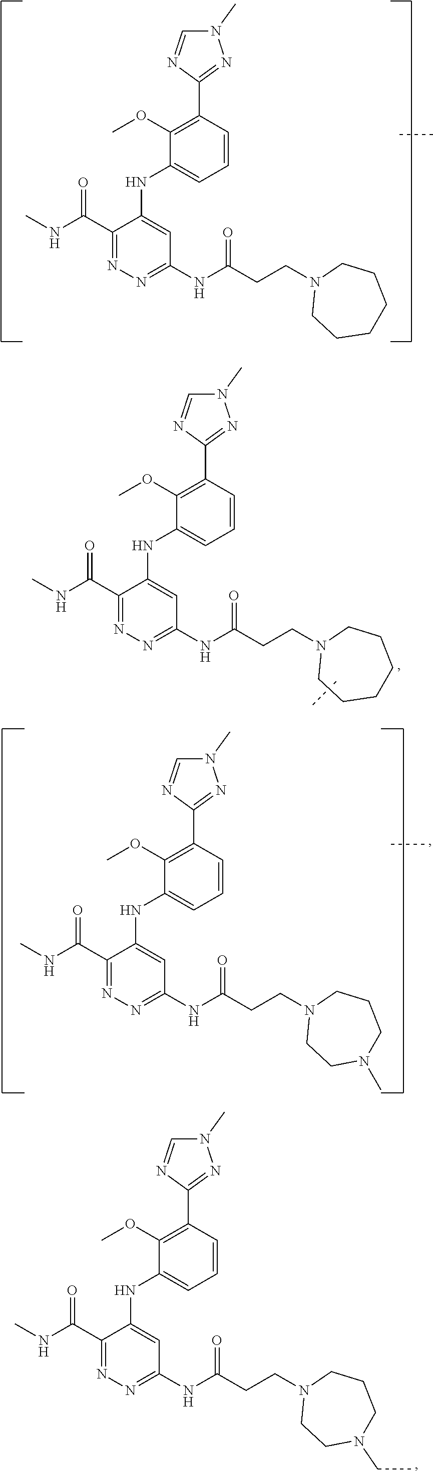 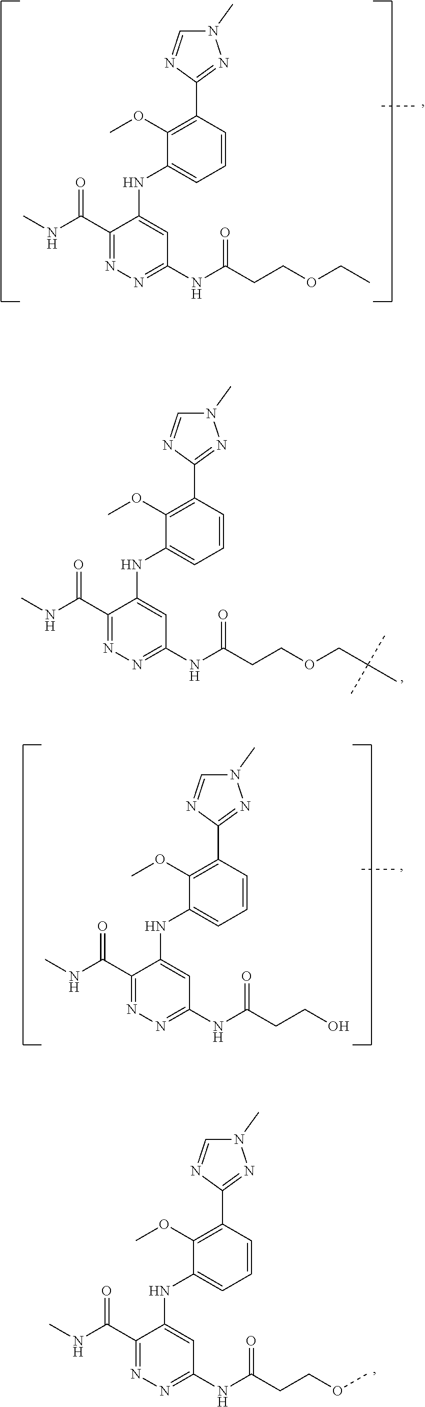 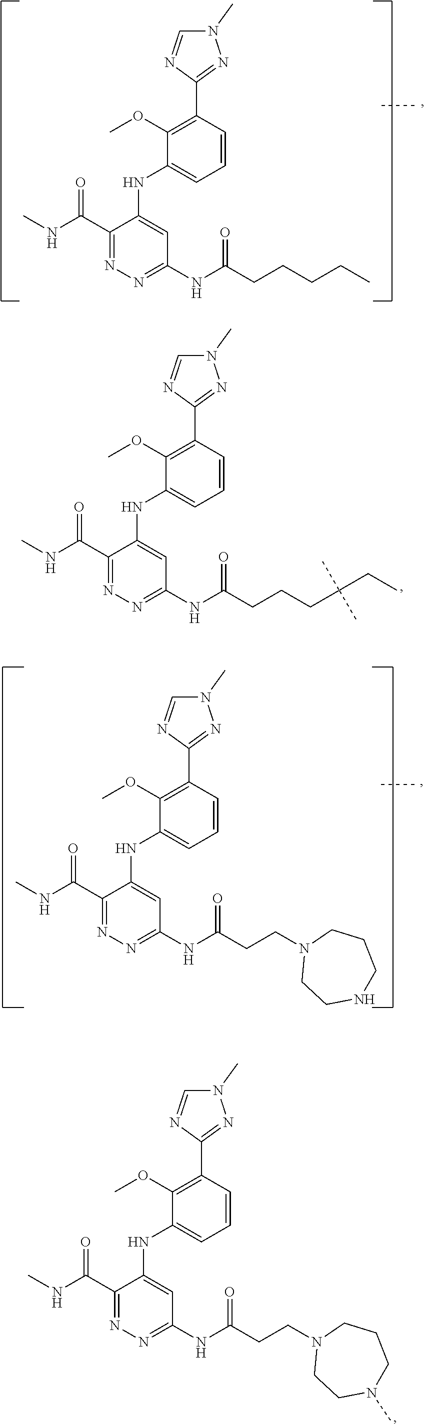
wherein the dashed line indicates a site of attachment of a linker or ULM, and wherein each PTM is coupled to at least one linker or ULM.

7. The compound of any of claims 1-6, wherein the ULM is a cereblon E3 ligase-binding moiety (CLM) selected from the group consisting of a thalidomide, lenalidomide, pomalidomide, analogs thereof, isosteres thereof, and derivatives thereof.

8. The compound of claim 1-7, wherein the CLM has a chemical structure represented by: 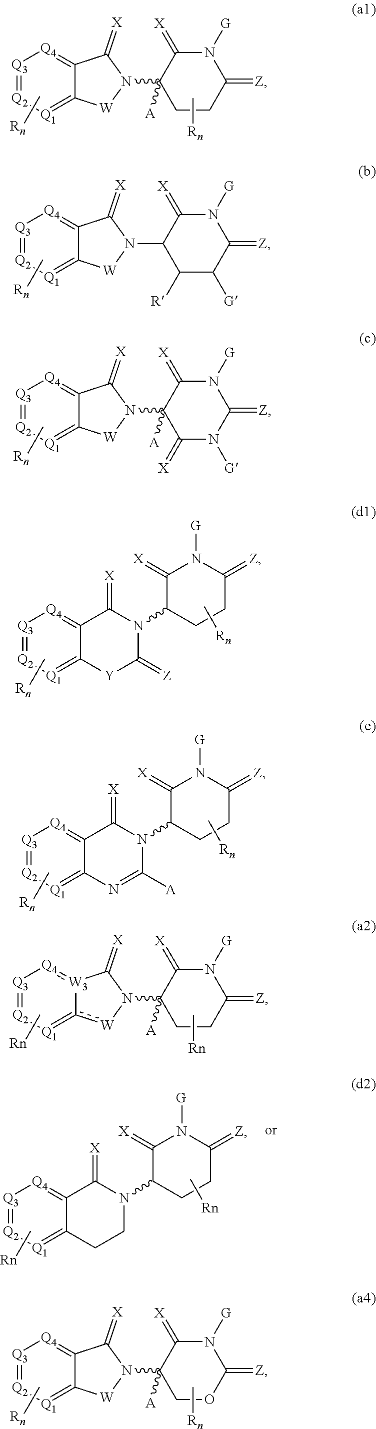
wherein:
W is selected from the group consisting of CH₂, O, CHR, C═O, SO₂, NH, N, optionally substituted cyclopropyl group, optionally substituted cyclobutyl group, and N-alkyl;
W₃ is selected from C or N;
each X is independently selected from the group consisting of absent, O, S, and CH₂;
Y is selected from the group consisting of CH₂, —C═CR′, NH, N-alkyl, N-aryl, N-heteroaryl, N-cycloalkyl, N-heterocyclyl, O, and S;
Z is selected from the group consisting of absent, O, S, and CH₂;
G and G′ are independently selected from the group consisting of H, unsubstituted or substituted linear or branched alkyl, OH, R′OCOOR, R′OCONRR″, CH₂-heterocyclyl optionally substituted with R′, and benzyl optionally substituted with R′;
Q₁, Q₂, Q₃, and Q₄ represent a carbon C or N substituted with a group independently selected from H, R, N or N-oxide;
A is independently selected from the group H, unsubstituted or substituted linear or branched alkyl, cycloalkyl, Cl and F;
n is an integer from 1 to 10 (e.g., 1-4, 1, 2, 3, 4, 5, 6, 7, 8, 9, or 10);
R comprises H, —CONR′R″, —C(═O)R′, —OR′, —NR′R″, —SR′, —SO₂R′, —SO₂NR′R″, —CR′R″—, —CR′NR′R″—, (—CR′O)_{n′}R″, optionally substituted heterocyclyl, -aryl (e.g., an optionally substituted C5-C7 aryl), optionally substituted alkyl-aryl (e.g., an alkyl-aryl comprising at least one of an optionally substituted C1-C6 alkyl, an optionally substituted C5-C7 aryl, or combinations thereof), -hetaryl, unsubstituted or substituted linear or branched alkyl (e.g., a C1-C6 linear or branched alkyl optionally substituted with one or more halogen, cycloalkyl (e.g., a C3-C6 cycloalkyl), or aryl (e.g., C5-C7 aryl)), optionally substituted alkoxyl group (e.g., a methoxy, ethoxy, butoxy, propoxy, pentoxy, or hexoxy; wherein the alkoxyl may be substituted with one or more halogen, alkyl, haloalky, fluoroalkyl, cycloalkyl (e.g., a C3-C6 cycloalkyl), or aryl (e.g., C5-C7 aryl)), optionally substituted heterocyclyl, —P(O)(OR′)R″, —P(O)R′R″, —OP(O)(OR′)R″, —OP(O)R′R″, —Cl, —F, —Br, —I, —CF₃, —CN, —NR′SO₂NR′R″, —NR′CONR′R″, —CONR′COR″, —NR′C(═N—CN)NR′R″, —C(═N—CN)NR′R″, —NR′C(═N—CN)R″, —NR′C(═C—NO₂)NR′R″, —SO₂NR′COR″, —NO₂, —CO₂R′, —C(C═N—OR′)R″, —CR′═CR′R″, —CCR′, —S(C═O)(C═N—R′)R″, —SF₅ and —OCF₃, wherein at least one R or W is modified to be covalently joined to a PTM, a chemical linker group (L), a ULM;
each of x, y, and z are independently 0, 1, 2, 3, 4, 5, or 6;
R′ and R″ are independently selected from the group consisting of H, optionally substituted alkyl, optionally substituted cycloalkyl, optionally substituted aryl, optionally substituted heteroaryl, optionally substituted heterocyclic, —C(═O)R, and optionally substituted heterocyclyl;
n is an integer from 1-10 (e.g., 1-4, 1, 2, 3, 4, 5, 6, 7, 8, 9, or 10); [symbol]
represents a single bond or a double bond; and [symbol]
represents a bond that may be stereospecific ((R) or (S)) or non-stereospecific.

9. The compound of any of claims 1-8, wherein the CLM has a chemical structure represented by: 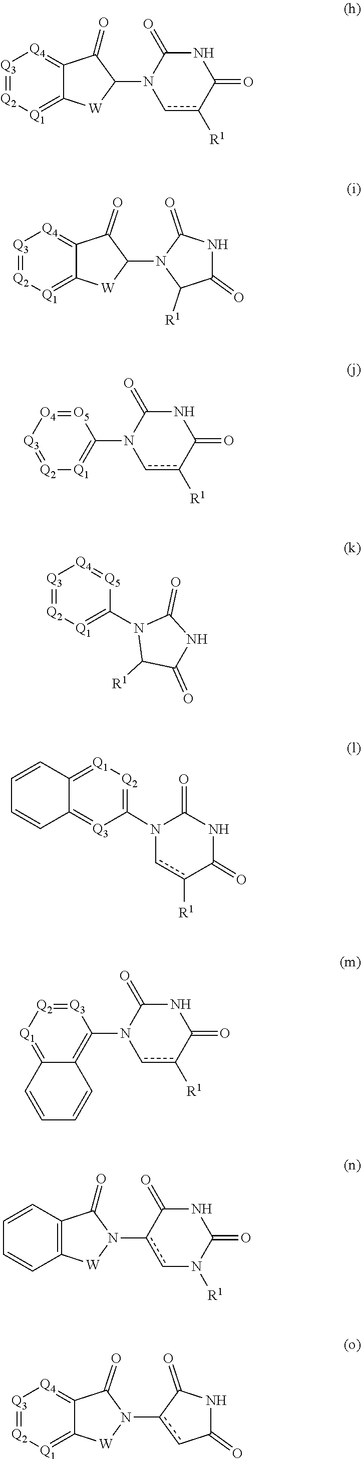 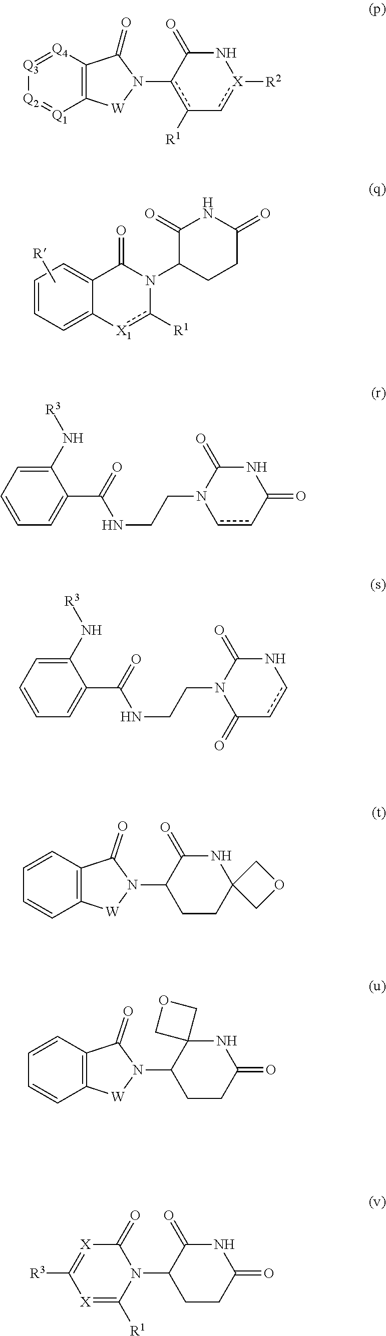 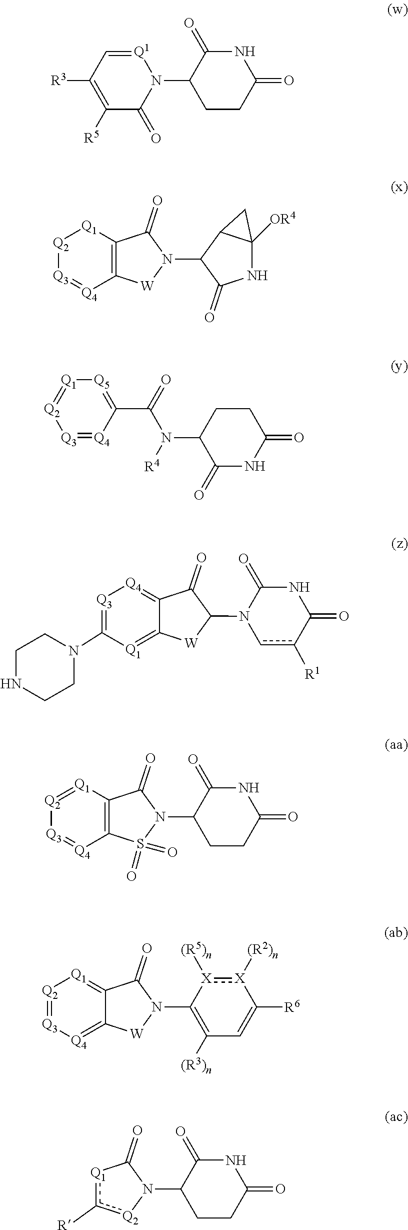 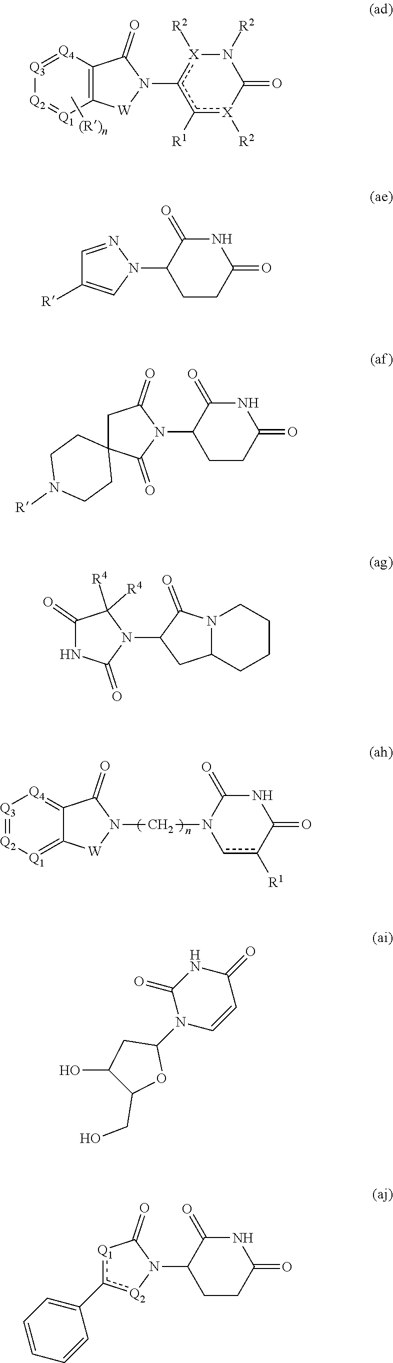 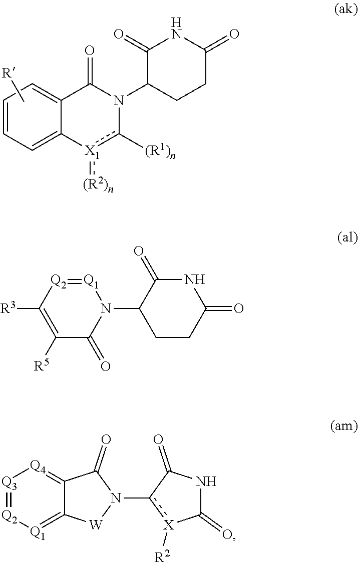
wherein:
W is selected from the group consisting of CH₂, O, CHR, C═O, SO₂, NH, N, optionally substituted cyclopropyl group, optionally substituted cyclobutyl group, and N-alkyl;
Q₁, Q₂, Q₃, Q₄, Q₅ each independently represent a carbon C or N substituted with a group independently selected from R′, N or N-oxide;
R¹ is selected from absent, H, OH, CN, C1-C3 alkyl, C═O;
R² is selected from the group absent, H, OH, CN, C1-C3 alkyl, CHF₂, CF₃, CHO, C(═O)NH₂;
R³ is selected from H, alkyl (e.g., C1-C6 or C1-C3 alkyl), substituted alkyl (e.g., substituted C1-C6 or C1-C3 alkyl), alkoxy (e.g., C1-C6 or C1-C3 alkoxyl), substituted alkoxy (e.g., substituted C1-C6 or C1-C3 alkoxyl);
R⁴ is selected from H, alkyl, substituted alkyl;
R⁵ and R⁶ are each independently H, halogen, C(═O)R′, CN, OH, CF₃;
X is C, CH, C═O, or N;
X₁ is C═O, N, CH, or CH₂;
R′ is selected from H, halogen, amine, alkyl (e.g., C1-C3 alkyl), substituted alkyl (e.g., substituted C1-C3 alkyl), alkoxy (e.g., C1-C3 alkoxyl), substituted alkoxy (e.g., substituted C1-C3 alkoxyl), NR²R³, C(═O)OR², optionally substituted phenyl;
n is 0-4; [symbol]
is a single or double bond; and
the CLM is covalently joined to a PTM, a chemical linker group (L), a ULM, or CLM).

10. The compound of any one of claims 1-9, wherein the linker (L) comprises a chemical structural unit represented by the formula: 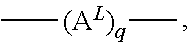
wherein:
-(A^{L})_{q}- is a group which is connected to at least one of ULM, PTM, or both;
q is an integer greater than or equal to 1;
each A is independently selected from the group consisting of CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L1} and/or R^{L2} groups, C₃₋₁₁ heterocyclyl optionally substituted with 0-6 R^{L1} and/or R^{L2} groups, spiroheterocyclyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R¹⁵ groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂.

11. The compound according to any one of claim 1-10, wherein the linker (L) includes an optionally substituted C₁-C₅₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkyl), wherein each carbon is optionally substituted or replaced with (1) a heteroatom selected from N, S, P, or Si atoms that has an appropriate number of hydrogens, substitutions, or both to complete valency, (2) an optionally substituted cycloalkyl or bicyclic cycloalkly, (3) an optionally substituted heterocyloalkyl or bicyclic heterocyloalkyl, (4) an optionally substituted aryl or bicyclic aryl, or (5) optionally substituted heteroaryl or bicyclic heteroaryl, with the proviso that there is no heteroatom-heteroatom bonding (e.g., no heteroatoms are covalently linked or adjacently located).

12. The compound according to any one of claim 1-10, wherein the linker (L) includes an optionally substituted C₁-C₅₀ alkyl (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ alkyl), wherein:
each carbon is optionally substituted with CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L1} and/or R^{L2} groups, C₃₋₁₁ heteocyclyl optionally substituted with 0-6 R^{L1} and/or R^{L2} groups, C₅₋₁₃ spiroheterocyclyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂.

13. The compound according to any of claims 1-14, wherein the linker (L) comprises the following chemical structure: 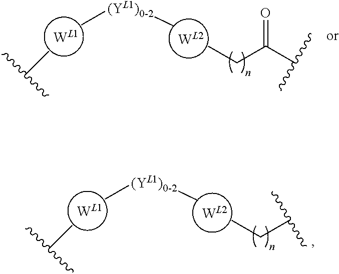
wherein:
W^{L1} and W^{L2} are each independently absent, a 4-8 membered ring with 0-4 heteroatoms, optionally substituted with RQ, each RQ is independently a H, halogen, OH, CN, CF₃, unsubstituted or substituted linear or branched C1-C6, unsubstituted or substituted linear or branched C1-C6 alkoxy, or 2 RQ groups taken together with the atom they are attached to form a 4-8 membered ring system containing 0-4 heteroatoms;
each Y^{L1} is independently a bond, or unsubstituted or substituted linear or branched C1-C6 alkoxy, or unsubstituted or substituted linear or branched C1-C6 alkyl and optionally one or more C atoms are replaced with 0;
n is an integer from 0 to 10; and 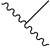
and [symbol] indicate the attachment point to the PTM or ULM moieties.

14. The compound according to any of claims 1-15, wherein the linker (L) comprises the following chemical structure: 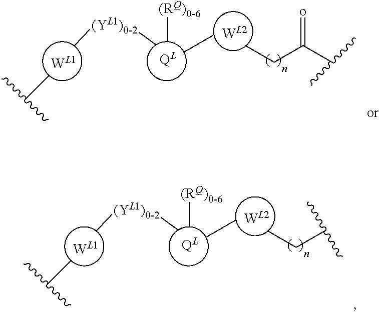
wherein:
W^{L1} and W^{L2} are each independently absent, aryl, heteroaryl, cyclic, heterocyclic, C₁₋₆ alkyl and optionally one or more C atoms are replaced with O, C₁₋₆ alkene and optionally one or more C atoms are replaced with O, C₁₋₆ alkyne and optionally one or more C atoms are replaced with O, bicyclic, biaryl, biheteroaryl, or biheterocyclic, each optionally substituted with RQ, each RQ is independently a H, halogen, OH, NH₂, NR^{Y1}R^{Y2}, CN, CF₃, hydroxyl, nitro, C≡CH, C₂₋₆ alkenyl, C₂₋₆ alkynyl, unsubstituted or substituted linear or branched C₁-C₆ alkyl, unsubstituted or substituted linear or branched C₁-C₆ alkoxy, OC₁₋₃alkyl optionally substituted by 1 or more —F, or 2 RQ groups taken together with the atom they are attached to form a 4-8 membered ring system containing 0-4 heteroatoms;
each Y^{L1} is independently: a bond; NR^{YL1}; O; S; NR^{YL2}; CR^{YL1}R^{YL2}; C═O; C═S; SO; SO₂; unsubstituted or substituted linear or branched C₁-C₆ alkoxy; or unsubstituted or substituted linear or branched C₁-C₆ alkyl with one or more C atoms are optionally replaced with O;
Q^{L} is a 3-6 membered alicyclic or aromatic ring with 0-4 heteroatoms, optionally bridged, optionally substituted with 0-6 R^{Q}, each R^{Q} is independently H, linear or branched C₁₋₆ alkyl optionally substituted by 1 or more halo or C₁₋₆ alkoxyl, or 2 R^{Q} groups taken together with the atom they are attached to form a 3-8 membered ring system containing 0-2 heteroatoms;
R^{YL1} and R^{YL2} are each independently: H; OH; linear or branched C₁₋₆ alkyl optionally substituted by 1 or more halo or C₁₋₆ alkoxyl; or R¹, R² together with the atom they are attached to form a 3-8 membered ring system containing 0-2 heteroatoms;
n is an integer from 0 to 10; and 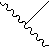
and [symbol] indicate the attachment point to the PTM or ULM.

15. The compound according to any one of claim 1-10, wherein the linker (L) includes an optionally substituted C₆-C₃₀ alkyl (e.g., C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, or C₃₀ alkyl), wherein each carbon is optionally substituted or replaced with (1) a heteroatom selected from N, S, P, or Si atoms that has an appropriate number of hydrogens, substitutions, or both to complete valency, (2) an optionally substituted cycloalkyl or bicyclic cycloalkly, (3) an optionally substituted heterocyloalkyl or bicyclic heterocyloalkyl, (4) an optionally substituted aryl or bicyclic aryl, or (5) optionally substituted heteroaryl or bicyclic heteroaryl, with the proviso that there is no heteroatom-heteroatom bonding (e.g., no heteroatoms are covalently linked or adjacently located).

16. The compound according to any one of claim 1-10, wherein the linker (L) is a bond or a chemical linker group represented by the formula -(A^{L})_{q}-, wherein A is a chemical moiety and q is an integer from 6-20 (e.g., 1, 2, 3, 4, 5, 6, 7, 8, 9, 10, 11, 12, 13, 14, 15, 16, 17, 18, 19, or 20), and wherein L is covalently bound to both the PTM and the ULM, and provides for binding of the PTM to the protein target and the ULM to an E3 ubiquitin ligase in sufficient proximity to result in target protein ubiquitination.

17. The compound according to any of claims 1-16, wherein L is a means for covalently coupling the PTM to the ULM.

18. The compound according to any of claims 1-17, wherein the unit A^{L} of the linker (L) is selected from the group consisting of: 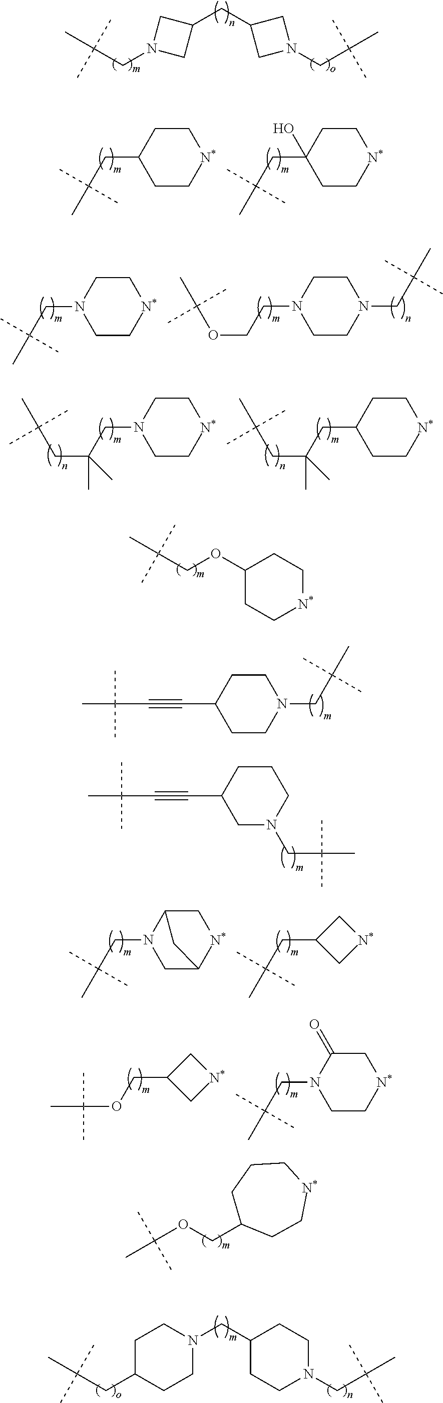 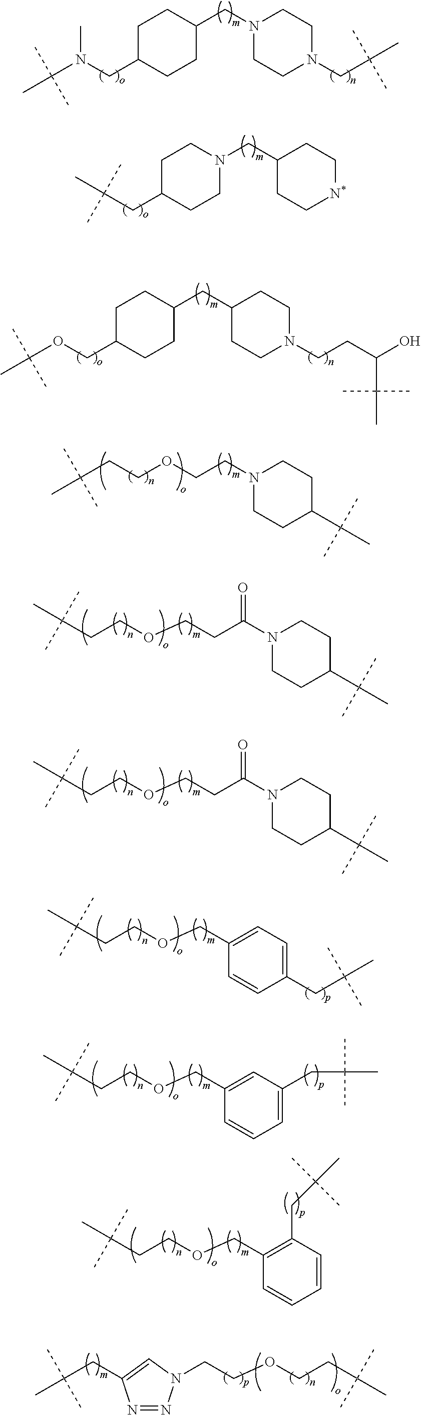 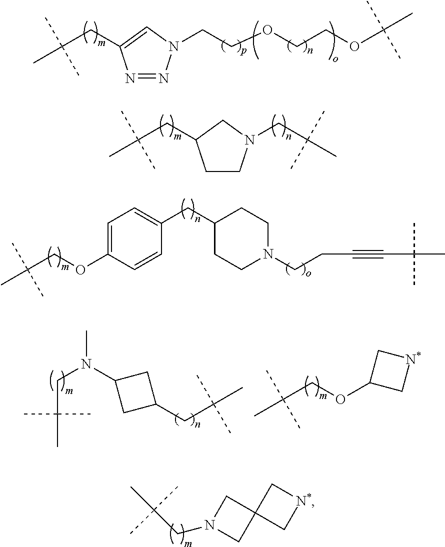
wherein:
N* is a nitrogen atom that is covalently linked to the ULM or PTM, or is shared with the ULM or PTM; and
each m, n, o, p, q, and r is independently 0, 1, 2, 3, 4, 5, 6, 7, 8, 9, 10, 11, 12, 13, 14, 15, 16, 17, 18, 19, or 20.

19. The compound according to any of claims 1-18, wherein at least one of:
(a) the ULM is represented by: 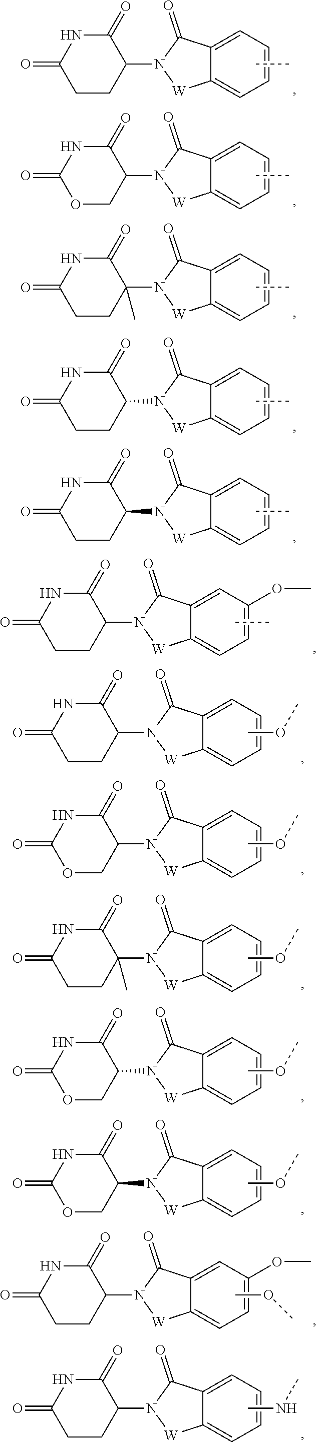 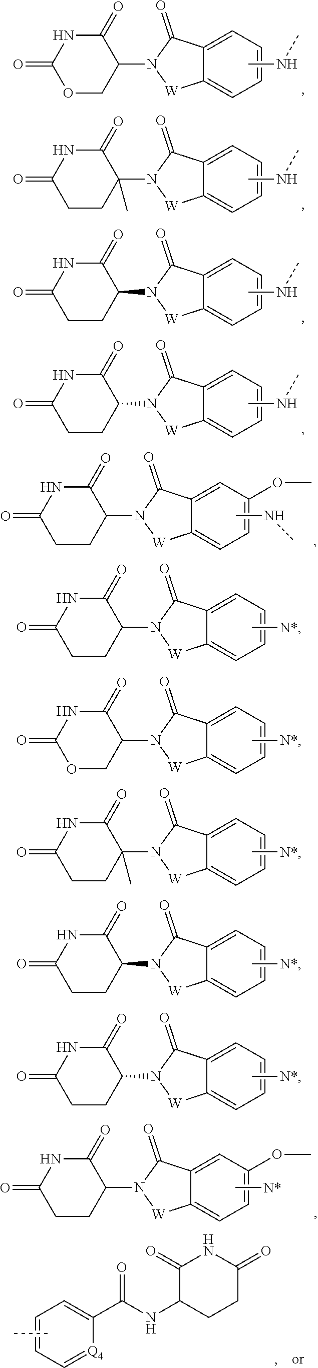 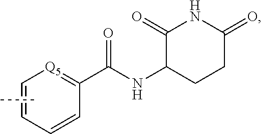
wherein: [symbol]
of the ULM indicates the point of attachment with a linker group or a PTM; and
N* is a nitrogen atom that is shared with the chemical linker group or PTM;
(b) the PTM is represented by: 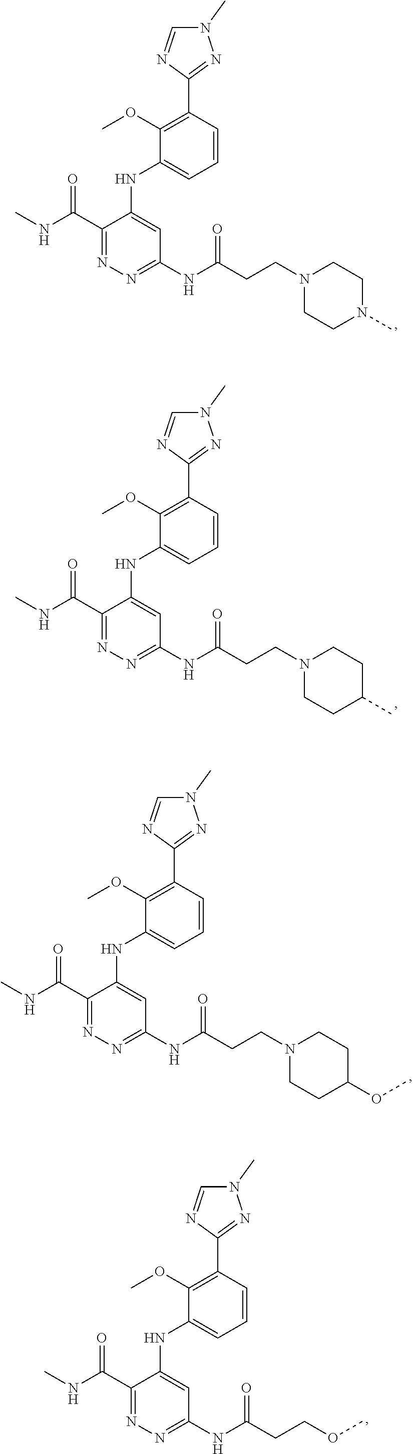 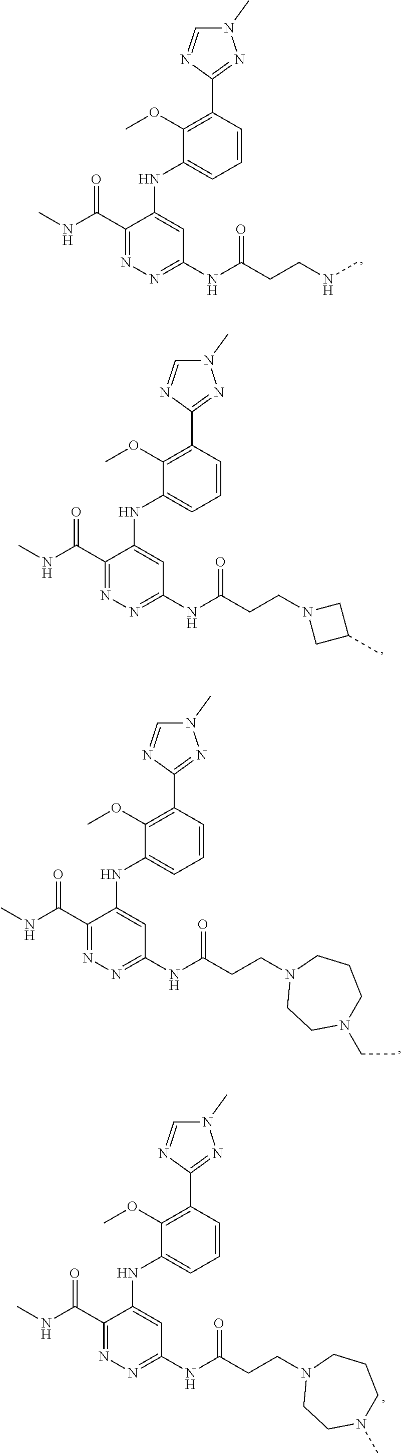 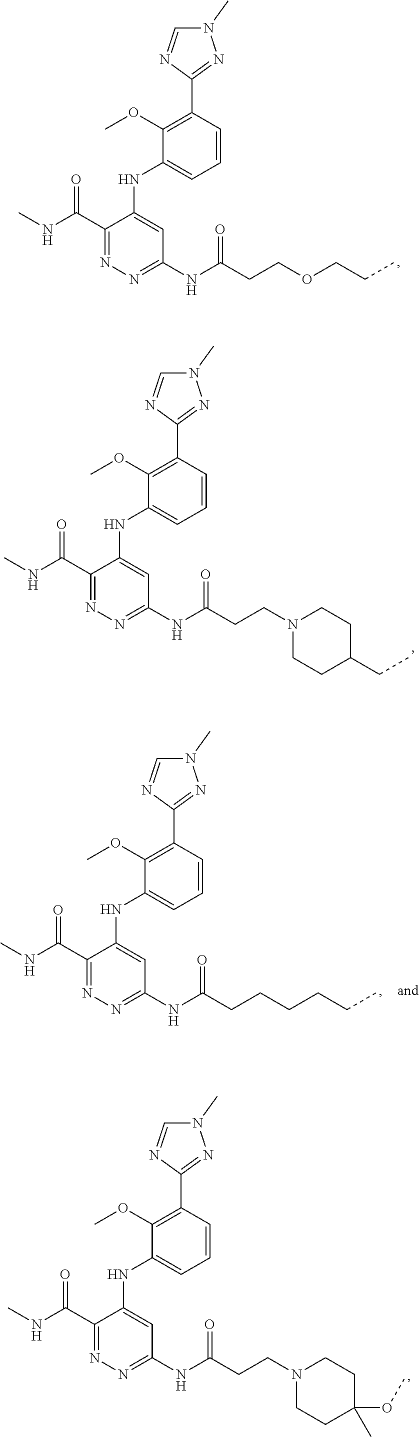
wherein [symbol] of the PTM indicates the point of attachment with a linker group (L) or a ULM, and [symbol] represents a bond that may be stereospecific ((R) or (S)) or non-stereospecific;
(c) the L is a linker group (L) selected from: 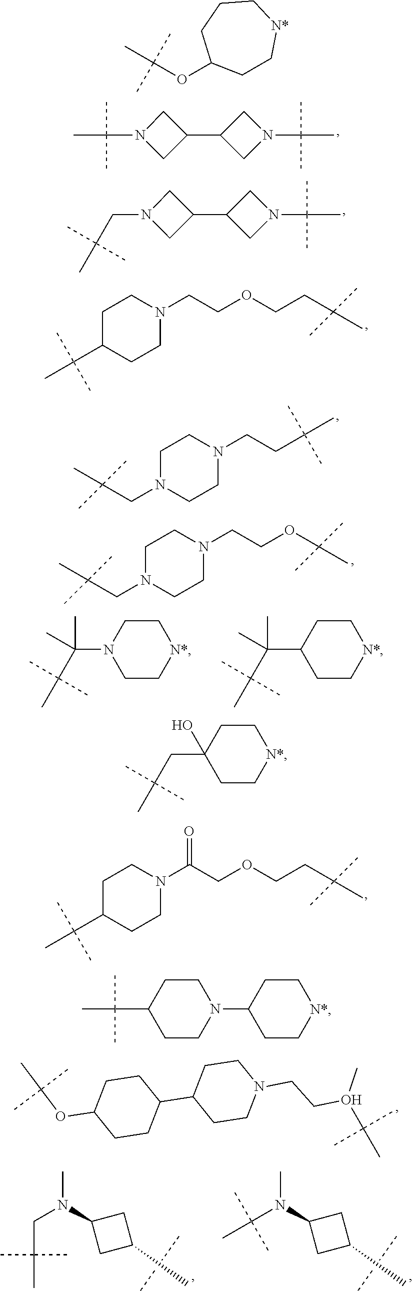 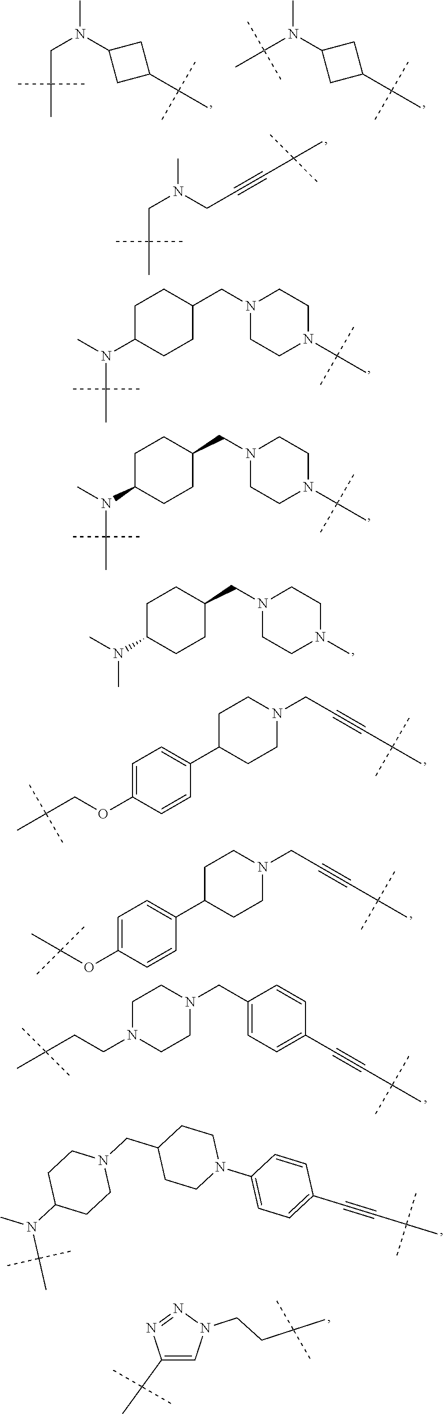 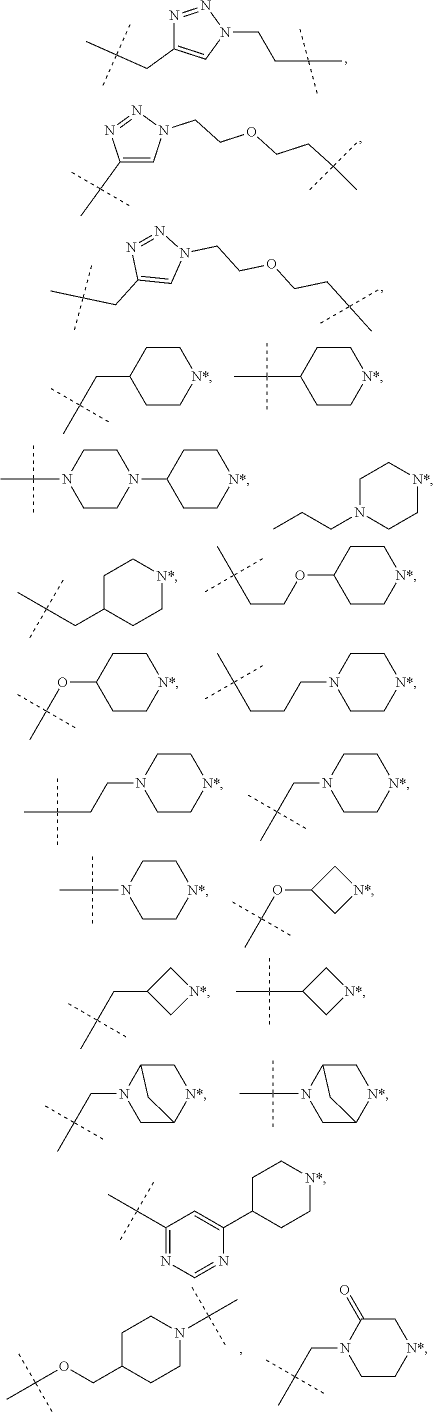 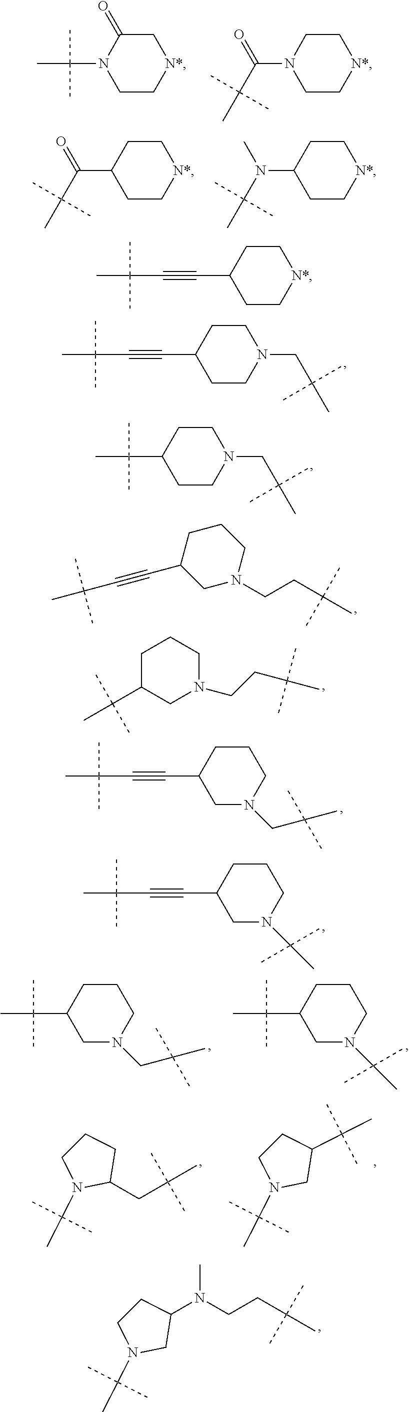 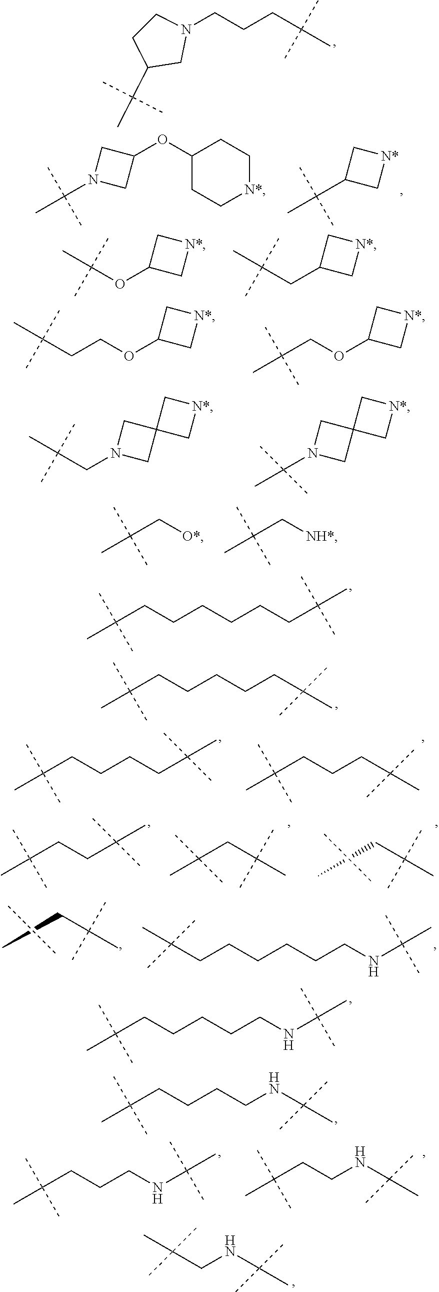
wherein N* is a nitrogen atom that is covalently linked to the ULM or PTM, or that is shared with the ULM or PTM; or
(d) a combination thereof.

20. The compound according to claim 1, wherein at least one of:
the PTM is a PTM selected from a compound of Table 1;
the ULM is a ULM selected from a compound of Table 1; and
the L is an L selected from a compound of Table 1.

21. The compound of claim 1, wherein the compound is selected from the group consisting of compounds 1-10 of Table 1.

| PROTAC # | Parent Mol Structure |
|---|---|
| 1 | 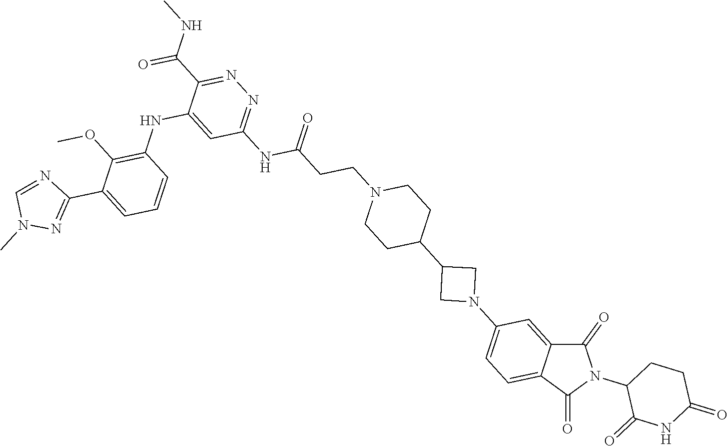 [SMILES: CNC(=O)c1nnc(NC(=O)CCN2CCC(C3CN(c4ccc5c(c4)C(=O)N(C4CCC(=O)NC4=O)C5=O)C3)CC2)cc1Nc1cccc(-c2ncn(C)n2)c1OC] |
| 2 | 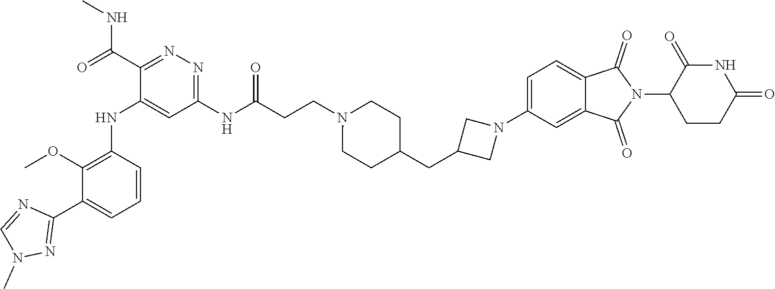 [SMILES: CNC(=O)c1nnc(NC(=O)CCN2CCC(CC3CN(c4ccc5c(c4)C(=O)N(C4CCC(=O)NC4=O)C5=O)C3)CC2)cc1Nc1cccc(-c2ncn(C)n2)c1OC] |
| 3 | 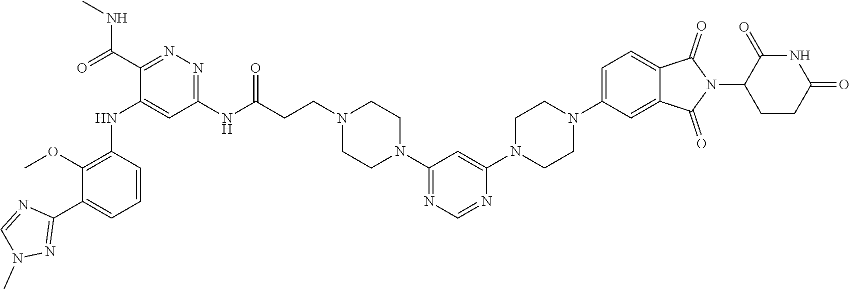 [SMILES: CNC(=O)c1nnc(NC(=O)CCN2CCN(c3cc(N4CCN(c5ccc6c(c5)C(=O)N(C5CCC(=O)NC5=O)C6=O)CC4)ncn3)CC2)cc1Nc1cccc(-c2ncn(C)n2)c1OC] |
| 4 | 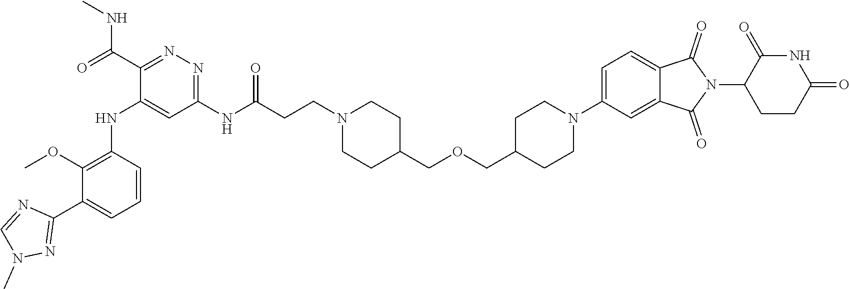 [SMILES: CNC(=O)c1nnc(NC(=O)CCN2CCC(COCC3CCN(c4ccc5c(c4)C(=O)N(C4CCC(=O)NC4=O)C5=O)CC3)CC2)cc1Nc1cccc(-c2ncn(C)n2)c1OC] |
| 5 | 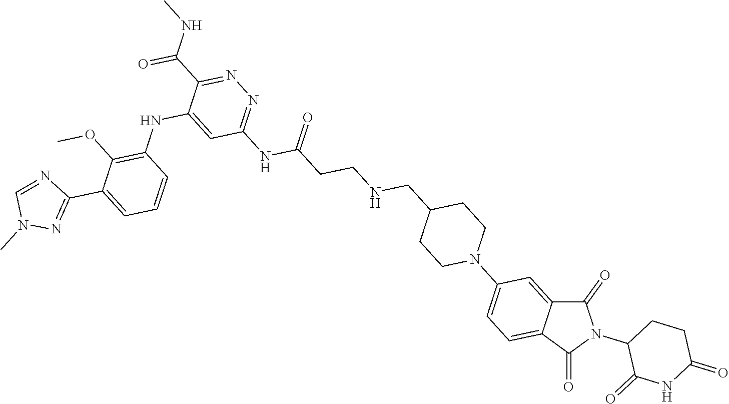 [SMILES: CNC(=O)c1nnc(NC(=O)CCNCC2CCN(c3ccc4c(c3)C(=O)N(C3CCC(=O)NC3=O)C4=O)CC2)cc1Nc1cccc(-c2ncn(C)n2)c1OC] |
| 6 | 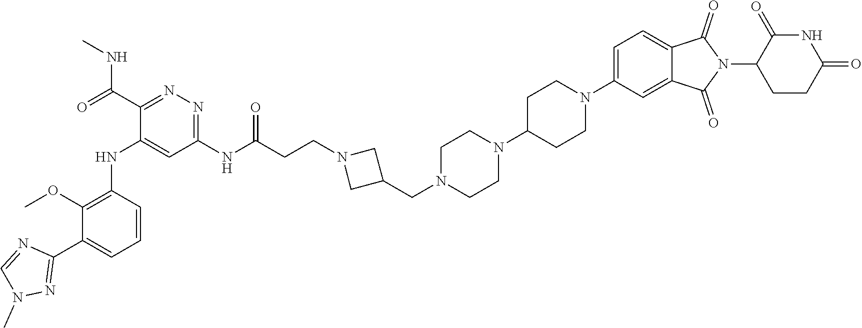 [SMILES: CNC(=O)c1nnc(NC(=O)CCN2CC(CN3CCN(C4CCN(c5ccc6c(c5)C(=O)N(C5CCC(=O)NC5=O)C6=O)CC4)CC3)C2)cc1Nc1cccc(-c2ncn(C)n2)c1OC] |
| 7 | 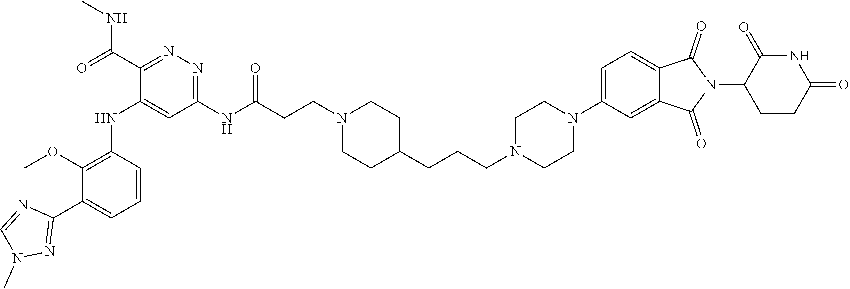 [SMILES: CNC(=O)c1nnc(NC(=O)CCN2CCC(CCCN3CCN(c4ccc5c(c4)C(=O)N(C4CCC(=O)NC4=O)C5=O)CC3)CC2)cc1Nc1cccc(-c2ncn(C)n2)c1OC] |
| 8 | 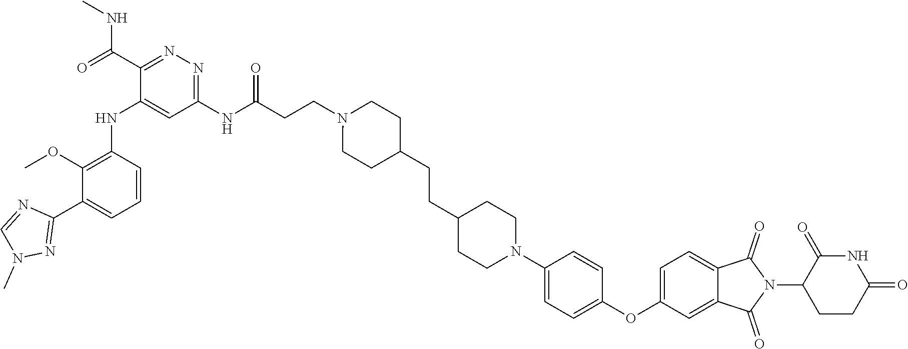 [SMILES: CNC(=O)c1nnc(NC(=O)CCN2CCC(CCC3CCN(c4ccc(Oc5ccc6c(c5)C(=O)N(C5CCC(=O)NC5=O)C6=O)cc4)CC3)CC2)cc1Nc1cccc(-c2ncn(C)n2)c1OC] |
| 9 | 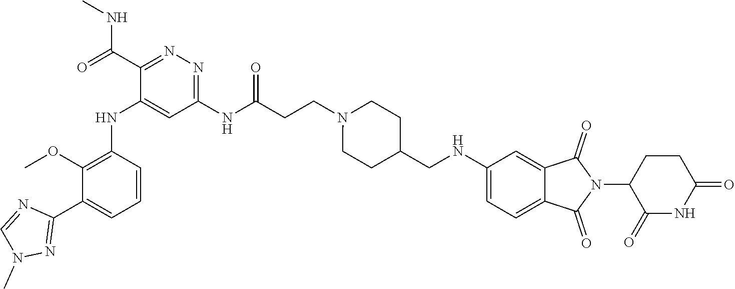 [SMILES: CNC(=O)c1nnc(NC(=O)CCN2CCC(CNc3ccc4c(c3)C(=O)N(C3CCC(=O)NC3=O)C4=O)CC2)cc1Nc1cccc(-c2ncn(C)n2)c1OC] |

22. A composition comprising an effective amount of a bifunctional compound of any of claims 1-21, and a pharmaceutically acceptable carrier.

23. The composition of claim 22, wherein the composition further comprises at least one of additional bioactive agent or a second bifunctional compound of any of claims 1-21.

24. The composition of claim 23, wherein the additional bioactive agent is an anti-inflammatory, a chemotherapy agent, or an immunomodulatory agent.

25. A composition comprising a pharmaceutically acceptable carrier and an effective amount of at least one compound of any of claims 1-21 for treating a disease, a disorder or a symptom casually related to TYK2 in a subject, wherein the composition is effective in treating or ameliorating the disease, disorder, or at least one symptom of the disease or disorder.

26. The composition of claim 25, wherein the disease or disorder is an immunological disorder, inflammatory disorder, or cancer.

27. A method of treating or preventing a disease, a disorder, or symptom associated with TYK2 comprising, providing a patient in need thereof, and administering an effective amount of a compound as described herein or composition comprising the same to the patient, wherein the compound or composition is effective in treating or ameliorating the disease, disorder, or at least one symptom of the disease or disorder.
